(19) United States
(12) Patent Application Publication
(10) Pub. No.: US 2022/0389398 A1
(43) Pub. Date: Dec. 8, 2022

(54) ENGINEERED CRISPR/CAS13 SYSTEM AND USES THEREOF

(71) Applicant: HuiGene Therapeutics Co., Ltd., Shanghai (CN)

(72) Inventors: Huawei Tong, Shanghai (CN); Xing Wang, Shanghai (CN); Shaoran Wang, Shanghai (CN)

(21) Appl. No.: 17/836,175

(22) Filed: Jun. 9, 2022

Related U.S. Application Data
(63) Continuation of application No. PCT/CN2021/121926, filed on Sep. 29, 2021.

(30) Foreign Application Priority Data
Sep. 30, 2020 (CN) PCT/CN2020/119559
Mar. 9, 2021 (CN) PCT/CN2021/079821

(51) Int. Cl. C12N 9/22; C12N 15/11; C12N 15/10; C12N 15/90

(52) U.S. Cl. CPC C12N 9/22; C12N 15/11; C12N 15/102; C12N 15/907; C12N 2310/20; C12N 2800/80; A61K 48/00

(57) ABSTRACT

The invention provides novel engineered CRISPR/Cas effector enzymes, such as Cas13 (e.g., Cas13d, Cas13e, or Cas13f) that substantially maintain guide-sequence-specific endonuclease activity and substantially lack guide-sequence-independent collateral endonuclease activity compared to the corresponding wild-type Cas. Also provided are polynucleotides encoding the same, vectors or host cells comprising the polynucleotides or engineered Cas, and method of use, such as in RNA-based target gene transcript knock down.

### REFERENCE TO RELATED APPLICATIONS

The instant application is a continuation application, filed under 35 U.S.C. 111(a), of International Patent Application No. PCT/CN2021/121926, filed on Sep. 29, 2021, which claims foreign priority under 35 U.S.C. 365(b), to International Patent Application No. PCT/CN2021/079821, filed on Mar. 9, 2021, and International Patent Application No. PCT/CN2020/119559, filed on Sep. 30, 2020, the entire contents of each of the above-referenced applications, including any sequence listing and drawings, are incorporated herein by reference.

### SEQUENCE LISTING

The instant application contains a Sequence Listing which has been submitted electronically in ASCII format and is hereby incorporated by reference in its entirety. Said ASCII copy, created on Jun. 9, 2022, is named 132045-00401_SL.txt and is 903,580 bytes in size.

### BACKGROUND OF THE INVENTION

CRISPR (clustered regularly interspaced short palindromic repeats) is a family of DNA sequences found within the genomes of prokaryotic organisms such as bacteria and archaea. These sequences are understood to be derived from DNA fragments of bacteriophages that have previously infected the prokaryote, and are used to detect and destroy DNA or RNA from similar bacteriophages during subsequent infections of the prokaryotes.

CRISPR-associated system is a set of homologous genes, or Cas genes, some of which encode Cas protein having helicase and nuclease activities. The Cas proteins are enzymes that utilize RNA derived from the CRISPR sequences (crRNA) as guide sequences to recognize and cleave specific strands of polynucleotide (e.g., DNA) that are complementary to the crRNA.

Together, the CRISPR-Cas system constitutes a primitive prokaryotic “immune system” that confers resistance or acquired immunity to foreign pathogenic genetic elements, such as those present within extrachromosomal DNA (e.g., plasmids) and bacteriophages, or foreign RNA encoded by foreign DNA.

In nature, the CRISPR/Cas system appears to be a widespread prokaryotic defense mechanism against foreign genetic materials, and is found in approximately 50% of sequenced bacterial genomes and nearly 90% of sequenced archaea. This prokaryotic system has since been developed to form the basis of a technology known as CRISPR-Cas that found extensive use in numerous eukaryotic organisms including human, in a wide variety of applications including basic biological research, development of biotechnology products, and disease treatment.

The prokaryotic CRISPR-Cas systems comprise an extremely diverse group of effector proteins, non-coding elements, as well as loci architectures, some examples of which have been engineered and adapted to produce important biotechnologies.

The CRISPR locus structure has been studied in many systems. In these systems, the CRISPR array in the genomic DNA typically comprises an AT-rich leader sequence, followed by short DR sequences separated by unique spacer sequences. These CRISPR DR sequences typically range in size from 28 to 37 bps, though the range can be 23-55 bps. Some DR sequences show dyad symmetry, implying the formation of a secondary structure such as a stem-loop (“hairpin”) in the RNA, while others appear unstructured. The size of spacers in different CRISPR arrays is typically 28-38 bps (with a range of 21-72 bps). There are usually fewer than 50 units of the repeat-spacer sequence in a CRISPR array.

Small clusters of cas genes are often found next to such CRISPR repeat-spacer arrays. So far, the 93 identified cas genes have been grouped into 35 families, based on sequence similarity of their encoded proteins. Eleven of the 35 families form the so-called cas core, which includes the protein families Cas1 through Cas9. A complete CRISPR-Cas locus has at least one gene belonging to the cas core.

CRISPR-Cas systems can be broadly divided into two classes—Class 1 systems use a complex of multiple Cas proteins to degrade foreign nucleic acids, while Class 2 systems use a single large Cas protein for the same purpose. The single-subunit effector compositions of the Class 2 systems provide a simpler component set for engineering and application translation, and has thus far been important sources of discovery, engineering, and optimization of novel powerful programmable technologies for genome engineering and beyond.

Class 1 system is further divided into types I, III, and IV; and Class 2 system is divided into types II, V, and VI. These 6 system types are additionally divided into 19 subtypes. Classification is also based on the complement of cas genes that are present. Most CRISPR-Cas systems have a Cas1 protein. Many prokaryotes contain multiple CRISPR-Cas systems, suggesting that they are compatible and may share components.

One of the first and best characterized Cas proteins—Cas9—is a prototypical member of Class 2, type II, and originates from Streptococcus pyogenes (SpCas9). Cas9 is a DNA endonuclease activated by a small crRNA molecule that complements a target DNA sequence, and a separate trans-activating CRISPR RNA (tracrRNA). The crRNA consists of a direct repeat (DR) sequence responsible for protein binding to the crRNA and a spacer sequence, which may be engineered to be complementary to any desired nucleic acid target sequence. In this way, CRISPR systems can be programmed to target DNA or RNA targets by modifying the spacer sequence of the crRNA. The crRNA and tracrRNA have been fused to form a single guide RNA (sgRNA) for better practical utility. When combined with Cas9, sgRNA hybridizes with its target DNA, and guides Cas9 to cut the target DNA. Other Cas9 effector protein from other species have also been identified and used similarly, including Cas9 from the S. thermophilus CRISPR system. These CRISPR/Cas9 systems have been widely used in numerous eukaryotic organisms, including baker's yeast (Saccharomyces cerevisiae), the opportunistic pathogen Candida albicans, zebrafish (Danio rerio), fruit flies (Drosophila melanogaster), ants (Harpegnathos saltator and Ooceraea biroi), mosquitoes (Aedes aegypti), nematodes (Caenorhabditis elegans), plants, mice, monkeys, and human embryos.

Another recently characterized Cas effector protein is Cas12a (formerly known as Cpf1). Cas12a, together with C2c1 and C2c3, are members belonging to Class 2, type V Cas proteins that lack HNH nuclease, but have RuvC nuclease activity. Cas12a which was initially characterized in the CRISPR/Cpf1 system of the bacterium Francisella novicida. Its original name reflects the prevalence of its CRISPR-Cas subtype in the Prevotella and Francisella lineages. Cas12a showed several key differences from Cas9, including: causing a “staggered” cut in double stranded DNA as opposed to the “blunt” cut produced by Cas9, relying on a “T rich” PAM sequence (which provides alternative targeting sites to Cas9) and requiring only a CRISPR RNA (crRNA) and no tracrRNA for successful targeting. Cas12a's small crRNAs are better suited than Cas9 for multiplexed genome editing, as more of them can be packaged in one vector than can Cas9's sgRNAs. Further, the sticky 5′ overhangs left by Cas12a can be used for DNA assembly that is much more target-specific than traditional Restriction Enzyme cloning. Finally, Cas12a cleaves DNA 18-23 base pairs downstream from its PAM site, which means no disruption to the nuclease recognition sequence after DNA repair following the creation of double stranded break (DSB) by the NHEJ system, thus Cas12a enables multiple rounds of DNA cleavage, as opposed to the likely one round after Cas9 cleavage because the Cas9 cleavage sequence is only 3 base pairs upstream of the PAM site, and the NHEJ pathway typically results in indel mutations which destroy the recognition sequence, thereby preventing further rounds of cutting. In theory, repeated rounds of DNA cleavage is associated with an increased chance for the desired genomic editing to occur.

More recently, several Class 2, type VI Cas proteins, including Cas13 (also known as C2c2), Cas13b, Cas13c, Cas13d (including the engineered variant CasRx), Cas13e, and Cas13f have been identified, each is an RNA-guided RNase (i.e., these Cas proteins use their crRNA to recognize target RNA sequences, rather than target DNA sequences in Cas9 and Cas12a). Overall, the CRISPR/Cas13 systems can achieve higher RNA digestion efficiency compared to the traditional RNAi and CRISPRi technologies, while simultaneously exhibiting much less off-target cleavage compared to RNAi.

CRISPR-Cas13 is quickly becoming a widely adopted RNA editing technology. This system can use its sequence specific guide RNA to selectively modify (e.g., cut or cleave via endonuclease activity) a target RNA, such as mRNA. Compared to the permanent genomic changes introduced by DNA-based editing, RNA controls gene expression at the transcription level, thus providing a safer and more controllable gene therapy approach. Because of the high RNA editing efficiency of the CRISPR/Cas13 systems, they have already been widely used in a number of organisms including yeast, plant, mammal, and zebra fish (see (Abudayyeh et al., 2017; Aman et al., 2018; Cox et al., 2017; Jing et al., 2018; Konermann et al., 2018). An ortholog of CRISPR-Cas13d, CasRx, could mediate RNA knockdown in vivo and effectively alleviate disease phenotypes in various mouse models (He et al., Protein Cell 11:518-524, 2020; Zhou et al., Cell 181:590-603 e516, 2020; and Zhou et al., National Science Review 7:835-837, 2020).

One drawback from these currently identified Cas13 proteins, however, is that they all have non-specific/collateral RNase activity upon activation by crRNA-based target sequence recognition. This activity is particularly strong in Cas13a and Cas13b, and still detectably exists in Cas13d and, to a lesser extent, in Cas13e, for example. While this property can be advantageously used in nucleic acid detection methods, the non-specific/collateral RNase activity of these Cas13 proteins also causes undesirable collateral degradation of bystander RNAs, and has imposed a major barrier for their in vivo application, such as in gene therapy.

On the other hand, for practical utilities such as SHERLOCK that relies on collateral activity for sensitive detection, it can be beneficial to have mutant Cas13 effector enzymes that exhibit even higher collateral activity compared to wild-type Cas13.

Thus, there is a need to further optimize wild-type Cas13 in the art for different purposes, e.g., either to lower collateral cleavage activity with acceptable on-target cleavage activity for certain uses such as therapeutical applications, or to enhance/increase collateral cleavage activity with acceptable on-target cleavage activity for certain other uses such as diagnostic applications.

### SUMMARY OF THE INVENTION

One aspect of the invention provides an engineered Clustered Regularly Interspaced Short Palindromic Repeat (CRISPR)-Cas13 effector enzyme, wherein the engineered Cas13: (1) comprises a mutation in a region spatially close to an endonuclease catalytic domain (e.g., a HEPN domain) of the corresponding wild-type Cas13 effector enzyme; (2) substantially preserves (e.g., retains at least 50%, 60%, 70%, 72.5%, 75%, 80%, 85%, 87.5%, 90%, 95%, 96%, 97%, 97.5%, 98%, 99% or more of) guide sequence-specific endonuclease cleavage activity of the wild-type Cas13 (or theoretical maximum thereof) towards a target RNA complementary to the guide sequence; and, (3) substantially lacks (e.g., retains less than 50%, 40%, 35%, 30%, 27.5%, 25%, 22.5%, 20%, 17.5%, 15%, 12.5%, 10%, 7.5%, 5%, 4%, 3%, 2.5%, 2%, 1% or less of) guide sequence-independent collateral endonuclease cleavage activity of the wild-type Cas13 (or theoretical maximum thereof) towards a non-target RNA that does not bind to the guide sequence.

Another aspect of the invention provides an engineered Clustered Regularly Interspaced Short Palindromic Repeat (CRISPR)-Cas13 effector enzyme, wherein the engineered Cas13: (1) comprises a mutation in a region spatially close to an endonuclease catalytic domain (e.g., a HEPN domain) of the corresponding wild-type Cas13 effector enzyme; (2) substantially preserves or has enhanced (e.g., retains at least 50%, 60%, 70%, 72.5%, 75%, 80%, 85%, 87.5%, 90%, 95%, 96%, 97%, 97.5%, 98%, 99%, 100%, 102%, 105%, 108%, 110% or more of) guide sequence-specific endonuclease cleavage activity of the wild-type Cas13 (or theoretical maximum thereof) towards a target RNA complementary to the guide sequence; and, (3) substantially enhances (e.g., has more than 100%, 110%, 120%, 130%, 140%, 150%, 160%, 170%, 180%, 190%, 200% or more of) guide sequence-independent collateral endonuclease cleavage activity of the wild-type Cas13 towards a non-target RNA that does not bind to the guide sequence.

In certain embodiments, the Cas13 is a Cas13a, a Cas13b, a Cas13c, a Cas13d (including CasRx), a Cas13e, or a Cas13f.

In certain embodiments, the Cas13e has the amino acid sequence of SEQ ID NO: 4, and/or wherein the Cas13d has the amino acid sequence of SEQ ID NO: 101, and/or wherein the Cas13f has the amino acid sequence of SEQ ID NO: 52.

In certain embodiments, the region includes residues within 130, 125, 120, 110, 100, 90, 80, 70, 60, 50, 40, 30, 20, or 10 amino acids from any residues of the endonuclease catalytic domain (e.g., an RXXXXH domain) in the primary sequence of the Cas13e, and residues within 170, 160, 150, 140, 130, 120, 110, 100, 90, 80, 70, 60, 50,40, 30, 20, or 10 amino acids from any residues of the endonuclease catalytic domain (e.g., an RXXXXH domain) in the primary sequence of the Cas13d; or residues within 140, 130, 120, 110, 100, 90, 80, 70, 60, 50, 40, 30, 20, or 10 amino acids from any residues of the endonuclease catalytic domain (e.g., an RXXXXH domain) in the primary sequence of the Cas13f.

In certain embodiments, the region includes residues more than 100, 110, 120, or 130 residues away from any residues of the endonuclease catalytic domain in the primary sequence of the Cas13, but are spatially within 1-10 or 5 angstrom of a residue of the endonuclease catalytic domain.

In certain embodiments, the endonuclease catalytic domain is a HEPN domain, optionally a HEPN domain comprising an RXXXXH motif.

In certain embodiments, the RXXXXH motif comprises a R{N/H/K/Q/R}X₁X₂X₃H sequence (SEQ ID NO: 1024).

In certain embodiments, in the R{N/H/K/Q/R}X₁X₂X₃H sequence (SEQ ID NO: 1025), X₁ is R, S, D, E, Q, N, G, or Y; X₂ is I, S, T, V, or L; and X₃ is L, F, N, Y, V, I, S, D, E, or A.

In certain embodiments, the RXXXXH motif is an N-terminal RXXXXH motif comprising an RNXXXH sequence, such as an RN{Y/F}{F/Y}SH sequence (SEQ ID NO: 64).

In certain embodiments, the N-terminal RXXXXH motif has a RNYFSH sequence (SEQ ID NO: 65).

In certain embodiments, the N-terminal RXXXXH motif has a RNFYSH sequence (SEQ ID NO: 66).

In certain embodiments, the RXXXXH motif is a C-terminal RXXXXH motif comprising an R{N/A/R}{A/K/S/F}{A/L/F}{F/H/L}H sequence (SEQ ID NO: 1026).

In certain embodiments, the C-terminal RXXXXH motif has a RN(A/K)ALH sequence (SEQ ID NO: 67).

In certain embodiments, the C-terminal RXXXXH motif has a RAFFHH (SEQ ID NO: 68) or RRAFFH sequence (SEQ ID NO: 69).

In certain embodiments, said region comprises, consists essentially of, or consists of: (i) residues corresponding to residues between residues 1-194, 2-187, 227-242, 620-775, or 634-755 of SEQ ID NO: 4; or, (ii) residues corresponding to the HEPN1-1 domain (e.g., residues 90-292), Helical2 domain (e.g., residues 536-690), and the HEPN2 domain (e.g., residues 690-967) of SEQ ID NO: 101; or, (iii) residues corresponding to the HEPN1 domain (e.g., residues 1-168), Helical1 domain, Helical2 domain (e.g., residues 346-477), and the HEPN2 domain (e.g., residues 644-790) of SEQ ID NO: 52.

In certain embodiments, said region comprises, consists essentially of, or consists of residues corresponding to residues between residues 35-51, 52-67, 156-171, 666-682, or 712-727 of SEQ ID NO: 4.

In certain embodiments, said mutation comprises, consists essentially of, or consists of substitutions, within a stretch of 15-20 consecutive amino acids within the region, (a) one or more charged, nitrogen-containing side chain group, bulky (such as F or Y), aliphatic, and/or polar residues to a charge-neutral short chain aliphatic residue (such as A, V, or I); (b) one or more I/L to A substitution(s); and/or (c) one or more A to V substitution(s).

In certain embodiments, said stretch is about 16 or 17 residues.

In certain embodiments, substantially all, except for up to 1, 2, or 3, charged and polar residues within the stretch are substituted.

In certain embodiments, a total of about 7, 8, 9, or 10 charged and polar residues within the stretch are substituted.

In certain embodiments, the N- and C-terminal 2 residues of the stretch are substituted to amino acids the coding sequences of which contain a restriction enzyme recognition sequence.

In certain embodiments, the N-terminal two residues are VF, and the C-terminal 2 residues are ED, and the restriction enzyme is BpiI.

In certain embodiments, the one or more charged or polar residues comprise N, Q, R, K, H, D, E, Y, S, and T residues.

In certain embodiments, the one or more charged or polar residues comprise R, K, H, N, Y, and/or Q residues.

In certain embodiments, one or more Y residue(s) within said stretch is substituted.

In certain embodiments, said one or more Y residues(s) correspond to Y672, Y676, and/or Y715 of wild-type Cas13e.1 (SEQ ID NO: 4).

In certain embodiments, said stretch is residues 35-51, 52-67, 156-171, 666-682, or 712-727 of SEQ ID NO: 4.

In certain embodiments, the mutation comprises Ala substitution(s) corresponding to any one or more of SEQ ID NOs: 37-39, 45, and 48.

In certain embodiments, the charge-neutral short chain aliphatic residue is Ala (A).

In certain embodiments, said mutation with reduced collateral activity comprises, consists essentially of, or consists of: (a) substitutions within 1, 2, 3, 4, or 5 of said stretches of 15-20 consecutive amino acids within the region; (b) a mutation corresponds to a Cas13d mutation of Example 4 that retains at least about 75% of guide RNA-specific cleavage of wild-type Cas13d (such as SEQ ID NO: 101) (or theoretical maximum thereof), and exhibits less than about 27.5% collateral effect of wild-type Cas13d (such as SEQ ID NO: 101) (or theoretical maximum thereof); (c) a mutation corresponds to the N1V7, N2V7, N2V8 (cfCas13d), N3V7, or N15V4 mutation of Cas13d mutation; (d) a mutation corresponds to a Cas13d mutation of Example 4 that retains between about 25-75% of guide RNA-specific cleavage of wild-type Cas13d (such as SEQ ID NO: 101) (or theoretical maximum thereof), and exhibits less than about 27.5% collateral effect of wild-type Cas13d (such as SEQ ID NO: 101) (or theoretical maximum thereof); (e) a mutation corresponds to the N2V4, N2V5, N4V3, N6V3, N10V6, N15V2, N20V6, or N20-Y910A mutation of Cas13d mutation; (f) a mutation corresponds to a Cas13e mutation of Example 1, 2, or 5 that retains at least about 75% of guide RNA-specific cleavage of wild-type Cas13e (such as SEQ ID NO: 4) (or theoretical maximum thereof), and exhibits less than about 25% collateral effect of wild-type Cas13e (such as SEQ ID NO: 4) (or theoretical maximum thereof); (g) a mutation corresponds to the M1V4, M2V2, M2V3, M2V4, M5V1, M6V2, M6V3, M6V4, M7V1, M7V2, M7V3, M7-Y55A, M7-Y61A, M11V1, M12V3, M15V1, M15V2, M15-Y643A, M15-Y647A, M16V1, M16V2, M17V2, M18V2, M18V3, M19V2, M19V3, or M19-IA mutation of Cas13e mutation; (h) a mutation corresponds to a Cas13e mutation of Example 5 that retains between about 25-75% of guide RNA-specific cleavage of wild-type Cas13e (such as SEQ ID NO: 4) (or theoretical maximum thereof), and exhibits less than about 25% collateral effect of wild-type Cas13e (such as SEQ ID NO: 4) (or theoretical maximum thereof); (i) a mutation corresponds to the M17YY (cfCas13e), M8V4, M9V1, M11V2, M11V3, M13V1, M13V2, M13V3, M15V3, or M20V2 mutation of Cas13e mutation; (j) a mutation corresponds to a Cas13f mutation (e.g., that of Example 12) that retains at least about 75% of guide RNA-specific cleavage of wild-type Cas13f (such as SEQ ID NO: 52) (or theoretical maximum thereof), and exhibits less than about 25 or 27.5% collateral effect of wild-type Cas13f (such as SEQ ID NO: 52) (or theoretical maximum thereof); (k) a mutation corresponds to the F7V2, F10V1, F10V4, F40V2, F40V4, F44V2, F10S19, F10S21, F10S24, F10S26, F10S27, F10S33, F10S34, F10S35, F10S36, F10S45, F10S46, F10S48, F10S49, F40S22, F40S23, F40S26, F40S27, OR F40S36 mutation of Cas13f mutation; (1) a mutation corresponds to a Cas13f mutation (e.g., that of Example 12) that retains between about 50-75% of guide RNA-specific cleavage of wild-type Cas13f (such as SEQ ID NO: 52) (or theoretical maximum thereof), and exhibits less than about 25 or 27.5% collateral effect of wild-type Cas13f (such as SEQ ID NO: 52) (or theoretical maximum thereof); and/or (m) a mutation corresponds to the F2V4, F3V1, F3V3, F3V4, F5V2, F5V3, F6V4, F7V1, F38V4, F40V1, F41V1, F41V3, F42V4, F43V1, F10S2, F10S11, F10S12, F10S18, F10S20, F10S23, F10S25, F10S28, F10S43, F10S44, F10S47, F10S50, F10S51, F10S52, F40S7, F40S9, F40S11, F40S21, F40S22, F40S24, F40S28, F40S29, F40S30, F40S35, OR F40S37 mutation of Cas13f mutation.

In certain embodiments, the mutation with enhanced collateral activity comprises, consists essentially of, or consists of: (a) substitutions within 1, 2, 3, 4, or 5 of said stretches of 15-20 consecutive amino acids within the region; (b) a mutation corresponds to a Cas13d mutation (e.g., that of Example 4) that retains at least about 75% of guide RNA-specific cleavage of wild-type Cas13d (such as SEQ ID NO: 101) (or theoretical maximum thereof), and exhibits more than about 110%, 115%, 120%, 125%, 130%, 135%, 140%, 145%, 150%, 155%, 160%, 165%, 170%, 175%, 180% or more collateral effect of wild-type Cas13d (such as SEQ ID NO: 101); (c) a mutation corresponds to the N2-Y142A, N4-Y193A, N12-Y604A, N21V7 mutation of Cas13d mutation in Example 4; (d) a mutation corresponds to a Cas13e mutation (e.g., that of Example 5) that retains at least about 75% of guide RNA-specific cleavage of wild-type Cas13e (such as SEQ ID NO: 4) (or theoretical maximum thereof), and exhibits more than about 110%, 115%, 120%, 125%, 130%, 135%, 140%, 145%, 150%, 155%, 160%, 165%, 170%, 175%, 180% or more collateral effect of wild-type Cas13e (such as SEQ ID NO: 4); (e) a mutation corresponds to the M4V2, M4V3, M4V4, M8V1, M8V2, M9V2, M9V3, M10V1, M10V2, M11V4, M12V2, M14V1, M14V2, M16V3, M18V1, M19-G712A, M19-C727A, M19T725A, or M21V2 mutation of Cas13e mutation; (f) a mutation corresponds to a Cas13f mutation (e.g., that of Example 12) that retains at least about 75% of guide RNA-specific cleavage of wild-type Cas13f (such as SEQ ID NO: 52) (or theoretical maximum thereof), and exhibits more than about 110%, 115%, 120%, 125%, 130%, 135%, 140%, 145%, 150%, 155%, 160%, 165%, 170%, 175%, 180% or more collateral effect of wild-type Cas13f (such as SEQ ID NO: 52); (g) a mutation corresponds to the F38V2, F42V1, F46V3, F38S2, F38S4, F38S5, F38S6, F38S7, F38S8, F38S9, F38S10, F38S11, F38S12, F38S13, F38S15, F38S16, F38S17, F40S1, F40S2, F40S3, F40S4, F40S5, F40S6, F40S8, F40S16, F40S18, F46S1, F46S4, F46S6, F46S7, F46S10, F46S14, F46S15, F10S4, F10S5, F10S6, F10S9, F10S10, F10S7, F38S1, F38S13, or F46S2 mutation of Cas13f mutation (e.g., that of Example 12).

In certain embodiments, the engineered Cas13 preserves at least about 50%, 60%, 70%, 80%, 85%, 90%, 95%, 96%, 97%, 98%, or 99% of the guide sequence-specific endonuclease cleavage activity of the wild-type Cas13 (or theoretical maximum thereof) towards the target RNA.

In certain embodiments, the engineered Cas13 lacks at least about 70%, 72.5%, 75%, 77.5%, 80%, 82.5%, 85%, 87.5%, 90%, 92.5%, 95%, 96%, 97%, 98%, 99%, or 100% of the guide sequence-independent collateral endonuclease cleavage activity of the wild-type Cas13 (or theoretical maximum thereof) towards the non-target RNA.

In certain embodiments, the engineered Cas13 preserves at least about 80-90% of the guide sequence-specific endonuclease cleavage activity of the wild-type Cas13 (or theoretical maximum thereof) towards the target RNA, and lacks at least about 95-100% of the guide sequence-independent collateral endonuclease cleavage activity of the wild-type Cas13 (or theoretical maximum thereof) towards the non-target RNA.

In certain embodiments, the engineered Cas13 of the invention has an amino acid sequence at least 80%, 85%, 90%, 95%, 96%, 97%, 98%, 99%, 99.1%, 99.2%, 99.3%, 99.4%, 99.5%, 99.6%, 99.7%, 99.8%, or 99.86% identical to any one of SEQ ID NOs: 6-10 and Cas13d (e.g., SEQ ID NO: 101), excluding any one or more of the regions defined by SEQ ID NOs: 16, 20, 24, 28, and 32, and any of the mutation regions in Example 4 or 5.

In certain embodiments, said amino acid sequence contains up to 1, 2, 3, 4, or 5 differences (a) in each of one or more regions defined by SEQ ID NO: 16, 20, 24, 28, and 32, as compared to SEQ ID NOs: 17, 21, 25, 29, and 33, respectively, or (b) in any of the desired mutations in Cas13d and Cas13e disclosed herein.

In certain embodiments, the engineered Cas13 of the invention has the amino acid sequence of any one of SEQ ID NOs: 6-10.

In certain embodiments, the engineered Cas13 of the invention has the amino acid sequence of SEQ ID NO: 9 or 10.

In certain embodiments, the engineered Cas13 of the invention further comprises a nuclear localization signal (NLS) sequence or a nuclear export signal (NES).

In certain embodiments, the engineered Cas13 comprises an N- and/or a C-terminal NLS.

Another aspect of the invention provides a polynucleotide encoding the engineered Cas13 of the invention.

In certain embodiments, the polynucleotide of the invention is codon-optimized for expression in a eukaryote, a mammal, such as a human or a non-human mammal, a plant, an insect, a bird, a reptile, a rodent (e.g., mouse, rat), a fish, a worm/nematode, or a yeast.

Another aspect of the invention provides A polynucleotide having (i) one or more (e.g., 1, 2, 3, 4, 5, 6, 7, 8, 9 or 10) nucleotides additions, deletions, or substitutions compared to the polynucleotide of the invention; (ii) at least 50%, 60%, 70%, 80%, 90%, 95%, or 97% sequence identity to the polynucleotide of the invention; (iii) hybridize under stringent conditions with the polynucleotide of the invention, or any of (i) and (ii); or (iv) is a complement of any of (i)-(iii).

Another aspect of the invention provides a vector comprising the polynucleotide of the invention.

In certain embodiments, the polynucleotide is operably linked to a promoter and optionally an enhancer.

In certain embodiments, the promoter is a constitutive promoter, an inducible promoter, a ubiquitous promoter, or a tissue specific promoter.

In certain embodiments, the vector is a plasmid.

In certain embodiments, the vector is a retroviral vector, a phage vector, an adenoviral vector, a herpes simplex viral (HSV) vector, an AAV vector, or a lentiviral vector.

In certain embodiments, the AAV vector is a recombinant AAV vector of the serotype AAV1, AAV2, AAV4, AAV5, AAV6, AAV7, AAVrh74, AAV8, AAV9, AAV10, AAV 11, AAV 12, or AAV 13.

Another aspect of the invention provides a delivery system comprising (1) a delivery vehicle, and (2) the engineered Cas13 of the invention, the polynucleotide of the invention, or the vector of the invention.

In certain embodiments, the delivery vehicle is a nanoparticle, a liposome, an exosome, a microvesicle, or a gene-gun.

Another aspect of the invention provides a cell or a progeny thereof, comprising the engineered Cas13 of the invention, the polynucleotide of the invention, or the vector of the invention.

In certain embodiments, the cell or progeny thereof is a eukaryotic cell (e.g., a non-human mammalian cell, a human cell, or a plant cell) or a prokaryotic cell (e.g., a bacteria cell).

Another aspect of the invention provides a non-human multicellular eukaryote comprising the cell of the invention.

In certain embodiments, the non-human multicellular eukaryote is an animal (e.g., rodent or primate) model for a human genetic disorder.

Another aspect of the invention provides a method of modifying a target RNA, the method comprising contacting the target RNA with a CRISPR-Cas13 complex comprising the engineered Cas13 of the invention, and a spacer sequence complementary to at least 15 nucleotides of the target RNA; wherein upon binding of the complex to the target RNA through the spacer sequence, engineered Cas13 modifies the target RNA.

In certain embodiments, the target RNA is modified by cleavage by the engineered Cas13.

In certain embodiments, the target RNA is an mRNA, a tRNA, an rRNA, a non-coding RNA, an lncRNA, or a nuclear RNA.

In certain embodiments, upon binding of the complex to the target RNA, the engineered Cas13 does not exhibit substantial (or detectable) collateral RNase activity.

In certain embodiments, the target RNA is within a cell.

In certain embodiments, the cell is a cancer cell.

In certain embodiments, the cell is infected with an infectious agent.

In certain embodiments, the infectious agent is a virus, a prion, a protozoan, a fungus, or a parasite.

In certain embodiments, the cell is a neuronal cell (e.g., astrocyte, glial cell (e.g., Muller glia cell, oligodendrocyte, ependymal cell, Schwan cell, NG2 cell, or satellite cell)).

In certain embodiments, the CRISPR-Cas13 complex is encoded by a first polynucleotide encoding the engineered Cas13 of the invention, and a second polynucleotide comprising or encoding a spacer RNA capable of binding to the target RNA, wherein the first and the second polynucleotides are introduced into the cell.

In certain embodiments, the first and the second polynucleotides are introduced into the cell by the same vector.

In certain embodiments, the method causes one or more of: (i) in vitro or in vivo induction of cellular senescence; (ii) in vitro or in vivo cell cycle arrest; (iii) in vitro or in vivo cell growth inhibition and/or cell growth inhibition; (iv) in vitro or in vitro induction of anergy; (v) in vitro or in vitro induction of apoptosis; and (vi) in vitro or in vitro induction of necrosis.

Another aspect of the invention provides a method of treating a condition or disease in a subject in need thereof, the method comprising administering to the subject a composition comprising a CRISPR-Cas complex comprising the engineered Cas13 of the invention or a polynucleotide encoding the same; and a spacer sequence complementary to at least 15 nucleotides of a target RNA associated with the condition or disease; wherein upon binding of the complex to the target RNA through the spacer sequence, the engineered Cas13 cleaves the target RNA, thereby treating the condition or disease in the subject.

In certain embodiments, the condition or disease is a neurological condition, a cancer or an infectious disease.

In certain embodiments, the cancer is Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, or urinary bladder cancer.

In certain embodiments, the neurological condition is glaucoma, age-related RGC loss, optic nerve injury, retinal ischemia, Leber's hereditary optic neuropathy, a neurological condition associated with degeneration of RGC neurons, a neurological condition associated with degeneration of functional neurons in the striatum of a subject in need thereof, Parkinson's disease, Alzheimer's disease, Huntington's disease, Schizophrenia, depression, drug addiction, movement disorder such as chorea, choreoathetosis, and dyskinesias, bipolar disorder, Autism spectrum disorder (ASD), or dysfunction.

In certain embodiments, the method is an in vitro method, an in vivo method, or an ex vivo method.

Another aspect of the invention provides A CRISPR-Cas complex comprising the engineered Cas13 of the invention, a guide RNA comprising a DR sequence that binds the engineered Cas13 and a spacer sequence designed to be complementary to and binds a target RNA.

In certain embodiments, the target RNA is encoded by a eukaryotic DNA.

In certain embodiments, the eukaryotic DNA is a non-human mammalian DNA, a non-human primate DNA, a human DNA, a plant DNA, an insect DNA, a bird DNA, a reptile DNA, a rodent DNA, a fish DNA, a worm/nematode DNA, a yeast DNA.

In certain embodiments, the target RNA is an mRNA.

In certain embodiments, the CRISPR-Cas complex further comprises a target RNA comprising a sequence capable of hybridizing to the spacer sequence.

Another aspect of the invention provides a method of identifying an engineered CRISPR/Cas effector enzyme of a corresponding wild-type Cas effector enzyme, wherein the engineered Cas substantially maintains guide-sequence-specific endonuclease activity and substantially lacks guide-sequence-independent collateral endonuclease activity, the method comprising: (1) in each of one or more regions of 15-20 consecutive polynucleotides (a) within 80, 90, 100, 110, 120, 130, 140, 150, 160, 170, or 180 residues of any residues of a endonuclease catalytic domain of the wild-type Cas effector enzyme or (b) spatially within 1-10 Ångström of any residues of the endonuclease catalytic domain of the wild-type Cas effector enzyme, substituting one or more (e.g., substantially all, except for up to 1, 2, 3, 4, or 5) polar and charged residues with a charge neutral aliphatic side-chain residue (such as A); and, (2) identifying engineered Cas substantially maintains guide-sequence-specific endonuclease activity and substantially lacks guide-sequence-independent collateral endonuclease activity compared to the corresponding wild-type Cas.

In certain embodiments, the wild-type Cas effector enzyme is a Cas13.

In certain embodiments, the Cas13 is a Cas13a, a Cas13b, a Cas13c, a Cas13d (e.g., CasRx), a Cas13e, or a Cas13f.

In certain embodiments, the Cas13e has the amino acid sequence of SEQ ID NO: 4; or wherein the Cas13d has the amino acid sequence of SEQ ID NO: 101; or wherein the Cas13f has the amino acid sequence of SEQ ID NO: 52.

Another aspect of the invention provides a method of identifying an engineered Clustered Regularly Interspaced Short Palindromic Repeat (CRISPR)-Cas13 effector enzyme with altered guide sequence-independent collateral nuclease activity, the method comprising: in a region spatially close to an endonuclease catalytic domain of the corresponding wild-type Cas13 effector enzyme, substituting one or more charged or polar residues to a charge-neutral short chain aliphatic residue (such as A), to determine whether the resulting variant Cas13 effector enzyme: (1) has substantially preserved guide sequence-specific endonuclease cleavage activity of the wild-type Cas13 (or theoretical maximum thereof) towards a target RNA complementary to the guide sequence; and, (2) either substantially lacks or has enhanced guide sequence-independent collateral endonuclease cleavage activity of the wild-type Cas13 (or theoretical maximum thereof) towards a non-target RNA that does not bind to the guide sequence, thereby identifying said engineered Cas13 effector enzyme with altered guide sequence-independent collateral nuclease activity.

In certain embodiments, the engineered Cas13 effector enzyme substantially lacks guide sequence-independent collateral nuclease activity.

In certain embodiments, the engineered Cas13 effector enzyme has enhanced guide sequence-independent collateral nuclease activity.

In certain embodiments, said one or more charged or polar residues are within a stretch of 15-20 (e.g., 16 or 17) consecutive amino acids within the region.

In certain embodiments, said one or more charged or polar residues comprise, consist essentially of, or consist of one or more (or all) Tyr (Y) residue(s) within the stretch.

It should be understood that any one embodiment of the invention described herein, including those described only in the examples or claims, or only in one aspects/sections below, can be combined with any other one or more embodiments of the invention, unless explicitly disclaimed or improper.

### BRIEF DESCRIPTION OF THE DRAWINGS

FIG. 1 is a schematic (not to scale) illustration of a possible mechanism of reduced collateral effect by a Cas13 (e.g., Cas13e) effector enzyme. The upper left panel shows a possible mechanism of sequence-specific targeting and cleavage of a target RNA by wild-type Cas13e. The upper right panel shows a possible mechanism of non-sequence-specific targeting and cleavage of non-target RNA by wild-type Cas13e. The lower left panel shows a possible mechanism of action by a subject engineered Cas13e with reduced affinity for non-target RNA and higher tendency to cleave target RNA in a sequence-specific manner.

FIG. 2 shows a predicted 3D structure of a Cas13e protein.

FIG. 3 shows the locations of the mutations in the engineered Cas13e mapped to the wild-type Cas13e sequence (SEQ ID NO: 4). The two HEPN sequences (HEPN1 and HEPN2) are also shown.

FIG. 4 is a schematic drawing (not to scale) of the double-fluorescent vector used to identify the subject engineered Cas13e effector proteins. The guide RNA (gRNA) encoded by the vector targets an EGFP reporter. Boxes with dashed lines include the two HEPN RXXXXH sequences (HEPN1 and HEPN2) and their respective nearby sequences (residues 2-187 and 634-755), as well as a sequence (residues 227-242) predicted to be spatially close to the HEPN sequences in Cas13e. Mutations with desired functional changes in those regions were identified in engineered Cas13e.

FIG. 5 shows the relative fluorescent intensity distribution among the various engineered Cas13e effector enzymes (Mut-1 to Mut-21) and Cas13e wild-type positive and negative controls, each shown as the intensity difference between the targeted (guide sequence-specific cleavage of) EGFP signal (left panel) and the control mCherry signal (right panel).

FIG. 6 shows the relative percentage of mCherry positive cells, upon comparing the various engineered Cas13e effector enzymes to wild-type or dCas13e (nuclease null mutant), after activating Cas13e nuclease activity using guide-sequence specific cleavage of EGFP. Engineered Cas13e effector enzymes with close to 100% relative percentage of mCherry positive cells have no or nearly no non-sequence-specific endonuclease activity, like dCas13e (which has neither sequence-specific nor non-sequence-specific endonuclease activity).

FIG. 7 shows the relative percentage of EGFP positive cells, upon comparing the various engineered Cas13e effector enzymes to wild-type or dCas13e (nuclease null mutant), after activating Cas13e nuclease activity using guide-sequence specific cleavage of EGFP. Engineered Cas13e effector enzymes with close to wild-type Cas13e relative percentage (e.g., about 20%) of EGFP positive cells have comparable level of sequence-specific endonuclease activity as wild-type Cas13e.

FIG. 8 shows the spacial distribution of the various mutations with reduced collateral effect, in the predicted Cas13e 3D structure.

FIG. 9 shows the sequences of several mutations in the Mut-17 region. FIG. 9 discloses SEQ ID NOs: 28, 29, and 36-43, respectively, in order of appearance.

FIG. 10 shows the relative percentage of mCherry positive cells, upon comparing the various engineered Cas13e effector enzymes to wild-type or dCas13e (nuclease null mutant), after activating Cas13e nuclease activity using guide-sequence specific cleavage of EGFP. Engineered Cas13e effector enzymes with close to 100% relative percentage of mCherry positive cells have no or nearly no non-sequence-specific endonuclease activity, like dCas13e (which has neither sequence-specific nor non-sequence-specific endonuclease activity).

FIG. 11 shows the relative percentage of EGFP positive cells, upon comparing the various engineered Cas13e effector enzymes to wild-type or dCas13e (nuclease null mutant), after activating Cas13e nuclease activity using guide-sequence specific cleavage of EGFP. Engineered Cas13e effector enzymes with close to wild-type Cas13e relative percentage (e.g., about 20%) of EGFP positive cells have comparable level of sequence-specific endonuclease activity as wild-type Cas13e.

FIG. 12 shows the sequences of the mutations in the Mut-19 region. FIG. 12 discloses SEQ ID NOs: 32 and 44-49, respectively, in order of appearance.

FIG. 13 shows the relative percentage of mCherry positive cells, upon comparing the various engineered Cas13e effector enzymes to wild-type or dCas13e (nuclease null mutant), after activating Cas13e nuclease activity using guide-sequence specific cleavage of EGFP. Engineered Cas13e effector enzymes with close to 100% relative percentage of mCherry positive cells have no or nearly no non-sequence-specific endonuclease activity, like dCas13e (which has neither sequence-specific nor non-sequence-specific endonuclease activity). M17.15-1 and M17.15-2 are the same, and are both double mutants with both Y-to-A mutations in M17.8 and M17.9 (see FIG. 9).

FIG. 14 shows the relative percentage of EGFP positive cells, upon comparing the various engineered Cas13e effector enzymes to wild-type or dCas13e (nuclease null mutant), after activating Cas13e nuclease activity using guide-sequence specific cleavage of EGFP. Engineered Cas13e effector enzymes with close to wild-type Cas13e relative percentage (e.g., about 20%) of EGFP positive cells have comparable level of sequence-specific endonuclease activity as wild-type Cas13e.

FIG. 15 is a schematic drawing showing the domain structures for representative Cas13a-Cas13f effector enzymes. The overall sizes, and the locations of the two RXXXXH motifs on each representative member of the representative Cas13 proteins are indicated.

FIGS. 16A-16D show the results of evaluating collateral effects in transiently transfected mammalian cell HEK293T using the dual-fluorescence reporter system of the invention.

FIG. 16A is a schematic drawing of the mammalian dual-fluorescence reporter system used to evaluate collateral effects induced by Cas13 (Cas13d/Cas13a)-mediated RNA knockdown. The exemplary dual-fluorescence reporter used herein contains one plasmid with coding sequences for Cas13 (with NLS) and EGFP under the transcription control of the strong CAG promoter, and another plasmid with coding sequences for the various gRNA targeting endogenous or exogenous targets (e.g., mCherry, NT, or RPL4, under the transcriptional control of the U6 promoter) and mCherry (under the transcriptional control of the EF1α promoter). NLS, nuclear localization signal; DR: direct repeat; P2A: 2A peptide from porcine teschovirus-1 promoter; and pA: polyA signal. HEK293T cells transfected by the dual-fluorescence reporter system plasmids are subjected to FACS analysis for EGFP (non-specific target) and mCherry (specific target) expression 48 hrs post transfection. Representative FACS analysis data of Cas13d/Cas13a-mediated mCherry and EGFP RNA knockdown with three different mCherry gRNAs in HEK293T cells, compared with NT, and representative FACS analysis data of mCherry and EGFP RNA knockdown induced by Cas13d with four different RPL4 gRNAs in HEK293T cells, compared with NT, are not shown.

FIG. 16B shows the bar graphs summarizing the relative knockdown of exogenous gRNA specific target mCherry and exogenous collateral target EGFP transcripts induced by Cas13d (left panel) or Cas13a (middle panel) with three different mCherry gRNAs, as well as the relative knockdown of the endogenous gRNA specific RPL4 and exogenous collateral target EGFP transcripts induced by Cas13d with four different RPL4 gRNAs (right panel). Knockdown relative to a NT gRNA was determined by qPCR. NT: non-targeting gRNA. All values are mean±s.e.m. (n=3), unless otherwise noted. Two-tailed unpaired two-sample t-test was used for statisticalal analysis. *P<0.05, **P<0.01, ***P<0.001, ns, no significance.

FIG. 16C shows FACS quantitative analysis of relative percentage of EGFP or mCherry positive cells from these experiments. NT: non-targeting gRNA. All values are mean±s.e.m. (n=3), unless otherwise noted. Two-tailed unpaired two-sample t-test was used for statisticalal analysis. *P<0.05, **P<0.01, ***P<0.001, ns, no significance.

FIG. 16D shows characteristics collateral effects of Cas13-mediated endogenous transcripts knockdown in HEK293T cells. Representative bright-field, fluorescence images, and flow cytometry images of cells with reduced mCherry and EGFP fluorescence intensity using Cas13d knockdown of three endogenous transcripts (RPL4, PFN1, PKM), each with four gRNAs, were not shown. However, differential decreases of relative percentage of EGFP or mCherry positive cells were induced by Cas13d targeting PFN1 (left panel) and PKM (right panel) transcript, with four gRNAs each transcript. NT: non-targeting gRNA. All values are mean±s.e.m. (n=3), unless otherwise noted. Two-tailed unpaired two-sample t-test was used for statisticalal analysis. *P<0.05, **P<0.01, ***P<0.001, ns, no significance.

FIGS. 17A-17H show results of rational mutagenesis of Cas13d to eliminate collateral activity. FIG. 17A is a schematic drawing of the mammalian dual-fluorescence reporter system used to screen on-target interference activity of Cas13 (shown as Cas13d but broadly represent all Cas13, including Cas13a, Cas13b, Cas13c, Cas13d, Cas13e, and Cas13f, etc.), with coding sequences for Cas13, EGFP (target in this experiment), mCherry (collateral target in this experiment) and EGFP gRNA all in one plasmid. Wild-type (wt) Cas13 cleaves the target EGFP mRNA via the gRNA-specific mechanism and the non-target mCherry mRNA via the collateral activity. dCas13 does not cleave either mCherry or EGFP mRNA for lack of endonuclease activity. The subject engineered Cas13 mutants/variants preserved gRNA-specific EGFP cleavage, but lost the collateral activity against mCherry mRNA. FIG. 17B shows a view of the predicted overall structure (by I-TASSER) of the RfxCas13d complex in ribbon representation. RXXXXH of HEPN domains are the catalytic sites. FIG. 17C shows the 21 regions in HEPN1 (including HEPN1-I and HEPN1-II), HEPN2, Helical2 and partial Helical1 domains of Cas13d selected for mutagenesis studies, with each spanning about 36-amino acids. FIG. 17D shows quantification of relative percentage of EGFP or mCherry positive cells among 118 Cas13d mutants targeting EGFP transcript. WT (wild-type Cas13d) and dead Cas13d (dCas13d) as controls, relative percentages of positive cell were all normalized to dCas13d. FIG. 17E shows quantification of relative percentage of EGFP or mCherry positive cells among Cas13d mutants with different combinations of mutation sites within or nearby N2V7 and N2V8. WT (wild-type Cas13d) and dead Cas13d (dCas13d) as controls, relative percentages of positive cell were all normalized to dCas13d. Representative FACS analysis of mCherry and EGFP knockdown induced by Cas13d mutants with EGFP gRNA is not shown. FIG. 17F shows differential changes of relative percentage of mCherry and EGFP positive cells were induced by cfCas13d with EGFP gRNAs in comparison with Cas13d, dCas13d as control. FIGS. 17G and 17H show kinetics of in vitro nuclease activity for Cas13 enzymes. In vitro collateral ribonuclease activity (FIG. 17G) analysis and target ribonuclease activity (FIG. 17H) analysis of Cas13d, cfCas13d, and dCas13d with off-target or on-target synthetic ssRNA fluorescence probes.

FIGS. 18A and 18B show the cartoon view (FIG. 18A) and opposing surface view (FIG. 18B) of the crystal structure of Cas13d, including the catalytic sites of the HEPN domains (labeled by RXXXXH), and effective mutated sites (labeled by the various NxVy mutations).

FIG. 18C shows mutated sequences of effective variants from Cas13d. FIG. 18C discloses SEQ ID NOs: 948, 949, 561, 950-955, 561, 950, 951, 601, 615, and 625, respectively, in order of columns.

FIGS. 19A-19I show results of rational mutagenesis of Cas13e to improve nuclease specificity. FIG. 19A shows a view of the predicted overall structure of the Cas13e complex in ribbon representation. RXXXXH of HEPN domains are catalytic sites. FIG. 19B shows a mutagenesis scheme according to which the HEPN1 and HEPN2 domains were mainly selected and divided into 21 mutant regions for further subsequent mutagenesis. FIG. 19C shows quantification of relative percentage of EGFP or mCherry positive cells among Cas13e mutants targeting EGFP transcript. WT (wild-type Cas13e) and dead Cas13e (dCas13e) were used as positive and negative controls, respectively, and the relative percentages of positive cell were all normalized to dCas13e. FIG. 19D shows quantification of relative percentage of EGFP or mCherry positive cells among Cas13e mutants from different combinations of mutation sites based on M17 targeting EGFP transcript. Cas13e and dCas13e as used as controls. FIGS. 19E and 19F show kinetics of in vitro nuclease activity for Cas13 enzymes. In vitro collateral ribonuclease activity (FIG. 19E) analysis and target ribonuclease activity (FIG. 19F) analysis of Cas13e, cfCas13e, and dCas13e with off-target or on-target synthetic ssRNA fluorescence probes. FIG. 19G shows differential changes of mCherry and EGFP fluorescence intensity induced by cfCas13e with EGFP gRNAs in comparison with Cas13e. FIG. 19H is a schematic diagram showing the AAV vector genome encoding cfCas13e (collateral activity free Cas13e) and guide RNAs targeting VEGFA, and results of target mRNA knock-down. FIG. 19I shows knock down of target mRNA using cfCas13e in a dose-dependent manner and results comparison with two comparator drugs.

FIGS. 20A-20I show efficient and specific interference activity of cfCas13d targeting endogenous genes in HEK293 cells. FIG. 20A shows relative expression level (as measured by CPM, counts per million) of 23 endogenous genes in HEK293 cells from RNA-seq of dCas13d groups. FIG. 20B shows differential decreases of relative percentage of EGFP or mCherry positive cells induced by Cas13d targeting 22 endogenous transcripts, with 1-7 gRNAs each transcript, compared with NT. FIG. 20C shows statisticalal quantification from FIG. 20B. FACS images of differential decreases of mCherry and EGFP fluorescence intensity induced by dCas13d/Cas13d/cfCas13d with gRNA targeting RPL4, PPIA or RPS5 transcripts were not shown, but FACS quantitative analysis of relative percentage of EGFP or mCherry positive cells from such FACS analysis is shown in FIGS. 20D-20G. FIG. 20H shows Cas13d and cfCas13d targeting of 14 endogenous transcripts in HEK293 cells. Transcript levels are relative to dCas13d as vehicle control. FIG. 20I shows statisticalal data analysis from FIG. 20H. NT: non-targeting gRNA. All values are mean±s.e.m. (n=3), unless otherwise noted. Two-tailed unpaired two-sample t-test was used. *P<0.05, **P<0.01, ***P<0.001, ns, no significance.

FIGS. 20J and 20K show differential gene expression of Cas13d/cfCas13d targeting CA2/B4GALNT1 transcripts by flow cytometry analysis. FACS images of differential decreases of mCherry and EGFP fluorescence intensity induced by dCas13d/Cas13d/cfCas13d with targeting CA2 or B4GALNT1 transcript gRNA were not shown, but FACS quantitative analysis of relative percentage of EGFP or mCherry positive cells were shown in FIGS. 20J and 20K. All values are mean±s.e.m. (n=3), unless otherwise noted. Two-tailed unpaired two-sample t-test was used for statisticalal analysis. *P<0.05, **P<0.01, ***P<0.001, ns, no significance.

FIGS. 21A-21E show the results of transcriptome-wide off-target edits analysis of Cas13d/cfCas13d targeting endogenous transcript. FIG. 21A shows characteristic of gRNA dependent off-target sites from RPL4-g3, PPIA-g1, CA2-g1 or PPARG-g1, measured in Cas13d and cfCas13d groups. MM #, mismatch number of off-target sites. FIG. 21A discloses SEQ ID NOs: 956, 956, 956-958, and 958-970, respectively, in order of appearance. FIG. 21B shows statisticalal data analysis from FIG. 21A, of which off-target sites with one or more mismatches were analyzed. FIGS. 21C-21D show biological process of significant down-regulated genes induced by Cas13d/cfCas13d-mediated RPL4 (FIG. 21C)/PPIA (FIG. 21D) knockdown. In FIGS. 21C and 21D, the relevant genes are 0008219 (cell death), 0007049 (cell cycle), 0009056 (catabolic process), 0007165 (signal tranduction), 0009058 (biosynthetic process), 0051716 (cellular response to stimulus), 0071704 (organic substance metabolic process), and 0071840 (cellular component organization or biogenesis). In FIG. 21E, characteristic of gRNA dependent off-target sites from RPL4-g1 or PPIA-g2 were measured in Cas13d and cfCas13d groups. MM #, mismatch number of off-target sites. FIG. 21E discloses SEQ ID NOs: 971 and 971-975, respectively, in order of appearance.

FIGS. 22A-22C show cellular consequences and working model of collateral effects and its elimination. FIG. 22A is a schematic drawing of the dox-inducible Cas13d/cfCas13d/dCas13d expression system with RPL4 gRNA1 used to examine collateral effects. Representative bright-field images of HEK293T cell clones with dox-inducible Cas13d/cfCas13d/dCas13d expression system during 5 days after dox treatment were not shown. FIG. 22B left panel shows relative RPL4 mRNA knockdown by dCas13d/Cas13d/cfCas13d with RPL4 gRNA in the presence or absence of dox during 5 days. The two middle panels show growth curve and MTT assay of dCas13d, Cas13d, or cfCas13d cell clones treated with/without dox during 5 or 6 days (n=3). The right panel shows statistical analysis from the first three panels. FIG. 22C is a model of Cas13 on-target and collateral cleavage activity. Once activated by target RNA, cfCas13 (e.g., cfCas13d and cfCas13e) with mutant sites maintains on-target cleavage activity but eliminates collateral cleavage activity, while wtCas13 exhibits both cleavage activity. All values are mean±s.e.m. (n=3), unless otherwise noted. Two-tailed unpaired two-sample t-test was used for statisticalal analysis. *P<0.05, **P<0.01, ***P<0.001, ns, no significance.

FIGS. 23A-23J is an exemplary multi-sequence alignment of several representative Cas13 family proteins (e.g., Cas13b, Cas13e and Cas13f), and the domain organizations including the HPEN domains. FIGS. 23A-23J disclose SEQ ID NOs: 4, and 976-994, respectively, in order of appearance.

FIGS. 24A-24M is an exemplary multi-sequence alignment of several representative Cas13 family proteins (e.g., Cas13d, Cas13a and Cas13c), and the domain organizations including the HPEN domains. FIGS. 24A-24M disclose SEQ ID NOs: 101, 995-1008, 1007, 1009-1023, and 855, respectively, in order of appearance.

FIG. 25 is a schematic drawing of the mammalian dual-fluorescence reporter system used to screen on-target interference activity of Cas13f, with the Cas13f coding sequences, the EGFP target, the mCherry collateral target, and the EGFP gRNA in one plasmid. Wild-type (wt) Cas13f cleaves the target EGFP mRNA via gRNA-specific mechanism, and the non-target mCherry mRNA via its collateral activity. dCas13f cleaves neither mCherry nor EGFP mRNA, for lack of endonuclease activity. The subject engineered Cas13f mutants/variants preserved gRNA-specific EGFP cleavage, but lost its collateral activity against the mCherry mRNA.

FIG. 26 shows a view of the predicted overall structure (by I-TASSER) of the Cas13f.1 complex in ribbon representation. RXXXXH motifs of the HEPN domains are the catalytic sites.

FIG. 27 shows the 47 regions in HEPN1, HEPN2, Helical1 (including Hel1-1, Hel1-2 and Hel1-3) and Helical2 domains of Cas13f selected for mutagenesis, with each spanning about 17-amino acids.

FIG. 28 shows quantification of the relative percentages of EGFP or mCherry⁺ cells among 75 Cas13f mutants targeting EGFP transcript. WT (wild-type) Cas13f and dead Cas13f (dCas13f) are controls. Relative percentages of positive cell were normalized to dCas13df.

FIG. 29 shows quantification of relative percentages of EGFP or mCherry⁺ cells among Cas13f mutants with different combinations of mutation sites within or nearby F10V1, F10V4, F38V2, F40V2, F40V4, F46V1 and F46V3. WT (wild-type) Cas13f and dead Cas13f (dCas13f) are controls. Relative percentages of positive cell were normalized to dCas13f. Representative FACS analysis of mCherry and EGFP knock-down induced by Cas13f mutants with EGFP gRNA is not shown.

### DETAILED DESCRIPTION OF THE INVENTION

### 1. Overview

A broad range of CRISPR-Cas systems has been discovered, and a classification system and a common nomenclature have been established for the associated Cas genes. Under such classification system, the CRISPR-Cas systems and the associated effector enzymes belong to two classes—Class 1 and Class 2—each further divided into three types and numerous subtypes based on their signature Cas genes. The Class 1 systems encompass types I, III, and IV systems, utilizing multisubunit RNA-Protein (RNP) complexes. The Class 2 systems encompass types II, V, and VI systems, utilizing single protein RNP complexes.

Cas9 is a Class 2, type II effector enzyme, while the recently discovered Cas13 enzymes, including Cas13a, Cas13b, Cas13c, Cas13d (including the engineered variant CasRx), Cas13e, and Cas13f are Class 2, type VI effector enzymes. Unlike any other CRISPR-Cas systems, Class 2 type VI effector proteins have been demonstrated to exclusively cleave RNA targets. Such Class 2 type VI effector enzymes have two distinct active sites, both conferring RNase activity: one involved in pre-crRNA processing, the other involved in target RNA degradation.

Several subtypes of Class 2 type VI exist, including at least subtype VI-A (Cas13a/C2c2), VI-B (Cas13b1 and Cas13b2), VI-C (Cas13c), VI-D (Cas13d, CasRx), VI-E (Cas13e), and VI-F (Cas13f). The Cas13 subtypes generally share very low sequence identity/similarity, but can all be classified as type VI Cas proteins (e.g., generally referred to herein as “Cas13”) based on the presence of two conserved HEPN-like RNase domains. See FIG. 15. Although these two domains appear to be a conserved feature of Cas13 enzymes and are typically located close to the two terminal ends, their spacing within the protein appears to be unique for each subtype. At least three crystal structures for type VI-A Cas13a proteins have been published, including Cas13a from Leptotrichia shahii (LshCas13a), Lachnospiraceae bacterium (LbaCas13a), and Leptotrichia buccalis (LbuCas13a). Similar to other Class 2 complexes, the crRNA-Cas13a complex is bi-lobed with a nuclease (NUC) lobe and a crRNA recognition (REC) lobe. The crRNA-bound form of Cas13a adopts a “clenched fist”-like structure, with the REC lobe being imperfectly stacked on top of the NUC lobe. The REC lobe has a variable N-terminal domain (NTD), followed by a helical domain (Helical-1). Meanwhile, the NUC lobe consists of the two HEPN domains (HEPN-1 and HEPN-2) separated by a linker domain (Helical-3). In addition, the HEPN-1 domain is split into two subdomains by another helical domain (Helical-2). The NTD, Helical-1, and HEPN2 domains form a narrow, positively charged cleft that anchors the 5′ repeat-derived end of the bound crRNA (the 5′-handle), whereas the 3′ end of the crRNA is bound by the Helical-2 domain.

The Cas13 CRISPR locus is initially transcribed into a long pre-crRNA transcript. The Cas13 proteins then cleave the pre-crRNA at fixed positions upstream of the stem-loop structure formed by the palindromic nature of the direct repeat (DR) sequences. Pre-crRNA processing in type VI involves metal-independent cleavages upstream of the stem-loop, and does not require a trans-activating crRNA (tracrRNA) or other host factors. The mature crRNA, which comprises a DR sequence and a guide sequence complementary to a target RNA, assembles with the Cas13 proteins to form a functional RNP complex, which then scans transcripts for the complementary RNA target. Once such RNA target is found and bound by the guide sequence, the RNA target is degraded by the Cas13 endonuclease.

The Cas13 effector enzymes display unprecedented sensitivity to recognize specific target RNAs within a heterogeneous population of non-target RNAs. It has been reported that Cas13 can detect target RNAs with femtomolar sensitivity. Thus on the one hand, the Class 2 type VI enzymes or Cas13 offer tremendous opportunity to knock down target gene products (e.g., mRNA) for gene therapy, yet on the other hand, such use is inherently limited by the co-called collateral activity that poses significant risk of cytotoxicity.

Specifically, in Class 2 type VI systems, a guide sequence non-specific RNA cleavage, referred to as “collateral activity,” is conferred by the higher eukaryotes and prokaryotes nucleotide-binding (HEPN) domain in Cas13 after target RNA binding. Binding of its cognate target ssRNA complementary to the bound crRNA causes substantial conformational changes in Cas13 effector enzyme, leading to the formation of a single, composite catalytic site for guide-sequence independent “collateral” RNA cleavage, thus converting Cas13 into a sequence non-specific ribonuclease. This newly formed highly accessible active site would not only degrade the target RNA in cis if the target RNA is sufficiently long to reach this new active site, but also degrade non-target RNAs in trans based on this promiscuous RNase activity.

Most RNAs appear to be vulnerable to this promiscuous RNAse activity of Cas13, and most (if not all) Cas13 effector enzymes possess this collateral endonuclease activity. It has been shown recently that the collateral effects by Cas13-mediated knockdown exist in mammalian cells and animals (manuscript submitted), suggesting that clinical application of Cas13-mediated target RNA knock down will face significant challenge in the presence of collateral effect.

The existence of substantial collateral effects of Cas13-mediated RNA knockdown has been demonstrated using a dual-fluorescent reporter system of the invention as described herein. Such collateral effects have been observed for both exogenous and endogenous genes in mammalian cells. In particular, wild-type Cas13d with this collateral effect was found to induce transcriptome-wide off-target editing and cell growth arrest.

Thus, in order to use the Cas13 enzymes for specifically knocking down a target RNA in gene therapy, it is evident that this guide-sequence non-specific collateral activity must be tightly controlled to prevent unwanted spontaneous cellular toxicity. Through unclear mechanism, subtype VI-B systems include a natural means to regulate the collateral activity of Cas13b via the type VI-associated genes csx27 and csx28, but such natural regulatory mechanism appears to be unique to subtype VI-B, as similar mechanism does not seem to exist in other subtypes such as type VI-A and VI-C.

Using this same reporter system of the invention, about 200 Cas13d and Cas13e variants obtained by structure-guided mutagenesis were screened. It was found that several variants with 2-4 mutations on the Higher Eukaryotes and Prokaryotes Nucleotide-binding (HEPN) domains retained undiminished on-target activity, but greatly reduced collateral effects. For the Cas13d variant with diminished collateral effect, the transcriptome-wide off-target editing and cell growth arrest observed in wild-type Cas13d were eliminated.

Interestingly, it was found that the majority of variants exhibited either low dual cleavage activity, or high on-target cleavage activity but low collateral cleavage activity. However, there is almost no variants showing low on-target cleavage activity but high collateral cleavage activity. These results suggest a distinct binding mechanism between on-target and collateral cleavage activity.

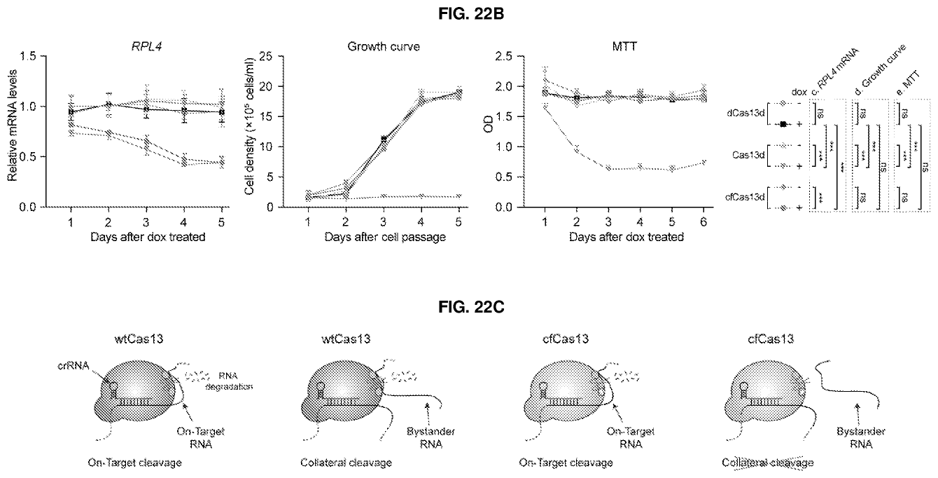

While not wishing to be bound by any particular theory, Applicant believes the following model of target (e.g., gRNA-specific) and collateral cleavage activity aids the rationale design of collateral effect-free variants of the Cas13 effector enzymes. Specifically, as shown in FIG. 22C, Cas13 is believed to contain two separated binding domains proximal to the HEPN domains—one is responsible for on-target cleavage, and both are required for collateral cleavage. Consistent with this model, mutations designed on the N1V7, N2V7, N2V8 and N15V4 regions, surrounding the cleavage site, cause steric hindrance effects or change in charge, leading to weakened interactions between activated Cas13 and promiscuous RNA, but not much (if any) effect between activated Cas13 and the on-target RNA. Thus, mutagenesis on these binding sites abolishes the collateral cleavage activity of Cas13, while retaining the on-target cleavage activity of the corresponding wild-type Cas13.

Thus, the invention described herein provides engineered high-fidelity Class 2 type VI or Cas13 (e.g., Cas13d, Cas13e, and Cas13f) effector enzyme variants with minimal residual collateral effects. These variants are useful, for example, in targeting degradation of RNAs in basic research and therapeutic applications.

On the other hand, multiple low-fidelity Cas13 variants exhibiting increased dual cleavage activity were identified. Such variants have utility for better nucleic acid detection application (such as those used in the SHERLOCK assay).

Specifically, in one aspect, the invention provides engineered Class 2 type VI or Cas13 (e.g., Cas13d, e, or f) effector enzymes that largely maintain their sequence-specific endonuclease activity against a target RNA, yet with diminished if not eliminated non-guide sequence-specific endonuclease activity against non-target RNAs. Such engineered Cas13 effector enzymes that substantially lack collateral effect pave the way for using Cas13 in target RNA-knock down-based utility, such as gene therapy. Such engineered Cas13 effector enzymes that substantially lack collateral effect are also useful for RNA-base editing, because a nuclease dead version (or “dCas13”) of such engineered Cas13 also has reduced off-target effect, which is still present in dCas13 without the mutations in the subject engineered Cas13.

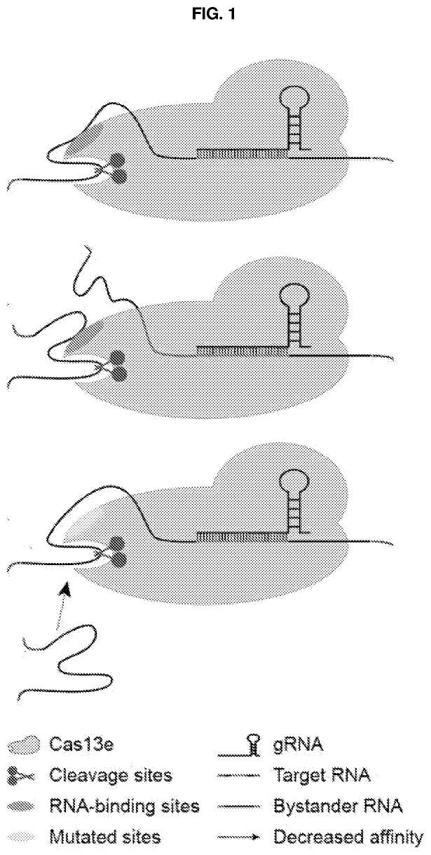

While not wishing to be bound by any particular theory, FIGS. 1 and 22C (see above) provide plausible mechanisms consistent with the data presented herein. In particular, in FIG. 1, a wild-type Cas13 not only possesses the ability to bind a target RNA through the guide sequence of the crRNA, but also possesses a non-specific RNA binding site (see the oval shaped motif around the catalytic site) for any RNA at the vicinity of the HEPN catalytic domains. Once the target RNA is recognized by the guide sequence, a conformation change of Cas13 activates its catalytic activity, and the target RNA, bound by both the complementary guide sequence and the non-specific RNA binding site, is cleaved. Once activated, Cas13 also non-specifically cleave non-target RNA that does not bind to the guide sequence, partly due to the binding of such non-target RNA to the non-specific RNA binding site on cas13. Mutations in the non-specific RNA binding motif (as signified by a different shade of the oval motif) reduces/eliminates (or in some cases enhances) the ability of Cas13 to bind RNA, thus collateral activity against non-target RNA is reduced/eliminated (or enhanced) without significantly affecting target RNA cleavage because the target RNA is still bound by the guide sequence.

According to this model, off-target effect in RNA-base editing using a nuclease-deficient (dCas13) version of the engineered Cas13 can also be reduced or eliminated, because the loss of non-specific RNA binding in the engineered dCas13 reduced/eliminates unintended RNA based editing due to the proximity of the RNA base editing domain (e.g., ADAR or CDAR) and an off-target RNA substrate.

In a related aspect, the invention also provides engineered Class 2 type VI or Cas13 (e.g., Cas13d, Cas13e, or Cas13f) effector enzymes that largely maintain their sequence-specific endonuclease activity against a target RNA, yet with enhanced non-guide sequence-specific endonuclease activity against non-target RNAs compared to the corresponding wild-type Cas13. Such engineered Cas13 with enhanced collateral effect provides a better (e.g., more sensitive) variant, compared to the wild-type, in nucleic acid detection assays such as SHERLOCK, which takes advantage of the collateral activity to provide an extreme sensitive assay for detecting very small quantities of a guide sequence-specific target RNA in a sample, with or without pre-amplification of the initial nucleic acids in the sample.

More specifically, one aspect of the invention provides an engineered Class 2 type VI Clustered Regularly Interspaced Short Palindromic Repeat (CRISPR)-Cas effector enzyme, such as Cas13 (e.g., Cas13d, Cas13e, or Cas13f) wherein the engineered Class 2 type VI Cas effector enzyme: (1) comprises a mutation in a region spatially close to an endonuclease catalytic domain of the corresponding wild-type effector enzyme; (2) substantially preserves guide sequence-specific endonuclease cleavage activity of the wild-type effector enzyme (or theoretical maximum thereof) towards a target RNA complementary to the guide sequence; and, (3) either substantially lacks or has enhanced guide sequence-independent collateral endonuclease cleavage activity of the wild-type effector enzyme (or theoretical maximum thereof) towards a non-target RNA that is substantially not complement to/does not bind to the guide sequence.

In certain embodiments, the guide sequence-specific endonuclease cleavage activity and the guide sequence-independent collateral endonuclease cleavage activity can both be measured as compared to the corresponding wild-type Cas13 effector enzymes (such as mutant Cas13e vs. wild-type Cas13e from which the mutant derives from), as normalized against a corresponding nuclease-deficient Cas13 (such as dCas13e).

The nuclease-deficient Cas13 may be lack of catalytic domain, motif, or key catalytic residues such that it exhibits no appreciable or detectable level of guide sequence-dependent target RNA endonuclease cleavage activity, as well as guide sequence-independent collateral endonuclease cleavage activity. Thus in the due reporter system described herein, dCas13 typically has 100% remaining/baseline EGFP signal as an indication of no appreciable or detectable level of guide sequence-dependent target RNA endonuclease cleavage activity, and has 100% remaining/baseline mCherry signal as an indication of no appreciable or detectable level of guide sequence-independent collateral endonuclease cleavage activity. Meanwhile, wild-type Cas13 typically exibit strong guide sequence-dependent target RNA endonuclease cleavage activity (as reflected by nearly 80%, 90%, 95%, or close to 100% reduction of the dCas13 EGFP reference signal). The theoretical maximum of such guide sequence-dependent target RNA endonuclease cleavage activity is 100%, which is equivalent to complete elimination of all dCas13 EGFP reference signal.

Wild-type Cas13 also typically exhibit various levels of guide sequence-independent collateral endonuclease cleavage activity, leading to about 50%-70% reduction of the dCas13 mCherry reference signal. The theoretical maximum of such guide sequence-independent collateral endonuclease cleavage activity is 100%, which is equivalent to complete elimination of all dCas13 mCherry reference signal.

In certain embodiments, the engineered Cas13 effector enzyme of the invention exhibits reduced or diminished guide sequence-independent collateral endonuclease cleavage activity compared to the corresponding wild-type Cas13 (or theoretical maximum thereof) from which the engineered Cas13 derives. For example, the engineered Cas13 effector enzyme may substantially lack (e.g., retains less than 50%, 40%, 35%, 30%, 27.5%, 25%, 22.5%, 20%, 17.5%, 15%, 12.5%, 10%, 7.5%, 5%, 4%, 3%, 2.5%, 2%, 1% or less of) guide sequence-independent collateral endonuclease cleavage activity of the wild-type Cas13 towards a non-target RNA that does not bind to the guide sequence. For example, if the wild-type Cas13 eliminates about 70% (with the theoretical maximum being 100% elimination) of the dCas13 mCherry baseline signal due to collateral activity, and the mutant Cas13 with diminished collateral activity only eliminates about 10% of the dCas13 mCherry baseline signal due to remaining collateral activity, the mutant only exhibits or retains about 1/7 (or about 15%) of the wild-type collateral activity (or 10% of the theoretical maximum).

In certain embodiments, the engineered Cas13 effector enzyme of the invention exhibits increased or enhanced guide sequence-independent collateral endonuclease cleavage activity compared to the corresponding wild-type Cas13 from which the engineered Cas13 derives. For example, the engineered Cas13 effector enzyme may have substantially enhanced or increased (e.g., has more than 100%, 110%, 120%, 130%, 140%, 150%, 160%, 170%, 180%, 190%, 200% or more of) guide sequence-independent collateral endonuclease cleavage activity of the wild-type Cas13 towards a non-target RNA that does not bind to the guide sequence. For example, if the wild-type Cas13 eliminates about 50% of the dCas13 mCherry baseline signal due to collateral activity, and the mutant Cas13 with enhanced collateral activity eliminates about 90% of the dCas13 mCherry baseline signal due to its enhanced collateral activity, the mutant exhibits about 90/50 (or about 180%) of the wild-type collateral activity.

In certain embodiments, the mutation occurs within a region, e.g., within one of two RNA binding domains at, near, or proximal to one of the HEPN-type catalytic domains, of a wild-type Cas13 (such as Cas13a, Cas13b, Cas13c, Cas13d, Cas13e, Cas13f etc). In certain embodiments, the mutation weakens (e.g., significantly weakens or eliminates) binding of the wild-type Cas13 to a non-specific RNA target (e.g., one not substantially complementary to a guide RNA), but substantially retains binding to a target RNA substantially complementary to the guide RNA. In certain embodiments, the mutation causes steric hindrance effects and/or change in charge, polarity, and/or size of the sidechain of the involved residues, leading to weakened interactions between activated Cas13 and promiscuous RNA, but not much (if any) effect between activated Cas13 and the on-target RNA.

As used herein, “Cas13” is a Class 2 type VI CRISPR-Cas effector enzyme that displays collateral activity as wild-type enzyme upon binding to a cognate target RNA complementary to a guide sequence of its crRNA. The collateral activity of a wild-type Class 2 type VI effector enzyme enables it to cleave RNase or endonuclease activity against a non-target RNA that does not or substantially does not complement with the guide sequence of the crRNA. The wild-type Class 2 type VI effector enzyme may also exhibit one or more of the following characteristics: having one or two conserved HEPN-like RNase domains, such as HEPN domains having the conserved RXXXXH motif (with X being any amino acid), e.g., the RXXXXH motifs described herein below; having a “clenched fist”-like structure when the Class 2 type VI effector enzyme (e.g., Cas13) binds a cognate crRNA; having a bi-lobed structure with a nuclease (NUC) lobe and a crRNA recognition (REC) lobe, optionally, the REC lobe has a variable N-terminal domain (NTD), followed by a helical domain (Helical-1), and/or optionally, the NUC lobe consists of the two HEPN domains (HEPN-1 and HEPN-2) separated by a linker domain (Helical-3), wherein the HEPN-1 domain is optionally split into two subdomains by another helical domain (Helical-2); processes pre-crRNA transcript into crRNA; does not require a trans-activating crRNA (tracrRNA) or other host factors for pre-crRNA processing; and exhibits femtomolar sensitivity to recognize guide sequence-specific target RNAs within a heterogeneous population of non-target RNAs.

In certain embodiments, the Class 2 type VI effector enzyme (e.g., Cas13) has one of the RXXXXN motifs in the HEPN-like domains located at or close to (e.g., within 50-160 residues, or within 10, 20, 30, 40, 50, 60, 70, 80, 90, 100, 110, 120, 130, 140, 150 or 160 residues of) the N-terminus. In certain embodiments, the Class 2 type VI effector enzyme (e.g., Cas13) has one of the RXXXXN motifs in the HEPN-like domains located at or close to (e.g., within 50-160 residues, or within 10, 20, 30, 40, 50, 60, 70, 80, 90, 100, 110, 120, 130, 140, 150 or 160 residues of) the C-terminus. In certain embodiments, the Class 2 type VI effector enzyme (e.g., Cas13) has one of the RXXXXN motifs of the HEPN-like domains located at or close to (e.g., within 50-160 residues, or within 10, 20, 30, 40, 50, 60, 70, 80, 90, 100, 110, 120, 130, 140, 150 or 160 residues of) the N-terminus, while the other of the RXXXXN of the HEPN-like domains is located at or close to (e.g., within 50-160 residues, or within 10, 20, 30, 40, 50, 60, 70, 80, 90, 100, 110, 120, 130, 140, 150 or 160 residues of) the C-terminus. An RXXXXN motif is “at or near” the N- or C-terminus, if either the R or the N residue of the RXXXXN motif is at or near the N- or C-terminus.

Based on biological and cellular experimental data, the engineered Class 2 type VI effector enzyme (e.g., Cas13 particularly Cas13e) effector enzymes have drastically reduced non-sequence-specific endonuclease activity against non-target RNAs, yet simultaneously exhibiting substantially the same if not higher sequence-specific endonuclease activity against a target RNA that substantially complements the guide sequence of the crRNA. The engineered effector enzymes enable high fidelity RNA targeting/editing.

In certain embodiments, the Class 2 type VI effector enzyme is Cas13a, Cas13b, Cas13c, Cas13d (including the engineered variant CasRx), Cas13e, or Cas13f, or an ortholog, paralog, homolog, natural or engineered variant thereof, or functional fragment thereof that substantially maintains the guide sequence-specific endonuclease activity.

In certain embodiments, the variant or functional fragment thereof maintains at least one function of the corresponding wild-type effector enzyme. Such functions include, but are not limited to, the ability to bind a guide RNA/crRNA of the invention (described herein below) to form a complex, the guide sequence-specific RNase activity, and the ability to bind to and cleave a target RNA at a specific site under the guidance of the crRNA that is at least partially complementary to the target RNA.

In certain embodiments, the Cas13 protein is a Cas13a protein. In some embodiments, the Cas13a protein is from a species of the genus Bacteroides, Blautia, Butyrivibrio, Carnobacterium, Chloroflexus, Clostridium, Demequina, Eubacterium, Herbinix, Insoliti spirillum, Lachnospiraceae, Leptotrichia, Listeria, Paludibacter, Porphyromonadaceae, Pseudobutyrivibrio, Rhodobacter, or Thalassospira. In certain embodiments, the Cas13a protein is from a species of Leptotrichia shahii, Listeria seeligeri, Lachnospiraceae bacterium (such as Lb MA2020, Lb NK4A179, Lb NK4A144), Clostridium aminophilum (such as Ca DSM 10710), Carnobacterium gallinarum (such as Cg DSM 4847), Paludibacter propionicigenes (such as Pp WB4), Listeria weihenstephanensis (such as Lw FSL R9-0317), Listeriaceae bacterium (such as Lb FSL M6-0635), Leptotrichia wadei (such as Lw F0279), Rhodobacter capsulatus (such as Rc SB 1003, Rc R121, Rc DE442), Leptotrichia buccalis (such as Lb C-1013-b), Herbinix hemicellulosilytica, Eubacteriaceae bacterium (such as Eb CHKCI004), Blautia. sp Marseille-P2398, Leptotrichia sp. oral taxon 879 str. F0557, Chloroflexus aggregans, Demequina aurantiaca, Thalassospira sp. TSLS-1, Pseudobutyrivibrio sp. OR37, Butyrivibrio sp. YAB3001, Leptotrichia sp. Marseille-P3007, Bacteroides ihuae, Porphyromonadaceae bacterium (such as Pb KH3CP3RA), Listeria riparia, or Insoliti spirillum peregrinum.

In certain embodiments, the Cas13a is any one of Cas13a disclosed in WO2020/028555 (incorporated herein by reference).

In some embodiments, the Cas13 protein is a Cas13b protein. In some embodiments, the Cas13b protein is from a species of the genus Alistipes, Bacteroides, Bacteroidetes, Bergeyella, Capnocytophaga, Chryseobacterium, Flavobacterium, Myroides, Paludibacter, Phaeodactylibacter, Porphyromonas, Prevotella, Psychroflexus, Reichenbachiella, Riemerella, or Sinomicrobium. In certain embodiments, the Cas13b protein is from a species Alistipes sp. ZOR0009, Bacteroides pyogenes (such as Bp F0041), Bacteroidetes bacterium (such as Bb GWA2319), Bergeyella zoohelcum (such as Bz ATCC 43767), Capnocytophaga canimorsus, Capnocytophaga cynodegmi, Chryseobacterium carnipullorum, Chryseobacterium jejuense, Chryseobacterium ureilyticum, Flavobacterium branchiophilum, Flavobacterium columnare, Flavobacterium sp. 316, Myroides odoratimimus (such as Mo CCUG 10230, Mo CCUG 12901, Mo CCUG 3837), Paludibacter propionicigenes, Phaeodactylibacter xiamenensis, Porphyromonas gingivalis (such as Pg F0185, Pg F0568, Pg JCVI SC001, Pg W4087, Porphyromonas gulae, Porphyromonas sp. COT-052 OH4946, Prevotella aurantiaca, Prevotella buccae (such as Pb ATCC 33574), Prevotella falsenii, Prevotella intermedia (such as Pi 17, Pi ZT), Prevotella pallens (such as Pp ATCC 700821), Prevotella pleuritidis, Prevotella saccharolytica (such as Ps F0055), Prevotella sp. MA2016, Prevotella sp. MSX73, Prevotella sp. P4-76, Prevotella sp. P5-119, Prevotella sp. P5-125, Prevotella sp. P5-60, Psychroflexus torquis, Reichenbachiella agariperforans, Riemerella anatipestifer, or Sinomicrobium oceani.

In certain embodiments, the Cas13b is any one of Cas13b disclosed in WO2020/028555 (incorporated herein by reference).

In some embodiments, the Cas13 protein is a Cas13c protein. In some embodiments, the Cas13c protein is from a species of the genus Fusobacterium or Anaerosalibacter. In certain embodiments, the Cas13c protein is from a species of Fusobacterium necrophorum (such as Fn subsp. funduliforme ATCC 51357, Fn DJ-2, Fn BFTR-1, Fn subsp. Funduliforme), Fusobacterium perfoetens (such as Fp ATCC 29250), Fusobacterium ulcerans (such as Fu ATCC 49185), or Anaerosalibacter sp. ND1.

In certain embodiments, the Cas13c is any one of Cas13c disclosed in WO2020/028555 (incorporated herein by reference).

In some embodiments, the Cas13 protein is a Cas13d protein. In some embodiments, the Cas13d protein is from a species of the genus Eubacterium or Ruminococcus. In certain embodiments, the Cas13d protein is from a species of Eubacterium siraeum, Ruminococcus flavefaciens (such as Rfx XPD3002), or Ruminococcus albus. In certain embodiments, Cas13d is CasRx. In certain embodiments, Cas13d has the amino acid sequence of SEQ ID NO: 101.

In certain embodiments, the Cas13d is any one of Cas13d disclosed in WO2020/028555 (incorporated herein by reference).

In some embodiments, the Cas13 protein is a Cas13e protein. In some embodiments, the Cas13e protein is from a species of the genus Planctomycetes. In certain embodiments, the Cas13e protein has an amino acid sequence of SEQ ID NO: 4, 50 or 51. The direct repeat (DR) sequences for the Cas13e of SEQ ID NOs: 50 and 51 are SEQ ID NOs: 57 and 58, respectively.

In some embodiments, the Cas13 protein is a Cas13f protein. In certain embodiments, the Cas13f protein has an amino acid sequence of any one of SEQ ID NOs: 52-56. The direct repeat (DR) sequences for the Cas13f of SEQ ID NOs: 52-56 are SEQ ID NOs: 59-63, respectively.

As used herein, “direct repeat sequence” may refer to the DNA coding sequence in the CRISPR locus, or to the RNA encoded by the same in crRNA. Thus when any of SEQ ID NOs: 57-63 is referred to in the context of an RNA molecule, such as crRNA, each T is understood to represent a U.

In certain embodiments, the wild-type Cas effector proteins of the invention can be: (i) any one of SEQ ID NOs: 50-56, such as SEQ ID NO: 50; (ii) an ortholog, paralog, homolog of any one of SEQ ID NOs: 50-56; or (iii) a Class 2 type VI effector enzyme having amino acid sequence identity of at least about 80%, 85%, 90%, 91%, 92%, 93%, 94%, 95%, 96%, 97%, 98%, or 99% compared to any one of SEQ ID NOs: 50-56.

In certain embodiments, the Cas13e and Cas13f effector proteins, orthologs, homologs, derivatives and functional fragments thereof are naturally existing. In certain other embodiments, the Cas13e and Cas13f effector proteins, orthologs, homologs, derivatives and functional fragments thereof are not naturally existing, e.g., having at least one amino acid difference compared to a naturally existing sequence.

In certain embodiments, the region spatially close to the endonuclease catalytic domain of the corresponding wild-type Cas13 effector enzyme includes residues within 120, 110, 100, 90, 80, 70, 60, 50, 40, 30, 20, or 10 amino acids from any residues of the endonuclease catalytic domain (e.g., an RXXXXH domain) in the primary sequence of the Cas13.

In certain embodiments, the region includes residues within 130, 125, 120, 110, 100, 90, 80, 70, 60, 50, 40, 30, 20, or 10 amino acids from any residues of the endonuclease catalytic domain (e.g., an RXXXXH domain) in the primary sequence of the Cas13e; residues within 170, 160, 150, 140, 130, 120, 110, 100, 90, 80, 70, 60, 50, 40, 30, 20, or 10 amino acids from any residues of the endonuclease catalytic domain (e.g., an RXXXXH domain) in the primary sequence of the Cas13d; or residues within 140, 130, 120, 110, 100, 90, 80, 70, 60, 50, 40, 30, 20, or 10 amino acids from any residues of the endonuclease catalytic domain (e.g., an RXXXXH domain) in the primary sequence of the Cas13f.

In certain embodiments, the region spatially close to the endonuclease catalytic domain of the corresponding wild-type Cas13 effector enzyme includes residues more than 100, 110, 120, or 130 residues away from any residues of the endonuclease catalytic domain in the primary sequence of the Cas13, but are spatially within 1-10 or 5 ångström of a residue of the endonuclease catalytic domain.

In certain embodiments, the endonuclease catalytic domain is a HEPN domain, optionally a HEPN domain comprising an RXXXXH motif.

In certain embodiments, the RXXXXH motif comprises a R{N/H/K/Q/R}X₁X₂X₃H sequence (SEQ ID NO: 1024).

In certain embodiments, in the R{N/H/K/Q/R}X₁X₂X₃H sequence (SEQ ID NO: 1025), X₁ is R, S, D, E, Q, N, G, or Y; X₂ is I, S, T, V, or L; and X₃ is L, F, N, Y, V, I, S, D, E, or A.

In certain embodiments, the RXXXXH motif is an N-terminal RXXXXH motif comprising an RNXXXH sequence, such as an RN{Y/F}{F/Y}SH sequence (SEQ ID NO: 64). In certain embodiments, the N-terminal RXXXXH motif has a RNYFSH sequence (SEQ ID NO: 65). In certain embodiments, the N-terminal RXXXXH motif has a RNFYSH sequence (SEQ ID NO: 66). In certain embodiments, the RXXXXH motif is a C-terminal RXXXXH motif comprising an R{N/A/R}{A/K/S/F}{A/L/F}{F/H/L}H sequence (SEQ ID NO: 1026). For example, the C-terminal RXXXXH motif may have a RN(A/K)ALH sequence (SEQ ID NO: 67), or a RAFFHH (SEQ ID NO: 68) or RRAFFH sequence (SEQ ID NO: 69).

In certain embodiments, region comprises, consists essentially of, or consists of: (a) residues corresponding to residues between residues 1-194, 2-187, 227-242, 620-775, or 634-755 of SEQ ID NO: 4. In certain embodiments, region comprises, consists essentially of, or consists of residues corresponding to residues between residues 35-51, 52-67, 156-171, 666-682, or 712-727 of SEQ ID NO: 4; (ii) residues corresponding to the HEPN1-1 domain (e.g., residues 90-292), Helical2 domain (e.g., residues 536-690), and the HEPN2 domain (e.g., residues 690-967) of SEQ ID NO: 101; or (iii) residues corresponding to the HEPN1 domain (e.g., residues 1-168), Helical1 domain, Helical2 domain (e.g., residues 346-477), and the HEPN2 domain (e.g., residues 644-790) of SEQ ID NO: 52.

In certain embodiments, the mutation comprises, consists essentially of, or consists of substitutions, within a stretch of 15-20 consecutive amino acids within the region, one or more charged or polar residues to a charge neutral short chain aliphatic residue (such as A). For example, in some embodiments, the stretch is about 16 or 17 residues.

In certain embodiments, the mutation comprises, consists essentially of, or consists of substitutions, within a stretch of 15-20 consecutive amino acids within the region, (a) one or more charged, nitrogen-containing side chain group, bulky (such as F or Y), aliphatic, and/or polar residues to a charge-neutral short chain aliphatic residue (such as A, V, or I); (b) one or more I/L to A substitution(s); and/or (c) one or more A to V substitution(s).

In certain embodiments, substantially all, except for up to 1, 2, or 3, charged and polar residues within the stretch are substituted.

In certain embodiments, a total of about 7, 8, 9, or 10 charged and polar residues within the stretch are substituted.

In certain embodiments, the N- and C-terminal 2 residues of the stretch are substituted to amino acids the coding sequences of which contain a restriction enzyme recognition sequence. For example, in some embodiments, the N-terminal two residues may be VF, and the C-terminal 2 residues may be ED, and the restriction enzyme is BpiI. Other suitable RE sites are readily envisioned. The RE sites for the N- and C-terminal ends can be, but need not be identical.

In certain embodiments, the one or more charged or polar residues comprise N, Q, R, K, H, D, E, Y, S, and T residues. In certain embodiments, the one or more charged or polar residues comprise R, K, H, N, Y, and/or Q residues.

In certain embodiments, one or more Y residue(s) within said stretch is substituted. In certain embodiments, said one or more Y residues(s) correspond to Y672, Y676, and/or Y715 of wild-type Cas13e.1 (SEQ ID NO: 4). In certain embodiments, said stretch is residues 35-51, 52-67, 156-171, 666-682, or 712-727 of SEQ ID NO: 4.

In certain embodiments, the mutation leads to reduction or elimination of guide sequence-independent collateral RNase activity. In certain embodiments, the mutation comprises charge-neutral short chain aliphatic residue substitution(s) corresponding to any one or more of SEQ ID NOs: 37-39, 45, and 48.

In certain embodiments, the mutation leads to enhanced guide sequence-independent collateral RNase activity compared to the wild-type Cas13. In certain embodiments, the mutation comprises charge-neutral short chain aliphatic residue substitution(s) corresponding to any one or more of SEQ ID NOs: 40-42.

In certain embodiments, the charge-neutral short chain aliphatic residue is A, I, L, V, or G.

In certain embodiments, the charge-neutral short chain aliphatic residue is Ala (A).

In certain embodiments, the mutation comprises, consists essentially of, or consists of substitutions within 2, 3, 4, or 5 said stretches of 15-20 consecutive amino acids within the region.

In certain embodiments, the mutation with reduced collateral activity comprises, consists essentially of, or consists of: (a) substitutions within 1, 2, 3, 4, or 5 of said stretches of 15-20 consecutive amino acids within the region; (b) a mutation corresponds to a Cas13d mutation (e.g., that of Example 4) that retains at least about 75% of guide RNA-specific cleavage of wild-type Cas13d (such as SEQ ID NO: 101) (or theoretical maximum thereof), and exhibits less than about 25% or 27.5% collateral effect of wild-type Cas13d (such as SEQ ID NO: 101) (or theoretical maximum thereof); (c) a mutation corresponds to the N1V7, N2V7, N2V8 (cfCas13d), N3V7, or N15V4 mutation of Cas13d mutation; (d) a mutation corresponds to a Cas13d mutation (e.g., that of Example 4) that retains between about 25-75% of guide RNA-specific cleavage of wild-type Cas13d (such as SEQ ID NO: 101) (or theoretical maximum thereof), and exhibits less than about 25% or 27.5% collateral effect of wild-type Cas13d (such as SEQ ID NO: 101) (or theoretical maximum thereof); (e) a mutation corresponds to the N2V4, N2V5, N4V3, N6V3, N10V6, N15V2, N20V6, or N20-Y910A mutation of Cas13d mutation; (f) a mutation corresponds to a Cas13e mutation (e.g., that of Example 1, 2, or 5) that retains at least about 75% of guide RNA-specific cleavage of wild-type Cas13e (such as SEQ ID NO: 4) (or theoretical maximum thereof), and exhibits less than about 25% collateral effect of wild-type Cas13e (such as SEQ ID NO: 4) (or theoretical maximum thereof); (g) a mutation corresponds to the M1V4, M2V2, M2V3, M2V4, M5V1, M6V2, M6V3, M6V4, M7V1, M7V2, M7V3, M7-Y55A, M7-Y61A, M11V1, M12V3, M15V1, M15V2, M15-Y643A, M15-Y647A, M16V1, M16V2, M17V2, M18V2, M18V3, M19V2, M19V3, or M19-IA mutation of Cas13e mutation; (h) a mutation corresponds to a Cas13e mutation (e.g., that of Example 5) that retains between about 25-75% of guide RNA-specific cleavage of wild-type Cas13e (such as SEQ ID NO: 4) (or theoretical maximum thereof), and exhibits less than about 25% collateral effect of wild-type Cas13e (such as SEQ ID NO: 4) (or theoretical maximum thereof); and/or (i) a mutation corresponds to the M17YY (cfCas13e), M8V4, M9V1, M11V2, M11V3, M13V1, M13V2, M13V3, M15V3, or M20V2 mutation of Cas13e mutation; (j) a mutation corresponds to a Cas13f mutation (e.g., that of Example 12) that retains at least about 75% of guide RNA-specific cleavage of wild-type Cas13f (such as SEQ ID NO: 52) (or theoretical maximum thereof), and exhibits less than about 25 or 27.5% collateral effect of wild-type Cas13f (such as SEQ ID NO: 52) (or theoretical maximum thereof); (k) a mutation corresponds to the F7V2, F10V1, F10V4, F40V2, F40V4, F44V2, F10S19, F10S21, F10S24, F10S26, F10S27, F10S33, F10S34, F10S35, F10S36, F10S45, F10S46, F10S48, F10S49, F40S22, F40S23, F40S26, F40S27, OR F40S36 mutation of Cas13f mutation; (1) a mutation corresponds to a Cas13f mutation (e.g., that of Example 12) that retains between about 50-75% of guide RNA-specific cleavage of wild-type Cas13f (such as SEQ ID NO: 52) (or theoretical maximum thereof), and exhibits less than about 25 or 27.5% collateral effect of wild-type Cas13f (such as SEQ ID NO: 52) (or theoretical maximum thereof); and/or (m) a mutation corresponds to the F2V4, F3V1, F3V3, F3V4, F5V2, F5V3, F6V4, F7V1, F38V4, F40V1, F41V1, F41V3, F42V4, F43V1, F10S2, F10S11, F10S12, F10S18, F10S20, F10S23, F10S25, F10S28, F10S43, F10S44, F10S47, F10S50, F10S51, F10S52, F40S7, F40S9, F40S11, F40S21, F40S22, F40S24, F40S28, F40S29, F40S30, F40S35, OR F40S37 mutation of Cas13f mutation.

In certain embodiments, the mutation with enhanced collateral activity comprises, consists essentially of, or consists of: (a) substitutions within 1, 2, 3, 4, or 5 of said stretches of 15-20 consecutive amino acids within the region; (b) a mutation corresponds to a Cas13d mutation (e.g., that of Example 4) that retains at least about 75% of guide RNA-specific cleavage of wild-type Cas13d (such as SEQ ID NO: 101) (or theoretical maximum thereof), and exhibits more than about 110%, 115%, 120%, 125%, 130%, 135%, 140%, 145%, 150%, 155%, 160%, 165%, 170%, 175%, 180% or more collateral effect of wild-type Cas13d (such as SEQ ID NO: 101); (c) a mutation corresponds to the N2-Y142A, N4-Y193A, N12-Y604A, N21V7 mutation of Cas13d mutation in Example 4; (d) a mutation corresponds to a Cas13e mutation (e.g., that of Example 5) that retains at least about 75% of guide RNA-specific cleavage of wild-type Cas13e (such as SEQ ID NO: 4) (or theoretical maximum thereof), and exhibits more than about 110%, 115%, 120%, 125%, 130%, 135%, 140%, 145%, 150%, 155%, 160%, 165%, 170%, 175%, 180% or more collateral effect of wild-type Cas13e (such as SEQ ID NO: 4); (e) a mutation corresponds to the M4V2, M4V3, M4V4, M8V1, M8V2, M9V2, M9V3, M10V1, M10V2, M11V4, M12V2, M14V1, M14V2, M16V3, M18V1, M19-G712A, M19-C727A, M19T725A, or M21V2 mutation of Cas13e mutation; (1) a mutation corresponds to a Cas13f mutation (e.g., that of Example 12) that retains at least about 75% of guide RNA-specific cleavage of wild-type Cas13f (such as SEQ ID NO: 52) (or theoretical maximum thereof), and exhibits more than about 110%, 115%, 120%, 125%, 130%, 135%, 140%, 145%, 150%, 155%, 160%, 165%, 170%, 175%, 180% or more collateral effect of wild-type Cas13f (such as SEQ ID NO: 52); (g) a mutation corresponds to the F38V2, F42V1, F46V3, F38S2, F38S4, F3855, F38S6, F38S7, F38S8, F38S9, F38S10, F38S11, F38S12, F38S13, F38S15, F38S16, F38S17, F40S1, F40S2, F40S3, F40S4, F40S5, F40S6, F40S8, F40S16, F40S18, F46S1, F46S4, F46S6, F46S7, F46S10, F46S14, F46S15, F10S4, F10S5, F10S6, F10S9, F10S10, F10S7, F38S1, F38S13, or F46S2 mutation of Cas13f mutation (e.g., that of Example 12).

The sequences of the mutations and/or variants referenced herein for Cas13d, Cas13e, and Cas13f are described in detail in the examples (such as examples 1, 2, 4, 5, and 12) and the associated sequence listing.

In certain embodiments, more than one (e.g., any combinations of two or more of) such mutations/variants may be present in the same engineered Cas13 effector enzyme.

In certain embodiments, the engineered Cas13 preserves at least about 50%, 60%, 70%, 72.5%, 75%, 80%, 85%, 87.5%, 90%, 95%, 96%, 97%, 97.5%, 98%, or 99% of the guide sequence-specific endonuclease cleavage activity of the wild-type Cas13 (or theoretical maximum thereof) towards the target RNA.

In certain embodiments, the engineered Cas13 has at least about 95%, 100%, 105%, 110%, 115%, 120%, 125%, 130%, 135%, 140%, 145%, 150%, 155%, 160% or more of the guide sequence-specific endonuclease cleavage activity of the wild-type Cas13 towards the target RNA. That is, the subject engineered Cas13 variant may have higher guide sequence-specific endonuclease cleavage activity towards the target RNA compared to the wild-type Cas13 from which the variant is derived.

In certain embodiments, the engineered Cas13 lacks at least about 70%, 72.5%, 75%, 77.5%, 80%, 82.5%, 85%, 87.5%, 90%, 92.5%, 95%, 96%, 97%, 98%, 99%, or 100% of the guide sequence-independent collateral endonuclease cleavage activity of the wild-type Cas13 (or theoretical maximum thereof) towards the non-target RNA.

In certain embodiments, the engineered Cas13 preserves at least about 80-90% of the guide sequence-specific endonuclease cleavage activity of the wild-type Cas13 (or theoretical maximum thereof) towards the target RNA, and lacks at least about 95-100% of the guide sequence-independent collateral endonuclease cleavage activity of the wild-type Cas13 (or theoretical maximum thereof) towards the non-target RNA.

In certain embodiments, the guide RNA-specific and collateral (gRNA-independent) cleavage activity by the engineered Cas13 effector enzymes are measured using methods substantially as described in any of the examples (such as Examples 1, 2, 4, 5 and 12).

In certain embodiments, the engineered Cas13 of the invention has an amino acid sequence at least 80%, 85%, 90%, 95%, 96%, 97%, 98%, 99%, 99.1%, 99.2%, 99.3%, 99.4%, 99.5%, 99.6%, 99.7%, 99.8%, or 99.86% identical to any one of SEQ ID NOs: 6-10, and Cas13d (such as SEQ ID NO: 101), excluding any one or more of the regions defined by SEQ ID NOs: 16, 20, 24, 28, and 32, and any of the mutation regions in Example 4 or 5. For example, in the regions outside or excluding SEQ ID NOs: 16, 20, 24, 28, and/or 32, the engineered Cas13 of the invention may differ from the engineered Cas13 of any one of SEQ ID NOs: 6-10 by 1, 2, 3, 4, 5, 6, 7, 8, 9, 10, or more residues, provided that such additional changes do not substantially negatively affect the guide sequence-specific endonuclease activity, and/or do not increase the guide sequence-independent collateral effect.

In certain embodiments, the amino acid sequence contains up to 1, 2, 3, 4, or 5 differences in each of one or more regions defined by SEQ ID NO: 16, 20, 24, 28, and 32, as compared to SEQ ID NOs: 17, 21, 25, 29, and 33, respectively. For example, additional changes in SEQ ID NOs: 17, 21, 25, 29, and/or 33 are possible without substantially negatively affect the guide sequence-specific endonuclease activity, and/or do not increase the guide sequence-independent collateral effect.

In certain embodiments, the engineered Cas13 of the invention has the amino acid sequence of any one of SEQ ID NOs: 6-10. In certain embodiments, the engineered Cas13 of the invention has the amino acid sequence of SEQ ID NO: 9 or 10.

In certain embodiments, the engineered Cas13 of the invention further comprises a nuclear localization signal (NLS) sequence or a nuclear export signal (NES). For example, in certain embodiments, the engineered Cas13 may comprise an N- and/or a C-terminal NLS.

In a related aspect, the invention provides additional derivatives of the subject engineered Cas13, such as those either substantially lacking or having enhanced collateral endonuclease activity, such as Cas13e and Cas13f effector proteins based on any one of SEQ ID NOs: 50-56 (e.g., SEQ ID NOs: 6-10), or the above orthologs, homologs, derivatives and functional fragments thereof, which comprises another covalently or non-covalently linked protein or polypeptide or other molecules (such as detection reagents or drug/chemical moieties). Such other proteins/polypeptides/other molecules can be linked through, for example, chemical coupling, gene fusion, or other non-covalent linkage (such as biotin-streptavidin binding). Such derived proteins do not affect the function of the original protein, such as the ability to bind a guide RNA/crRNA of the invention (described herein below) to form a complex, the RNase activity, and the ability to bind to and cleave a target RNA at a specific site, under the guidance of the crRNA that is at least partially complementary to the target RNA. In addition, such derived proteins do retain the characteristics of the subject engineered Cas13 either lacking or having enhanced collateral endonuclease activity.

That is, in certain embodiments, upon binding of the RNP complex of the subject engineered Cas13 (or derivative thereof) to the target RNA, the engineered Cas13 either does not exhibit substantial (or detectable) or has enhanced collateral RNase activity.

Such derivation may be used, for example, to add a nuclear localization signal (NLS, such as SV40 large T antigen NLS) to enhance the ability of the subject Cas13, e.g., Cas13e and Cas13f effector proteins, to enter cell nucleus. Such derivation can also be used to add a targeting molecule or moiety to direct the subject Cas13, e.g., Cas13e and Cas13f effector proteins, to specific cellular or subcellular locations. Such derivation can also be used to add a detectable label to facilitate the detection, monitoring, or purification of the subject Cas13, e.g., Cas13e and Cas13f effector proteins. Such derivation can further be used to add a deamination enzyme moiety (such as one with adenine or cytosine deamination activity) to facilitate RNA base editing.

The derivation can be through adding any of the additional moieties at the N- or C-terminal of the subject Cas13 effector proteins, or internally (e.g., internal fusion or linkage through side chains of internal amino acids).

In a related aspect, the invention provides conjugates of the subject engineered Cas13, such as those either substantially lacking or having enhanced substantially lacking collateral endonuclease activity, such as Cas13e and Cas13f effector proteins based on any one of SEQ ID NOs: 50-56 (e.g., SEQ ID NOs: 6-10), or the above orthologs, homologs, derivatives and functional fragments thereof, which are conjugated with moieties such as other proteins or polypeptides, detectable labels, or combinations thereof. Such conjugated moieties may include, without limitation, localization signals, reporter genes (e.g., GST, HRP, CAT, GFP, HcRed, DsRed, CFP, YFP, BFP), labels (e.g., fluorescent dye such as FITC, or DAPI), NLS, targeting moieties, DNA binding domains (e.g., MBP, Lex A DBD, Gal4 DBD), epitope tags (e.g., His, myc, V5, FLAG, HA, VSV-G, Trx, etc), transcription activation domains (e.g., VP64 or VPR), transcription inhibition domains (e.g., KRAB moiety or SID moiety), nucleases (e.g., FokI), deamination domain (e.g., ADAR1, ADAR2, APOBEC, AID, or TAD), methylase, demethylase, transcription release factor, HDAC, ssRNA cleavage activity, dsRNA cleavage activity, ssDNA cleavage activity, dsDNA cleavage activity, DNA or RNA ligase, any combination thereof, etc.

For example, the conjugate may include one or more NLSs, which can be located at or near N-terminal, C-terminal, internally, or combination thereof. The linkage can be through amino acids (such as D or E, or S or T), amino acid derivatives (such as Ahx, β-Ala, GABA or Ava), or PEG linkage.

In certain embodiments, conjugations do not affect the function of the original engineered protein, such as those either substantially lacking or having enhanced collateral effect, such as the ability to bind a guide RNA/crRNA of the invention (described herein below) to form a complex, and the ability to bind to and cleave a target RNA at a specific site, under the guidance of the crRNA that is at least partially complementary to the target RNA.

In a related aspect, the invention provides fusions of the subject engineered Cas13, such as those either substantially lacking or having enhanced collateral endonuclease activity, such as Cas13e and Cas13f effector proteins based on any one of SEQ ID NOs: 50-56 (e.g., SEQ ID NOs: 6-10), or the above orthologs, homologs, derivatives and functional fragments thereof, which fusions are with moieties such as localization signals, reporter genes (e.g., GST, HRP, CAT, GFP, HcRed, DsRed, CFP, YFP, BFP), NLS, protein targeting moieties, DNA binding domains (e.g., MBP, Lex A DBD, Gal4 DBD), epitope tags (e.g., His, myc, V5, FLAG, HA, VSV-G, Trx, etc), transcription activation domains (e.g., VP64 or VPR), transcription inhibition domains (e.g., KRAB moiety or SID moiety), nucleases (e.g., FokI), deamination domain (e.g., ADAR1, ADAR2, APOBEC, AID, or TAD), methylase, demethylase, transcription release factor, HDAC, ssRNA cleavage activity, dsRNA cleavage activity, ssDNA cleavage activity, dsDNA cleavage activity, DNA or RNA ligase, any combination thereof, etc.

For example, the fusion may include one or more NLSs, which can be located at or near N-terminal, C-terminal, internally, or combination thereof. In certain embodiments, conjugations do not affect the function of the original engineered Cas13 protein, such as those either substantially lacking or having enhanced collateral activity, such as the ability to bind a guide RNA/crRNA of the invention (described herein below) to form a complex, the RNase activity, and the ability to bind to and cleave a target RNA at a specific site, under the guidance of the crRNA that is at least partially complementary to the target RNA.

In another aspect, the invention provides a polynucleotide encoding the engineered Cas13 of the invention. The polynucleotide may comprise: (i) a polynucleotide encoding any one of the engineered Cas13, such as those either substantially lacking or having enhanced collateral effect, e.g., those based on Cas13e or Cas13f effector proteins of SEQ ID NOs: 50-56 (such as SEQ ID NOs: 6-10), or orthologs, homologs, derivatives, functional fragments, fusions thereof; (ii) a polynucleotide of any one of SEQ ID NOs: 11-15; or (iii) a polynucleotide comprising (i) and (ii).

In certain embodiments, the polynucleotide of the invention is codon-optimized for expression in a eukaryote, a mammal (such as a human or a non-human mammal), a plant, an insect, a bird, a reptile, a rodent (e.g., mouse, rat), a fish, a worm/nematode, or a yeast.

In a related aspect, the invention provides a polynucleotide having (i) one or more (e.g., 1, 2, 3, 4, 5, 6, 7, 8, 9 or 10) nucleotides additions, deletions, or substitutions compared to the subject polynucleotide described above; (ii) at least 50%, 60%, 70%, 80%, 90%, 95%, or 97% sequence identity to the subject polynucleotide described above; (iii) hybridize under stringent conditions with the subject polynucleotide described above or any of (i) and (ii); or (iv) is a complement of any of (i)-(iii).

In another related aspect, the invention provides a vector comprising or encompassing any one of the polynucleotides of the invention described herein. The vector can be a cloning vector, or an expression vector. The vector can be a plasmid, phagemid, or cosmid, just to name a few. In certain embodiments, the vector can be used to express the polynucleotide in a mammalian cell, such as a human cell, any one of the engineered Cas13, such as those either substantially lacking or having enhanced collateral activity, e.g., the subject engineered Cas13e or Cas13f effector proteins based on SEQ ID NOs: 50-56 (such as SEQ ID NOs: 6-10), or orthologs, homologs, derivatives, functional fragments, fusions thereof; or any of the polynucleotide of the invention; or any of the complex of the invention.

In certain embodiments, the polynucleotide is operably linked to a promoter and optionally an enhancer. For example, in some embodiments, the promoter is a constitutive promoter, an inducible promoter, a ubiquitous promoter, or a tissue specific promoter. In certain embodiments, the vector is a plasmid. In certain embodiments, the vector is a retroviral vector, a phage vector, an adenoviral vector, a herpes simplex viral (HSV) vector, an AAV vector, or a lentiviral vector. In certain embodiments, the AAV vector is a recombinant AAV vector of the serotype AAV1, AAV2, AAV4, AAV5, AAV6, AAV7, AAVrh74, AAV8, AAV9, AAV10, AAV 11, AAV 12, or AAV 13. In certain embodiments.

Another aspect of the invention provides a delivery system comprising (1) a delivery vehicle, and (2) the engineered Cas13 of the invention, the polynucleotide of the invention, or the vector of the invention.

In certain embodiments, the delivery vehicle is a nanoparticle, a liposome, an exosome, a microvesicle, or a gene-gun.

A further aspect of the invention provides a cell or a progeny thereof, comprising the engineered Cas13 of the invention, the polynucleotide of the invention, or the vector of the invention. The cell can be a prokaryote such as E. coli, or a cell from a eukaryote such as yeast, insect, plant, animal (e.g., mammal including human and mouse). The cell can be isolated primary cell (such as bone marrow cells for ex vivo therapy), or established cell lines such as tumor cell lines, 293T cells, or stem cells, iPCs, etc.

In certain embodiments, the cell or progeny thereof is a eukaryotic cell (e.g., a non-human mammalian cell, a human cell, or a plant cell) or a prokaryotic cell (e.g., a bacteria cell).

A further aspect of the invention provides a non-human multicellular eukaryote comprising the cell of the invention.

In certain embodiments, the non-human multicellular eukaryote is an animal (e.g., rodent or primate) model for a human genetic disorder.

In another aspect, the invention provides a complex comprising: (i) a protein composition of any one of the subject engineered Cas13, such as those either substantially lacking or having enhanced collateral endonuclease activity, e.g., engineered Cas13e or Cas13f effector protein, or orthologs, homologs, derivatives, conjugates, functional fragments thereof, conjugates thereof, or fusions thereof; and (ii) a polynucleotide composition, comprising an isolated polynucleotide comprising a cognate DR sequence for said engineered Cas13 effector enzyme, and a spacer/guide sequence complementary to at least a portion of a target RNA.

In certain embodiments, the DR sequence is at the 3′ end of the spacer sequence.

In certain embodiments, the DR sequence is at the 5′ end of the spacer sequence.

In some embodiments, the polynucleotide composition is the guide RNA/crRNA of the subject engineered Cas13, such as those either substantially lacking or having enhanced collateral activity, e.g., engineered Cas13e or Cas13f system, which does not include a tracrRNA.

In certain embodiments, for use with the subject engineered Cas13, such as those either substantially lacking or having enhanced collateral activity, e.g., the subject engineered Cas13e and Cas13f effector proteins, homologs, orthologs, derivatives, fusions, conjugates, or functional fragments thereof having guide sequence-specific RNase activity, the spacer sequence is at least about 10 nucleotides, or between 10-60, 15-50, 20-50, 25-40, 25-50, or 19-50 nucleotides.

In a related aspect, the invention provides a eukaryotic cell comprising a subject complex comprising a subject engineered Cas13, said complex comprising: (1) an RNA guide sequence comprising a spacer sequence capable of hybridizing to a target RNA, and a direct repeat (DR) sequence 5′ or 3′ to the spacer sequence; and, (2) a subject engineered Cas13, such as those either substantially lacking or having enhanced collateral activity, such as a subject engineered Cas13e or Cas13f effector enzyme based on a wild-type having an amino acid sequence of any one of SEQ ID NOs: 50-56 (such as SEQ ID NOs: 6-10), or a derivative or functional fragment of said Cas; wherein the Cas, the derivative, and the functional fragment of said Cas, are capable of (i) binding to the RNA guide sequence and (ii) targeting the target RNA.

In another aspect, the invention provides a composition comprising: (i) a first (protein) composition selected from any one of the engineered Cas13, such as those either substantially lacking or having enhanced collateral activity, e.g., engineered Cas13e or Cas13f effector proteins based on SEQ ID NOs: 50-56 (such as SEQ ID NOs: 6-10), or orthologs, homologs, derivatives, conjugates, functional fragments, fusions thereof; and (ii) a second (nucleotide) composition comprising an RNA encompassing a guide RNA/crRNA, particularly a spacer sequence, or a coding sequence for the same. The guide RNA may comprise a DR sequence, and a spacer sequence which can complement or hybridize with a target RNA. The guide RNA can form a complex with the first (protein) composition of (i). In some embodiment, the DR sequence can be the polynucleotide of the invention. In some embodiment, the DR sequence can be at the 5- or 3′-end of the guide RNA. In some embodiments, the composition (such as (i) and/or (ii)) is non-naturally occurring or modified from a naturally occurring composition. In some embodiments, the target sequence is an RNA from a prokaryote or a eukaryote, such as a non-naturally existing RNA. The target RNA may be present inside a cell, such as in the cytosol or inside an organelle. In some embodiments, the protein composition may have an NLS that can be located at its N- or C-terminal, or internally.

In another aspect, the invention provides a composition comprising one or more vectors of the invention, said one or more vectors comprise: (i) a first polynucleotide that encodes any one of the engineered Cas13, such as those either substantially lacking or having enhanced collateral activity, such as a subject engineered Cas13e or Cas13f effector proteins based on SEQ ID NOs: 50-56 (such as SEQ ID NOs: 6-10), or orthologs, homologs, derivatives, functional fragments, fusions thereof; optionally operably linked to a first regulatory element; and (ii) a second polynucleotide that encodes a guide RNA of the invention; optionally operably linked to a second regulatory element. The first and the second polynucleotides can be on different vectors, or on the same vector. The guide RNA can form a complex with the protein product encoded by the first polynucleotide, and comprises a DR sequence (such as any one of the 4th aspect) and a spacer sequence that can bind to/complement with a target RNA. In some embodiments, the first regulatory element is a promoter, such as an inducible promoter. In some embodiments, the second regulatory element is a promoter, such as an inducible promoter. In some embodiments, the target sequence is an RNA from a prokaryote or a eukaryote, such as a non-naturally existing RNA. The target RNA may be present inside a cell, such as in the cytosol or inside an organelle. In some embodiments, the protein composition may have an NLS that can be located at its N- or C-terminal, or internally.

In some embodiments, the vector is a plasmid. In some embodiment, the vector is a viral vector based on a retrovirus, a replication incompetent retrovirus, adenovirus, replication incompetent adenovirus, or AAV. In some embodiments, the vector can self-replicate in a host cell (e.g., having a bacterial replication origin sequence). In some embodiments, the vector can integrate into a host genome and be replicated therewith. In some embodiment, the vector is a cloning vector. In some embodiment, the vector is an expression vector.

The invention further provides a delivery composition for delivering any of the engineered Cas13, such as those either substantially lacking or having enhanced collateral activity, e.g., a subject engineered Cas13e or Cas13f effector proteins based on SEQ ID NOs: 50-56 (such as SEQ ID NOs: 6-10), or orthologs, homologs, derivatives, conjugates, functional fragments, fusions thereof of the invention; the polynucleotide of the invention; the complex of the invention; the vector of the invention; the cell of the invention, and the composition of the invention. The delivery can be through any one known in the art, such as transfection, lipofection, electroporation, gene gun, microinjection, sonication, calcium phosphate transfection, cation transfection, viral vector delivery, etc., using vehicles such as liposome(s), nanoparticle(s), exosome(s), microvesicle(s), a gene-gun or one or more viral vector(s).

The invention further provides a kit comprising any one or more of the following: any of the engineered Cas13, such as those either substantially lacking or having enhanced collateral activity, e.g., a subject engineered Cas13e or Cas13f effector proteins based on SEQ ID NOs: 50-56 (such as SEQ ID NOs: 6-10), or orthologs, homologs, derivatives, conjugates, functional fragments, fusions thereof of the invention; the polynucleotide of the invention; the complex of the invention; the vector of the invention; the cell of the invention, and the composition of the invention. In some embodiments, the kit may further comprise an instruction for how to use the kit components, and/or how to obtain additional components from 3^{rd} party for use with the kit components. Any component of the kit can be stored in any suitable container.

Another aspect of the invention provides an engineered Cas13 effector enzyme comprising any one or more mutations as described in any of the Examples, such as Example 1, 2, 4, 5, or 12.

In certain embodiments, the engineered Cas13 effector enzyme exhibits about the same or enhanced guide-RNA-mediated cleavage of a target RNA complementary to the guide RNA, as compared to that of the wild-type Cas13 effector enzyme from which the engineered Cas13 effector enzyme derives (or theoretical maximum thereof).

In certain embodiments, the engineered Cas13 effector enzyme exhibits reduced or diminished guide-RNA independent or collateral cleavage of a non-specific RNA (e.g., one not substantially complementary to the guide RNA), as compared to that of the wild-type Cas13 effector enzyme (or theoretical maximum thereof) from which the engineered Cas13 effector enzyme derives. For example, the engineered Cas13 effector enzyme exhibits about 50%, 40%, 30%, 20%, 15%, 10% or less collateral cleavage compared to that of the wild-type Cas13 effector enzyme (or theoretical maximum thereof) from which the engineered Cas13 effector enzyme derives.

In certain embodiments, the engineered Cas13 effector enzyme exhibits increased guide-RNA independent or collateral cleavage of a non-specific RNA (e.g., one not substantially complementary to the guide RNA), as compared to that of the wild-type Cas13 effector enzyme from which the engineered Cas13 effector enzyme derives. For example, the engineered Cas13 effector enzyme exhibits about 105%, 110%, 120%, 130%, 140%, 150%, 160%, 170%, 180%, 190%, 200% or more collateral cleavage compared to that of the wild-type Cas13 effector enzyme from which the engineered Cas13 effector enzyme derives.

With the inventions generally described herein above, more detailed descriptions for the various aspects of the invention are provided in separate sections below. However, it should be understood that, for simplicity and to reduce redundancy, certain embodiments of the invention are only described under one section or only described in the claims or examples. Thus it should also be understood that any one embodiment of the invention, including those described only under one aspect, section, or only in the claims or examples, can be combined with any other embodiment of the invention, unless specifically disclaimed or the combination is improper.

### 2. Representative Engineered Class 2 Type VI Cas and Derivatives Thereof

One aspect of the invention provides engineered Cas13, such as those either substantially lacking or having enhanced collateral activity.

In certain embodiments, the Cas13 effector enzyme is a Class 2, type VI effector enzyme having two strictly conserved RX4-6H (RXXXXH)-like motifs, characteristic of Higher Eukaryotes and Prokaryotes Nucleotide-binding (HEPN) domains. In certain embodiments, the CRISPR Class 2, type VI effectors that contain two HEPN domains have been previously characterized and include, for example, CRISPR Cas13a (C2c2), Cas13b, Cas13c, Cas13d (including the engineered variant CasRx), Cas13e, and Cas13f.

HEPN domains have been shown to be RNase domains and confer the ability to bind to and cleave target RNA molecule. The target RNA may be any suitable form of RNA, including but not limited to mRNA, tRNA, ribosomal RNA, non-coding RNA, lncRNA (long non-coding RNA), and nuclear RNA. For example, in some embodiments, the engineered Cas13 proteins recognize and cleave RNA targets located on the coding strand of open reading frames (ORFs).

In one embodiment, the Class 2 type VI Cas13 effector enzyme is of the subtype Type VI-E and VI-F, or Cas13e or Cas13f (such as SEQ ID NOs: 50-56). Direct comparison of the wild-type Type VI-E and VI-F CRISPR-Cas effector proteins with the effector of these other systems shows that Type VI-E and VI-F CRISPR-Cas effector proteins are significantly smaller (e.g., about 20% fewer amino acids) than even the smallest previously identified Type VI-D/Cas13d effectors (see FIG. 15), and have less than 30% sequence similarity in one to one sequence alignments to other previously described effector proteins, including the phylogenetically closest relatives Cas13b.

Class 2, subtypes VI-E and VI-F effectors, like other Cas13 proteins, can be used in a variety of applications, and are particularly suitable for therapeutic applications since they are significantly smaller than other effectors (e.g., CRISPR Cas13a, Cas13b, Cas13c, and Cas13d/CasRx effectors) which allows for the packaging of the nucleic acids encoding the effectors and their guide RNA coding sequences into delivery systems having size limitations, such as the AAV vectors. Further, the lack of detectable collateral/non-specific RNase activity of the subject engineered Cas13, upon activation of the guide sequence-specific RNase activity, makes these engineered Cas13 effectors less prong to (if not immune from) potentially dangerous generalized off-target RNA digestion in target cells that are desirably not destroyed.

Exemplary Type VI-D CRISPR-Cas effector proteins include Cas13d, such as SEQ ID NO: 101. Exemplary Type VI-E and VI-F CRISPR-Cas effector proteins are provided in the table below.

| | |
|---|---|
| Cas13e.l | MAQVSKQTSKKRELSIDEYQGARKWCFTIAFNKALVNRDKNDGLFVESLLRHEKYSKHDWY |
| | DEDTRALIKCSTOAANAKAEALRNYFSHYRHSPGCLTFTAEDELRTIMERAYERAIFECRR |
| | RETEVIIEFPSLFEGDRITTAGVVFFVSFFVERRVLDRLYGAVSGLKKNEGQYKLTRKALS |
| | MYCLKDSRFTKAWDKRVLLFRDILAQLGRIPAEAYEYYHGEQGDKKRANDNEGTNPKRHKD |
| | KFIEFALHYLEAQHSEICFGRRHIVREEAGAGDEHKKHRTKGKVVVDFSKKDEDQSYYISK |
| | NNVIVRIDKNAGPRSYRMGLNELKYLVLLSLQGKGDDAIAKLYRYRQHVENILDVVKVTDK |
| | DNHVFLPRFVLEQHGIGRKAFKQRIDGRVKHVRGVWEKKKAATNEMTLHEKARDILQYVNE |
| | NCTRSFNPGEYNRLLVCLVGKDVENFQAGLKRLQLAERIDGRVYSIFAQTSTINEMHQVVC |
| | DQILNRLCRIGDQKLYDYVGLGKKDEIDYKQKVAWFKEHISIRRGFLRKKFWYDSKKGFAK |
| | LVEEHLESGGGQRDVGLDKKYYHIDAIGRFEGANPALYETLARDRLCLMMAQYFLGSVRKE |
| | LGNKIVWSNDSIELPVEGSVGNEKSIVFSVSDYGKLYVLDDAEFLGRICEYFMPHEKGKIR |
| | YHTVYEKGFRAYNDLQKKCVEAVLAFEEKVVKAKKMSEKEGAHYIDFREILAQTMCKEAEK |
| | TAVNKVRRAFFHHHLKFVIDEFGLFSDVMKKYGIEKEWKFPVK* (SEQ ID NO: 50) |
| Cas13e.2 | MKVENIKEKSKKAMYLINHYEGPKKWCFAIVLNRACDNYEDNPHLFSKSLLEFEKTSRKDW |
| | FDEETRELVEQADTEIQPNPNLKPNTTANRKLKDIRNYFSHHYHKNECLYFKNDDPIRCIM |
| | EAAYEKSKIYIKGKQIEQSDIPLPELFESSGWITPAGILLLASFFVERGILHRLMGNIGGF |
| | KDNRGEYGLTHDIFTTYCLKGSYSIRAQDHDAVMFRDILGYLSRVPTESFQRIKQPQIRKE |
| | GQLSERKTDKFITFALNYLEDYGLKDLEGCKACFARSKIVREQENVESINDKEYKPHENKK |
| | KVEIHFDQSKEDRFYINRNNVILKIQKKDGHSNIVRMGVYELKYLVLMSLVGKAKEAVEKI |
| | DNYIQDLRDQLPYIEGKNKEEIKEYVRFFPRFIRSHLGLLQINDEEKIKARLDYVKTKWLD |
| | KKEKSKELELHKKGRDILRYINERCDRELNRNVYNRILELLVSKDLTGFYRELEELKRTRR |
| | IDKNIVQNLSGQKTINALHEKVCDLVLKEIESLDTENLRKYLGLIPKEEKEVTFKEKVDRI |
| | LKQPVIYKGFLRYQFFKDDKKSFVLLVEDALKEKGGGCDVPLGKEYYKIVSLDKYDKENKT |
| | LCETLAMDRLCLMMARQYYLSLNAKLAQEAQQIEWKKEDSIELIIFTLKNPDQSKQSFSIR |
| | FSVRDFTKLYVTDDPEFLARLCSYFFPVEKEIEYHKLYSEGINKYTNLQKEGIEAILELEK |
| | KLIERNRIQSAKNYLSFNEIMNKSGYNKDEQDDLKKVRNSLLHYKLIFEKEHLKKFYEVMR |
| | GEGIEKKWSLIV* (SEQ ID NO: 51) |
| Cas13f.l | MNGIELKKEEAAFYFNQAELNLKAIEDNIFDKERRKTLLNNPQILAKMENFIFNFRDVTKN |
| | AKGEIDCLLLKLRELRNFYSHYVHKRDVRELSKGEKPILEKYYQFAIESTGSENVKLEIIE |
| | NDAWLADAGVLFFLCIFLKKSQANKLISGISGFKRNDDTGQPRRNLFTYFSIREGYKVVPE |
| | MQKHFLLFSLVNHLSNQDDYIEKAHQPYDIGEGLFFHRIASTFLNISGILRNMKFYTYQSK |
| | RLVEQRGELKREKDIFAWEEPFQGNSYFEINGHKGVIGEDELKELCYAFLIGNQDANKVEG |
| | RITQFLEKFRNANSVQQVKDDEMLKPEYFPANYFAESGVGRIKDRVLNRLNKAIKSNKAKK |
| | GEIIAYDKMREVMAFINNSLPVDEKLKPKDYKRYLGMVRFWDREKDNIKREFETKEWSKYL |
| | PSNFWTAKNLERVYGLAREKNAELFNKLKADVEKMDERELEKYQKINDAKDLANLRRLASD |
| | FGVKWEEKDWDEYSGQIKKQITDSQKLTIMKQRITAGLKKKHGIENLNLRITIDINKSRKA |
| | VLNRIAIPRGFVKRHILGWQESEKVSKKIREAECEILLSKEYEELSKQFFQSKDYDKMTRI |
| | NGLYEKNKLIALMAVYLMGQLRILFKEHTKLDDITKTTVDFKISDKVTVKIPFSNYPSLVY |
| | TMSSKYVDNIGNYGFSNKDKDKPILGKIDVIEKQRMEFIKEVLGFEKYLFDDKIIDKSKFA |
| | DTATHISFAEIVEELVEKGWDKDRLTKLKDARNKALHGEILTGTSFDETKSLINELKK* |
| | (SEQ ID NO: 52) |
| Cas13f.2 | MSPDFIKLEKQEAAFYFNQTELNLKAIESNILDKQQRMILLNNPRILAKVGNFIFNFRDVT |
| | KNAKGEIDCLLFKLEELRNFYSHYVHTDNVKELSNGEKPLLERYYQIAIQATRSEDVKFEL |
| | FETRNENKITDAGVLFFLCMFLKKSQANKLISGISGFKRNDPTGQPRRNLFTYFSAREGYK |
| | ALPDMQKHFLLFTLVNYLSNQDEYISELKQYGEIGQGAFFNRIASTFLNISGISGNTKFYS |
| | YQSKRIKEQRGELNSEKDSFEWIEPFQGNSYFEINGHKGVIGEDELKELCYALLVAKQDIN |
| | AVEGKIMQFLKKFRNTGNLQQVKDDEMLEIEYFPASYFNESKKEDIKKEILGRLDKKIRSC |
| | SAKAEKAYDKMKEVMEFINNSLPAEEKLKRKDYRRYLKMVRFWSREKGNIEREFRTKEWSK |
| | YFSSDFWRKNNLEDVYKLATQKNAELFKNLKAAAEKMGETEFEKYQQINDVKDLASLRRLT |
| | QDFGLKWEEKDWEEYSEQIKKQITDRQKLTIMKQRVTAELKKKHGIENLNLRITIDSNKSR |
| | KAVLNRIAIPRGFVKKHILGWQGSEKISKNIREAECKILLSKKYEELSRQFFEAGNFDKLT |
| | QINGLYEKNKLTAFMSVYLMGRLNIQLNKHTELGNLKKTEVDFKISDKVTEKIPFSQYPSL |
| | VYAMSRKYVDNVDKYKFSHQDKKKPFLGKIDSIEKERIEFIKEVLDFEEYLFKNKVIDKSK |
| | FSDTATHISFKEICDEMGKKGCNRNKLTELNNARNAALHGEIPSETSFREAKPLINELKK* |
| | (SEQ ID NO: 53) |
| Cas13f.3 | MSPDFIKLEKQEAAFYFNQTELNLKAIESNIFDKQQRVILLNNPQILAKVGDFIFNFRDVT |
| | KNAKGEIDCLLLKLRELRNFYSHYVYTDDVKILSNGERPLLEKYYOFAIEATGSENVKLEI |
| | IESNNRLTEAGVLFFLCMFLKKSQANKLISGISGFKRNDPTGQPRRNLFTYFSVREGYKVV |
| | PDMQKHFLLFVLVNHLSGQDDYIEKAQKPYDIGEGLFFHRIASTFLNISGILRNMEFYIYQ |
| | SKRLKEQQGELKREKDIFPWIEPFQGNSYFEINGNKGIIGEDELKELCYALLVAGKDVRAV |
| | EGKITQFLEKFKNADNAQQVEKDEMLDRNNFPANYFAESNIGSIKEKILNRLGKTDDSYNK |
| | TGTKIKPYDMMKEVMEFINNSLPADEKLKRKDYRRYLKMVRIWDSEKDNIKREFESKEWSK |
| | YFSSDFWMAKNLERVYGLAREKNAELFNKLKAVVEKMDEREFEKYRLINSAEDLASLRRLA |
| | KDFGLKWEEKDWQEYSGQIKKQISDRQKLTIMKQRITAELKKKHGIENLNLRITIDSNKSR |
| | KAVLNRIAVPRGFVKEHILGWQGSEKVSKKTREAKCKILLSKEYEELSKQFFQTRNYDKMT |
| | QVNGLYEKNKLLAFMVVYLMERLNILLNKPTELNELEKAEVDFKISDKVMAKIPFSQYPSL |
| | VYAMSSKYADSVGSYKFENDEKNKPFLGKIDTIEKQRMEFIKEVLGFEEYLFEKKIIDKSE |
| | FADTATHISFDEICNELIKKGWDKDKLTKLKDARNAALHGEIPAETSFREAKPLINGLKK* |
| | (SEQ ID NO: 54) |
| Cas13f.4 | MNIIKLKKEEAAFYFNQTILNLSGLDEIIEKQIPHIISNKENAKKVIDKIFNNRLLLKSVE |
| | NYIYNFKDVAKNARTEIEAILLKLVELRNFYSHYVHNDTVKILSNGEKPILEKYYQIAIEA |
| | TGSKNVKLVIIENNNCLTDSGVLFLLCMFLKKSQANKLISSVSGFKRNDKEGQPRRNLFTY |
| | YSVREGYKVVPDMQKHFLLFALVNHLSEQDDHIEKQQQSDELGKGLFFHRIASTFLNESGI |
| | FNKMQFYTYQSNRLKEKRGELKHEKDTFTWIEPFQGNSYFTLNGHKGVISEDQLKELCYTI |
| | LIEKQNVDSLEGKIIQFLKKFQNVSSKQQVDEDELLKREYFPANYFGRAGTGTLKEKILNR |
| | LDKRMDPTSKVTDKAYDKMIEVMEFINMCLPSDEKLRQKDYRRYLKMVRFWNKEKHNIKRE |
| | FDSKKWTRFLPTELWNKRNLEEAYQLARKENKKKLEDMRNQVRSLKENDLEKYQQINYVND |
| | LENLRLLSQELGVKWQEKDWVEYSGQIKKQISDNQKLTIMKQRITAELKKMHGIENLNLRI |
| | SIDTNKSRQTVMNRIALPKGFVKNHIQQNSSEKISKRIREDYCKIELSGKYEELSRQFFDK |
| | KNFDKMTLINGLCEKNKLIAFMVIYLLERLGFELKEKTKLGELKQTRMTYKISDKVKEDIP |
| | LSYYPKLVYAMNRKYVDNIDSYAFAAYESKKAILDKVDIIEKQRMEFIKQVLCFEEYIFEN |
| | RIIEKSKFNDEETHISFTQIHDELIKKGRDTEKLSKLKHARNKALHGEIPDGTSFEKAKLL |
| | INEIKK* (SEQ ID NO: 55) |
| Cas13f.5 | MNAIELKKEEAAFYFNQARLNISGLDEIIEKQLPHIGSNRENAKKTVDMILDNPEVLKKME |
| | NYVFNSRDIAKNARGELEALLLKLVELRNFYSHYVHKDDVKTLSYGEKPLLDKYYEIAIEA |
| | TGSKDVRLEIIDDKNKLTDAGVLFLLCMFLKKSEANKLISSIRGFKRNDKEGQPRRNLFTY |
| | YSVREGYKVVPDMQKHFLLFTLVNHLSNQDEYISNLRPNQEIGQGGFFHRIASKFLSDSGI |
| | LHSMKFYTYRSKRLTEQRGELKPKKDHFTWIEPFQGNSYFSVQGQKGVIGEEQLKELCYVL |
| | LVAREDFRAVEGKVTQFLKKFQNANNVQQVEKDEVLEKEYFPANYFENRDVGRVKDKILNR |
| | LKKITESYKAKGREVKAYDKMKEVMEFINNCLPTDENLKLKDYRRYLKMVRFWGREKENIK |
| | REFDSKKWERFLPRELWQKRNLEDAYQLAKEKNTELFNKLKTTVERMNELEFEKYQQINDA |
| | KDLANLRQLARDFGVKWEEKDWQEYSGQIKKQITDRQKLTIMKQRITAALKKKQGIENLNL |
| | RITTDTNKSRKVVLNRIALPKGFVRKHILKTDIKISKQIRQSQCPIILSNNYMKLAKEFFE |
| | ERNFDKMTQINGLFEKNVLIAFMIVYLMEQLNLRLGKNTELSNLKKTEVNFTITDKVTEKV |
| | QISQYPSLVFAINREYVDGISGYKLPPKKPKEPPYTFFEKIDAIEKERMEFIKQVLGFEEH |
| | LFEKNVIDKTRFTDTATHISFNEICDELIKKGWDENKIIKLKDARNAALHGKIPEDTSFDE |
| | AKVLINELKK* (SEQ ID NO: 56) |

In the sequences above, the two RX4-6H (RXXXXH) motifs in each effector are double-underlined. In Cas13e.1, the C-terminal motif may have two possibilities due to the RR and HH sequences flanking the motif. Mutations at one or both such domains may create an RNase dead version (or “dCas) of the Cas13e and Cas13f effector proteins, homologs, orthologs, fusions, conjugates, derivatives, or functional fragments thereof, while substantially maintaining their ability to bind the guide RNA and the target RNA complementary to the guide RNA.

The corresponding DR coding sequences for the Cas effectors are listed below:

| | |
|---|---|
| Cas13e.1 | GCTGGAGCAGCCCCCGATTTGTGGGGTGATTACAGC |
| | (SEQ ID NO: 57) |
| Cas13e.2 | GCTGAAGAAGCCTCCGATTTGAGAGGTGATTACAGC |
| | (SEQ ID NO: 58) |
| Cas13f.l | GCTGTGATAGACCTCGATTTGTGGGGTAGTAACAGC |
| | (SEQ ID NO: 59) |
| Cas13f.2 | GCTGTGATAGACCTCGATTTGTGGGGTAGTAACAGC |
| | (SEQ ID NO: 60) |
| Cas13f.3 | GCTGTGATAGACCTCGATTTGTGGGGTAGTAACAGC |
| | (SEQ ID NO: 61) |
| Cas13f.4 | GCTGTGATGGGCCTCAATTTGTGGGGAAGTAACAGC |
| | (SEQ ID NO: 62) |
| Cas13f.5 | GCTGTGATAGGCCTCGATTTGTGGGGTAGTAACAGC |
| | (SEQ ID NO: 63) |

In some embodiments, a subject engineered Cas13 effector enzyme, such as those either substantially lacking or having enhanced collateral activity is based on a “derivative” of a wild-type Type VI-D, Type VI-E and VI-F CRISPR-Cas effector proteins, said derivative having an amino acid sequence with at least about 80% sequence identity to the amino acid sequence of any one of SEQ ID NOs: 50-56 and 101 above (e.g., 81%, 82%, 83%, 84%, 85%, 86%, 87% 88%, 89%, 90%, 91%, 92%, 93%, 94%, 95%, 96%, 97%, 98%, 99%, or 100%). Such derivative Cas effectors sharing significant protein sequence identity to any one of SEQ ID NOs: 50-56 and 101 have retained at least one of the functions of the Cas of SEQ ID NOs: 50-56 and 101 (see below), such as the ability to bind to and form a complex with a crRNA comprising at least one of the DR sequences of Cas13d, and SEQ ID NOs: 57-63. For example, a Cas13e.1 derivative may share 85% amino acid sequence identity to SEQ ID NO: 50, 51, 52, 53, 54, 55, or 56, respectively, and retains the ability to bind to and form a complex with a crRNA having a DR sequence of SEQ ID NO: 57, 58, 59, 60, 61, 62, or 63, respectively.

In certain embodiments, the sequence identity between the derivative and the wild-type Cas13 is based on regions outside the regions defined by the mutant regions in Examples 1, 2, 4 and 5, such as SEQ ID NOs: 16, 20, 24, 28, and 32.

In some embodiments, the derivative comprises conserved amino acid residue substitutions. In some embodiments, the derivative comprises only conserved amino acid residue substitutions (i.e., all amino acid substitutions in the derivative are conserved substitutions, and there is no substitution that is not conserved).

In some embodiments, the derivative comprises no more than 1, 2, 3, 4, 5, 6, 7, 8, 9, or 10 amino acid insertions or deletions into any one of the wild-type sequences of Cas13d, and SEQ ID NOs: 50-56. The insertion and/or deletion maybe clustered together, or separated throughout the entire length of the sequences, so long as at least one of the functions of the wild-type sequence is preserved. Such functions may include the ability to bind the guide/crRNA, the RNase activity, the ability to bind to and/or cleave the target RNA complementary to the guide/crRNA. In some embodiments, the insertions and/or deletions are not present in the RXXXXH motifs, or within 5, 10, 15, or 20 residues from the RXXXXH motifs.

In some embodiments, the derivative has retained the ability to bind guide RNA/crRNA.

In some embodiments, the derivative has retained the guide/crRNA-activated RNase activity.

In some embodiments, the derivative has retained the ability to bind target RNA and/or cleave the target RNA in the presence of the bound guide/crRNA that is complementary in sequence to at least a portion of the target RNA.

In other embodiments, the derivative has completely or partially lost the guide/crRNA-activated RNase activity, due to, for example, mutations in one or more catalytic residues of the RNA-guided RNase. Such derivatives are sometimes referred to as dCas, such as dCas13d and dCas13e.1.

Thus in certain embodiments, the derivative may be modified to have diminished nuclease/RNase activity, e.g., nuclease inactivation of at least 50%, at least 60%, at least 70%, at least 80%, at least 90%, at least 95%, at least 97%, or 100% as compared with the counterpart wild type proteins. The nuclease activity can be diminished by several methods known in the art, e.g., introducing mutations into the nuclease (catalytic) domains of the proteins. In some embodiments, catalytic residues for the nuclease activities are identified, and these amino acid residues can be substituted by different amino acid residues (e.g., glycine or alanine) to diminish the nuclease activity. In some embodiments, the amino acid substitution is a conservative amino acid substitution. In some embodiments, the amino acid substitution is a non-conservative amino acid substitution.

In some embodiments, the modification comprises one or more mutations (e.g., amino acid deletions, insertions, or substitutions) in at least one HEPN domain. In some embodiments, there is one, two, three, four, five, six, seven, eight, nine, or more amino acid substitutions in at least one HEPN domain.

For example, in some embodiments, the one or more mutations comprise a substitution (e.g., an alanine substitution) at an amino acid residue corresponding to R84, H89, R739, H744, R740, H745 of SEQ ID NO: 50 or R97, H102, R770, H775 of SEQ ID NO: 51 or R77, H82, R764, H769 of SEQ ID NO: 52, or R79, H84, R766A, H771 of SEQ ID NO: 53, or R79, H84, R766, H771 of SEQ ID NO: 54, or R89, H94, R773, H778 of SEQ ID NO: 55, or R89, H94, R777, H782 of SEQ ID NO: 56.

In certain embodiments, the one or more mutations comprises, consists essentially of, or consists of: (a) substitutions within 1, 2, 3, 4, or 5 of said stretches of 15-20 consecutive amino acids within the region; (b) a mutation corresponds to a Cas13d mutation of Example 4 that retains at least about 75% of guide RNA-specific cleavage of wild-type Cas13d (such as SEQ ID NO: 101), and exhibits less than about 27.5% collateral effect of wild-type Cas13d (such as SEQ ID NO: 101); (c) a mutation corresponds to the N1V7, N2V7, N2V8 (cfCas13d), N3V7, or N15V4 mutation of Cas13d mutation; (d) a mutation corresponds to a Cas13d mutation of Example 4 that retains between about 25-75% of guide RNA-specific cleavage of wild-type Cas13d (such as SEQ ID NO: 101), and exhibits less than about 27.5% collateral effect of wild-type Cas13d (such as SEQ ID NO: 101); (e) a mutation corresponds to the N2V4, N2V5, N4V3, N6V3, N10V6, N15V2, N20V6, or N20-Y910A mutation of Cas13d mutation; (f) a mutation corresponds to a Cas13e mutation of Example 1, 2, or 5 that retains at least about 75% of guide RNA-specific cleavage of wild-type Cas13e (such as SEQ ID NO: 4), and exhibits less than about 25% collateral effect of wild-type Cas13e (such as SEQ ID NO: 4); (f) a mutation corresponds to the M1V4, M2V2, M2V3, M2V4, M5V1, M6V2, M6V3, M6V4, M7V1, M7V2, M7V3, M7-Y55A, M7-Y61A, M11V1, M12V3, M15V1, M15V2, M15-Y643A, M15-Y647 A, M16V1, M16V2, M17V2, M18V2, M18V3, M19V2, M19V3, or M19-IA mutation of Cas13e mutation; (g) a mutation corresponds to a Cas13e mutation of Example 5 that retains between about 25-75% of guide RNA-specific cleavage of wild-type Cas13e (such as SEQ ID NO: 4), and exhibits less than about 25% collateral effect of wild-type Cas13e (such as SEQ ID NO: 4); and/or (h) a mutation corresponds to the M17YY (cfCas13e), M8V4, M9V1, M11V2, M11V3, M13V1, M13V2, M13V3, M15V3, or M20V2 mutation of Cas13e mutation.

In certain embodiments, the one or more mutations or the two or more mutations may be in a catalytically active domain of the effector protein comprising a HEPN domain, or a catalytically active domain which is homologous to a HEPN domain. In certain embodiments, the effector protein comprises one or more of the following mutations: R84A, H89A, R739A, H744A, R740A, H745A (wherein amino acid positions correspond to amino acid positions of Cas13e.1).

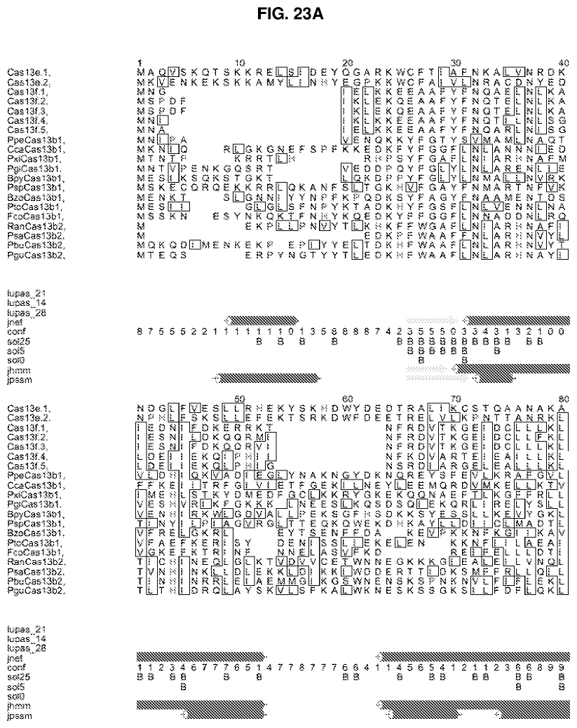

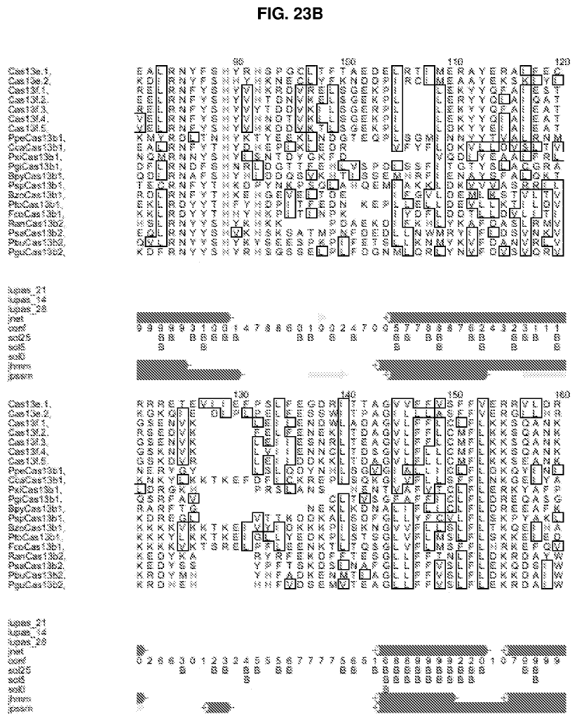

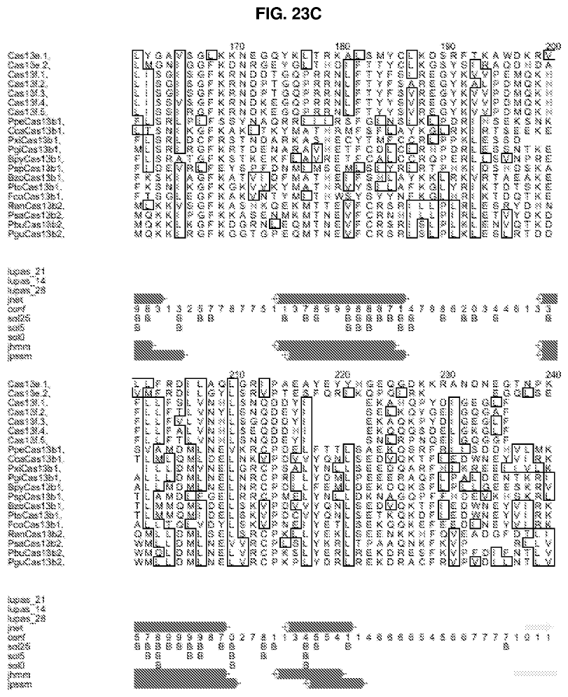

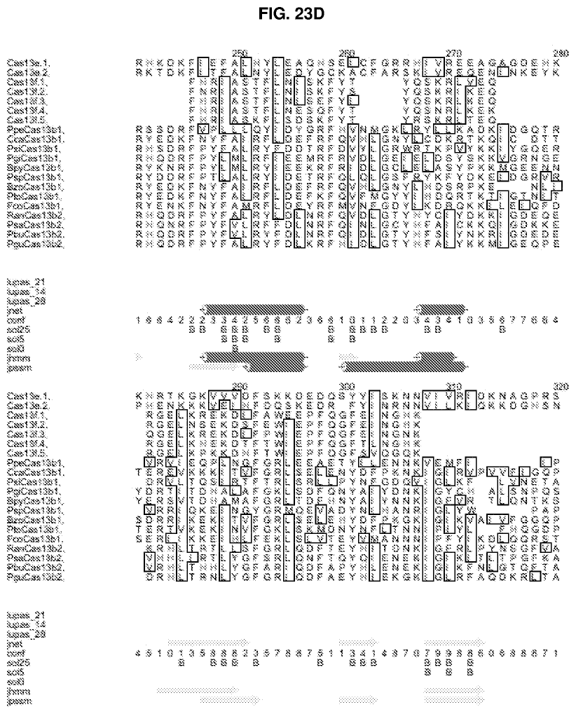

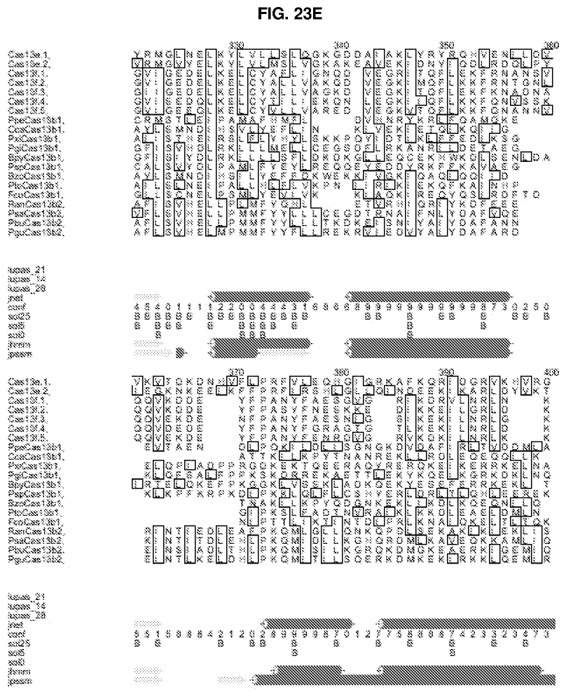

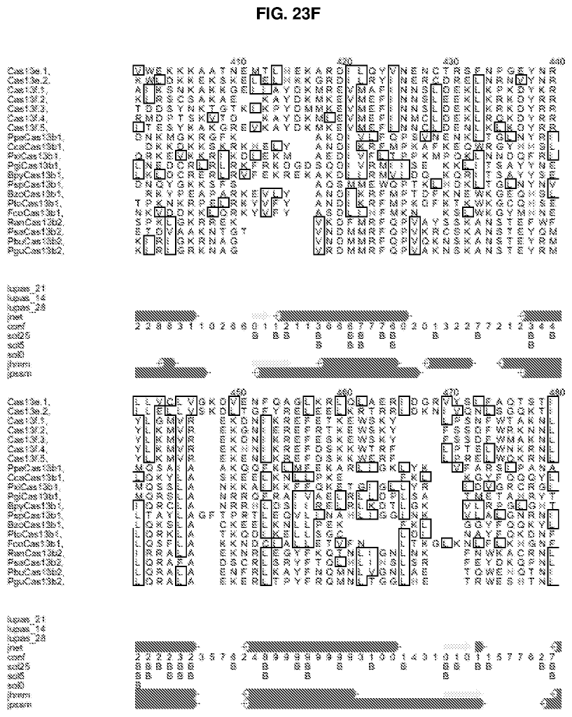

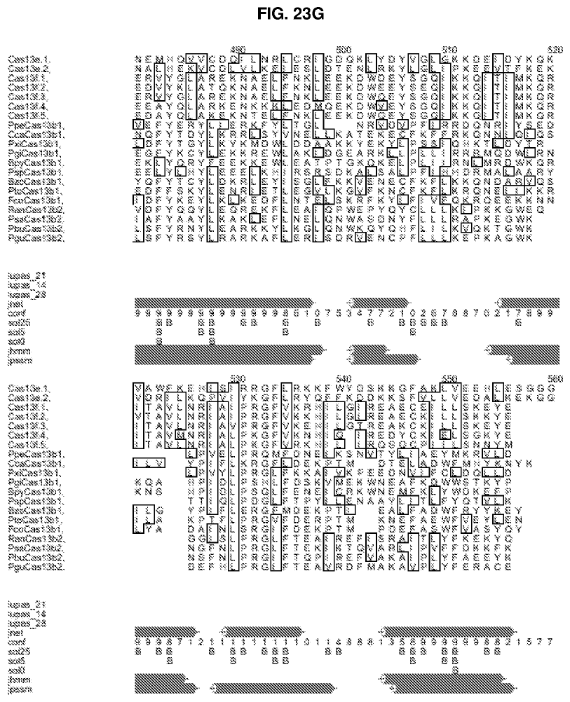

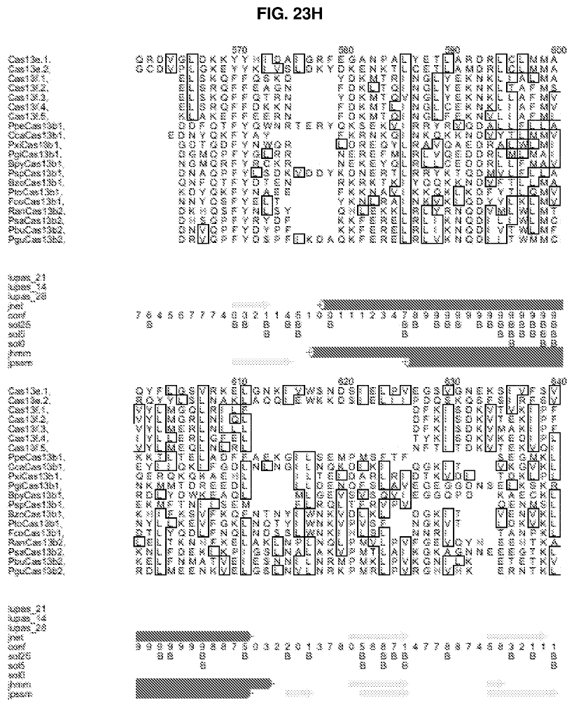

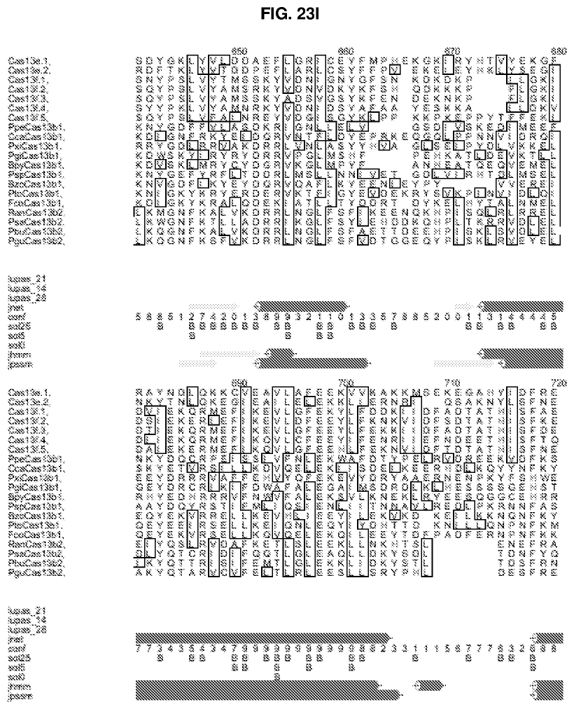

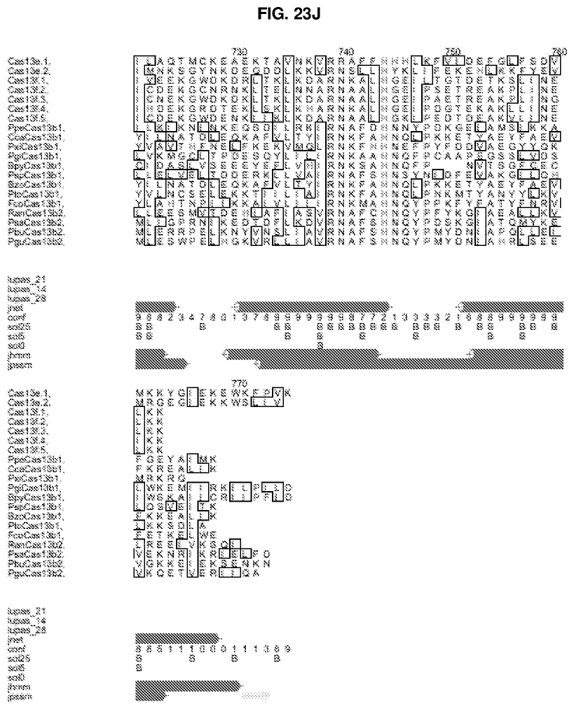

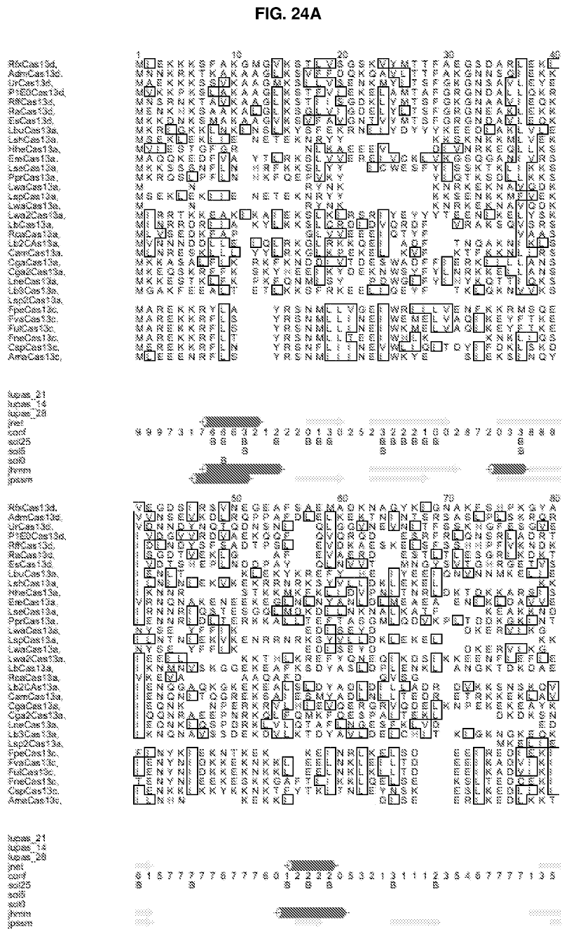

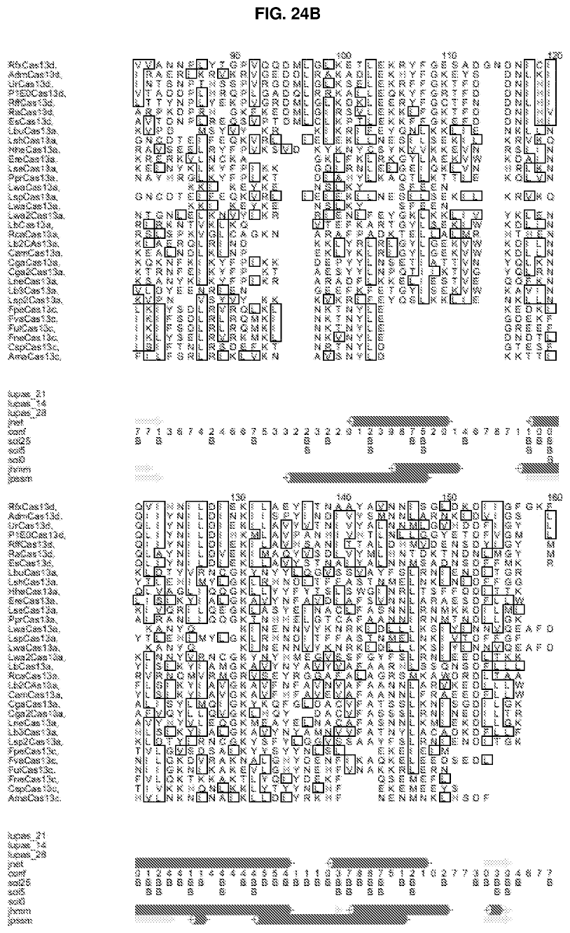

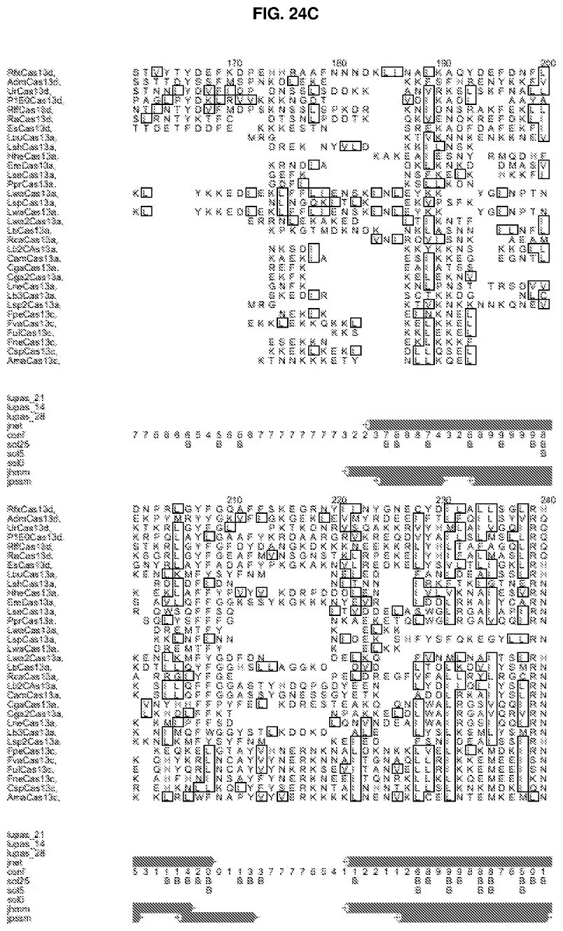

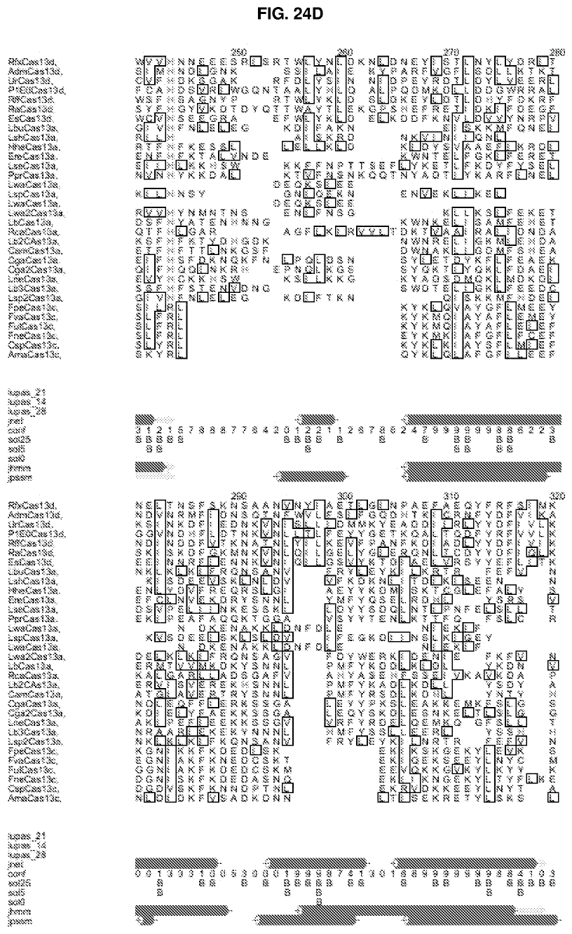

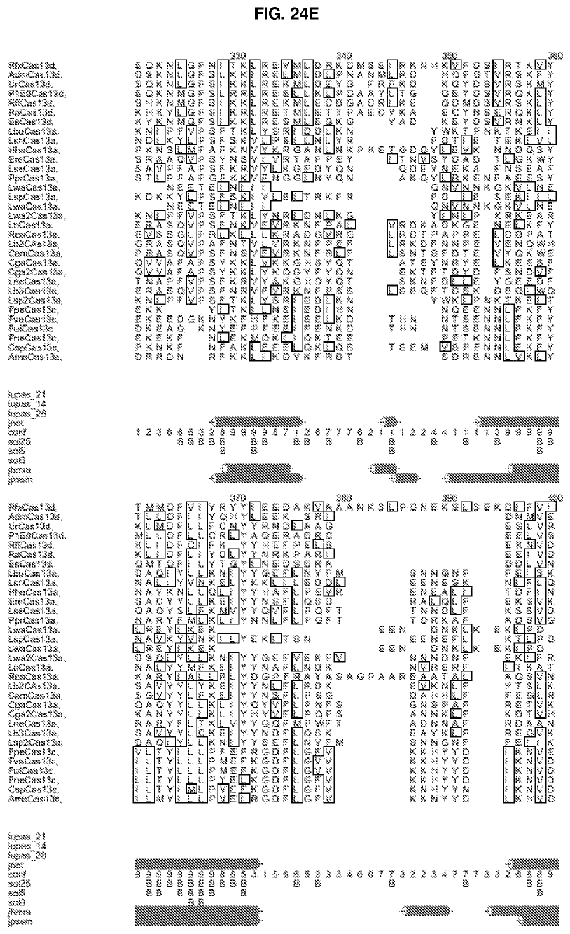

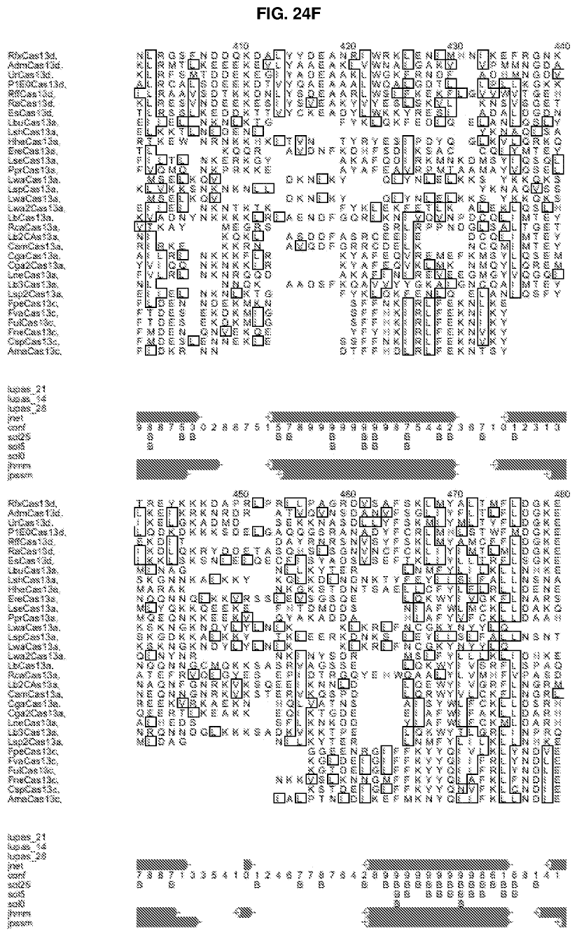

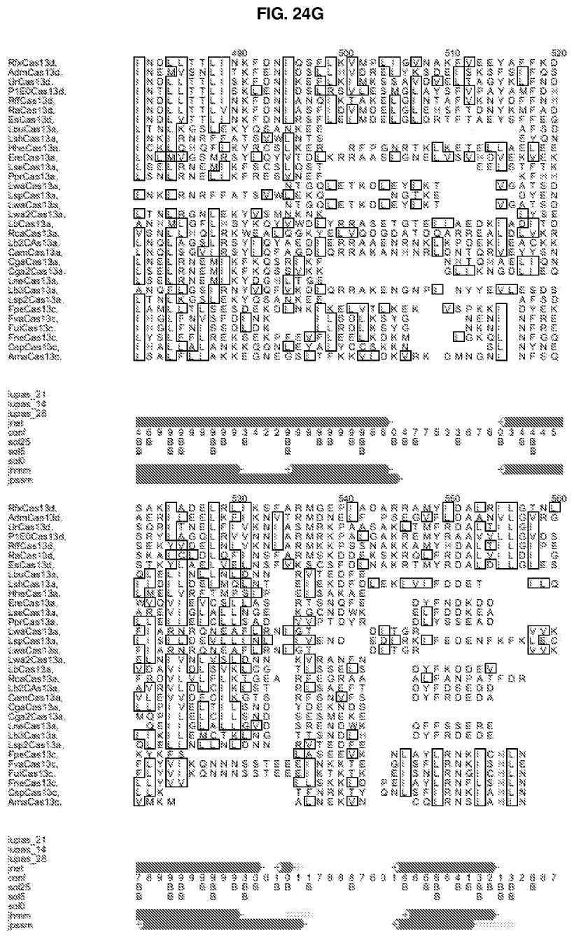

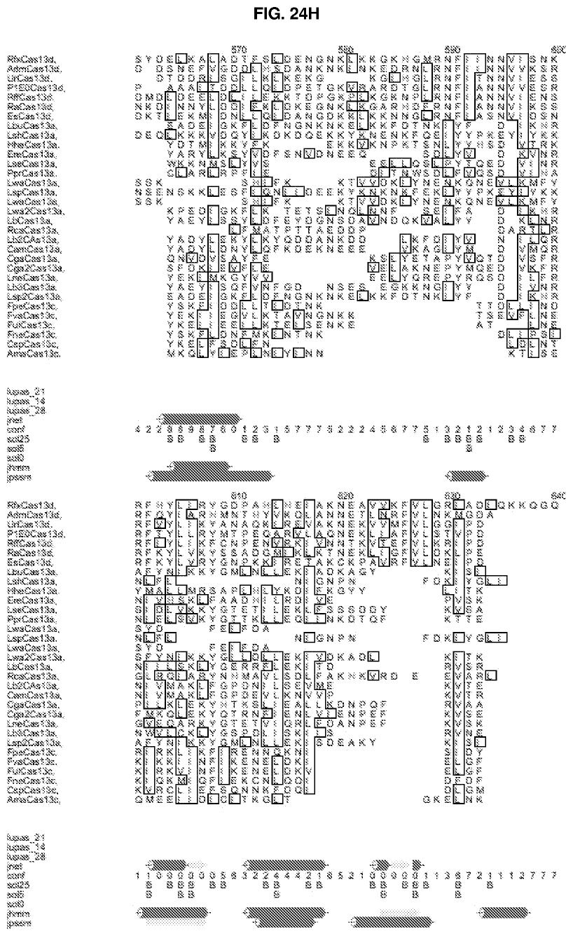

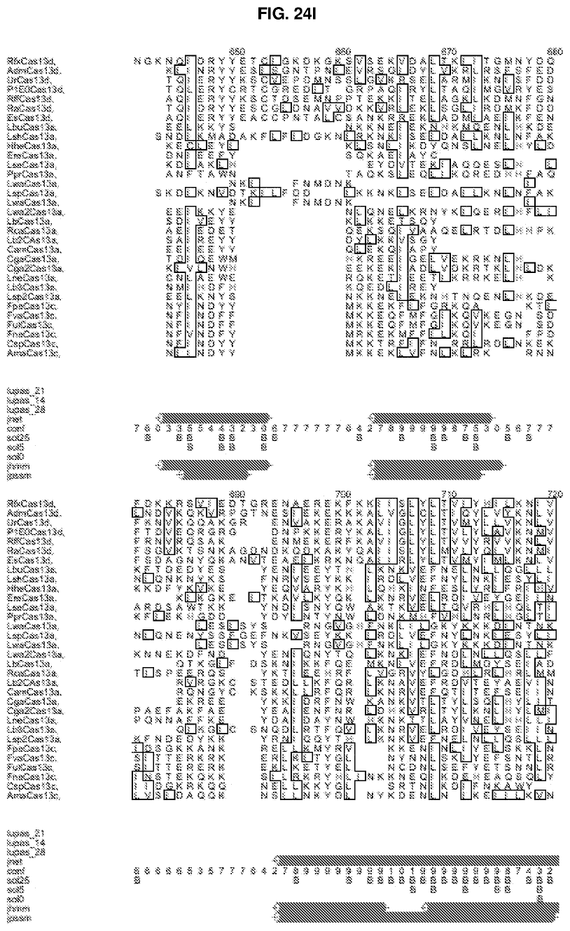

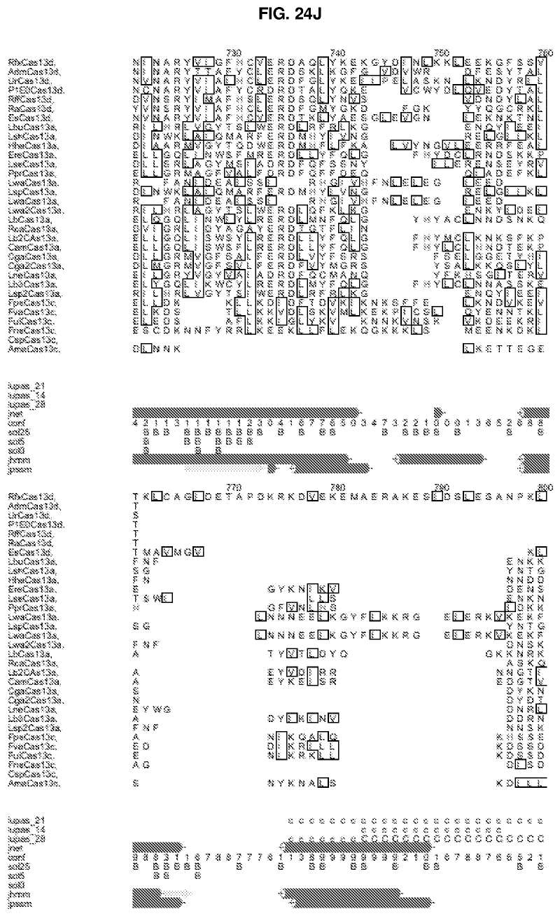

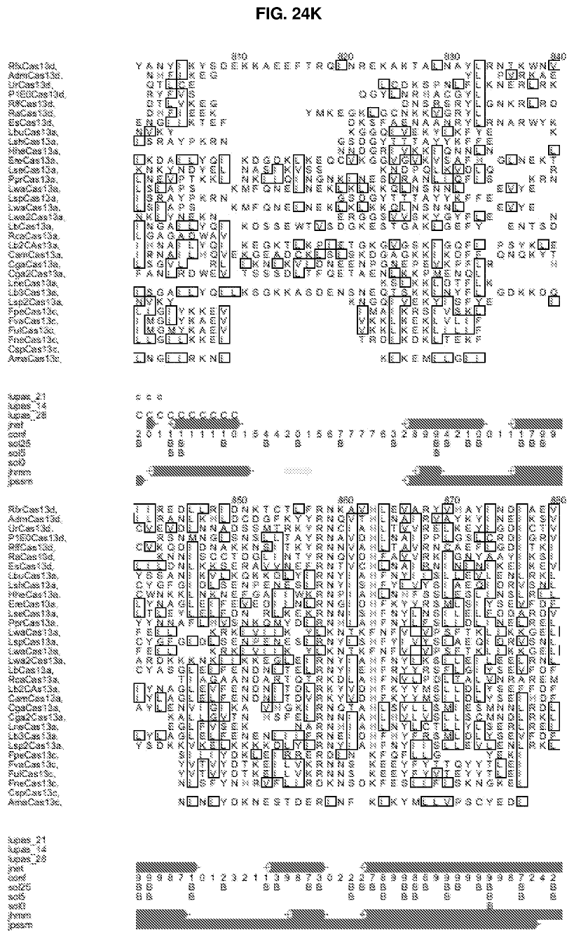

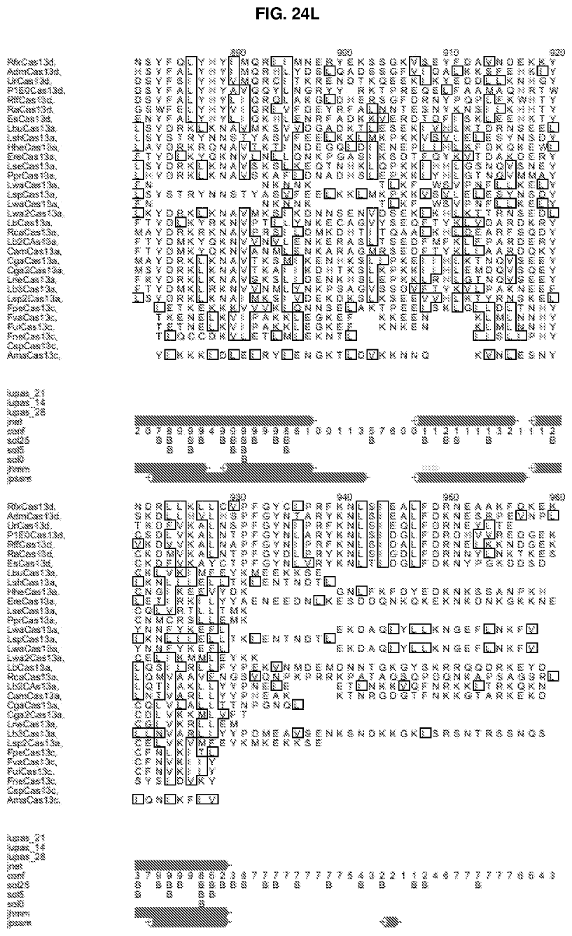

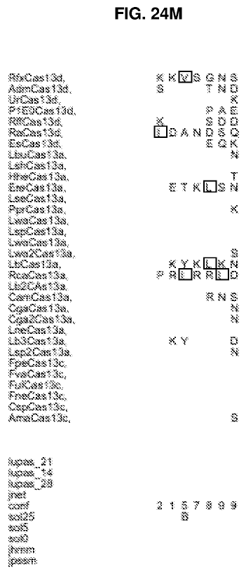

The skilled person will understand that corresponding amino acid positions in different Cas13 proteins, such as different Cas13d, Cas13e and Cas13f proteins, may be mutated to the same effect. In this regard, FIGS. 23A-23J provides an exemplary multisequence alignment of several representative Cas13 family enzymes. One of skill in the art can readily map the mutations in any Cas13 family protein sharing substantial sequence homology/identical to any of the sequences in FIGS. 23A-23J and 24A-24M, in order to determine the mutations “corresponding to” the exemplified Cas13d and Cas13e mutations described herein.

In certain embodiments, one or more mutations abolishes catalytic activity of the protein completely or partially (e.g. altered cleavage rate, altered specificity, etc.).

Other exemplary (catalytic) residue mutations include: R97A, H102A, R770A, H775A of Cas13e.2, or R77A, H82A, R764A, H769A of Cas13f.1, or R79A, H84A, R766A, H771A of Cas13f.2, or R79A, H84A, R766A, H771A of Cas13f.3, or R89A, H94A, R773A, H778A of Cas13f.4, or R89A, H94A, R777A, H782A of Cas13f.5. In certain embodiments, any of the R and/or H residues herein may be replaced not be A but by G, V, or I.

The presence of at least one of these mutations results in a derivative having reduced or diminished guide sequence-dependent RNase activity as compared to the corresponding wild-type protein lacking the mutations. The additional presence of any one of the mutations in the subject engineered Cas13 substantially lacking collateral effect can reduce/eliminate off-target effect resulting from non-specific RNA binding.

In certain embodiments, the effector protein as described herein is a “dead” effector protein, such as a dead Cas13e or Cas13f effector protein (i.e. dCas13e and dCas13f). In certain embodiments, the effector protein has one or more mutations in HEPN domain 1 (N-terminal). In certain embodiments, the effector protein has one or more mutations in HEPN domain 2 (C-terminal). In certain embodiments, the effector protein has one or more mutations in HEPN domain 1 and HEPN domain 2.

The inactivated Cas or derivative or functional fragment thereof can be fused or associated with one or more heterologous/functional domains (e.g., via fusion protein, linker peptides, “GS” linkers, etc.). These functional domains can have various activities, e.g., methylase activity, demethylase activity, transcription activation activity, transcription repression activity, transcription release factor activity, histone modification activity, RNA cleavage activity, DNA cleavage activity, nucleic acid binding activity, base-editing activity, and switch activity (e.g., light inducible). In some embodiments, the functional domains are Krüppel associated box (KRAB), SID (e.g. SID4X), VP64, VPR, VP16, FokI, P65, HSF1, MyoD1, Adenosine Deaminase Acting on RNA such as ADAR1, ADAR2, APOBEC, cytidine deaminase (AID), TAD, mini-SOG, APEX, and biotin-APEX.

In some embodiments, the functional domain is a base editing domain, e.g., ADAR1 (including wild-type or ADAR2DD version thereof, with or without the E1008Q and/or the E488Q mutation(s)), ADAR2 (including wild-type or ADAR2DD version thereof, with or without the E1008Q and/or the E488Q mutation(s)), APOBEC, or AID.

In some embodiments, the functional domain may comprise one or more nuclear localization signal (NLS) domains. The one or more heterologous functional domains may comprise at least two or more NLS domains. The one or more NLS domain(s) may be positioned at or near or in proximity to a terminus of the effector protein (e.g., Cas13e/Cas13f effector proteins) and if two or more NLSs, each of the two may be positioned at or near or in proximity to a terminus of the effector protein (e.g., Cas13e/Cas13f effector proteins).

In some embodiments, at least one or more heterologous functional domains may be at or near the amino-terminus of the effector protein and/or wherein at least one or more heterologous functional domains is at or near the carboxy-terminus of the effector protein. The one or more heterologous functional domains may be fused to the effector protein. The one or more heterologous functional domains may be tethered to the effector protein. The one or more heterologous functional domains may be linked to the effector protein by a linker moiety.

In some embodiments, multiple (e.g., two, three, four, five, six, seven, eight, or more) identical or different functional domains are present.

In some embodiments, the functional domain (e.g., a base editing domain) is further fused to an RNA-binding domain (e.g., MS2).

In some embodiments, the functional domain is associated to or fused via a linker sequence (e.g., a flexible linker sequence or a rigid linker sequence). Exemplary linker sequences and functional domain sequences are provided in table below.

Amino Acid Sequences of Motifs and Functional
Domains in Engineered Variants of Type VI-D,
Type VI E and VI-F CRISPR Cas Effectors

| | |
|---|---|
| Linker 1 | GS |
| Linker 2 | GSGGGGS (SEQ ID NO: 70) |
| Linker 3 | GGGGSGGGGSGGGGS |
| | (SEQ ID NO: 71) |
| ADAR1DD-WT | SEQ ID NO: 72 |
| ADAR1DD-E10080 | SEQ ID NO: 73 |
| ADAR2DD-WT | SEQ ID NO: 74 |
| ADAR2DD-E4880 | SEQ ID NO: 75 |
| AID-APOBEOl | SEQ ID NO: 76 |
| Lamprey_AID-APOBEC1 | SEQ ID NO: 77 |
| APOBEC1_BE1 | SEQ ID NO: 78 |

The positioning of the one or more functional domains on the inactivated Cas proteins is one that allows for correct spatial orientation for the functional domain to affect the target with the attributed functional effect. For example, if the functional domain is a transcription activator (e.g., VP16, VP64, or p65), the transcription activator is placed in a spatial orientation that allows it to affect the transcription of the target. Likewise, a transcription repressor is positioned to affect the transcription of the target, and a nuclease (e.g., FokI) is positioned to cleave or partially cleave the target. In some embodiments, the functional domain is positioned at the N-terminus of the Cas/dCas. In some embodiments, the functional domain is positioned at the C-terminus of the Cas/dCas. In some embodiments, the inactivated CRISPR-associated protein (dCas) is modified to comprise a first functional domain at the N-terminus and a second functional domain at the C-terminus.

Various examples of inactivated CRISPR-associated proteins fused with one or more functional domains and methods of using the same are described, e.g., in International Publication No. WO 2017/219027, which is incorporated herein by reference in its entirety, and in particular with respect to the features described herein.

In some embodiments, instead of using full-length wild-type (SEQ ID NOs: 50-56) or derivative Type VI-E and VI-F Cas effectors, “functional fragments” thereof can be used.

A “functional fragment,” as used herein, refers to a fragment of a wild-type Cas13 protein such as any one of SEQ ID NOs: 50-56 and 101, or a derivative thereof, that has less-than full-length sequence. The deleted residues in the functional fragment can be at the N-terminus, the C-terminus, and/or internally. The functional fragment retains at least one function of the wild-type VI-D, VI-E or VI-F Cas, or at least one function of its derivative. Thus a functional fragment is defined specifically with respect to the function at issue. For example, a functional fragment, wherein the function is the ability to bind crRNA and target RNA, may not be a functional fragment with respect to the RNase function, because losing the RXXXXH motifs at both ends of the Cas may not affect its ability to bind a crRNA and target RNA, but may eliminate/destroy the RNase activity. In certain embodiments, the engineered Cas13 of the invention including a functional fragment of an engineered Cas13 that substantially retains the corresponding wild-type Cas13's guide sequence-dependent RNase activity, but substantially lacks collateral activity.

In some embodiments, compared to full-length wild-type sequences, the engineered Class 2 type VI effector proteins or derivatives thereof or functional fragments thereof lacks about 30, 60, 90, 120, 150, or about 180 residues from the N-terminus.

In some embodiments, compared to full-length wild-type sequences, the engineered Class 2 type VI effector proteins or derivatives thereof or functional fragments thereof lacks about 30, 60, 90, 120, or about 150 residues from the C-terminus.

In some embodiments, compared to full-length wild-type sequences, the engineered Class 2 type VI effector proteins or derivatives thereof or functional fragments thereof lacks about 30, 60, 90, 120, 150, or about 180 residues from the N-terminus, and lacks about 30, 60, 90, 120, or about 150 residues from the C-terminus.

In some embodiments, the engineered Class 2 Type VI Cas13 effector proteins or derivatives thereof or functional fragments thereof have RNase activity, e.g., guide/crRNA-activated specific RNase activity.

In some embodiments, the engineered Class 2 Type VI Cas13 effector proteins or derivatives thereof or functional fragments thereof have no substantial/detectable collateral RNase activity.

The present disclosure also provides a split version of the engineered Class 2 type VI Cas13 effector enzyme described herein (e.g., a Type VI-D, VI-E or VI-F CRISPR-Cas effector protein). The split version of the engineered Cas13 may be advantageous for delivery. In some embodiments, the engineered Cas13 is split into two parts of the enzyme, which together substantially comprise a functioning engineered Class 2 type VI Cas13.

The split can be done in a way that the catalytic domain(s) are unaffected. The CRISPR-associated protein may function as a nuclease or may be an inactivated enzyme, which is essentially a RNA-binding protein with very little or no catalytic activity (e.g., due to mutation(s) in its catalytic domains). Split enzymes are described, e.g., in Wright et al., “Rational design of a split-Cas9 enzyme complex,” Proc. Nat'l. Acad. Sci. 112(10): 2984-2989, 2015, which is incorporated herein by reference in its entirety.

For example, in some embodiments, the nuclease lobe and a-helical lobe are expressed as separate polypeptides. Although the lobes do not interact on their own, the crRNA recruits them into a ternary complex that recapitulates the activity of full-length CRISPR-associated proteins and catalyzes site-specific cleavage. The use of a modified crRNA abrogates split-enzyme activity by preventing dimerization, allowing for the development of an inducible dimerization system.

In some embodiments, the split CRISPR-associated protein can be fused to a dimerization partner, e.g., by employing rapamycin sensitive dimerization domains. This allows the generation of a chemically inducible CRISPR-associated protein for temporal control of the activity of the protein. The CRISPR-associated protein can thus be rendered chemically inducible by being split into two fragments and rapamycin-sensitive dimerization domains can be used for controlled re-assembly of the protein.

The split point is typically designed in silico and cloned into the constructs. During this process, mutations can be introduced to the split CRISPR-associated protein and non-functional domains can be removed.

In some embodiments, the two parts or fragments of the split CRISPR-associated protein (i.e., the N-terminal and C-terminal fragments), can form a full CRISPR-associated protein, comprising, e.g., at least 70%, at least 80%, at least 90%, at least 95%, or at least 99% of the sequence of the wild-type CRISPR-associated protein.

The CRISPR-associated proteins described herein (e.g., a Type VI-D, VI-E or VI-F CRISPR-Cas effector protein) can be designed to be self-activating or self-inactivating. For example, the target sequence can be introduced into the coding construct of the CRISPR-associated protein. Thus, the CRISPR-associated protein can cleave the target sequence, as well as the construct encoding the protein thereby self-inactivating their expression. Methods of constructing a self-inactivating CRISPR system are described, e.g., in Epstein and Schaffer, Mol. Ther. 24: S50, 2016, which is incorporated herein by reference in its entirety.

In some other embodiments, an additional crRNA, expressed under the control of a weak promoter (e.g., 7SK promoter), can target the nucleic acid sequence encoding the CRISPR-associated protein to prevent and/or block its expression (e.g., by preventing the transcription and/or translation of the nucleic acid). The transfection of cells with vectors expressing the CRISPR-associated protein, the crRNAs, and crRNAs that target the nucleic acid encoding the CRISPR-associated protein can lead to efficient disruption of the nucleic acid encoding the CRISPR-associated protein and decrease the levels of CRISPR-associated protein, thereby limiting its activity.

In some embodiments, the activity of the CRISPR-associated protein can be modulated through endogenous RNA signatures (e.g., miRNA) in mammalian cells. A CRISPR-associated protein switch can be made by using a miRNA-complementary sequence in the 5′-UTR of mRNA encoding the CRISPR-associated protein. The switches selectively and efficiently respond to miRNA in the target cells. Thus, the switches can differentially control the Cas activity by sensing endogenous miRNA activities within a heterogeneous cell population. Therefore, the switch systems can provide a framework for cell-type selective activity and cell engineering based on intracellular miRNA information (see, e.g., Hirosawa et al., Nucl. Acids Res. 45(13): e118, 2017).

The engineered Class 2 type VI Cas13 effectors, such as those either substantially lacking or having enhanced collateral activity (e.g., engineered Type VI-D, VI-E and VI-F CRISPR-Cas effector proteins) can be inducibly expressed, e.g., their expression can be light-induced or chemically-induced. This mechanism allows for activation of the functional domain in the CRISPR-associated proteins. Light inducibility can be achieved by various methods known in the art, e.g., by designing a fusion complex wherein CRY2 PHR/CIBN pairing is used in split CRISPR-associated proteins (see, e.g., Konermann et al., “Optical control of mammalian endogenous transcription and epigenetic states,” Nature 500:7463, 2013.

Chemical inducibility can be achieved, e.g., by designing a fusion complex wherein FKBP/FRB (FK506 binding protein/FKBP rapamycin binding domain) pairing is used in split CRISPR-associated proteins. Rapamycin is required for forming the fusion complex, thereby activating the CRISPR-associated proteins (see, e.g., Zetsche et al., “A split-Cas9 architecture for inducible genome editing and transcription modulation,” Nature Biotech. 33:2:139-42, 2015).

Furthermore, expression of the engineered Class 2 type VI Cas13 effectors, such as those either substantially lacking or having enhanced collateral activity can be modulated by inducible promoters, e.g., tetracycline or doxycycline controlled transcriptional activation (Tet-On and Tet-Off expression system), hormone inducible gene expression system (e.g., an ecdysone inducible gene expression system), and an arabinose-inducible gene expression system. When delivered as RNA, expression of the RNA targeting effector protein can be modulated via a riboswitch, which can sense a small molecule like tetracycline (see, e.g., Goldfless et al., “Direct and specific chemical control of eukaryotic translation with a synthetic RNA-protein interaction,” Nucl. Acids Res. 40:9: e64-e64, 2012).

Various embodiments of inducible CRISPR-associated proteins and inducible CRISPR systems are described, e.g., in U.S. Pat. No. 8,871,445, US Publication No. 2016/0208243, and International Publication No. WO 2016/205764, each of which is incorporated herein by reference in its entirety.

In some embodiments, the engineered Class 2 type VI Cas13 effectors, such as those either substantially lacking or having enhanced collateral activity include at least one (e.g., 1, 2, 3, 4, 5, 6, 7, 8, 9, or 10) Nuclear Localization Signal (NLS) attached to the N-terminal or C-terminal of the protein. Non-limiting examples of NLSs include an NLS sequence derived from: the NLS of the SV40 virus large T-antigen, having the amino acid sequence of SEQ ID NO: 79; the NLS from nucleoplasmin (e.g., the nucleoplasmin bipartite NLS with the sequence of SEQ ID NO: 80); the c-myc NLS having the amino acid sequence of SEQ ID NO: 81 or 82; the hRNPA1 M9 NLS having the sequence of SEQ ID NO: 83; the sequence of SEQ ID NO: 84 of the IBB domain from importin-alpha; the sequences of SEQ ID NO: 85 or 86 of the myoma T protein; the sequence of SEQ ID NO: 87 of human p53; the sequence of SEQ ID NO: 88 of mouse c-abl IV; the sequences of SEQ ID NO: 89 or 90 of the influenza virus NS1; the sequence of SEQ ID NO: 91 of the Hepatitis virus delta antigen; the sequence of SEQ ID NO: 92 of the mouse Mx1 protein; the sequence of SEQ ID NO: 93 of the human poly(ADP-ribose) polymerase; and the sequence of SEQ ID NO: 94 of the human glucocorticoid receptor. In some embodiments, the CRISPR-associated protein comprises at least one (e.g., 1, 2, 3, 4, 5, 6, 7, 8, 9, or 10) Nuclear Export Signal (NES) attached the N-terminal or C-terminal of the protein. In a preferred embodiment a C-terminal and/or N-terminal NLS or NES is attached for optimal expression and nuclear targeting in eukaryotic cells, e.g., human cells.

In some embodiments, the engineered Class 2 type VI Cas13 effectors, such as those either substantially lacking or having enhanced collateral activity are mutated at one or more amino acid residues to alter one or more functional activities.

For example, in some embodiments, the engineered Class 2 type VI Cas13 effectors, such as those either substantially lacking or having enhanced collateral activity is mutated at one or more amino acid residues to alter its helicase activity.

In some embodiments, the engineered Class 2 type VI Cas13 effectors, such as those either substantially lacking or having enhanced collateral activity is mutated at one or more amino acid residues to alter its nuclease activity (e.g., endonuclease activity or exonuclease activity), such as the collateral nuclease activity that is not dependent on guide sequence.

In some embodiments, the engineered Class 2 type VI Cas13 effectors, such as those either substantially lacking or having enhanced collateral activity is mutated at one or more amino acid residues to alter its ability to functionally associate with a guide RNA.

In some embodiments, the engineered Class 2 type VI Cas13 effectors, such as those either substantially lacking or having enhanced collateral activity is mutated at one or more amino acid residues to alter its ability to functionally associate with a target nucleic acid.

In some embodiments, the engineered Class 2 type VI Cas13 effectors, such as those either substantially lacking or having enhanced collateral activity described herein are capable of cleaving a target RNA molecule.

In some embodiments, the engineered Class 2 type VI Cas13 effectors, such as those either substantially lacking or having enhanced collateral activity is mutated at one or more amino acid residues to alter its cleaving activity. For example, in some embodiments, the engineered Class 2 type VI Cas13 effectors, such as those either substantially lacking or having enhanced collateral activity may comprise one or more mutations that render the enzyme incapable of cleaving a target nucleic acid.

In some embodiments, the engineered Class 2 type VI Cas13 effectors, such as those either substantially lacking or having enhanced collateral activity is capable of cleaving the strand of the target nucleic acid that is complementary to the strand to which the guide RNA hybridizes.

In some embodiments, a engineered Class 2 type VI Cas13 effectors, such as those either substantially lacking or having enhanced collateral activity described herein can be engineered to have a deletion in one or more amino acid residues to reduce the size of the enzyme while retaining one or more desired functional activities (e.g., nuclease activity and the ability to interact functionally with a guide RNA). The truncated engineered Class 2 type VI Cas13 effectors, such as those either substantially lacking or having enhanced collateral activity can be advantageously used in combination with delivery systems having load limitations.

In some embodiments, the engineered Class 2 type VI Cas13 effectors, such as those either substantially lacking or having enhanced collateral activity described herein can be fused to one or more peptide tags, including a His-tag, GST-tag, a V5-tag, FLAG-tag, HA-tag, VSV-G-tag, Trx-tag, or myc-tag.

In some embodiments, the engineered Class 2 type VI Cas13 effectors, such as those either substantially lacking or having enhanced collateral activity described herein can be fused to a detectable moiety such as GST, a fluorescent protein (e.g., GFP, HcRed, DsRed, CFP, YFP, or BFP), or an enzyme (such as HRP or CAT).

In some embodiments, the engineered Class 2 type VI Cas13 effectors, such as those either substantially lacking or having enhanced collateral activity described herein can be fused to MBP, LexA DNA binding domain, or Gal4 DNA-binding domain.

In some embodiments, the engineered Class 2 type VI Cas13 effectors, such as those either substantially lacking or having enhanced collateral activity described herein can be linked to or conjugated with a detectable label such as a fluorescent dye, including FITC and DAPI.

In any of the embodiments herein, the linkage between the engineered Class 2 type VI Cas13 effectors, such as those either substantially lacking or having enhanced collateral activity described herein and the other moiety can be at the N- or C-terminal of the CRISPR-associated proteins, and sometimes even internally via covalent chemical bonds. The linkage can be affected by any chemical linkage known in the art, such as peptide linkage, linkage through the side chain of amino acids such as D, E, S, T, or amino acid derivatives (Ahx, β-Ala, GABA or Ava), or PEG linkage.

### 3. Polynucleotides

The invention also provides nucleic acids encoding the proteins described herein (e.g., an engineered Class 2 type VI Cas13 protein, such as those either substantially lacking or having enhanced collateral activity).

In some embodiments, the nucleic acid is a synthetic nucleic acid. In some embodiments, the nucleic acid is a DNA molecule. In some embodiments, the nucleic acid is an RNA molecule (e.g., an mRNA molecule encoding the engineered Class 2 type VI Cas13 protein, such as those either substantially lacking or having enhanced collateral activity, derivative or functional fragment thereof). In some embodiments, the mRNA is capped, polyadenylated, substituted with 5-methyl cytidine, substituted with pseudouridine, or a combination thereof.

In some embodiments, the nucleic acid (e.g., DNA) is operably linked to a regulatory element (e.g., a promoter) in order to control the expression of the nucleic acid. In some embodiments, the promoter is a constitutive promoter. In some embodiments, the promoter is an inducible promoter. In some embodiments, the promoter is a cell-specific promoter. In some embodiments, the promoter is an organism-specific promoter.

Suitable promoters are known in the art and include, for example, a pol I promoter, a pol II promoter, a pol III promoter, a T7 promoter, a U6 promoter, a H1 promoter, retroviral Rous sarcoma virus LTR promoter, a cytomegalovirus (CMV) promoter, a SV40 promoter, a dihydrofolate reductase promoter, and a β-actin promoter. For example, a U6 promoter can be used to regulate the expression of a guide RNA molecule described herein.

In some embodiments, the nucleic acid(s) are present in a vector (e.g., a viral vector or a phage). The vector can be a cloning vector, or an expression vector. The vectors can be plasmids, phagemids, Cosmids, etc. The vectors may include one or more regulatory elements that allow for the propagation of the vector in a cell of interest (e.g., a bacterial cell or a mammalian cell). In some embodiments, the vector includes a nucleic acid encoding a single component of a CRISPR-associated (Cas) system described herein. In some embodiments, the vector includes multiple nucleic acids, each encoding a component of a CRISPR-associated (Cas) system described herein.

In one aspect, the present disclosure provides nucleic acid sequences that are at least 50%, 55%, 60%, 65%, 70%, 75%, 80%, 85%, 90%, 91%, 92%, 93%, 94%, 95%, 96%, 97%, 98%, 99%, or 100% identical to the nucleic acid sequences described herein, i.e., nucleic acid sequences encoding the engineered Class 2 type VI Cas13 protein substantially lacking collateral activity, derivatives, functional fragments, or guide/crRNA, including the DR sequences.

In another aspect, the present disclosure also provides nucleic acid sequences encoding amino acid sequences that are at least 50%, 55%, 60%, 65%, 70%, 75%, 80%, 85%, 90%, 91%, 92%, 93%, 94%, 95%, 96%, 97%, 98%, 99%, or 100% identical to the amino acid sequences of the subject engineered Class 2 type VI Cas13 protein substantially lacking collateral activity.

In some embodiments, the nucleic acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as the sequences described herein. In some embodiments, the nucleic acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from the sequences described herein.

In related embodiments, the invention provides amino acid sequences having at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as the sequences described herein. In some embodiments, the amino acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from the sequences described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In general, the length of a reference sequence aligned for comparison purposes should be at least 80% of the length of the reference sequence, and in some embodiments is at least 90%, 95%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

The proteins described herein (e.g., an engineered Class 2 type VI Cas13 protein substantially lacking collateral activity) can be delivered or used as either nucleic acid molecules or polypeptides.

In certain embodiments, the nucleic acid molecule encoding the engineered Class 2 type VI Cas13 protein, such as those either substantially lacking or having enhanced collateral activity, derivatives or functional fragments thereof are codon-optimized for expression in a host cell or organism. The host cell may include established cell lines (such as 293T cells) or isolated primary cells. The nucleic acid can be codon optimized for use in any organism of interest, in particular human cells or bacteria. For example, the nucleic acid can be codon-optimized for any prokaryotes (such as E. coli), or any eukaryotes such as human and other non-human eukaryotes including yeast, worm, insect, plants and algae (including food crop, rice, corn, vegetables, fruits, trees, grasses), vertebrate, fish, non-human mammal (e.g., mice, rats, rabbits, dogs, birds (such as chicken), livestock (cow or cattle, pig, horse, sheep, goat etc.), or non-human primates). Codon usage tables are readily available, for example, at the “Codon Usage Database” available at www.kazusa.orjp/codon/, and these tables can be adapted in a number of ways. See Nakamura et al., Nucl. Acids Res. 28:292, 2000 (incorporated herein by reference in its entirety). Computer algorithms for codon optimizing a particular sequence for expression in a particular host cell are also available, such as Gene Forge (Aptagen; Jacobus, Pa.).

An example of a codon optimized sequence, is in this instance a sequence optimized for expression in a eukaryote, e.g., humans (i.e. being optimized for expression in humans), or for another eukaryote, animal or mammal as herein discussed; see, e.g., SaCas9 human codon optimized sequence in WO 2014/093622 (PCT/US2013/074667). Whilst this is preferred, it will be appreciated that other examples are possible and codon optimization for a host species other than human, or for codon optimization for specific organs is known. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at http://www.kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g., 1, 2, 3, 4, 5, 10, 15, 20, 25, 50, or more, or all codons) in a sequence encoding a Cas correspond to the most frequently used codon for a particular amino acid.

4. RNA Guides or crRNA

In some embodiments, the CRISPR systems described herein include at least RNA guide (e.g., a gRNA or a crRNA).

The architecture of multiple RNA guides is known in the art (see, e.g., International Publication Nos. WO 2014/093622 and WO 2015/070083, the entire contents of each of which are incorporated herein by reference).

In some embodiments, the CRISPR systems described herein include multiple RNA guides (e.g., one, two, three, four, five, six, seven, eight, or more RNA guides).

In some embodiments, the RNA guide includes a crRNA. In some embodiments, the RNA guide includes a crRNA but not a tracrRNA.

Sequences for guide RNAs from multiple CRISPR systems are generally known in the art, see, for example, Grissa et al. (Nucleic Acids Res. 35 (web server issue): W52-7, 2007; Grissa et al., BMC Bioinformatics 8:172, 2007; Grissa et al., Nucleic Acids Res. 36 (web server issue): W145-8, 2008; and Moller and Liang, PeerJ 5: e3788, 2017; the CRISPR database at: crispr.i2bc.paris-saclayfr/crispr/BLAST/CRISPRsBlast.php; and MetaCRAST available at: github.com/molleraj/MetaCRAST). All incorporated herein by reference.

In some embodiments, the crRNA includes a direct repeat (DR) sequence and a spacer sequence. In certain embodiments, the crRNA comprises, consists essentially of, or consists of a direct repeat sequence linked to a guide sequence or spacer sequence, preferably at the 3′-end of the spacer sequence.

In general, an engineered Class 2 type VI Cas13 protein, such as those either substantially lacking or having enhanced collateral activity forms a complex with the mature crRNA, which spacer sequence directs the complex to a sequence-specific binding with the target RNA that is complementary to the spacer sequence, and/or hybridizes to the spacer sequence. The resulting complex comprises the engineered Class 2 type VI Cas13 protein, such as those either substantially lacking or having enhanced collateral activity and the mature crRNA bound to the target RNA.

The direct repeat sequences for the Cas13 systems are generally well conserved, especially at the ends, with, for example, a GCTG for Cas13e and GCTGT for Cas13f at the 5′-end, reverse complementary to a CAGC for Cas13e and ACAGC for Cas13f at the 3′ end. This conservation suggests strong base pairing for an RNA stem-loop structure that potentially interacts with the protein(s) in the locus.

In some embodiments, the direct repeat sequence, when in RNA, comprises the general secondary structure of 5′-S1a-Ba-S2a-L-S2b-Bb-S1b-3′, wherein segments S1a and S1b are reverse complement sequences and form a first stem (S1) having 4 nucleotides in Cas13e and 5 nucleotides in Cas13f; segments Ba and Bb do not base pair with each other and form a symmetrical or nearly symmetrical bulge (B), and have 5 nucleotides each in Cas13e, and 5 (Ba) and 4 (Bb) or 6 (Ba) and 5 (Bb) nucleotides respectively in Cas13f; segments S2a and S2b are reverse complement sequences and form a second stem (S2) having 5 base pairs in Cas13e and either 6 or 5 base pairs in Cas13f; and L is an 8-nucleotide loop in Cas13e and a 5-nucleotide loop in Cas13f.

In certain embodiments, S1a has a sequence of GCUG in Cas13e and GCUGU in Cas13f.

In certain embodiments, S2a has a sequence of GCCCC in Cas13e and A/G CCUC G/A in Cas13f (wherein the first A or G may be absent).

In some embodiments, the direct repeat sequence comprises or consists of a nucleic acid sequence of SEQ ID NOs: 57-63.

As used herein, “direct repeat sequence” may refer to the DNA coding sequence in the CRISPR locus, or to the RNA encoded by the same in crRNA. Thus when any of SEQ ID NOs: 57-63 is referred to in the context of an RNA molecule, such as crRNA, each T is understood to represent a U.

In some embodiments, the direct repeat sequence comprises or consists of a nucleic acid sequence having up to 1, 2, 3, 4, 5, 6, 7, or 8 nucleotides of deletion, insertion, or substitution of SEQ ID NOs: 57-63. In some embodiments, the direct repeat sequence comprises or consists of a nucleic acid sequence having at least 80%, 85%, 90%, 95%, or 97% of sequence identity with SEQ ID NOs: 57-63 (e.g., due to deletion, insertion, or substitution of nucleotides in SEQ ID NOs: 57-63). In some embodiments, the direct repeat sequence comprises or consists of a nucleic acid sequence that is not identical to any one of SEQ ID NOs: 57-63, but can hybridize with a complement of any one of SEQ ID NOs: 57-63 under stringent hybridization conditions, or can bind to a complement of any one of SEQ ID NOs: 57-63 under physiological conditions.

In certain embodiments, the deletion, insertion, or substitution does not change the overall secondary structure of that of SEQ ID NOs: 57-63 (e.g., the relative locations and/or sizes of the stems and bulges and loop do not significantly deviate from that of the original stems, bulges, and loop). For example, the deletion, insert, or substitution may be in the bulge or loop region so that the overall symmetry of the bulge remains largely the same. The deletion, insertion, or substitution may be in the stems so that the length of the stems do not significantly deviate from that of the original stems (e.g., adding or deleting one base pair in each of the two stems correspond to 4 total base changes).

In certain embodiments, the deletion, insertion, or substitution results in a derivative DR sequence that may have ±1 or 2 base pair(s) in one or both stems, have ±1, 2, or 3 bases in either or both of the single strands in the bulge, and/or have ±1, 2, 3, or 4 bases in the loop region.

In certain embodiments, any of the above direct repeat sequences that is different from any one of SEQ ID NOs: 57-63 retains the ability to function as a direct repeat sequence in the Cas13e or Cas13f proteins, as the DR sequence of SEQ ID NOs: 57-63.

In some embodiments, the direct repeat sequence comprises or consists of a nucleic acid having a nucleic acid sequence of any one of SEQ ID NOs: 57-63, with a truncation of the initial three, four, five, six, seven, or eight 3′ nucleotides.

In classic CRISPR systems, the degree of complementarity between a guide sequence (e.g., a crRNA) and its corresponding target sequence can be about 50%, 60%, 75%, 80%, 85%, 90%, 95%, 97.5%, 99%, or 100%. In some embodiments, the degree of complementarity is 90-100%.

The guide RNAs can be about 5, 10, 11, 12, 13, 14, 15, 16, 17, 18, 19, 20, 21, 22, 23, 24, 25, 26, 27, 28, 29, 30, 35, 40, 45, 50, 75, 100, 125, 150, 175, 200 or more nucleotides in length. For example, for use in a functional engineered Cas13e or Cas13f effector protein, or homologs, orthologs, derivatives, fusions, conjugates, or functional fragment thereof, the spacer can be between 10-60 nucleotides, 20-50 nucleotides, 25-45 nucleotides, 25-35 nucleotides, or about 27, 28, 29, 30, 31, 32, or 33 nucleotides. For use in dCas version of any of the above, however, the spacer can be between 10-200 nucleotides, 20-150 nucleotides, 25-100 nucleotides, 25-85 nucleotides, 35-75 nucleotides, 45-60 nucleotides, or about 46, 47, 48, 49, 50, 51, 52, 53, 54, or 55 nucleotides.

To reduce off-target interactions, e.g., to reduce the guide interacting with a target sequence having low complementarity, mutations can be introduced to the CRISPR systems so that the CRISPR systems can distinguish between target and off-target sequences that have greater than 80%, 85%, 90%, or 95% complementarity. In some embodiments, the degree of complementarity is from 80% to 95%, e.g., about 83%, 84%, 85%, 86%, 87%, 88%, 89%, 90%, 91%, 92%, 93%, 94%, or 95% (for example, distinguishing between a target having 18 nucleotides from an off-target of 18 nucleotides having 1, 2, or 3 mismatches). Accordingly, in some embodiments, the degree of complementarity between a guide sequence and its corresponding target sequence is greater than 94.5%, 95%, 95.5%, 96%, 96.5%, 97%, 97.5%, 98%, 98.5%, 99%, 99.5%, or 99.9%. In some embodiments, the degree of complementarity is 100%.

It is known in the field that complete complementarity is not required, provided there is sufficient complementarity to be functional. Modulations of cleavage efficiency can be exploited by introduction of mismatches, e.g., one or more mismatches, such as 1 or 2 mismatches between spacer sequence and target sequence, including the position of the mismatch along the spacer/target. The more central (i.e., not at the 3′ or 5′-ends) a mismatch, e.g., a double mismatch, is located; the more cleavage efficiency is affected. Accordingly, by choosing mismatch positions along the spacer sequence, cleavage efficiency can be modulated. For example, if less than 100% cleavage of targets is desired (e.g., in a cell population), 1 or 2 mismatches between spacer and target sequence can be introduced in the spacer sequences.

Type VI CRISPR-Cas effectors have been demonstrated to employ more than one RNA guide, thus enabling the ability of these effectors, and systems and complexes that include them, to target multiple nucleic acids. In some embodiments, the CRISPR systems comprising the engineered Class 2 type VI Cas13 protein, such as those either substantially lacking or having enhanced collateral activity, as described herein, include multiple RNA guides (e.g., two, three, four, five, six, seven, eight, nine, ten, fifteen, twenty, thirty, forty, or more) RNA guides. In some embodiments, the CRISPR systems described herein include a single RNA strand or a nucleic acid encoding a single RNA strand, wherein the RNA guides are arranged in tandem. The single RNA strand can include multiple copies of the same RNA guide, multiple copies of distinct RNA guides, or combinations thereof. The processing capability of the Type VI-E and VI-F CRISPR-Cas effector proteins described herein enables these effectors to be able to target multiple target nucleic acids (e.g., target RNAs) without a loss of activity. In some embodiments, the Type VI-E and VI-F CRISPR-Cas effector proteins may be delivered in complex with multiple RNA guides directed to different target RNA. In some embodiments, the engineered Class 2 type VI Cas13 protein, such as those either substantially lacking or having enhanced collateral activity may be co-delivered with multiple RNA guides, each specific for a different target nucleic acid. Methods of multiplexing using CRISPR-associated proteins are described, for example, in U.S. Pat. No. 9,790,490 B2, and EP3009511 B1, the entire contents of each of which are expressly incorporated herein by reference.

The spacer length of crRNAs can range from about 10-50 nucleotides, such as 15-50 nucleotides, 20-50 nucleotides, 25-50 nucleotide, or 19-50 nucleotides. In some embodiments, the spacer length of a guide RNA is at least 16 nucleotides, at least 17 nucleotides, at least 18 nucleotides, at least 19 nucleotides, at least 20 nucleotides, at least 21 nucleotides, or at least 22 nucleotides. In some embodiments, the spacer length is from 15 to 17 nucleotides (e.g., 15, 16, or 17 nucleotides), from 17 to 20 nucleotides (e.g., 17, 18, 19, or 20 nucleotides), from 20 to 24 nucleotides (e.g., 20, 21, 22, 23, or 24 nucleotides), from 23 to 25 nucleotides (e.g., 23, 24, or 25 nucleotides), from 24 to 27 nucleotides, from 27 to 30 nucleotides, from 30 to 45 nucleotides (e.g., 30, 31, 32, 33, 34, 35, 36, 37, 38, 39, 40, 41, 42, 43, 44, or 45 nucleotides), from 30 or 35 to 40 nucleotides, from 41 to 45 nucleotides, from 45 to 50 nucleotides (e.g., 45, 46, 47, 48, 49, or 50 nucleotides), or longer. In some embodiments, the spacer length is from about 15 to about 42 nucleotides.

In some embodiments, the direct repeat length of the guide RNA is 15-36 nucleotides, is at least 16 nucleotides, is from 16 to 20 nucleotides (e.g., 16, 17, 18, 19, or 20 nucleotides), is from 20-30 nucleotides (e.g., 20, 21, 22, 23, 24, 25, 26, 27, 28, 29, or 30 nucleotides), is from 30-40 nucleotides (e.g., 30, 31, 32, 33, 34, 35, 36, 37, 38, 39, or 40 nucleotides), or is about 36 nucleotides (e.g., 33, 34, 35, 36, 37, 38, or 39 nucleotides). In some embodiments, the direct repeat length of the guide RNA is 36 nucleotides.

In some embodiments, the overall length of the crRNA/guide RNA is about 36 nucleotides longer than any one of the spacer sequence length described herein above. For example, the overall length of the crRNA/guide RNA may be between 45-86 nucleotides, or 60-86 nucleotides, 62-86 nucleotides, or 63-86 nucleotides.

The crRNA sequences can be modified in a manner that allows for formation of a complex between the crRNA and the engineered Class 2 type VI Cas13 protein, such as those either substantially lacking or having enhanced collateral activity, and successful binding to the target, while at the same time not allowing for successful nuclease activity (i.e., without nuclease activity/without causing indels). These modified guide sequences are referred to as “dead crRNAs,” “dead guides,” or “dead guide sequences.” These dead guides or dead guide sequences may be catalytically inactive or conformationally inactive with regard to nuclease activity. Dead guide sequences are typically shorter than respective guide sequences that result in active RNA cleavage. In some embodiments, dead guides are 5%, 10%, 20%, 30%, 40%, or 50%, shorter than respective guide RNAs that have nuclease activity. Dead guide sequences of guide RNAs can be from 13 to 15 nucleotides in length (e.g., 13, 14, or 15 nucleotides in length), from 15 to 19 nucleotides in length, or from 17 to 18 nucleotides in length (e.g., 17 nucleotides in length).

Thus, in one aspect, the disclosure provides non-naturally occurring or engineered CRISPR systems including a functional engineered Class 2 type VI Cas13 protein, such as those either substantially lacking or having enhanced collateral activity as described herein, and a crRNA, wherein the crRNA comprises a dead crRNA sequence whereby the crRNA is capable of hybridizing to a target sequence such that the CRISPR system is directed to a target RNA of interest in a cell without detectable nuclease activity (e.g., RNase activity).

A detailed description of dead guides is described, e.g., in International Publication No. WO 2016/094872, which is incorporated herein by reference in its entirety.

Guide RNAs (e.g., crRNAs) can be generated as components of inducible systems. The inducible nature of the systems allows for spatio-temporal control of gene editing or gene expression. In some embodiments, the stimuli for the inducible systems include, e.g., electromagnetic radiation, sound energy, chemical energy, and/or thermal energy.

In some embodiments, the transcription of guide RNA (e.g., crRNA) can be modulated by inducible promoters, e.g., tetracycline or doxycycline controlled transcriptional activation (Tet-On and Tet-Off expression systems), hormone inducible gene expression systems (e.g., ecdysone inducible gene expression systems), and arabinose-inducible gene expression systems. Other examples of inducible systems include, e.g., small molecule two-hybrid transcription activations systems (FKBP, ABA, etc.), light inducible systems (Phytochrome, LOV domains, or cryptochrome), or Light Inducible Transcriptional Effector (LITE). These inducible systems are described, e.g., in WO 2016205764 and U.S. Pat. No. 8,795,965, both of which are incorporated herein by reference in the entirety.

Chemical modifications can be applied to the crRNA's phosphate backbone, sugar, and/or base. Backbone modifications such as phosphorothioates modify the charge on the phosphate backbone and aid in the delivery and nuclease resistance of the oligonucleotide (see, e.g., Eckstein, “Phosphorothioates, essential components of therapeutic oligonucleotides,” Nucl. Acid Ther., 24, pp. 374-387, 2014); modifications of sugars, such as 2′-O-methyl (2′-OMe), 2′-F, and locked nucleic acid (LNA), enhance both base pairing and nuclease resistance (see, e.g., Allerson et al. “Fully 2′-modified oligonucleotide duplexes with improved in vitro potency and stability compared to unmodified small interfering RNA,” J. Med. Chem. 48.4: 901-904, 2005). Chemically modified bases such as 2-thiouridine or N6-methyladenosine, among others, can allow for either stronger or weaker base pairing (see, e.g., Bramsen et al., “Development of therapeutic-grade small interfering RNAs by chemical engineering,” Front. Genet., 2012 Aug. 20; 3:154). Additionally, RNA is amenable to both 5′ and 3′ end conjugations with a variety of functional moieties including fluorescent dyes, polyethylene glycol, or proteins.

A wide variety of modifications can be applied to chemically synthesized crRNA molecules. For example, modifying an oligonucleotide with a 2′-OMe to improve nuclease resistance can change the binding energy of Watson-Crick base pairing. Furthermore, a 2′-OMe modification can affect how the oligonucleotide interacts with transfection reagents, proteins or any other molecules in the cell. The effects of these modifications can be determined by empirical testing.

In some embodiments, the crRNA includes one or more phosphorothioate modifications. In some embodiments, the crRNA includes one or more locked nucleic acids for the purpose of enhancing base pairing and/or increasing nuclease resistance.

A summary of these chemical modifications can be found, e.g., in Kelley et al., “Versatility of chemically synthesized guide RNAs for CRISPR-Cas9 genome editing,” J. Biotechnol. 233:74-83, 2016; WO 2016205764; and U.S. Pat. No. 8,795,965 B2; each which is incorporated by reference in its entirety.

The sequences and the lengths of the RNA guides (e.g., crRNAs) described herein can be optimized. In some embodiments, the optimized length of an RNA guide can be determined by identifying the processed form of crRNA (i.e., a mature crRNA), or by empirical length studies for crRNA tetraloops.

The crRNAs can also include one or more aptamer sequences. Aptamers are oligonucleotide or peptide molecules have a specific three-dimensional structure and can bind to a specific target molecule. The aptamers can be specific to gene effectors, gene activators, or gene repressors. In some embodiments, the aptamers can be specific to a protein, which in turn is specific to and recruits and/or binds to specific gene effectors, gene activators, or gene repressors. The effectors, activators, or repressors can be present in the form of fusion proteins. In some embodiments, the guide RNA has two or more aptamer sequences that are specific to the same adaptor proteins. In some embodiments, the two or more aptamer sequences are specific to different adaptor proteins. The adaptor proteins can include, e.g., MS2, PP7, Qβ, F2, GA, fr, JP501, M12, R17, BZ13, JP34, JP500, KU1, M11, MX1, TW18, VK, SP, FI, ID2, NL95, TW19, AP205, ϕkCb5, ϕkCb8r, ϕkCb12r, ϕkCb23r, 7s, and PRR1. Accordingly, in some embodiments, the aptamer is selected from binding proteins specifically binding any one of the adaptor proteins as described herein. In some embodiments, the aptamer sequence is a MS2 binding loop (SEQ ID NO: 95). In some embodiments, the aptamer sequence is a QBeta binding loop (SEQ ID NO: 96). In some embodiments, the aptamer sequence is a PP7 binding loop (SEQ ID NO: 97). A detailed description of aptamers can be found, e.g., in Nowak et al., “Guide RNA engineering for versatile Cas9 functionality,” Nucl. Acid. Res., 44(20):9555-9564, 2016; and WO 2016205764, which are incorporated herein by reference in their entirety.

In certain embodiments, the methods make use of chemically modified guide RNAs. Examples of guide RNA chemical modifications include, without limitation, incorporation of 2′-O-methyl (M), 2′-O-methyl 3′-phosphorothioate (MS), or 2′-O-methyl 3′-thioPACE (MSP) at one or more terminal nucleotides. Such chemically modified guide RNAs can comprise increased stability and increased activity as compared to unmodified guide RNAs, though on-target vs. off-target specificity is not predictable. See, Hendel, Nat Biotechnol. 33(9):985-9, 2015, incorporated by reference). Chemically modified guide RNAs may further include, without limitation, RNAs with phosphorothioate linkages and locked nucleic acid (LNA) nucleotides comprising a methylene bridge between the 2′ and 4′ carbons of the ribose ring.

The invention also encompasses methods for delivering multiple nucleic acid components, wherein each nucleic acid component is specific for a different target locus of interest thereby modifying multiple target loci of interest. The nucleic acid component of the complex may comprise one or more protein-binding RNA aptamers. The one or more aptamers may be capable of binding a bacteriophage coat protein. The bacteriophage coat protein may be selected from the group comprising Qβ, F2, GA, fr, JP501, MS2, M12, R17, BZ13, JP34, JP500, KU1, M11, MX1, TW18, VK, SP, FI, ID2, NL95, TW19, AP205, ϕCb5, ϕCb8r, ϕCb12r, ϕCb23r, 7s and PRR1. In certain embodiments, the bacteriophage coat protein is MS2.

### 5. Target RNA

The target RNA can be any RNA molecule of interest, including naturally-occurring and engineered RNA molecules. The target RNA can be an mRNA, a tRNA, a ribosomal RNA (rRNA), a microRNA (miRNA), an interfering RNA (siRNA), a ribozyme, a riboswitch, a satellite RNA, a microswitch, a microzyme, or a viral RNA.

In some embodiments, the target nucleic acid is associated with a condition or disease (e.g., an infectious disease or a cancer).

Thus, in some embodiments, the systems described herein can be used to treat a condition or disease by targeting these nucleic acids. For instance, the target nucleic acid associated with a condition or disease may be an RNA molecule that is overexpressed in a diseased cell (e.g., a cancer or tumor cell). The target nucleic acid may also be a toxic RNA and/or a mutated RNA (e.g., an mRNA molecule having a splicing defect or a mutation). The target nucleic acid may also be an RNA that is specific for a particular microorganism (e.g., a pathogenic bacteria).

### 6. Complex and Cell

One aspect of the invention provides a complex of an engineered Class 2 type VI Cas13 protein, such as those either substantially lacking or having enhanced collateral activity, such as CRISPR/Cas13e or CRISPR/Cas13f complex, comprising (1) any of the engineered Class 2 type VI Cas13 protein, such as those either substantially lacking or having enhanced collateral activity (e.g., engineered Cas13e/Cas13f effector proteins, homologs, orthologs, fusions, derivative, conjugates, or functional fragments thereof as described herein), and (2) any of the guide RNA described herein, each including a spacer sequence designed to be at least partially complementary to a target RNA, and a DR sequence compatible with the engineered Class 2 type VI Cas13 protein, such as those either substantially lacking or having enhanced collateral activity (e.g., Cas13d, Cas13e/Cas13f effector proteins), homologs, orthologs, fusions, derivatives, conjugates, or functional fragments thereof.

In certain embodiments, the complex further comprises the target RNA bound by the guide RNA.

In a related aspect, the invention also provides a cell comprising any of the complex of the invention. In certain embodiments, the cell is a prokaryote. In certain embodiments, the cell is a eukaryote.

### 7. Methods of Using CRISPR Systems

The CRISPR/Cas systems having the engineered Cas13, e.g., an engineered Class 2 type VI Cas13 protein, such as those either substantially lacking or having enhanced collateral activity, as described herein, have a wide variety of utilities like the corresponding wild-type Cas13-based systems, including modifying (e.g., deleting, inserting, translocating, inactivating, or activating) a target polynucleotide or nucleic acid in a multiplicity of cell types. The CRISPR systems have a broad spectrum of applications in, e.g., tracking and labeling of nucleic acids, enrichment assays (extracting desired sequence from background), controlling interfering RNA or miRNA, detecting circulating tumor DNA, preparing next generation library, drug screening, disease diagnosis and prognosis, and treating various genetic disorders.

Certain engineered Cas13 effector enzymes, as described herein, have enhanced collateral effect compared to the wild-type, and thus may be better alternatives than the wild-type Cas13 effector enzymes for utilities that take advantage of the enhanced collateral activity, such as DNA/RNA detection (e.g., specific high sensitivity enzymatic reporter unlocking (SHERLOCK)). Such engineered Cas13 effector enzymes with enhanced collateral activity is within the scope of one aspect of the invention.

### RNA Detection

In one aspect, the CRISPR systems described herein can be used in RNA detection. As shown in the examples, wild-type Cas13 such as Cas13e of the invention exhibit non-specific/collateral RNase activity upon activation of its guide RNA-dependent specific RNase activity when the spacer sequence is about 30 nucleotides. Thus the engineered CRISPR-associated proteins of the invention with enhanced collateral activity (compared to the wild-type) can be reprogrammed with CRISPR RNAs (crRNAs) to provide a platform for specific RNA sensing. Further, by choosing specific spacer sequence length, and upon recognition of its RNA target, activated CRISPR-associated proteins engage in enhanced collateral cleavage of nearby non-targeted RNAs. This crRNA-programmed collateral cleavage activity allows the CRISPR systems to detect the presence of a specific RNA by triggering programmed cell death or by nonspecific degradation of labeled RNA.

The SHERLOCK method (Specific High Sensitivity Enzymatic Reporter UnLOCKing) provides an in vitro nucleic acid detection platform with attomolar sensitivity based on nucleic acid amplification and collateral cleavage of a reporter RNA, allowing for real-time detection of the target. To achieve signal detection, the detection can be combined with different isothermal amplification steps. For example, recombinase polymerase amplification (RPA) can be coupled with T7 transcription to convert amplified DNA to RNA for subsequent detection. The combination of amplification by RPA, T7 RNA polymerase transcription of amplified DNA to RNA, and detection of target RNA by collateral RNA cleavage-mediated release of reporter signal is referred as SHERLOCK. Methods of using CRISPR in SHERLOCK are described in detail, e.g., in Gootenberg, et al. “Nucleic acid detection with CRISPR-Cas13a/C2c2,” Science, 2017 Apr. 28; 356(6336):438-442, which is incorporated herein by reference in its entirety.

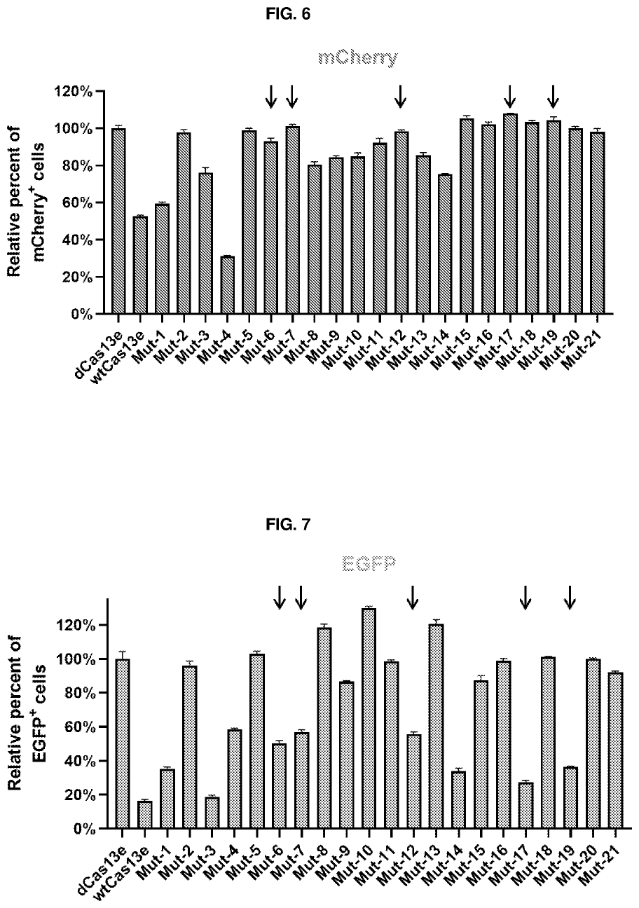

The invention described herein provides mutant/variant Class 2, Type VI CRISPR/Cas effector enzymes, especially Type VI-D, -E, and -F Cas mutants/variants having enhanced collateral effect, such that they can be more effective in nucleic acid detection assays based on the collateral effect, such as the SHERLOCK assay. Such mutants include any one described in Examples 1, 2, 4, and 5, as well as FIGS. 6, 7, 9-14, 17D, 17E, 19C, and 19D, having at least 80%, 85%, or 87.5% or more collateral cleavage efficiency, and optionally better gRNA-guided cleavage compared to a corresponding wild-type Cas13.

In certain embodiments, such Cas13 mutants have enhanced collateral effect comprises, consists essentially of, or consists of a mutation corresponding to the N2-Y142A, N4-Y193A, N12-Y604A, or N21V7 mutation of Cas13d, or to the M14V2, M16V3, M18V1, M19-G712A, M19-T725A, or M19-C727A mutation of Cas13e.

The CRISPR-associated proteins can be used in Northern blot assays, which use electrophoresis to separate RNA samples by size. The CRISPR-associated proteins can be used to specifically bind and detect the target RNA sequence. The CRISPR-associated proteins can also be fused to a fluorescent protein (e.g., GFP) and used to track RNA localization in living cells. More particularly, the CRISPR-associated proteins can be inactivated in that they no longer cleave RNAs as described above. Thus, CRISPR-associated proteins can be used to determine the localization of the RNA or specific splice variants, the level of mRNA transcripts, up- or down-regulation of transcripts and disease-specific diagnosis. The CRISPR-associated proteins can be used for visualization of RNA in (living) cells using, for example, fluorescent microscopy or flow cytometry, such as fluorescence-activated cell sorting (FACS), which allows for high-throughput screening of cells and recovery of living cells following cell sorting. A detailed description regarding how to detect DNA and RNA can be found, e.g., in International Publication No. WO 2017/070605, which is incorporated herein by reference in its entirety.

In some embodiments, the CRISPR systems described herein can be used in multiplexed error-robust fluorescence in situ hybridization (MERFISH). These methods are described in, e.g., Chen et al., “Spatially resolved, highly multiplexed RNA profiling in single cells,” Science, 2015 Apr. 24; 348(6233):aaa6090, which is incorporated herein by reference herein in its entirety.

In some embodiments, the CRISPR systems described herein can be used to detect a target RNA in a sample (e.g., a clinical sample, a cell, or a cell lysate). The collateral RNase activity of the engineered Cas13, e.g., Type VI-E and/or VI-F CRISPR-Cas effector proteins described herein, is activated when the effector proteins bind to a target nucleic acid when the spacer sequence is of a specific chosen length (such as about 30 nucleotides). Upon binding to the target RNA of interest, the effector protein cleaves a labeled detector RNA to generate a signal (e.g., an increased signal or a decreased signal) thereby allowing for the qualitative and quantitative detection of the target RNA in the sample. The specific detection and quantification of RNA in the sample allows for a multitude of applications including diagnostics. In some embodiments, the methods include contacting a sample with: i) an RNA guide (e.g., crRNA) and/or a nucleic acid encoding the RNA guide, wherein the RNA guide consists of a direct repeat sequence and a spacer sequence capable of hybridizing to the target RNA; (ii) an engineered Class 2 type VI Cas13 protein with enhanced collateral activity compared to wild-type Cas13, such as a subject engineered Type VI-E or VI-F CRISPR-Cas effector protein (Cas13e or Cas13f) and/or a nucleic acid encoding the effector protein; and (iii) a labeled detector RNA; wherein the effector protein associates with the RNA guide to form a complex; wherein the RNA guide hybridizes to the target RNA; and wherein upon binding of the complex to the target RNA, the effector protein exhibits collateral RNase activity and cleaves the labeled detector RNA; and b) measuring a detectable signal produced by cleavage of the labeled detector RNA, wherein said measuring provides for detection of the single-stranded target RNA in the sample. In some embodiments, the methods further comprise comparing the detectable signal with a reference signal and determining the amount of target RNA in the sample.

In some embodiments, the measuring is performed using gold nanoparticle detection, fluorescence polarization, colloid phase transition/dispersion, electrochemical detection, and semiconductor based-sensing. In some embodiments, the labeled detector RNA includes a fluorescence-emitting dye pair, a fluorescence resonance energy transfer (FRET) pair, or a quencher/fluor pair. In some embodiments, upon cleavage of the labeled detector RNA by the effector protein, an amount of detectable signal produced by the labeled detector RNA is decreased or increased. In some embodiments, the labeled detector RNA produces a first detectable signal prior to cleavage by the effector protein and a second detectable signal after cleavage by the effector protein. In some embodiments, a detectable signal is produced when the labeled detector RNA is cleaved by the effector protein. In some embodiments, the labeled detector RNA comprises a modified nucleobase, a modified sugar moiety, a modified nucleic acid linkage, or a combination thereof. In some embodiments, the methods include the multi-channel detection of multiple independent target RNAs in a sample (e.g., two, three, four, five, six, seven, eight, nine, ten, fifteen, twenty, thirty, forty, or more target RNAs) by using multiple engineered Cas13, such as the engineered Type VI-E and/or VI-F CRISPR-Cas (Cas13e and/or Cas130 systems of the invention, each including a distinct orthologous effector protein and corresponding RNA guides, allowing for the differentiation of multiple target RNAs in the sample. In some embodiments, the methods include the multi-channel detection of multiple independent target RNAs in a sample, with the use of multiple instances of engineered Cas13, such as engineered Type VI-E and/or VI-F CRISPR-Cas systems of the invention, each containing an orthologous effector protein with differentiable collateral RNase substrates. Methods of detecting an RNA in a sample using CRISPR-associated proteins are described, for example, in U.S. Patent Publication No. 2017/0362644, the entire contents of which are incorporated herein by reference.

### Tracking and Labeling of Nucleic Acids

Cellular processes depend on a network of molecular interactions among proteins, RNAs, and DNAs. Accurate detection of protein-DNA and protein-RNA interactions is key to understanding such processes. In vitro proximity labeling techniques employ an affinity tag combined with, a reporter group, e.g., a photoactivatable group, to label polypeptides and RNAs in the vicinity of a protein or RNA of interest in vitro. After UV irradiation, the photoactivatable groups react with proteins and other molecules that are in close proximity to the tagged molecules, thereby labelling them. Labelled interacting molecules can subsequently be recovered and identified. The CRISPR-associated proteins can for instance be used to target probes to selected RNA sequences. These applications can also be applied in animal models for in vivo imaging of diseases or difficult-to culture cell types. The methods of tracking and labeling of nucleic acids are described, e.g., in U.S. Pat. No. 8,795,965, WO 2016205764, and WO 2017070605; each of which is incorporated herein by reference herein in its entirety.

RNA Isolation, Purification, Enrichment, and/or Depletion

The CRISPR systems (e.g., CRISPR-associated proteins) described herein can be used to isolate and/or purify the RNA. The CRISPR-associated proteins can be fused to an affinity tag that can be used to isolate and/or purify the RNA-CRISPR-associated protein complex. These applications are useful, e.g., for the analysis of gene expression profiles in cells.

In some embodiments, the CRISPR-associated proteins can be used to target a specific noncoding RNA (ncRNA) thereby blocking its activity. In some embodiments, the CRISPR-associated proteins can be used to specifically enrich a particular RNA (including but not limited to increasing stability, etc.), or alternatively, to specifically deplete a particular RNA (e.g., particular splice variants, isoforms, etc.).

These methods are described, e.g., in U.S. Pat. No. 8,795,965, WO 2016205764, and WO 2017070605; each of which is incorporated herein by reference herein in its entirety.

### High-Throughput Screening

The CRISPR systems described herein can be used for preparing next generation sequencing (NGS) libraries. For example, to create a cost-effective NGS library, the CRISPR systems can be used to disrupt the coding sequence of a target gene product, and the CRISPR-associated protein transfected clones can be screened simultaneously by next-generation sequencing (e.g., on the Ion Torrent PGM system). A detailed description regarding how to prepare NGS libraries can be found, e.g., in Bell et al., “A high-throughput screening strategy for detecting CRISPR-Cas9 induced mutations using next-generation sequencing,” BMC Genomics, 15.1 (2014): 1002, which is incorporated herein by reference in its entirety.

### Engineered Microorganisms

Microorganisms (e.g., E. coli, yeast, and microalgae) are widely used for synthetic biology. The development of synthetic biology has a wide utility, including various clinical applications. For example, the programmable CRISPR systems can be used to split proteins of toxic domains for targeted cell death, e.g., using cancer-linked RNA as target transcript. Further, pathways involving protein-protein interactions can be influenced in synthetic biological systems with, e.g., fusion complexes with the appropriate effectors such as kinases or enzymes.

In some embodiments, crRNAs that target phage sequences can be introduced into the microorganism. Thus, the disclosure also provides methods of vaccinating a microorganism (e.g., a production strain) against phage infection.

In some embodiments, the CRISPR systems provided herein can be used to engineer microorganisms, e.g., to improve yield or improve fermentation efficiency. For example, the CRISPR systems described herein can be used to engineer microorganisms, such as yeast, to generate biofuel or biopolymers from fermentable sugars, or to degrade plant-derived lignocellulose derived from agricultural waste as a source of fermentable sugars. More particularly, the methods described herein can be used to modify the expression of endogenous genes required for biofuel production and/or to modify endogenous genes, which may interfere with the biofuel synthesis. These methods of engineering microorganisms are described e.g., in Verwaal et al., “CRISPR/Cpf1 enables fast and simple genome editing of Saccharomyces cerevisiae,” Yeast doi: 10.1002/yea.3278, 2017; and Hlavova et al., “Improving microalgae for biotechnology-from genetics to synthetic biology,” Biotechnol. Adv., 33:1194-203, 2015, both of which are incorporated herein by reference in the entirety.

In some embodiments, the CRISPR systems provided herein can be used to induce death or dormancy of a cell (e.g., a microorganism such as an engineered microorganism). These methods can be used to induce dormancy or death of a multitude of cell types including prokaryotic and eukaryotic cells, including, but not limited to mammalian cells (e.g., cancer cells, or tissue culture cells), protozoans, fungal cells, cells infected with a virus, cells infected with an intracellular bacteria, cells infected with an intracellular protozoan, cells infected with a prion, bacteria (e.g., pathogenic and non-pathogenic bacteria), protozoans, and unicellular and multicellular parasites. For instance, in the field of synthetic biology it is highly desirable to have mechanisms of controlling engineered microorganisms (e.g., bacteria) in order to prevent their propagation or dissemination. The systems described herein can be used as “kill-switches” to regulate and/or prevent the propagation or dissemination of an engineered microorganism. Further, there is a need in the art for alternatives to current antibiotic treatments. The systems described herein can also be used in applications where it is desirable to kill or control a specific microbial population (e.g., a bacterial population). For example, the systems described herein may include an RNA guide (e.g., a crRNA) that targets a nucleic acid (e.g., an RNA) that is genus-, species-, or strain-specific, and can be delivered to the cell. Upon complexing and binding to the target nucleic acid, the collateral RNase activity of the Type VI-E and/or VI-F CRISPR-Cas effector proteins is activated leading to the cleavage of non-target RNA within the microorganisms, ultimately resulting in dormancy or death. In some embodiments, the methods comprise contacting the cell with a system described herein including a Type VI-E and/or VI-F CRISPR-Cas effector proteins or a nucleic acid encoding the effector protein, and a RNA guide (e.g., a crRNA) or a nucleic acid encoding the RNA guide, wherein the spacer sequence is complementary to at least 15 nucleotides (e.g., 16, 17, 18, 19, 20, 21, 22, 23, 24, 25, 26, 27, 28, 29, 30, 35, 40, 45, 50 or more nucleotides) of a target nucleic acid (e.g., a genus-, strain-, or species-specific RNA guide). Without wishing to be bound by any particular theory, the cleavage of non-target RNA by the Type VI-E and/or VI-F CRISPR-Cas effector proteins may induce programmed cell death, cell toxicity, apoptosis, necrosis, necroptosis, cell death, cell cycle arrest, cell anergy, a reduction of cell growth, or a reduction in cell proliferation. For example, in bacteria, the cleavage of non-target RNA by the Type VI-E and/or VI-F CRISPR-Cas effector proteins may be bacteriostatic or bactericidal.

### Application in Plants

The CRISPR systems described herein have a wide variety of utility in plants. In some embodiments, the CRISPR systems can be used to engineer transcriptome of plants (e.g., improving production, making products with desired post-translational modifications, or introducing genes for producing industrial products). In some embodiments, the CRISPR systems can be used to introduce a desired trait to a plant (e.g., without heritable modifications to the genome), or regulate expression of endogenous genes in plant cells or whole plants.

In some embodiments, the CRISPR systems can be used to identify, edit, and/or silence genes encoding specific proteins, e.g., allergenic proteins (e.g., allergenic proteins in peanuts, soybeans, lentils, peas, green beans, and mung beans). A detailed description regarding how to identify, edit, and/or silence genes encoding proteins is described, e.g., in Nicolaou et al., “Molecular diagnosis of peanut and legume allergy,” Curr. Opin. Allergy Clin. Immunol. 11(3):222-8, 2011, and WO 2016205764 A1; both of which are incorporated herein by reference in the entirety.

### Pooled-Screening

As described herein, pooled CRISPR screening is a powerful tool for identifying genes involved in biological mechanisms such as cell proliferation, drug resistance, and viral infection. Cells are transduced in bulk with a library of guide RNA (gRNA)-encoding vectors described herein, and the distribution of gRNAs is measured before and after applying a selective challenge. Pooled CRISPR screens work well for mechanisms that affect cell survival and proliferation, and they can be extended to measure the activity of individual genes (e.g., by using engineered reporter cell lines). Arrayed CRISPR screens, in which only one gene is targeted at a time, make it possible to use RNA-seq as the readout. In some embodiments, the CRISPR systems as described herein can be used in single-cell CRISPR screens. A detailed description regarding pooled CRISPR screenings can be found, e.g., in Datlinger et al., “Pooled CRISPR screening with single-cell transcriptome read-out,” Nat. Methods. 14(3):297-301, 2017, which is incorporated herein by reference in its entirety.

### Saturation Mutagenesis (Bashing)

The CRISPR systems described herein can be used for in situ saturating mutagenesis. In some embodiments, a pooled guide RNA library can be used to perform in situ saturating mutagenesis for particular genes or regulatory elements. Such methods can reveal critical minimal features and discrete vulnerabilities of these genes or regulatory elements (e.g., enhancers). These methods are described, e.g., in Canver et al., “BCL11A enhancer dissection by Cas9-mediated in situ saturating mutagenesis,” Nature 527(7577):192-7, 2015, which is incorporated herein by reference in its entirety.

### RNA-Related Applications

The CRISPR systems described herein can have various RNA-related applications, e.g., modulating gene expression, degrading a RNA molecule, inhibiting RNA expression, screening RNA or RNA products, determining functions of lincRNA or non-coding RNA, inducing cell dormancy, inducing cell cycle arrest, reducing cell growth and/or cell proliferation, inducing cell anergy, inducing cell apoptosis, inducing cell necrosis, inducing cell death, and/or inducing programmed cell death. A detailed description of these applications can be found, e.g., in WO 2016/205764 A1, which is incorporated herein by reference in its entirety. In different embodiments, the methods described herein can be performed in vitro, in vivo, or ex vivo.

For example, the CRISPR systems described herein can be administered to a subject having a disease or disorder to target and induce cell death in a cell in a diseased state (e.g., cancer cells or cells infected with an infectious agent). For instance, in some embodiments, the CRISPR systems described herein can be used to target and induce cell death in a cancer cell, wherein the cancer cell is from a subject having a Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, or urinary bladder cancer.

### Modulating Gene Expression

The CRISPR systems described herein can be used to modulate gene expression. The CRISPR systems can be used, together with suitable guide RNAs, to target gene expression, via control of RNA processing. The control of RNA processing can include, e.g., RNA processing reactions such as RNA splicing (e.g., alternative splicing), viral replication, and tRNA biosynthesis. The RNA targeting proteins in combination with suitable guide RNAs can also be used to control RNA activation (RNAa). RNA activation is a small RNA-guided and Argonaute (Ago)-dependent gene regulation phenomenon in which promoter-targeted short double-stranded RNAs (dsRNAs) induce target gene expression at the transcriptional/epigenetic level. RNAa leads to the promotion of gene expression, so control of gene expression may be achieved that way through disruption or reduction of RNAa. In some embodiments, the methods include the use of the RNA targeting CRISPR as substitutes for e.g., interfering ribonucleic acids (such as siRNAs, shRNAs, or dsRNAs). The methods of modulating gene expression are described, e.g., in WO 2016205764, which is incorporated herein by reference in its entirety.

### Controlling RNA Interference

Control over interfering RNAs or microRNAs (miRNA) can help reduce off-target effects by reducing the longevity of the interfering RNAs or miRNAs in vivo or in vitro. In some embodiments, the target RNAs can include interfering RNAs, i.e., RNAs involved in the RNA interference pathway, such as small hairpin RNAs (shRNAs), small interfering (siRNAs), etc. In some embodiments, the target RNAs include, e.g., miRNAs or double stranded RNAs (dsRNA).

In some embodiments, if the RNA targeting protein and suitable guide RNAs are selectively expressed (for example spatially or temporally under the control of a regulated promoter, for example a tissue- or cell cycle-specific promoter and/or enhancer), this can be used to protect the cells or systems (in vivo or in vitro) from RNA interference (RNAi) in those cells. This may be useful in neighboring tissues or cells where RNAi is not required or for the purposes of comparison of the cells or tissues where the CRISPR-associated proteins and suitable crRNAs are and are not expressed (i.e., where the RNAi is not controlled and where it is, respectively). The RNA targeting proteins can be used to control or bind to molecules comprising or consisting of RNAs, such as ribozymes, ribosomes, or riboswitches. In some embodiments, the guide RNAs can recruit the RNA targeting proteins to these molecules so that the RNA targeting proteins are able to bind to them. These methods are described, e.g., in WO 2016205764 and WO 2017070605, both of which are incorporated herein by reference in the entirety.

### Modifying Riboswitches and Controlling Metabolic Regulations

Riboswitches are regulatory segments of messenger RNAs that bind small molecules and in turn regulate gene expression. This mechanism allows the cell to sense the intracellular concentration of these small molecules. A specific riboswitch typically regulates its adjacent gene by altering the transcription, the translation or the splicing of this gene. Thus, in some embodiments, the riboswitch activity can be controlled by the use of the RNA targeting proteins in combination with suitable guide RNAs to target the riboswitches. This may be achieved through cleavage of, or binding to, the riboswitch. Methods of using CRISPR systems to control riboswitches are described, e.g., in WO 2016205764 and WO 2017070605, both of which are incorporated herein by reference in their entireties.

### RNA Modification

In some embodiments, the CRISPR-associated proteins described herein can be fused to a base-editing domain, such as ADAR1, ADAR2, APOBEC, or activation-induced cytidine deaminase (AID), and can be used to modify an RNA sequence (e.g., an mRNA). In some embodiments, the CRISPR-associated protein includes one or more mutations (e.g., in a catalytic domain), which renders the subject CRISPR-associated protein incapable of cleaving RNA (e.g., the dCas13 version of the engineered Class 2 type VI Cas13 protein described herein).

In some embodiments, such CRISPR-associated proteins can be used with an RNA-binding fusion polypeptide comprising a base-editing domain (e.g., ADAR1, ADAR2, APOBEC, or AID) fused to an RNA-binding domain, such as MS2 (also known as MS2 coat protein), Qbeta (also known as Qbeta coat protein), or PP7 (also known as PP7 coat protein). The amino acid sequences of the RNA-binding domains MS2, Qbeta, and PP7 are provided below:

MS2 (MS2 coat protein) (SEQ ID NO: 98)

Qbeta (Qbeta coat protein) (SEQ ID NO: 99)

PP7 (PP7 coat protein) (SEQ ID NO: 100)

In some embodiments, the RNA binding domain can bind to a specific sequence (e.g., an aptamer sequence) or secondary structure motifs on a crRNA of the system described herein (e.g., when the crRNA is in an effector-crRNA complex), thereby recruiting the RNA binding fusion polypeptide (which has a base-editing domain) to the effector complex. For example, in some embodiments, the CRISPR system includes a CRISPR associated protein, a crRNA having an aptamer sequence (e.g., an MS2 binding loop, a QBeta binding loop, or a PP7 binding loop), and a RNA-binding fusion polypeptide having a base-editing domain fused to an RNA-binding domain that specifically binds to the aptamer sequence. In this system, the CRISPR-associated protein forms a complex with the crRNA having the aptamer sequence. Further the RNA-binding fusion polypeptide binds to the crRNA (via the aptamer sequence) thereby forming a tripartite complex that can modify a target RNA.

Methods of using CRISPR systems for base editing are described, e.g., in International Publication No. WO 2017/219027, which is incorporated herein by reference in its entirety, and in particular with respect to its discussion of RNA modification.

### RNA Splicing

In some embodiments, an inactivated or dCas13 version of the engineered Class 2 type VI Cas13 protein substantially lacking collateral activity described herein (e.g., an engineered CRISPR associated protein having one or more further mutations in a catalytic domain) can be used to target and bind to specific splicing sites on RNA transcripts. Binding of the inactivated CRISPR-associated protein to the RNA may sterically inhibit interaction of the spliceosome with the transcript, enabling alteration in the frequency of generation of specific transcript isoforms. Such method can be used to treat disease through exon skipping such that an exon having a mutation may be skipped in a mature protein. Methods of using CRISPR systems to alter splicing are described, e.g., in International Publication No. WO 2017/219027, which is incorporated herein by reference in its entirety, and in particular with respect to its discussion of RNA splicing.

### Therapeutic Applications

The CRISPR systems described herein can have various therapeutic applications. Such applications may be based on one or more of the abilities below, both in vitro and in vivo, of the subject engineered Cas13, e.g., engineered CRISPR/Cas13e or Cas13f systems: induce cellular senescence, induce cell cycle arrest, inhibit cell growth and/or proliferation, induce apoptosis, induce necrosis, etc.

In some embodiments, the new engineered CRISPR systems can be used to treat various diseases and disorders, e.g., genetic disorders (e.g., monogenetic diseases), diseases that can be treated by nuclease activity (e.g., Pcsk9 targeting, Duchenne Muscular Dystrophy (DMD), BCL11a targeting), and various cancers, etc.

In some embodiments, the CRISPR systems described herein can be used to edit a target nucleic acid to modify the target nucleic acid (e.g., by inserting, deleting, or mutating one or more nucleic acid residues).

In one aspect, the CRISPR systems described herein can be used for treating a disease caused by overexpression of RNAs, toxic RNAs, and/or mutated RNAs (e.g., splicing defects or truncations). For example, expression of toxic RNAs may be associated with the formation of nuclear inclusions and late-onset degenerative changes in brain, heart, or skeletal muscle. In some embodiments, the disorder is myotonic dystrophy. In myotonic dystrophy, the main pathogenic effect of the toxic RNAs is to sequester binding proteins and compromise the regulation of alternative splicing (see, e.g., Osborne et al., “RNA-dominant diseases,” Hum. Mol. Genet., 2009 Apr. 15; 18(8):1471-81). Myotonic dystrophy (dystrophia myotonica (DM)) is of particular interest to geneticists because it produces an extremely wide range of clinical features. The classical form of DM, which is now called DM type 1 (DM1), is caused by an expansion of CTG repeats in the 3′-untranslated region (UTR) of DMPK, a gene encoding a cytosolic protein kinase. The CRISPR systems as described herein can target overexpressed RNA or toxic RNA, e.g., the DMPK gene or any of the mis-regulated alternative splicing in DM1 skeletal muscle, heart, or brain.

The CRISPR systems described herein can also target trans-acting mutations affecting RNA-dependent functions that cause various diseases such as, e.g., Prader Willi syndrome, Spinal muscular atrophy (SMA), and Dyskeratosis congenita. A list of diseases that can be treated using the CRISPR systems described herein is summarized in Cooper et al., “RNA and disease,” Cell, 136.4 (2009): 777-793, and WO 2016/205764 A1, both of which are incorporated herein by reference in the entirety. Those of skill in this field will understand how to use the new CRISPR systems to treat these diseases.

The CRISPR systems described herein can also be used in the treatment of various tauopathies, including, e.g., primary and secondary tauopathies, such as primary age-related tauopathy (PART)/Neurofibrillary tangle (NFT)-predominant senile dementia (with NFTs similar to those seen in Alzheimer Disease (AD), but without plaques), dementia pugilistica (chronic traumatic encephalopathy), and progressive supranuclear palsy. A useful list of tauopathies and methods of treating these diseases are described, e.g., in WO 2016205764, which is incorporated herein by reference in its entirety.

The CRISPR systems described herein can also be used to target mutations disrupting the cis-acting splicing codes that can cause splicing defects and diseases. These diseases include, e.g., motor neuron degenerative disease that results from deletion of the SMN1 gene (e.g., spinal muscular atrophy), Duchenne Muscular Dystrophy (DMD), frontotemporal dementia, and Parkinsonism linked to chromosome 17 (FTDP-17), and cystic fibrosis.

The CRISPR systems described herein can further be used for antiviral activity, in particular against RNA viruses. The CRISPR-associated proteins can target the viral RNAs using suitable guide RNAs selected to target viral RNA sequences.

The CRISPR systems described herein can also be used to treat a cancer in a subject (e.g., a human subject). For example, the CRISPR-associated proteins described herein can be programmed with crRNA targeting a RNA molecule that is aberrant (e.g., comprises a point mutation or are alternatively-spliced) and found in cancer cells to induce cell death in the cancer cells (e.g., via apoptosis).

The CRISPR systems described herein can also be used to treat an autoimmune disease or disorder in a subject (e.g., a human subject). For example, the CRISPR-associated proteins described herein can be programmed with crRNA targeting a RNA molecule that is aberrant (e.g., comprises a point mutation or are alternatively-spliced) and found in cells responsible for causing the autoimmune disease or disorder.

Further, the CRISPR systems described herein can also be used to treat an infectious disease in a subject. For example, the CRISPR-associated proteins described herein can be programmed with crRNA targeting a RNA molecule expressed by an infectious agent (e.g., a bacteria, a virus, a parasite or a protozoan) in order to target and induce cell death in the infectious agent cell. The CRISPR systems may also be used to treat diseases where an intracellular infectious agent infects the cells of a host subject. By programming the CRISPR-associated protein to target a RNA molecule encoded by an infectious agent gene, cells infected with the infectious agent can be targeted and cell death induced.

Furthermore, in vitro RNA sensing assays can be used to detect specific RNA substrates. The CRISPR-associated proteins can be used for RNA-based sensing in living cells. Examples of applications are diagnostics by sensing of, for examples, disease-specific RNAs.

A detailed description of therapeutic applications of the CRISPR systems described herein can be found, e.g., in U.S. Pat. No. 8,795,965, EP3009511, WO 2016205764, and WO 2017070605; each of which is incorporated herein by reference in its entirety.

### Cells and Progenies Thereof

In certain embodiments, the methods of the invention can be used to introduce the CRISPR systems described herein into a cell, and cause the cell and/or its progeny to alter the production of one or more cellular produces, such as antibody, starch, ethanol, or any other desired products. Such cells and progenies thereof are within the scope of the invention.

In certain embodiments, the methods and/or the CRISPR systems described herein lead to modification of the translation and/or transcription of one or more RNA products of the cells. For example, the modification may lead to increased transcription/translation/expression of the RNA product. In other embodiments, the modification may lead to decreased transcription/translation/expression of the RNA product.

In certain embodiments, the cell is a prokaryotic cell.

In certain embodiments, the cell is a eukaryotic cell, such as a mammalian cell, including a human cell (a primary human cell or an established human cell line). In certain embodiments, the cell is a non-human mammalian cell, such as a cell from a non-human primate (e.g., monkey), a cow/bull/cattle, sheep, goat, pig, horse, dog, cat, rodent (such as rabbit, mouse, rat, hamster, etc). In certain embodiments, the cell is from fish (such as salmon), bird (such as poultry bird, including chick, duck, goose), reptile, shellfish (e.g., oyster, claim, lobster, shrimp), insect, worm, yeast, etc. In certain embodiments, the cell is from a plant, such as monocot or dicot. In certain embodiment, the plant is a food crop such as barley, cassava, cotton, groundnuts or peanuts, maize, millet, oil palm fruit, potatoes, pulses, rapeseed or canola, rice, rye, sorghum, soybeans, sugar cane, sugar beets, sunflower, and wheat. In certain embodiment, the plant is a cereal (barley, maize, millet, rice, rye, sorghum, and wheat). In certain embodiment, the plant is a tuber (cassava and potatoes). In certain embodiment, the plant is a sugar crop (sugar beets and sugar cane). In certain embodiment, the plant is an oil-bearing crop (soybeans, groundnuts or peanuts, rapeseed or canola, sunflower, and oil palm fruit). In certain embodiment, the plant is a fiber crop (cotton). In certain embodiment, the plant is a tree (such as a peach or a nectarine tree, an apple or pear tree, a nut tree such as almond or walnut or pistachio tree, or a citrus tree, e.g., orange, grapefruit or lemon tree), a grass, a vegetable, a fruit, or an algae. In certain embodiment, the plant is a nightshade plant; a plant of the genus Brassica; a plant of the genus Lactuca; a plant of the genus Spinacia; a plant of the genus Capsicum; cotton, tobacco, asparagus, carrot, cabbage, broccoli, cauliflower, tomato, eggplant, pepper, lettuce, spinach, strawberry, blueberry, raspberry, blackberry, grape, coffee, cocoa, etc.

A related aspect provides cells or progenies thereof modified by the methods of the invention using the CRISPR systems described herein.

In certain embodiments, the cell is modified in vitro, in vivo, or ex vivo.

In certain embodiments, the cell is a stem cell.

### 8. Delivery

Through this disclosure and the knowledge in the art, the CRISPR systems described herein comprising an engineered Class 2 type VI Cas13 protein, such as those either substantially lacking or having enhanced collateral activity (such as Cas13e or Cas13f), or any of the components thereof described herein (Cas13 proteins, derivatives, functional fragments or the various fusions or adducts thereof, and guide RNA/crRNA), nucleic acid molecules thereof, and/or nucleic acid molecules encoding or providing components thereof, can be delivered by various delivery systems such as vectors, e.g., plasmids and viral delivery vectors, using any suitable means in the art. Such methods include (and are not limited to) electroporation, lipofection, microinjection, transfection, sonication, gene gun, etc.

In certain embodiments, the CRISPR-associated proteins and/or any of the RNAs (e.g., guide RNAs or crRNAs) and/or accessory proteins can be delivered using suitable vectors, e.g., plasmids or viral vectors, such as adeno-associated viruses (AAV), lentiviruses, adenoviruses, retroviral vectors, and other viral vectors, or combinations thereof. The proteins and one or more crRNAs can be packaged into one or more vectors, e.g., plasmids or viral vectors. For bacterial applications, the nucleic acids encoding any of the components of the CRISPR systems described herein can be delivered to the bacteria using a phage. Exemplary phages, include, but are not limited to, T4 phage, Mu, λ phage, T5 phage, T7 phage, T3 phage, Φ29, M13, MS2, Qβ, and Φ174.

In some embodiments, the vectors, e.g., plasmids or viral vectors, are delivered to the tissue of interest by, e.g., intramuscular injection, intravenous administration, transdermal administration, intranasal administration, oral administration, or mucosal administration. Such delivery may be either via a single dose, or multiple doses. One skilled in the art understands that the actual dosage to be delivered herein may vary greatly depending upon a variety of factors, such as the vector choices, the target cells, organisms, tissues, the general conditions of the subject to be treated, the degrees of transformation/modification sought, the administration routes, the administration modes, the types of transformation/modification sought, etc.

In certain embodiments, the delivery is via adenoviruses, which can be at a single dose containing at least 1×10⁵ particles (also referred to as particle units, pu) of adenoviruses. In some embodiments, the dose preferably is at least about 1×10⁶ particles, at least about 1×10⁷ particles, at least about 1×10⁸ particles, and at least about 1×10⁹ particles of the adenoviruses. The delivery methods and the doses are described, e.g., in WO 2016205764 A1 and U.S. Pat. No. 8,454,972 B2, both of which are incorporated herein by reference in the entirety.

In some embodiments, the delivery is via plasmids. The dosage can be a sufficient number of plasmids to elicit a response. In some cases, suitable quantities of plasmid DNA in plasmid compositions can be from about 0.1 to about 2 mg. Plasmids will generally include (i) a promoter; (ii) a sequence encoding a nucleic acid-targeting CRISPR-associated proteins and/or an accessory protein, each operably linked to a promoter (e.g., the same promoter or a different promoter); (iii) a selectable marker; (iv) an origin of replication; and (v) a transcription terminator downstream of and operably linked to (ii). The plasmids can also encode the RNA components of a CRISPR complex, but one or more of these may instead be encoded on different vectors. The frequency of administration is within the ambit of the medical or veterinary practitioner (e.g., physician, veterinarian), or a person skilled in the art.

In another embodiment, the delivery is via liposomes or lipofection formulations and the like, and can be prepared by methods known to those skilled in the art. Such methods are described, for example, in WO 2016205764 and U.S. Pat. Nos. 5,593,972; 5,589,466; and 5,580,859; each of which is incorporated herein by reference in its entirety.

In some embodiments, the delivery is via nanoparticles or exosomes. For example, exosomes have been shown to be particularly useful in delivery RNA.

Further means of introducing one or more components of the new CRISPR systems to the cell is by using cell penetrating peptides (CPP). In some embodiments, a cell penetrating peptide is linked to the CRISPR-associated proteins. In some embodiments, the CRISPR-associated proteins and/or guide RNAs are coupled to one or more CPPs to effectively transport them inside cells (e.g., plant protoplasts). In some embodiments, the CRISPR-associated proteins and/or guide RNA(s) are encoded by one or more circular or non-circular DNA molecules that are coupled to one or more CPPs for cell delivery.

CPPs are short peptides of fewer than 35 amino acids derived either from proteins or from chimeric sequences capable of transporting biomolecules across cell membrane in a receptor independent manner. CPPs can be cationic peptides, peptides having hydrophobic sequences, amphipathic peptides, peptides having proline-rich and anti-microbial sequences, and chimeric or bipartite peptides. Examples of CPPs include, e.g., Tat (which is a nuclear transcriptional activator protein required for viral replication by HIV type 1), penetratin, Kaposi fibroblast growth factor (FGF) signal peptide sequence, integrin β3 signal peptide sequence, polyarginine peptide Args sequence, Guanine rich-molecular transporters, and sweet arrow peptide. CPPs and methods of using them are described, e.g., in Hällbrink et al., “Prediction of cell-penetrating peptides,” Methods Mol. Biol., 2015; 1324:39-58; Ramakrishna et al., “Gene disruption by cell-penetrating peptide-mediated delivery of Cas9 protein and guide RNA,” Genome Res., 2014 June; 24(6):1020-7; and WO 2016205764 A1; each of which is incorporated herein by reference in its entirety.

Various delivery methods for the CRISPR systems described herein are also described, e.g., in U.S. Pat. No. 8,795,965, EP3009511, WO 2016205764, and WO 2017070605; each of which is incorporated herein by reference in its entirety.

### 9. Kits

Another aspect of the invention provides a kit, comprising any two or more components of the subject CRISPR/Cas system described herein comprising an engineered Class 2 type VI Cas13 protein, such as those either substantially lacking or having enhanced collateral activity, such as the Cas13e and Cas13f proteins, derivatives, functional fragments or the various fusions or adducts thereof, guide RNA/crRNA, complexes thereof, vectors encompassing the same, or host encompassing the same.

In certain embodiments, the kit further comprises an instruction to use the components encompassed therein, and/or instructions for combining with additional components that may be available elsewhere.

In certain embodiments, the kit further comprises one or more nucleotides, such as nucleotide(s) corresponding to those useful to insert the guide RNA coding sequence into a vector and operably linking the coding sequence to one or more control elements of the vector.

In certain embodiments, the kit further comprises one or more buffers that may be used to dissolve any of the components, and/or to provide suitable reaction conditions for one or more of the components. Such buffers may include one or more of PBS, HEPES, Tris, MOPS, Na₂CO₃, NaHCO₃, NaB, or combinations thereof. In certain embodiments, the reaction condition includes a proper pH, such as a basic pH. In certain embodiments, the pH is between 7-10.

In certain embodiments, any one or more of the kit components may be stored in a suitable container.

### EXAMPLES

### Example 1 Identification and Characterization of Engineered Cas13e Effector Enzymes Having Reduced Collateral Effect

This example demonstrates that collateral effect or non-sequence-specific endonuclease activity of the Cas13 enzymes (e.g., Cas13e) can be largely reduced by introducing mutations that reduce the affinity between Cas13e and potential RNA targets (sequence specific or non-sequence specific targets), thus disproportionally reducing collateral non-sequence-specific endonuclease activity, while substantially maintaining sequence-specific endonuclease activity against the target RNA, partly due to the binding between the guide sequence and the target RNA. See FIG. 1.

Using the I-TASSER website (zhanglab.ccmb.med.umich.edu/I-TASSER), the 3D structure of Cas13e was predicted. Further, using the NCBI web tool (ncbi.nlm.nih.gov/Structure/icn3d/full.html), or PyMOL, the predicted structure was visualized. Based on the relevant sequence information, sequences that are spatially close to the two HEPN RXXXXH sequences were analyzed in Cas13e. See FIG. 2. These spatially close sequences were predicted to participate in binding of the target RNAs (both guide-sequence-specific and non-guide sequence-specific target RNAs) by the Cas13e effector enzyme, before the target RNA molecules were cleaved in the catalytic domain of the Cas13e endonuclease.

Based on this theory, sequences that are spatially close to the two HEPN domains in Cas13e, e.g., residues 2-187 and 634-755 that are around the two HEPN domains, respectively, as well as the spatially close region between residues 227-242, were systematically mutated (see FIG. 3) over the entire regions of interest. Within each region, mutations were focused on those residues that likely participate in RNA binding (or RNA binding hotspots), namely those with nitrogen-containing and/or positively charged side chain groups such as R, K, H, N, or Q residues. These mutation hotspot residues were systematically changed to Ala to avoid catastrophic disruption of the overall protein folding, based on the principle of Ala scanning mutagenesis.

In order to facilitate further screening and selection, to the ends of each selected mutagenesis region (see FIG. 3), a BpiI recognition sequence was introduced, i.e., GTCTTC on one end (corresponding to the di-peptide sequence of ValPhe or VF), and GAAGAC on the other end (corresponding to the di-peptide sequence of GluAsp or ED). In general, 5-8 mutations were introduced between each pair of BpiI recognition sequences. In some mutation regions, Y/S/T>A style mutants were introduced.

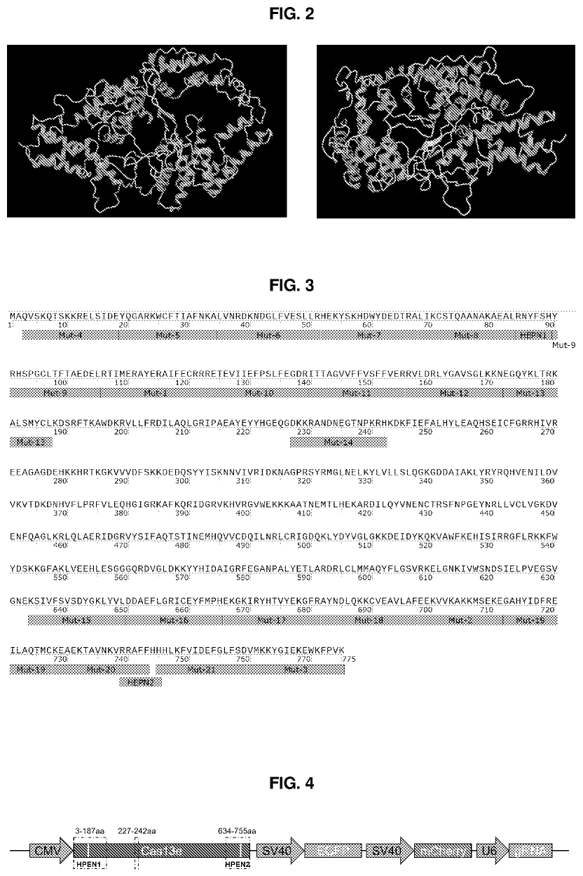

To facilitate further characterization, an EGFP-mCherry double fluorescent reporting system was constructed (see FIG. 4). In that system, expression of EGFP and mCherry were under the separate but identical control of their respective SV40 promoters, in order to ensure that their mRNA ratio was relatively stably maintained in transfected cells. The gRNA of this system specifically targeted EGFP coding sequence (mRNA). In addition, each tested engineered Cas13e has a NLS (nuclear localization sequence) at the N-terminus, as well as the C-terminus. The CMV promoter was used to drive the expression of the engineered Cas13e.

The sequences of the EGFP and mCherry reporters are in SEQ ID NOs: 1 and 2. The gRNA is SEQ ID NO: 3. Wild type Cas13e protein is SEQ ID NO: 4, and its codon-optimized polynucleotide coding sequence is SEQ ID NO: 5.

Human HEK293T cells were cultured in 24-well tissue culture plates according to standard methods, before the double-fluorescent reporting system plasmid was transfected into the cells using standard polyethylenimine (PEI) transfection. Transfected cells were then cultured at 37° C. under CO₂ for 48 hrs. EGFP and mCherry signals were detected using FACS.

The standard for selecting engineered Cas13e with reduced collateral effect, using the double-fluorescent reporting system, was following:

1) mutant/engineered Cas13e has similar/equivalent EGFP signal compared to the wild-type Cas13e, indicating that the guide-sequence-specific cleavage of the target RNA (EGFP) was not/little affected by the mutations in the engineered Cas13e;

2) mutant/engineered Cas13e has similar/equivalent mCherry signal compared to the nuclease dead dCas13e, indicating that the non-sequence-specific cleavage of the non-target RNA (mCherry) was non-existing in the engineered Cas13e, just like dCas13e that is unable to cleave mCherry mRNA.

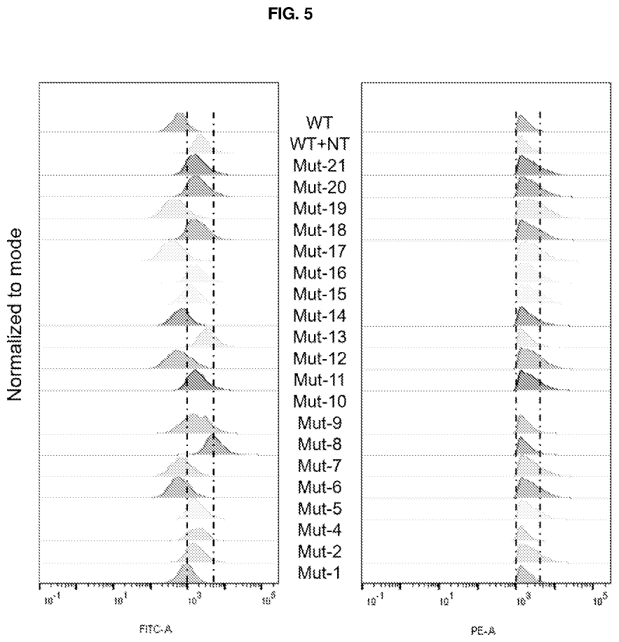

Based on the above standard and further characterization, 5 distinct engineered Cas13e were identified, each with much reduced collateral effect compared to wild-type Cas13e (see FIGS. 5-7), including Mut-6, -7, -12, -17, and -19. The complete protein sequences of these engineered Cas13e are in SEQ ID NOs: 6-10, respectively. The coding sequences are SEQ ID NOs: 11-15, respectively.

For comparison, in the Mut-6 mutation region, the corresponding wild-type sequence and mutant sequence listed below in SEQ ID NOs: 16 and 17, with changed sequences double underlined. The corresponding nucleotide sequences are in SEQ ID NOs: 18 and 19.

(SEQ ID NO: 16)
LVNRDKNDGLFVESLLR
(SEQ ID NO: 17)
VFAAAAAAGLFVASLED
(SEQ ID NO:18)
CTGGTGAACCGGGACAAGAACGACGGCCTG
TTCGTGGAAAGCCTGCTGAGA
(SEQ ID NO: 19)
gtcttcgcCgccGcCgccgccGcC
GGCCTGTTCGTGGccAGCCTGgaagac

In the Mut-7 mutation region, the corresponding wild-type sequence and mutant sequence listed below in SEQ ID NOs: 20 and 21, with changed sequences double underlined. The corresponding nucleotide sequences are in SEQ ID NOs: 22 and 23.

(SEQ ID NO: 20)
HEKYSKHDWYDEDTRA
(SEQ ID NO: 21)
VFAYSAAAWYAAATED
(SEQ ID NO: 22)
CACGAGAAGTACAGCAAGCACGACTG
GTACGACGAAGATACCCGGGCC
(SEQ ID NO: 23)
gtcttcgccTACAGCgccgccgccTGGT
ACGccgcccgccACCgaagac

In the Mut-12 mutation region, the corresponding wild-type sequence and mutant sequence listed below in SEQ ID NOs: 22 and 23, with changed sequences double underlined. The corresponding nucleotide sequences are in SEQ ID NOs: 24 and 25.

(SEQ ID NO: 24)
RVLDRLYGAVSGLKKN
(SEQ ID NO: 25)
VFLAALAGAVAGLAED
(SEQ ID NO: 26)
AGAGTGCTGGATCGGCTGTATGGAGCCGTG
TCCGGCCTGAAGAAGAAT
(SEQ ID NO: 27
gtcttcCTGGccgccCTGgccG
GAGCCGTGgCCGGCCTGgccgaagac

In the Mut-17 mutation region, the corresponding wild-type sequence and mutant sequence listed below in SEQ ID NOs: 28 and 29, with changed sequences double underlined. The corresponding nucleotide sequences are in SEQ ID NOs: 30 and 31.

(SEQ ID NO: 28)
EKGKIRYHTVYEKGFR
(SEQ ID NO: 29)
VFGAIAAATVYAAGED
(SEQ ID NO: 30)
GAAAAGGGCAAGATCCGGTACCACACAGTGTACGAAAAGGGCTTTAGA
(SEQ ID NO: 31)
gtcttcGGCgccATCgccgccgccA
CAGTGTACgccgccGGCgaagac

In the Mut-19 mutation region, the corresponding wild-type sequence and mutant sequence listed below in SEQ ID NOs: 32 and 33, with changed sequences double underlined. The corresponding nucleotide sequences are in SEQ ID NOs: 34 and 35.

(SEQ ID NO: 32)
GAHYIDFREILAQTMC
(SEQ ID NO: 33)
VFAAIAFAAILAQAED
(SEQ ID NO: 34)
GGCGCCCACTACATCGACTTCCGGGAGATCCTGGCCCAGACCATGTGC
(SEQ ID NO: 35)
GtcttcgcCgcCATCGcCTTCgccGccATCCTGGCCCAGgCCgaagaC

Based on further characterization, Mut-17 and Mut-19 essentially eliminated collateral effect of wild-type Cas13e, while maintained relatively high guide-sequence specific endonuclease activity.

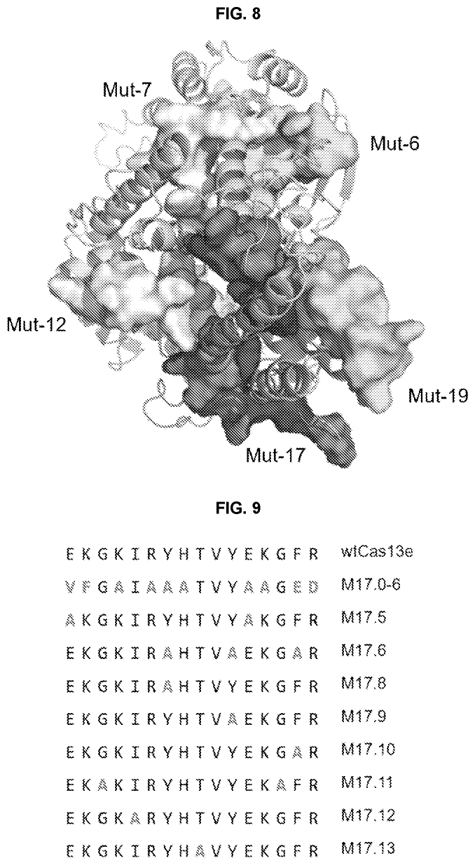

Further, the method described herein has been shown to be able to identify residues for engineering even though these residues are far away from the HEPN domains in primary sequence, but can be shown to be spatially close to the HEPN domains based on predicted 3D structure (using commonly available tools such as PyMOL or I-TASSER). See FIG. 8.

### Example 2 Identification and Characterization of Further Engineered Cas13e Effector Enzymes Point Mutations Having Reduced Collateral Effect

In order to narrow down the key amino acids in the Mut-17 region that affect the bystander effect, a series of 8 mutations in the Mut-17 region were constructed and tested, including M17.5, M17.6, M17.8, M17.9, M17.10, M17.11, M17.12, and M17.13 (see FIG. 9). M17.0-6 is the same as Mut-17.

For comparison, in the M17.5 mutation region, the corresponding wild-type sequence and mutant sequence are listed below in SEQ ID NOs: 28 and 36, with changed sequences double underlined.

(SEQ ID NO: 28)
EKGKIRYHTVYEKGFR
(SEQ ID NO: 36)
AKGKIRYHTVYAKGFR

In the M17.6 mutation region, the corresponding wild-type sequence and mutant sequence are listed below in SEQ ID NOs: 28 and 37, with changed sequences double underlined.

(SEQ ID NO: 28)
EKGKIRYHTVYEKGFR
(SEQ ID NO: 37)
EKGKIRAHTVAEKGAR

In the M17.8 mutation region, the corresponding wild-type sequence and mutant sequence are listed below in SEQ ID NOs: 28 and 38, with changed sequences double underlined.

(SEQ ID NO: 28)
EKGKIRYHTVYEKGFR
(SEQ ID NO: 38)
EKGKIRAHTVYEKGFR

In the M17.9 mutation region, the corresponding wild-type sequence and mutant sequence are listed below in SEQ ID NOs: 28 and 39, with changed sequences double underlined.

(SEQ ID NO: 28)
EKGKIRYHTVYEKGFR
(SEQ ID NO: 39)
EKGKIRYHTVAEKGFR

In the M17.10 mutation region, the corresponding wild-type sequence and mutant sequence are listed below in SEQ ID NOs: 28 and 40, with changed sequences double underlined.

| (SEQ ID NO: 28) | |
|---|---|
| | EKGKIRYHTVYEKGFR |
| (SEQ ID NO: 40) | |
| | EKGKIRYHTVYEKGAR |

In the M17.11 mutation region, the corresponding wild-type sequence and mutant sequence are listed below in SEQ ID NOs: 28 and 41, with changed sequences double underlined.

| (SEQ ID NO: 28) | |
|---|---|
| | EKGKIRYHTVYEKGFR |
| (SEQ ID NO: 41) | |
| | EKAKIRYHTVYEKAFR |

In the M17.12 mutation region, the corresponding wild-type sequence and mutant sequence are listed below in SEQ ID NOs: 28 and 42, with changed sequences double underlined.

| (SEQ ID NO: 28) | |
|---|---|
| | EKGKIRYHTVYEKGFR |
| (SEQ ID NO: 42) | |
| | EKGKARYHTVYEKGFR |

In the M17.13 mutation region, the corresponding wild-type sequence and mutant sequence are listed below in SEQ ID NOs: 28 and 43, with changed sequences double underlined.

| (SEQ ID NO: 28) | |
|---|---|
| | EKGKIRYHTVYEKGFR |
| (SEQ ID NO: 43) | |
| | EKGKIRYHAVYEKGFR |

Based on this further characterization, and consistent with previous results, most tested point mutations within the Mut-17 region do not have significant effect on the guide sequence-dependent RNase activity (see FIG. 11)—most mutants have comparable levels of guide sequence-dependent RNase activity compared to wild-type Cas13e.1.

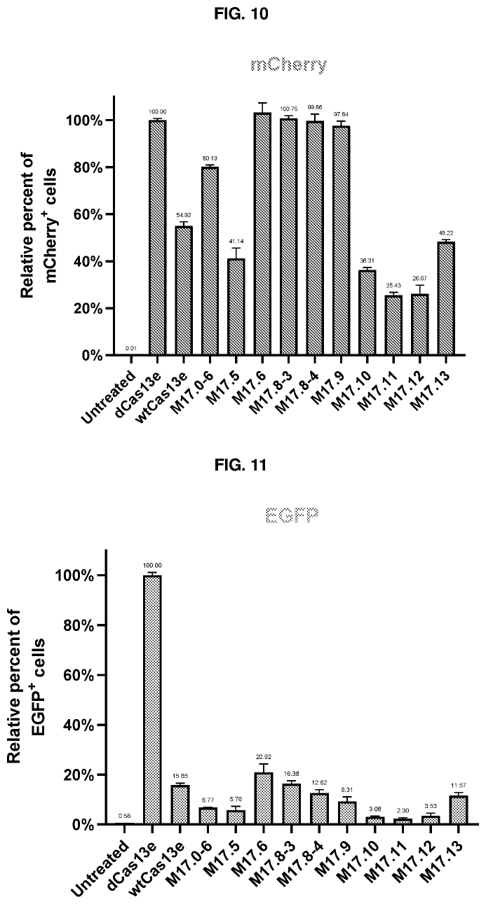

In contrast, point mutations M17.6, M17.8 and M17.9 (SEQ ID NOs: 37-39) essentially eliminated collateral effect of wild-type Cas13e to dCas13e.1 level, while the other point mutations retained different degrees of collateral effect compared to wild-type Cas13e.1, including in some cases enhanced collateral effect (see FIG. 10). Therefore, residues Y672 and Y676 in the Mut-17 region of wtCas13e.1 appear to be two key residues that affect the collateral circumcision effect of wild-type Cas13e.1.

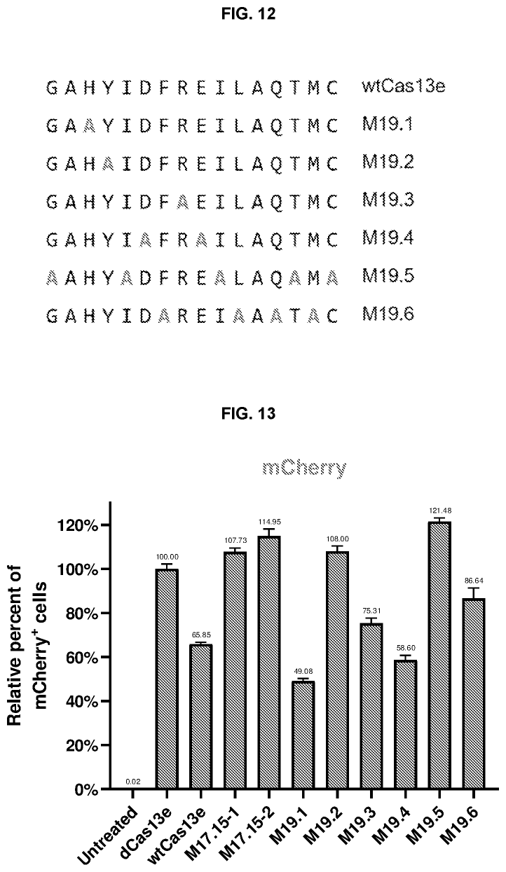

Similarly, in order to narrow down the key amino acid residues in the Mut-19 region that affect the collateral activity, a series of 6 mutants in the Mut-19 region were constructed and tested (see FIG. 12), including M19.1, M19.2, M19.3, M19.4, M19.5, and M19.6.

For comparison, in the M19.1 mutation region, the corresponding wild-type Cas13e.1 sequence and mutant sequences are listed below in SEQ ID NOs: 32 and 44, with changed sequences double underlined.

| (SEQ ID NO: 32) | |
|---|---|
| | GAHYIDFREILAQTMC |
| (SEQ ID NO: 44) | |
| | GAAYIDFREILAQTMC |

In the M19.2 mutation region, the corresponding wild-type Cas13e.1 sequence and mutant sequences are listed below in SEQ ID NOs: 32 and 45, with changed sequences double underlined.

| (SEQ ID NO: 32) | |
|---|---|
| | GAHYIDFREILAQTMC |
| (SEQ ID NO: 45) | |
| | GAHAIDFREILAQTMC |

In the M19.3 mutation region, the corresponding wild-type Cas13e.1 sequence and mutant sequences are listed below in SEQ ID NOs: 32 and 46, with changed sequences double underlined.

| (SEQ ID NO: 32) | |
|---|---|
| | GAHYIDFREILAQTMC |
| (SEQ ID NO: 46) | |
| | GAHYIDFAEILAQTMC |

In the M19.4 mutation region, the corresponding wild-type Cas13e.1 sequence and mutant sequences are listed below in SEQ ID NOs: 32 and 47, with changed sequences double underlined.

| (SEQ ID NO: 32) | |
|---|---|
| | GAHYIDFREILAQTMC |
| (SEQ ID NO: 47) | |
| | GAHYIAFRAILAQTMC |

In the M19.5 mutation region, the corresponding wild-type Cas13e.1 sequence and mutant sequences are listed below in SEQ ID NOs: 32 and 48, with changed sequences double underlined.

| (SEQ ID NO: 32) | |
|---|---|
| | GAHYIDFREILAQTMC |
| (SEQ ID NO: 48) | |
| | AAHYADFREALAQAMA |

In the M19.6 mutation region, the corresponding wild-type Cas13e.1 sequence and mutant sequences are listed below in SEQ ID NOs: 32 and 49, with changed sequences double underlined.

| (SEQ ID NO: 32) | |
|---|---|
| | GAHYIDFREILAQTMC |
| (SEQ ID NO: 49) | |
| | GAHYIDAREIAAATAC |

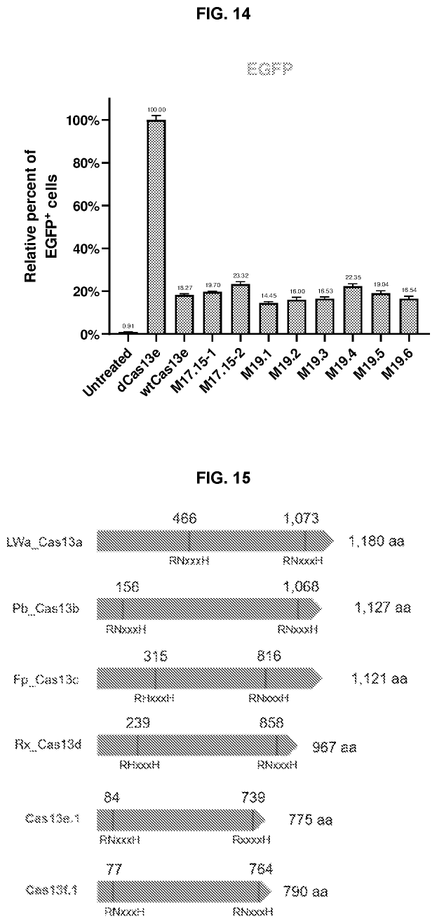

Based on this further characterization, and consistent with previous results, most tested point mutations within the Mut-19 region do not have significant effect on the guide sequence-dependent RNase activity (see FIG. 14) — most mutants have comparable levels of guide sequence-dependent RNase activity compared to wild-type Cas13e.1.

In contrast, point mutations M19.2 and M19.5 (SEQ ID NOs: 45 and 48) essentially eliminated collateral effect of wild-type Cas13e to dCas13e.1 level, while the other point mutations retained different degrees of collateral effect compared to wild-type Cas13e.1 (see FIG. 13). Therefore, residues Y715 in the Mut-19 region of wtCas13e.1 appear to be a key residue that affects the collateral circumcision effect of wild-type Cas13e.1.

### Example 3 Collateral Effects of Cas13 in Mammalian Cells

Collateral RNA degradation by the Cas13 family of effector enzymes has previously been found in glioma cells and flies, but its presence in mammalian cells has not been definitively demonstrated. Based on the fast and sensitive dual-fluorescence reporter system for detecting collateral effects as described herein, this example demonstrates that Cas13 could indeed induce substantial collateral effects in HEK293T cells when targeting either exogenous and endogenous genes. In particular, Cas13d was shown to mediate transcriptome-wide RNA off-target editing, causing cell growth arrest and reducing cell viability.

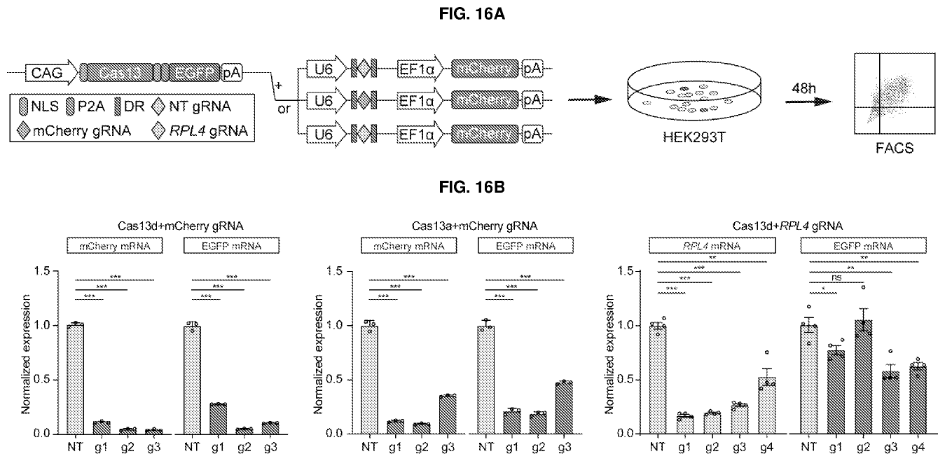

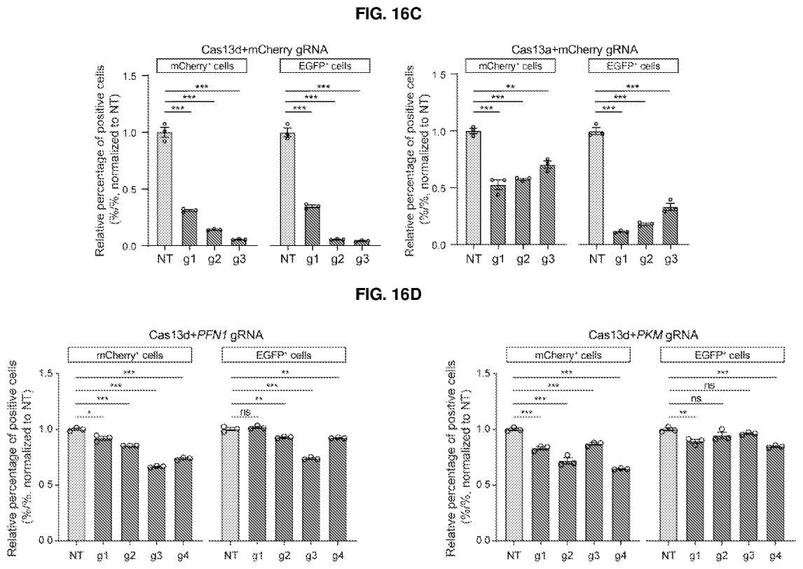

Specifically, to evaluate the collateral effects of Cas13 in mammalian cells, Cas13 (Cas13a or Cas13d) were co-transfected with EGFP and mCherry coding sequences, together with targeted (against mCherry) or non-targeted (NT, control) guide RNA (gRNA) into HEK293T cells. Expression levels of the targeted mCherry and the non-targeted EGFP were measured 48 hrs after transfection (FIG. 16A).

It was found that, with three different mCherry gRNAs, both Cas13a and Cas13d not only mediated expected decrease of mCherry fluorescence intensity, but also caused significant decrease of EGFP fluorescence intensity, as compared to NT gRNA (FIG. 16C). This result was further confirmed by EGFP and mCherry transcripts analysis with qPCR (FIG. 16B).

Together, these findings showed that collateral effects of Cas13-mediated RNA reduction were detectable in the mammalian HEK293T cells when targeting transiently overexpressed exogenous genes.

However, the collateral effects are not limited to transiently overexpressed exogenous genes. The data presented herein also demonstrates that Cas13d could induce collateral effects when targeting endogenous genes in HEK293T.

Flow cytometry experiments showed that Cas13d-mediated knockdown induced a substantial collateral cleavage (as indicated by the reduction of EGFP and mCherry fluorescence) when targeting the endogenous RPL4 gene (FIG. 16B), and a slight collateral cleavage when targeting the endogenous PKM and PFN1 genes (FIG. 16D).

Furthermore, by determining the RNA-targeting efficiency on RPL4 with four different gRNAs (gRNA-1 to gRNA-4), consistently robust knockdown for RPL4 with each gRNA by Cas13 targeting was observed, along with notable knockdown of EGFP transcript with RPL4 gRNA-1, gRNA-3 and gRNA-4, but not gRNA-2 (FIG. 16B, right panel). This observation is consistent with previous reports that different gRNAs exhibited different extent of collateral effects when targeting the same or different transcripts, probably due to the stability of Cas13/gRNA complexes.

Regardless, these findings convincingly demonstrate that Cas13-mediated RNA knockdown results in substantial collateral effects in mammalian cells, when targeting either exogenous or endogenous genes.

### Example 4 Eliminating Collateral Effects of Cas13d through Mutagenesis

Consistent with what has been shown in Examples 1 and 2 concerning Cas13e, the example demonstrates that the collateral effects of other Cas13 (e.g., Cas13d or CasRx) can also be diminished (even if not completely eliminated) via mutagenesis, based on the hypothesis that changing RNA-binding cleft proximal to catalytic sites RXXXXH in HEPN domains may selectively decrease promiscuous RNA binding and non-target cleavage while maintain on-target RNA cleavage.

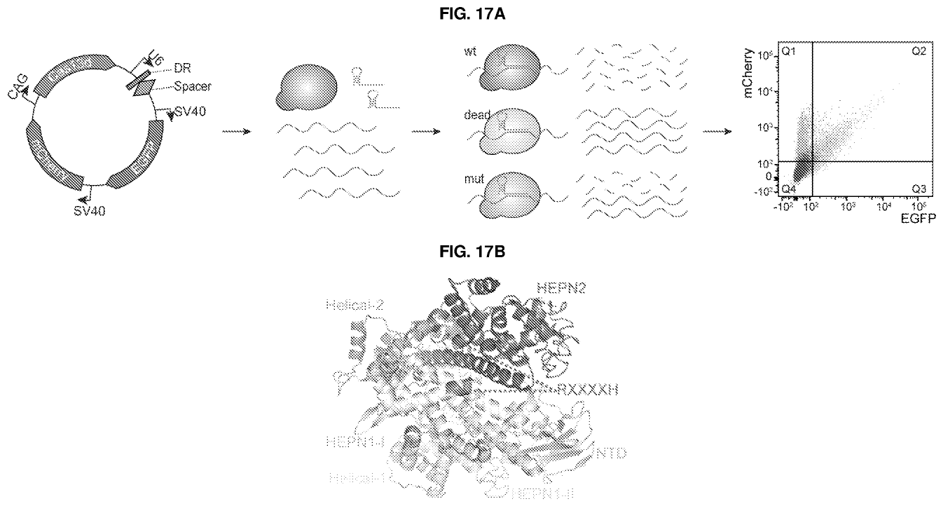

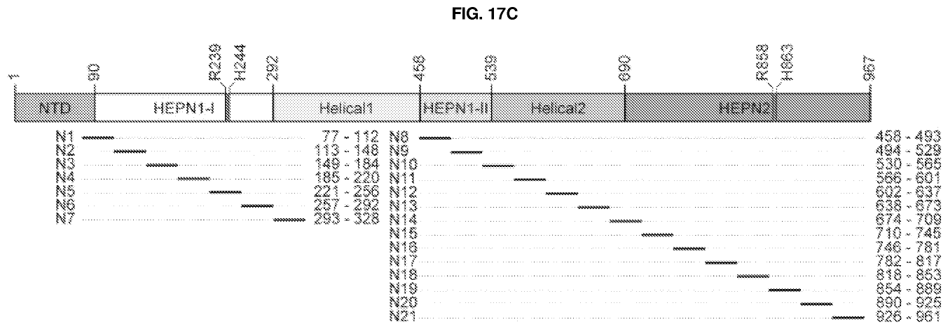

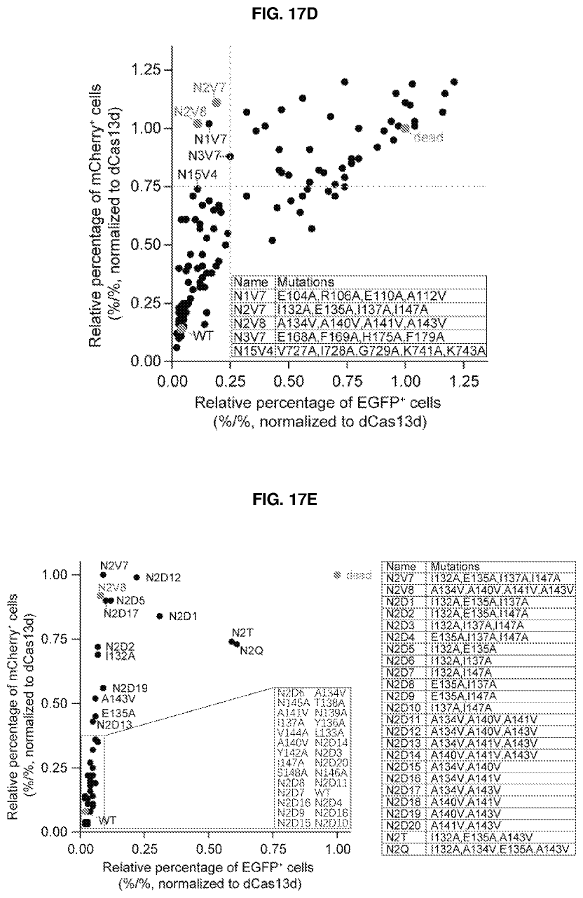

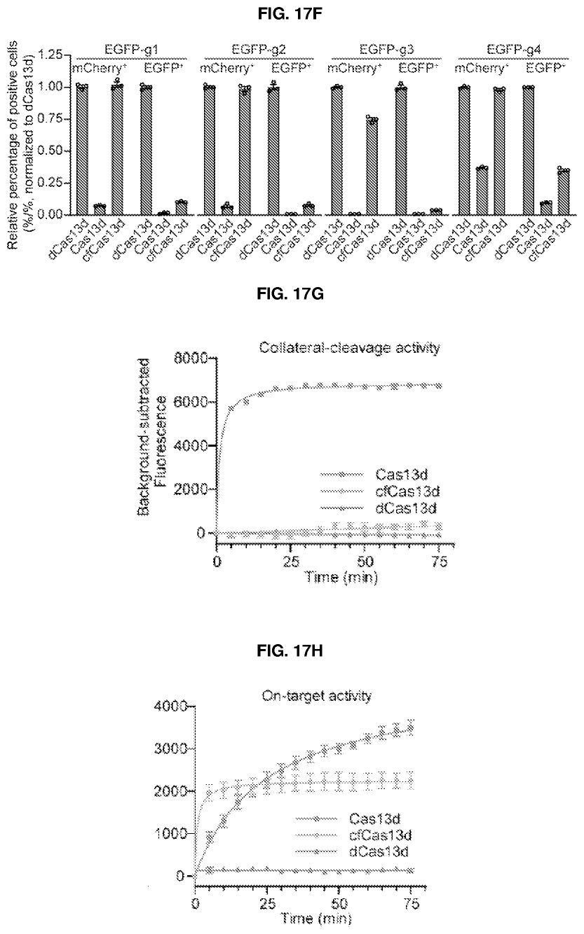

Specifically, as before, a publicly available online tool TASSER was used to predict the 3D structure of Cas13d, and the predicted structure was visualized with PyMOL in order to determine the position of the various structual domains in 3D (see FIGS. 17B and 17C).

Then an unbiased screening system was designed based on the dual-fluorescence approach described above, in which coding sequences for EGFP, mCherry, EGFP-targeting gRNA, together with each Cas13 variants, were inserted into one plasmid for expression in 293T cells. In this system, expression of EGFP and expression of mCherry were driven by the same SV40 promoter, in order to ensure roughly equally stable expression of the reporter genes in the transfected host cell. The gRNA was chosen to be specific for EGFP mRNA. Each coding sequence for Cad13d and variants has an N-terminal and a C-terminal nuclear localization signal (NLS), and expression of Cas13d and variants/mutants was driven by the strong CAG promoter.

The EGFP and mCherry coding sequences are SEQ ID NOs: 1 and 2, respectively. The corresponding DNA sequence of the gRNA is SEQ ID NO: 3. The wild-type Cas13d protein sequence is SEQ ID NO: 101. The coding sequence for the wild-type Cas13d is SEQ ID NO: 102. The CAG promoter sequence is SEQ ID NO: 103. The SV40 promoter sequence is SEQ ID NO: 104.

The HEPN1-I, HEPN1-II, and HEPN2 domains of Cas13d, corresponding to residues 77-328 and 458-961, were chosen for generating a Cas13d mutagenesis library. First, these regions were divided into 21 small segments (N1-N21), each with about 36 residues. More specifically, these 21 mutated regions cover HEPN1-I (N1-N6), HEPN1-II (N8-N10), HEPN2 (N14-N21), Helical-1 (N7) and Helical-2 (N10-N14) domains (FIG. 17C).

To facilitate subsequent selection, a BpiI restriction enzyme recognition site (GTCTTC, corresponding to encoded residues VF; reverse complement GAAGAC, corresponding to encoded residues ED) was introduced at each end of the segments. When producing mutants, all non-Ala residues were substituted by Ala, and all Ala residues were substituted by Val (e.g., replacing all non-alanine to alanine, X>A, and alanine to valine, A>V). About 4-5 total mutations were introduced between the two BpiI sites flanking each segment. The various mutants so generated and their corresponding wild-type sequences (N1L1-N21L, N1R-N21R) are provided below.

| | | SEQ | | SEQ |
|---|---|---|---|---|
| | | ID | | ID |
| Variants | Amino Acids | NO: | DNA sequence | NO: |
| N1L | KGYAVVANNPLYTGPVQ | 105 | AAGGGCTACGCCGTGGTGGCTAACAACCCACTGTACACCGGACCAGTGCAG | 106 |
| N1V1 | AAYAVVAANPLYAAPVQ | 107 | gccgccTACGCCGTGGTGGCTgccAACCCACTGTACgccgccCCAGTGCAG | 108 |
| N1V2 | KGYAAAANAPLYTGPAQ | 109 | AAGGGCTACGCCgccgccGCTAACgccCCACTGTACACCGGACCAgccCAG | 110 |
| N1V3 | KGYVVVVNNPAYTGPVA | 111 | AAGGGCTACgtgGTGGTGgtgAACAACCCAgccTACACCGGACCAGTGgcc | 112 |
| N1-Y79A | KGAAVVANNPLYTGPVQ | 113 | AAGGGCgccGCCGTGGTGGCTAACAACCCACTGTACACCGGACCAGTGCAG | 114 |
| N1-Y88A | KGYAWANNPLATGPVQ | 115 | AAGGGCTACGCCGTGGTGGCTAACAACCCACTGgccACCGGACCAGTGCAG | 116 |
| NIR | QDMLGLKETLEKRYFGESA | 117 | CAGGACATGCTGGGACTGAAGGAGACACTGGAGAAGAGGTACTTCGGCGAGTCCGCC | 118 |
| N1V4 | AAMLGLAETLEARYFGEAA | 119 | gccgccATGCTGGGACTGgccGAGACACTGGAGgccAGGTACTTCGGCGAGgccGCC | 120 |
| N1V5 | QDMAAAKEALEKRYFAESA | 121 | CAGGACATGgccgccgccAAGGAGgccCTGGAGAAGAGGTACTTCgccGAGTCCGCC | 122 |
| N1V6 | QDALGLKATAEKRYAGESA | 123 | CAGGACgccCTGGGACTGAAGgccACAgccGAGAAGAGGTACgccGGCGAGTCCGCC | 124 |
| N1V7 | QDMLGLKETLAKAYFGASV | 125 | CAGGACATGCTGGGACTGAAGGAGACACTGgccAAGgccTACTTCGGCgccTCCgtg | 126 |
| N1-Y107A | QDMLGLKETLEKRAFGESA | 127 | CAGGACATGCTGGGACTGAAGGAGACACTGGAGAAGAGGgccTTCGGCGAGTCCGCC | 128 |
| N2L | DGNDNICIQVIHNILDI | 129 | GACGGAAACGATAACATCTGCATCCAGGTCATCCACAACATCCTGGATATC | 130 |
| N2V1 | AAAANICIQVIHNILAI | 131 | gccgccgccgccAACATCTGCATCCAGGTCATCCACAACATCCTGgccATC | 132 |
| N2V2 | DGNDAICIAAIHAILDI | 133 | GACGGAAACGATgccATCTGCATCgccgccATCCACgccATCCTGGATATC | 134 |
| N2V3 | DGNDNAAIQVIANIADI | 135 | GACGGAAACGATAACgccgccATCCAGGTCATCgccAACATCgccGATATC | 136 |
| N2V4 | DGNDNICAQVAHNALDA | 137 | GACGGAAACGATAACATCTGCgccCAGGTCgccCACAACgccCTGGATgcc | 138 |
| N2R | EKILAEYITNAAYAVNNIS | 139 | GAGAAGATCCTGGCTGAGTACATCACAAACGCCGCTTACGCCGTGAACAACATCTCC | 140 |
| N2V5 | AAILAEYIAAAAYAVNNIA | 141 | gccgccATCCTGGCTGAGTACATCgccgccGCCGCTTACGCCGTGAACAACATCgcc | 142 |
| N2V6 | EKIAAEYITNAAYAAAAIS | 143 | GAGAAGATCgccGCTGAGTACATCACAAACGCCGCTTACGCCgccgccgccATCTCC | 144 |
| N2V7 | EKALAAYATNAAYAVNNAS | 145 | GAGAAGgccCTGGCTgccTACgccACAAACGCCGCTTACGCCGTGAACAACgccTCC | 146 |
| N2V8 | EKILVEYITNVVYVVNNIS | 147 | GAGAAGATCCTGgtgGAGTACATCACAAACgtggtgTACgtgGTGAACAACATCTCC | 148 |
| N2-Y136A | EKILAEAITNAAYAVNNIS | 149 | GAGAAGATCCTGGCTGAGgccATCACAAACGCCGCTTACGCCGTGAACAACATCTCC | 150 |
| N2-Y142A | EKILAEYITNAAAAVNNIS | 151 | GAGAAGATCCTGGCTGAGTACATCACAAACGCCGCTgccGCCGTGAACAACATCTCC | 152 |
| N3L | GLDKDIIGFGKFSTVYT | 153 | GGCCTGGACAAGGATATCATCGGCTTCGGAAAGTTTTCTACCGTGTACACA | 154 |
| N3V1 | ALDADIIGFGAFATVYT | 155 | gccCTGGACgccGATATCATCGGCTTCGGAgccTTTgccACCGTGTACACA | 156 |
| N3V2 | GLDKDIIAFAKFSAVYA | 157 | GGCCTGGACAAGGATATCATCgccTTCgccAAGTTTTCTgccGTGTACgcc | 158 |
| N3V3 | GAAKAIIGFGKFSTAYT | 159 | GGCgccgccAAGgccATCATCGGCTTCGGAAAGTTTTCTACCgccTACACA | 160 |
| N3V4 | GLDKDAAGAGKASTVYT | 161 | GGCCTGGACAAGGATgccgccGGCgccGGAAAGgccTCTACCGTGTACACA | 162 |
| N3-Y164A | GLDKD11GFGKFSTVAT | 163 | GGCCTGGACAAGGATATCATCGGCTTCGGAAAGTTTTCTACCGTGgccACA | 164 |
| N3R | YDEFKDPEHHRAAFNNNDK | 165 | TACGACGAGTTCAAGGATCCAGAGCACCACCGGGCCGCTTTTAACAACAACGACAAG | 166 |
| N3V5 | AAEFAAPEHHRAAFNNNDA | 167 | gccgccGAGTTCgccgccCCAGAGCACCACCGGGCCGCTTTTAACAACAACGACgcc | 168 |
| N3V6 | YDEFKDPEAHRAAFAAAAK | 169 | TACGACGAGTTCAAGGATCCAGAGgccCACCGGGCCGCTTTTgccgccgccgccAAG | 170 |
| N3V7 | YDAAKDPEHARAAANNNDK | 171 | TACGACgccgccAAGGATCCAGAGCACgccCGGGCCGCTgccAACAACAACGACAAG | 172 |
| N3V8 | YDEFKDPAHHAVVFNNNDK | 173 | TACGACGAGTTCAAGGATCCAgccCACCACgccgtggtgTTTAACAACAACGACAAG | 174 |
| N3-Y166A | ADEFKDPEHHRAAFNNNDK | 175 | gccGACGAGTTCAAGGATCCAGAGCACCACCGGGCCGCTTTTAACAACAACGACAAG | 176 |
| N4L | LINAIKAQYDEFDNFLD | 177 | CTGATCAACGCCATCAAGGCTCAGTACGACGAGTTCGATAACTTTCTGGAT | 178 |
| N4V1 | LINAIAAQYAEFANFLA | 179 | CTGATCAACGCCATCgccGCTCAGTACgccGAGTTCgccAACTTTCTGgcc | 180 |
| N4V2 | AIAAIKAAYDEFDAFLD | 181 | gccATCgccGCCATCAAGGCTgccTACGACGAGTTCGATgccTTTCTGGAT | 182 |
| N4V3 | LANAAKAQYDEADNFAD | 183 | CTGgccAACGCCgccAAGGCTCAGTACGACGAGgccGATAACTTTgccGAT | 184 |
| N4V4 | LINVIKVQYDAFDNALD | 185 | CTGATCAACgtgATCAAGgtgCAGTACGACgccTTCGATAACgccCTGGAT | 186 |
| N4-Y193A | LINAIKAQADEFDNFLD | 187 | CTGATCAACGCCATCAAGGCTCAGgccGACGAGTTCGATAACTTTCTGGAT | 188 |
| N4R | NPRLGYFGQAFFSKEGRNY | 189 | AACCCCAGGCTGGGCTACTTCGGACAGGCTTTCTTTTCTAAGGAGGGCAGAAACTAC | 190 |
| N4V5 | AARLAYFGQAFFAAEGRNY | 191 | gccgccAGGCTGgccTACTTCGGACAGGCTTTCTTTgccgccGAGGGCAGAAACTAC | 192 |
| N4V6 | NPRLGYFAAAFFSKEARAY | 193 | AACCCCAGGCTGGGCTACTTCgccgccGCTTTCTTTTCTAAGGAGgccAGAgccTAC | 194 |
| N4V7 | NPRAGYAGQAAASKEGRNY | 195 | AACCCCAGGgccGGCTACgccGGACAGGCTgccgccTCTAAGGAGGGCAGAAACTAC | 196 |
| N4V8 | NPALGYFGQVFFSKAGANY | 197 | AACCCCgccCTGGGCTACTTCGGACAGgtgTTCTTTTCTAAGgccGGCgccAACTAC | 198 |
| N4-Y207A | NPRLGAFGQAFFSKEGRNY | 199 | AACCCCAGGCTGGGCgccTTCGGACAGGCTTTCTTTTCTAAGGAGGGCAGAAACTAC | 200 |
| N4-Y220A | NPRLGYFGQAFFSKEGRNA | 201 | AACCCCAGGCTGGGCTACTTCGGACAGGCTTTCTTTTCTAAGGAGGGCAGAAACgcc | 202 |
| N5L | IINYGNECYDILALLSG | 203 | ATCATCAACTACGGAAACGAGTGTTACGACATCCTGGCCCTGCTGAGCGGA | 204 |
| N5V1 | IIAYANECYAILALLAA | 205 | ATCATCgccTACgccAACGAGTGTTACgccATCCTGGCCCTGCTGgccgcc | 206 |
| N5V2 | IINYGAEAYDIAAAASG | 207 | ATCATCAACTACGGAgccGAGgccTACGACATCgccGCCgccgccAGCGGA | 208 |
| N5V3 | AANYGNACYDALVLLSG | 209 | gccgccAACTACGGAAACgccTGTTACGACgccCTGgtgCTGCTGAGCGGA | 210 |
| N5R | LRHWVVHNNEEESRISRTW | 211 | CTGAGGCACTGGGTGGTGCACAACAACGAGGAGGAGTCTCGGATCAGCCGCACCTGG | 212 |
| N5V4 | AAHWVVHNNEEEARIARAW | 213 | gccgccCACTGGGTGGTGCACAACAACGAGGAGGAGgccCGGATCgccCGCgccTGG | 214 |
| N5V5 | LRHWVVHAAAAESRASRTW | 215 | CTGAGGCACTGGGTGGTGCACgccgccgccgccGAGTCTCGGgccAGCCGCACCTGG | 216 |
| N5V6 | LRHWVVHNNEEASAISATA | 217 | CTGAGGCACTGGGTGGTGCACAACAACGAGGAGgccTCTgccATCAGCgccACCgcc | 218 |
| N6L | LYNLDKNLDNEYISTLN | 219 | CTGTACAACCTGGACAAGAACCTGGATAACGAGTACATCTCCACACTGAAC | 220 |
| N6V1 | LYNLAANLANEYIAALN | 221 | CTGTACAACCTGgccgccAACCTGgccAACGAGTACATCgccgccCTGAAC | 222 |
| N6V2 | AYALDKALDAEYISTLA | 223 | gccTACgccCTGGACAAGgccCTGGATgccGAGTACATCTCCACACTGgcc | 224 |
| N6V3 | LYNADKNADNAYASTAN | 225 | CTGTACAACgCCGACAAGAACgCCGATAACgCCTACgCCTCCACAgcCAAC | 226 |
| N6-Y258A | LANLDKNLDNEYISTLN | 227 | CTGgccAACCTGGACAAGAACCTGGATAACGAGTACATCTCCACACTGAAC | 228 |
| N6-Y268A | LYNLDKNLDNEAISTLN | 229 | CTGTACAACCTGGACAAGAACCTGGATAACGAGgccATCTCCACACTGAAC | 230 |
| N6R | YLYDRITNELTNSFSKNSA | 231 | TACCTGTACGACAGGATCACCAACGAGCTGACAAACAGCTTCTCCAAGAACTCTGCC | 232 |
| N6V4 | AAYDRITNELTNAFAANSA | 233 | gccgccTACGACAGGATCACCAACGAGCTGACAAACgccTTCgccgccAACTCTGCC | 234 |
| N6V5 | YLYARIAAELANSFSKNAA | 235 | TACCTGTACgccAGGATCgccgccGAGCTGgccAACAGCTTCTCCAAGAACgccGCC | 236 |
| N6V6 | YLYDRATNEATASFSKASA | 237 | TACCTGTACGACAGGgccACCAACGAGgccACAgccAGCTTCTCCAAGgccTCTGCC | 238 |
| N6V7 | YLYDAITNALTNSASKNSV | 239 | TACCTGTACGACgccATCACCAACgccCTGACAAACAGCgccTCCAAGAACTCTgtg | 240 |
| N6-Y274A | ALYDRITNELTNSFSKNSA | 241 | gccCTGTACGACAGGATCACCAACGAGCTGACAAACAGCTTCTCCAAGAACTCTGCC | 242 |
| N6-Y276A | YLADRITNELTNSFSKNSA | 243 | TACCTGgccGACAGGATCACCAACGAGCTGACAAACAGCTTCTCCAAGAACTCTGCC | 244 |
| N7L | ANVNYIAETLGINPAEF | 245 | GCTAACGTGAACTACATCGCTGAGACCCTGGGCATCAACCCAGCTGAGTTC | 246 |
| N7V1 | AAVAYIAEALAIAPAEF | 247 | GCTgccGTGgccTACATCGCTGAGgccCTGgccATCgccCCAGCTGAGTTC | 248 |
| N7V2 | ANANYAAETAGANPAEA | 249 | GCTAACgccAACTACgccGCTGAGACCgccGGCgccAACCCAGCTGAGgcc | 250 |
| N7V3 | VNVNYIVATLGINPVAF | 251 | gtgAACGTGAACTACATCgtggccACCCTGGGCATCAACCCAgtggccTTC | 252 |
| N7-Y297A | ANVNAIAETLGINPAEF | 253 | GCTAACGTGAACgccATCGCTGAGACCCTGGGCATCAACCCAGCTGAGTTC | 254 |
| N7R | AEQYFRFSIMKEQKNLGFN | 255 | GCTGAGCAGTACTTCAGATTTTCCATCATGAAGGAGCAGAAGAACCTGGGCTTCAAC | 256 |
| N7V4 | VAQYFRFAIMAEQANLGFN | 257 | gtggccCAGTACTTCAGATTTgccATCATGgccGAGCAGgccAACCTGGGCTTCAAC | 258 |
| N7V5 | AEAYFRFSIMKEAKALAFA | 259 | GCTGAGgccTACTTCAGATTTTCCATCATGAAGGAGgccAAGgccCTGgccTTCgcc | 260 |
| N7V6 | AEQYARFSAAKEQKNAGFN | 261 | GCTGAGCAGTACgccAGATTTTCCgccgccAAGGAGCAGAAGAACgccGGCTTCAAC | 262 |
| N7V7 | AEQYFAASIMKAQKNLGAN | 263 | GCTGAGCAGTACTTCgccgccTCCATCATGAAGgccCAGAAGAACCTGGGCgccAAC | 264 |
| N7-Y313A | AEQAFRFSIMKEQKNLGFN | 265 | GCTGAGCAGgccTTCAGATTTTCCATCATGAAGGAGCAGAAGAACCTGGGCTTCAAC | 266 |
| N8L | AGRDVSAFSKLMYALTM | 267 | GCTGGAAGGGACGTGAGCGCCTTCAGCAAGCTGATGTACGCCCTGACAATG | 268 |
| N8V1 | AARDVAAFAALMYALTM | 269 | GCTgccAGGGACGTGgccGCCTTCgccgccCTGATGTACGCCCTGACAATG | 270 |
| N8V2 | AGRAASAFSKAMYALAM | 271 | GCTGGAAGGgccgccAGCGCCTTCAGCAAGgccATGTACGCCCTGgccATG | 272 |
| N8V3 | AGRDVSAASKLAYAATA | 273 | GCTGGAAGGGACGTGAGCGCCgccAGCAAGCTGgccTACGCCgccACAgcc | 274 |
| N8V4 | VGADVSVFSKLMYVLTM | 275 | gtgGGAgccGACGTGAGCgtgTTCAGCAAGCTGATGTACgtgCTGACAATG | 276 |
| N8-Y470A | AGRDVSAFSKLMAALTM | 277 | GCTGGAAGGGACGTGAGCGCCTTCAGCAAGCTGATGgccGCCCTGACAATG | 278 |
| N8R | FLDGKEINDLLTTLINKFD | 279 | TTTCTGGACGGAAAGGAGATCAACGATCTGCTGACCACACTGATCAACAAGTTCGAC | 280 |
| N8V5 | AADAAEINDLLTTLINAFD | 281 | gccgccGACgccgccGAGATCAACGATCTGCTGACCACACTGATCAACgccTTCGAC | 282 |
| N8V6 | FLAGKEINALLAALINKFA | 283 | TTTCTGgccGGAAAGGAGATCAACgccCTGCTGgccgccCTGATCAACAAGTTCgcc | 284 |
| N8V7 | FLDGKEIADAATTAIAKFD | 285 | TTTCTGGACGGAAAGGAGATCgccGATgccgccACCACAgccATCgccAAGTTCGAC | 286 |
| N8V8 | FLDGKAANDLLTTLANKAD | 287 | TTTCTGGACGGAAAGgccgccAACGATCTGCTGACCACACTGgccAACAAGgccGAC | 288 |
| N9L | NIQSFLKVMPLIGVNAK | 289 | AACATCCAGTCTTTTCTGAAAGTGATGCCTCTGATCGGCGTGAACGCTAAG | 290 |
| N9V1 | NIQAFLAVMPLIAVNAA | 291 | AACATCCAGgccTTTCTGgccGTGATGCCTCTGATCgccGTGAACGCTgcc | 292 |
| N9V2 | AIQSFLKAMPLIGAAAK | 293 | gccATCCAGTCTTTTCTGAAAgccATGCCTCTGATCGGCgccgccGCTAAG | 294 |
| N9V3 | NIASFAKVAPAIGVNAK | 295 | AACATCgccTCTTTTgccAAAGTGgccCCTgccATCGGCGTGAACGCTAAG | 296 |
| N9V4 | NAQSALKVMPLAGVNVK | 297 | AACgccCAGTCTgccCTGAAAGTGATGCCTCTGgccGGCGTGAACgtgAAG | 298 |
| N9R | FVEEYAFFKDSAKIADELR | 299 | TTCGTGGAGGAGTACGCCTTCTTTAAGGACAGCGCCAAGATCGCTGATGAGCTGCGG | 300 |
| N9V5 | AAEEYAFFADAAAIADELR | 301 | gccgccGAGGAGTACGCCTTCTTTgccGACgccGCCgccATCGCTGATGAGCTGCGG | 302 |
| N9V6 | FVEEYAAFKASAKAAAEAR | 303 | TTCGTGGAGGAGTACGCCgccTTTAAGgccAGCGCCAAGgccGCTgccGAGgccCGG | 304 |
| N9V7 | FVAAYAFAKDSAKIADALR | 305 | TTCGTGgccgccTACGCCTTCgccAAGGACAGCGCCAAGATCGCTGATgccCTGCGG | 306 |
| N9V8 | FVEEYVFFKDSVKIVDELA | 307 | TTCGTGGAGGAGTACgtgTTCTTTAAGGACAGCgtgAAGATCgtgGATGAGCTGgcc | 308 |
| N9-Y515A | FVEEAAFFKDSAKIADELR | 309 | TTCGTGGAGGAGgccGCCTTCTTTAAGGACAGCGCCAAGATCGCTGATGAGCTGCGG | 310 |
| N10L | LIKSFARMGEPIADARR | 311 | CTGATCAAGTCCTTTGCCAGGATGGGAGAGCCAATCGCTGACGCTAGGAGA | 312 |
| N10V1 | LIAAFARMAEPIAAARR | 313 | CTGATCgccgccTTTGCCAGGATGgccGAGCCAATCGCTgccGCTAGGAGA | 314 |
| N10V2 | AAKSFARAGEPAADARR | 315 | gccgccAAGTCCTTTGCCAGGgccGGAGAGCCAgccGCTGACGCTAGGAGA | 316 |
| N10V3 | LIKSAVRMGAPIVDARR | 317 | CTGATCAAGTCCgccgtgAGGATGGGAgccCCAATCgtgGACGCTAGGAGA | 318 |
| N10V4 | LIKSFAAMGEPIADVAA | 319 | CTGATCAAGTCCTTTGCCgccATGGGAGAGCCAATCGCTGACgtggccgcc | 320 |
| N10R | AMYIDAIRILGTNLSYDEL | 321 | GCTATGTACATCGATGCCATCCGGATCCTGGGAACCAACCTGTCTTACGACGAGCTG | 322 |
| N10V5 | VAYIDAIRILAANLAYDEL | 323 | gtggccTACATCGATGCCATCCGGATCCTGgccgccAACCTGgccTACGACGAGCTG | 324 |
| N10V6 | AMYIAAIRIAGTALSYAEL | 325 | GCTATGTACATCgccGCCATCCGGATCgccGGAACCgccCTGTCTTACgccGAGCTG | 326 |
| N10V7 | AMYADAARILGTNASYDEA | 327 | GCTATGTACgccGATGCCgccCGGATCCTGGGAACCAACgccTCTTACGACGAGgcc | 328 |
| N10V8 | AMYIDVIAALGTNLSYDAL | 329 | GCTATGTACATCGATgtgATCgccgccCTGGGAACCAACCTGTCTTACGACgccCTG | 330 |
| N10-Y549A | AMAIDAIRILGTNLSYDEL | 331 | GCTATGgccATCGATGCCATCCGGATCCTGGGAACCAACCTGTCTTACGACGAGCTG | 332 |
| N10-Y562A | AMYIDAIRILGTNLSADEL | 333 | GCTATGTACATCGATGCCATCCGGATCCTGGGAACCAACCTGTCTgccGACGAGCTG | 334 |
| NHL | KALADTFSLDENGNKLK | 335 | AAGGCTCTGGCCGACACCTTCAGCCTGGATGAGAACGGCAACAAGCTGAAG | 336 |
| N11V1 | AALADTFALDENANALA | 337 | gccGCTCTGGCCGACACCTTCgccCTGGATGAGAACgccAACgccCTGgcc | 338 |
| NHV2 | KALAAAFSLAEAGNKLK | 339 | AAGGCTCTGGCCgccgccTTCAGCCTGgccGAGgccGGCAACAAGCTGAAG | 340 |
| NHV3 | KAAADTFSADENGAKAK | 341 | AAGGCTgccGCCGACACCTTCAGCgccGATGAGAACGGCgccAAGgccAAG | 342 |
| NHV4 | KVLVDTASLDANGNKLK | 343 | AAGgtgCTGgtgGACACCgccAGCCTGGATgccAACGGCAACAAGCTGAAG | 344 |
| NHR | KGKHGMRNFIINNVISNKR | 345 | AAGGGCAAGCACGGAATGCGCAACTTCATCATCAACAACGTGATCAGCAACAAGCGG | 346 |
| NHV5 | AAAHGMRNFIINNVIANAR | 347 | gccgccgccCACGGAATGCGCAACTTCATCATCAACAACGTGATCgccAACgccCGG | 348 |
| N11V6 | KGKHAMRAFIIAAVISAKR | 349 | AAGGGCAAGCACgccATGCGCgccTTCATCATCgccgccGTGATCAGCgccAAGCGG | 350 |
| N11V7 | KGKAGARNFAANNAISNKR | 351 | AAGGGCAAGgccGGAgccCGCAACTTCgccgccAACAACgccATCAGCAACAAGCGG | 352 |
| N11V8 | KGKHGMANAIINNVASNKA | 353 | AAGGGCAAGCACGGAATGgccAACgccATCATCAACAACGTGgccAGCAACAAGgcc | 354 |
| N12L | FHYLIRYGDPAHLHEIA | 355 | TTTCACTACCTGATCAGATACGGCGACCCAGCTCACCTGCACGAGATCGCT | 356 |
| N12V1 | FAYAIRYAAPAHAHEIA | 357 | TTTgccTACgccATCAGATACgccgccCCAGCTCACgccCACGAGATCGCT | 358 |
| N12V2 | AHYLARYGDPAALAEAA | 359 | gccCACTACCTGgccAGATACGGCGACCCAGCTgccCTGgccGAGgccGCT | 360 |
| N12V3 | FHYLIAYGDPVHLHAIV | 361 | TTTCACTACCTGATCgccTACGGCGACCCAgtgCACCTGCACgccATCgtg | 362 |
| N12-Y604A | FHALIRYGDPAHLHEIA | 363 | TTTCACgccCTGATCAGATACGGCGACCCAGCTCACCTGCACGAGATCGCT | 364 |
| N12-Y608A | FHYLIRAGDPAHLHEIA | 365 | TTTCACTACCTGATCAGAgccGGCGACCCAGCTCACCTGCACGAGATCGCT | 366 |
| N12R | KNEAVVKFVLGRIADIQKK | 367 | AAGAACGAGGCCGTGGTGAAGTTCGTGCTGGGACGGATCGCCGATATCCAGAAGAAG | 368 |
| N12V4 | AAEAVVAFVLGRIADIQAA | 369 | gccgccGAGGCCGTGGTGgccTTCGTGCTGGGACGGATCGCCGATATCCAGgccgcc | 370 |
| N12V5 | KNEAAAKFALARIAAIQKK | 371 | AAGAACGAGGCCgccgccAAGTTCgccCTGgccCGGATCGCCgccATCCAGAAGAAG | 372 |
| N12V6 | KNEAVVKAVAGRAADAAKK | 373 | AAGAACGAGGCCGTGGTGAAGgccGTGgccGGACGGgccGCCGATgccgccAAGAAG | 374 |
| N12V7 | KNAVVVKFVLGAIVDIQKK | 375 | AAGAACgccgtgGTGGTGAAGTTCGTGCTGGGAgccATCgtgGATATCCAGAAGAAG | 376 |
| N13L | QGQNGKNQIDRYYETCI | 377 | CAGGGCCAGAACGGAAAGAACCAGATCGACCGCTACTACGAGACCTGCATC | 378 |
| N13V1 | QAQNAANQIARYYEACI | 379 | CAGgccCAGAACgccgccAACCAGATCgccCGCTACTACGAGgccTGCATC | 380 |
| N13V2 | AGAAGKAAIDRYYETCI | 381 | gccGGCgccgccGGAAAGgccgccATCGACCGCTACTACGAGACCTGCATC | 382 |
| N13V3 | QGQNGKNQADAYYATAA | 383 | CAGGGCCAGAACGGAAAGAACCAGgccGACgccTACTACgccACCgccgcc | 384 |
| N13-Y649A | QGQNGKNQIDRAYETCI | 385 | CAGGGCCAGAACGGAAAGAACCAGATCGACCGCgccTACGAGACCTGCATC | 386 |
| N13-Y650A | QGQNGKNQIDRYAETCI | 387 | CAGGGCCAGAACGGAAAGAACCAGATCGACCGCTACgccGAGACCTGCATC | 388 |
| N13R | GKDKGKSVSEKVDALTKII | 389 | GGCAAGGATAAGGGAAAGTCCGTGTCTGAGAAGGTGGACGCTCTGACCAAGATCATC | 390 |
| N13V4 | AADAGASVSEAVDALTKII | 391 | gccgccGATgccGGAgccTCCGTGTCTGAGgccGTGGACGCTCTGACCAAGATCATC | 392 |
| N13V5 | GKDKAKAVAEKVDALAAII | 393 | GGCAAGGATAAGgccAAGgccGTGgccGAGAAGGTGGACGCTCTGgccgccATCATC | 394 |
| N13V6 | GKAKGKSASEKAAAATKII | 395 | GGCAAGgccAAGGGAAAGTCCgccTCTGAGAAGgccgccGCTgccACCAAGATCATC | 396 |
| N13V7 | GKDKGKSVSAKVDVLTKAA | 397 | GGCAAGGATAAGGGAAAGTCCGTGTCTgccAAGGTGGACgtgCTGACCAAGgccgcc | 398 |
| N14L | TGMNYDQFDKKRSVIED | 399 | ACAGGCATGAACTACGACCAGTTCGATAAGAAGAGATCTGTGATCGAGGAC | 400 |
| N14V1 | TAMNYDQFDAARAVIED | 401 | ACAgccATGAACTACGACCAGTTCGATgccgccAGAgccGTGATCGAGGAC | 402 |
| N14V2 | AGMNYAQFAKKRSVIEA | 403 | gccGGCATGAACTACgccCAGTTCgccAAGAAGAGATCTGTGATCGAGgcc | 404 |
| N14V3 | TGAAYDAFDKKRSAIED | 405 | ACAGGCgccgccTACGACgccTTCGATAAGAAGAGATCTgccATCGAGGAC | 406 |
| N14V4 | TGMNYDQADKKASVAAD | 407 | ACAGGCATGAACTACGACCAGgccGATAAGAAGgccTCTGTGgccgccGAC | 408 |
| N14-Y678A | TGMNADQFDKKRSVIED | 409 | ACAGGCATGAACgccGACCAGTTCGATAAGAAGAGATCTGTGATCGAGGAC | 410 |
| N14R | TGRENAEREKFKKIISLYL | 411 | ACCGGAAGGGAGAACGCCGAGAGAGAGAAGTTTAAGAAGATCATCAGCCTGTACCTG | 412 |
| N14V5 | AARENAEREAFAAIISLYL | 413 | gccgccAGGGAGAACGCCGAGAGAGAGgccTTTgccgccATCATCAGCCTGTACCTG | 414 |
| N14V6 | TGREAAEREKFKKAIAAYA | 415 | ACCGGAAGGGAGgccGCCGAGAGAGAGAAGTTTAAGAAGgccATCgccgccTACgcc | 416 |
| N14V7 | TGRANAAREKAKKIASLYL | 417 | ACCGGAAGGgccAACGCCgccAGAGAGAAGgccAAGAAGATCgccAGCCTGTACCTG | 418 |
| N14V8 | TGAENVEAAKFKKIISLYL | 419 | ACCGGAgccGAGAACgtgGAGgccgccAAGTTTAAGAAGATCATCAGCCTGTACCTG | 420 |
| N14-Y708A | TGRENAEREKFKKIISLAL | 421 | ACCGGAAGGGAGAACGCCGAGAGAGAGAAGTTTAAGAAGATCATCAGCCTGgccCTG | 422 |
| N15L | TVIYHILKNIVNINARY | 423 | ACAGTGATCTACCACATCCTGAAGAACATCGTGAACATCAACGCTAGATAC | 424 |
| N15V1 | AVIYHILAAIVAIAARY | 425 | gccGTGATCTACCACATCCTGgccgccATCGTGgccATCgccGCTAGATAC | 426 |
| N15V2 | TAAYAIAKNIANINARY | 427 | ACAgcCgCCTACgCCATCgCCAAGAACATCgCCAACATCAACGCTAGATAC | 428 |
| N15V3 | TVIYHALKNAVNANVAY | 429 | ACAGTGATCTACCACgccCTGAAGAACgccGTGAACgccAACgtggccTAC | 430 |
| N15R | VIGFHCVERDAQLYKEKGY | 431 | GTGATCGGCTTCCACTGCGTGGAGCGCGATGCCCAGCTGTACAAGGAGAAGGGATAC | 432 |
| N15V4 | AAAFHCVERDAQLYAEAGY | 433 | gccgccgccTTCCACTGCGTGGAGCGCGATGCCCAGCTGTACgccGAGgccGGATAC | 434 |
| N15V5 | VIGFHCAERAAALYKEKAY | 435 | GTGATCGGCTTCCACTGCgccGAGCGCgccGCCgccCTGTACAAGGAGAAGgccTAC | 436 |
| N15V6 | VIGAAAVERDAQAYKEKGY | 437 | GTGATCGGCgccgccgccGTGGAGCGCGATGCCCAGgccTACAAGGAGAAGGGATAC | 438 |
| N15V7 | VIGFHCVAADVQLYKAKGY | 439 | GTGATCGGCTTCCACTGCGTGgccgccGATgtgCAGCTGTACAAGgccAAGGGATAC | 440 |
| N16L | DINLKKLEEKGFSSVTK | 441 | GACATCAACCTGAAGAAGCTGGAGGAGAAGGGCTTTAGCTCCGTGACCAAG | 442 |
| N16V1 | DINLAALEEAGFASVTA | 443 | GACATCAACCTGgccgccCTGGAGGAGgccGGCTTTgccTCCGTGACCgcc | 444 |
| N16V2 | AINLKKLEEKAFSAVAK | 445 | gccATCAACCTGAAGAAGCTGGAGGAGAAGgccTTTAGCgccGTGgccAAG | 446 |
| N16V3 | DIAAKKAEEKGFSSATK | 447 | GACATCgccgccAAGAAGgccGAGGAGAAGGGCTTTAGCTCCgccACCAAG | 448 |
| N16V4 | DANLKKLAAKGASSVTK | 449 | GACgccAACCTGAAGAAGCTGgccgccAAGGGCgccAGCTCCGTGACCAAG | 450 |
| N16R | LCAGIDETAPDKRKDVEKE | 451 | CTGTGCGCTGGAATCGACGAGACAGCCCCCGACAAGAGGAAGGATGTGGAGAAGGAG | 452 |
| N16V5 | AAAGIDETAPDARADVEAE | 453 | gccgccGCTGGAATCGACGAGACAGCCCCCGACgccAGGgccGATGTGGAGgccGAG | 454 |
| N16V6 | LCAAIAEAAPAKRKAVEKE | 455 | CTGTGCGCTgccATCgccGAGgccGCCCCCgccAAGAGGAAGgccGTGGAGAAGGAG | 456 |
| N16V7 | LCAGADATAPDKRKDAAKE | 457 | CTGTGCGCTGGAgccGACgccACAGCCCCCGACAAGAGGAAGGATgccgccAAGGAG | 458 |
| N16V8 | LCVGIDETVPDKAKDVEKA | 459 | CTGTGCgtgGGAATCGACGAGACAgtgCCCGACAAGgccAAGGATGTGGAGAAGgcc | 460 |
| N17L | MAERAKESIDSLESANP | 461 | ATGGCCGAGAGAGCTAAGGAGAGCATCGACTCCCTGGAGTCTGCTAACCCT | 462 |
| N17V1 | MAERAAEAIDALEAANP | 463 | ATGGCCGAGAGAGCTgccGAGgccATCGACgccCTGGAGgccGCTAACCCT | 464 |
| N17V2 | AAERAKESIASAESAAP | 465 | gccGCCGAGAGAGCTAAGGAGAGCATCgccTCCgccGAGTCTGCTgccCCT | 466 |
| N17V3 | MAARAKASADSLASANP | 467 | ATGGCCgccAGAGCTAAGgccAGCgccGACTCCCTGgccTCTGCTAACCCT | 468 |
| N17V4 | MVEAVKESIDSLESVNP | 469 | ATGgtgGAGgccgtgAAGGAGAGCATCGACTCCCTGGAGTCTgtgAACCCT | 470 |
| N17R | KLYANYIKYSDEKKAEEFT | 471 | AAGCTGTACGCCAACTACATCAAGTACTCCGATGAGAAGAAGGCCGAGGAGTTCACC | 472 |
| N17V5 | AAYANYIAYSDEAKAEEFT | 473 | gccgccTACGCCAACTACATCgccTACTCCGATGAGgccAAGGCCGAGGAGTTCACC | 474 |
| N17V6 | KLYANYIKYAAEKAAEEFA | 475 | AAGCTGTACGCCAACTACATCAAGTACgccgccGAGAAGgccGCCGAGGAGTTCgcc | 476 |
| N17V7 | KLYAAYAKYSDAKKAEEAT | 477 | AAGCTGTACGCCgccTACgccAAGTACTCCGATgccAAGAAGGCCGAGGAGgccACC | 478 |
| N17V8 | KLYVNYIKYSDEKKVAAFT | 479 | AAGCTGTACgtgAACTACATCAAGTACTCCGATGAGAAGAAGgtggccgccTTCACC | 480 |
| N18L | RQINREKAKTALNAYLR | 481 | AGGCAGATCAACAGAGAGAAGGCCAAGACCGCTCTGAACGCCTACCTGAGG | 482 |
| N18V1 | RQIAREAAAAALNAYLR | 483 | AGGCAGATCgccAGAGAGgccGCCgccgccGCTCTGAACGCCTACCTGAGG | 484 |
| N18V2 | RAINREKAKTAAAAYAR | 485 | AGGgccATCAACAGAGAGAAGGCCAAGACCGCTgccgccGCCTACgccAGG | 486 |
| N18V3 | RQANRAKVKTVLNAYLR | 487 | AGGCAGgccAACAGAgccAAGgtgAAGACCgtgCTGAACGCCTACCTGAGG | 488 |
| N18V4 | AQINAEKAKTALNVYLA | 489 | gccCAGATCAACgccGAGAAGGCCAAGACCGCTCTGAACgtgTACCTGgcc | 490 |
| N18R | NTKWNVIIREDLLRIDNKT | 491 | AACACAAAGTGGAACGTGATCATCCGGGAGGACCTGCTGCGCATCGATAACAAGACC | 492 |
| N18V5 | AAAWNVIIREDLLRIDNAA | 493 | gccgccgccTGGAACGTGATCATCCGGGAGGACCTGCTGCGCATCGATAACgccgcc | 494 |
| N18V6 | NTKWAAIIREALLRIAAKT | 495 | AACACAAAGTGGgccgccATCATCCGGGAGgccCTGCTGCGCATCgccgccAAGACC | 496 |
| N18V7 | NTKWNVAAREDAARADNKT | 497 | AACACAAAGTGGAACGTGgccgccCGGGAGGACgccgccCGCgccGATAACAAGACC | 498 |
| N18V8 | NTKANVIIAADLLAIDNKT | 499 | AACACAAAGgccAACGTGATCATCgccgccGACCTGCTGgccATCGATAACAAGACC | 500 |
| N19L | CTLFRNKAVHLEVARYV | 501 | TGTACACTGTTCCGGAACAAGGCTGTGCACCTGGAGGTGGCTCGCTACGTG | 502 |
| N19V1 | CAAFRNKAVHAEAARYA | 503 | TGTgccgccTTCcggaacaaggctgtgcacgccGAGgccGCTCGCTACgcc | 504 |
| N19V2 | ATLARNKAVHLAVVAYV | 505 | gccACACTGgcccggaacaaggctgtgcacCTGgccGTGgtggccTACGTG | 506 |
| N19R | HAYINDIAEVNSYFQLYHY | 507 | CACGCCTACATCAACGACATCGCCGAGGTGAACTCCTACTTTCAGCTGTACCACTAC | 508 |
| N19V3 | AVYIAAIAEVNAYFQLYHY | 509 | gccgtgTACATCgccgccATCGCCGAGGTGAACgccTACTTTCAGCTGTACCACTAC | 510 |
| N19V4 | HAYINDIAEAASYFAAYAY | 511 | CACGCCTACATCAACGACATCGCCGAGgccgccTCCTACTTTgccgccTACgccTAC | 512 |
| N19V5 | HAYANDAVAVNSYAQLYHY | 513 | CACGCCTACgccAACGACgccgtggccGTGAACTCCTACgccCAGCTGTACCACTAC | 514 |
| N20L | IMQRIIMNERYEKSSGK | 515 | ATCATGCAGAGGATCATCATGAACGAGAGATACGAGAAGTCTAGCGGCAAG | 516 |
| N20V1 | IMQRIIMNERYEAAAGA | 517 | ATCATGCAGAGGATCATCATGAACGAGAGATACGAGgccgccgccGGCgcc | 518 |
| N20V2 | IAARIIMAERYEKSSAK | 519 | ATCgccgccAGGATCATCATGgccGAGAGATACGAGAAGTCTAGCgccAAG | 520 |
| N20V3 | AMQRAAANERYEKSSGK | 521 | gccATGCAGAGGgccgccgccAACGAGAGATACGAGAAGTCTAGCGGCAAG | 522 |
| N20V4 | IMQAIIMNAAYAKSSGK | 523 | ATCATGCAGgccATCATCATGAACgccgccTACgccAAGTCTAGCGGCAAG | 524 |
| N20-Y900A | IMQRIIMNERAEKSSGK | 525 | ATCATGCAGAGGATCATCATGAACGAGAGAgccGAGAAGTCTAGCGGCAAG | 526 |
| N20R | VSEYFDAVNDEKKYNDRLL | 527 | GTGTCTGAGTACTTCGACGCCGTGAACGATGAGAAGAAGTACAACGATAGACTGCTG | 528 |
| N20V5 | AAEYFAAVNDEAAYNDRLL | 529 | gccgccGAGTACTTCgccGCCGTGAACGATGAGgccgccTACAACGATAGACTGCTG | 530 |
| N20V6 | VSEYFDAVAAEKKYAARLL | 531 | GTGTCTGAGTACTTCGACGCCGTGgccgccGAGAAGAAGTACgccgccAGACTGCTG | 532 |
| N20V7 | VSEYADAANDEKKYNDRAA | 533 | GTGTCTGAGTACgccGACGCCgccAACGATGAGAAGAAGTACAACGATAGAgccgcc | 534 |
| N20V8 | VSAYFDVVNDAKKYNDALL | 535 | GTGTCTgccTACTTCGACgtgGTGAACGATgccAAGAAGTACAACGATgccCTGCTG | 536 |
| N20-Y910A | VSEAFDAVNDEKKYNDRLL | 537 | GTGTCTGAGgccTTCGACGCCGTGAACGATGAGAAGAAGTACAACGATAGACTGCTG | 538 |
| N20-Y920A | VSEYFDAVNDEKKANDRLL | 539 | GTGTCTGAGTACTTCGACGCCGTGAACGATGAGAAGAAGgccAACGATAGACTGCTG | 540 |
| N21L | KLLCVPFGYCIPRFKNL | 541 | AAGCTGCTGTGCGTGCCTTTCGGATACTGTATCCCACGGTTTAAGAACCTG | 542 |
| N21V1 | ALLCAPFAYCIPRFAAL | 543 | gccCTGCTGTGCgccCCTTTCgccTACTGTATCCCACGGTTTgccgccCTG | 544 |
| N21V2 | KAAAVPFGYAIPRFKNA | 545 | AAGgccgccgccGTGCCTTTCGGATACgccATCCCACGGTTTAAGAACgcc | 546 |
| N21V3 | KLLCVPAGYCAPAAKNL | 547 | AAGCTGCTGTGCGTGCCTgccGGATACTGTgccCCAgccgccAAGAACCTG | 548 |
| N21-Y934A | KLLCVPFGACIPRFKNL | 549 | AAGCTGCTGTGCGTGCCTTTCGGAgccTGTATCCCACGGTTTAAGAACCTG | 550 |
| N21R | SIEALFDRNEAAKFDKEKK | 551 | AGCATCGAGGCCCTGTTCGACCGCAACGAGGCTGCCAAGTTTGATAAGGAGAAGAAG | 552 |
| N21V4 | AAEALFDRNEAAAFDAEAK | 553 | gccgccGAGGCCCTGTTCGACCGCAACGAGGCTGCCgccTTTGATgccGAGgccAAG | 554 |
| N21V5 | SIEAAFARAEAAKFAKEKA | 555 | AGCATCGAGGCCgccTTCgccCGCgccGAGGCTGCCAAGTTTgccAAGGAGAAGgcc | 556 |
| N21V6 | SIAALADRNAAAKADKAKK | 557 | AGCATCgccGCCCTGgccGACCGCAACgccGCTGCCAAGgccGATAAGgccAAGAAG | 558 |
| N21V7 | SIEVLFDANEVVKFDKEKK | 559 | AGCATCGAGgtgCTGTTCGACgccAACGAGgtggtgAAGTTTGATAAGGAGAAGAAG | 560 |

Using the EGFP-mCherry dual-fluorescence reporter system of the invention, these Cas13d mutants were functionally screened to assess their collateral vs. gRNA-guided cleavage activities. Specifically, according to standard cell culture methods, human HEK293 cells were grown in 24-well tissue culture plates to a suitable density before the cells were transfected with PEI reagents and plasmids that express each mutant Cas13d and the reporter system fluorescent proteins. Transfected cells were cultured at 37° C. in incubator under 5% CO₂ for about 48 hours, before measuring EGFP and mCherry signals in the cells with FACS. Mutants leading to low percentage of the gRNA-targeted EGFP signal (lower percentage of EGFP cells, as a readout for preserved gRNA-guided cleavage) and high percentage of non-targeted mCherry signal (higher percentage of mCherry⁺ cells, as a readout for lacking collateral effect) were selected.

In this experiment, dCas13d with no gRNA-guided cleavage was used as a negative control, and the results (mean±s.e.m.) were normalized against that of dCas13d and listed below. Cas13d mutants located at the upper left area of FIG. 17D had low collateral effect (high mCherry signal) and high gRNA-guided cleavage activity (low EGFP signal), and were selected as the desired low/no collateral effect mutants.

| Variants | % mCherry | S.E.M. | % EGFP | S.E.M. |
|---|---|---|---|---|
| dead | 1.0000 | 0.0425 | 1.0000 | 0.0886 |
| N1V5 | 0.6944 | 0.0030 | 0.5137 | 0.0136 |
| N1V6 | 0.7138 | 0.0350 | 0.6954 | 0.0302 |
| N1V7 | 1.0235 | 0.0119 | 0.1633 | 0.0044 |
| N1-Y79A | 0.3946 | 0.0098 | 0.0612 | 0.0029 |
| N1-Y107A | 0.6085 | 0.0196 | 0.0399 | 0.0044 |
| N2V1 | 1.0472 | 0.0355 | 0.6814 | 0.0165 |
| N2V4 | 1.0681 | 0.0834 | 0.3169 | 0.0355 |
| N2V5 | 1.0812 | 0.0220 | 0.4743 | 0.0129 |
| N2V6 | 0.7921 | 0.0201 | 0.7409 | 0.0159 |
| N2V7 | 1.1137 | 0.0111 | 0.1874 | 0.0039 |
| N2V8 | 1.0213 | 0.0197 | 0.1071 | 0.0023 |
| N2-Y136A | 0.2408 | 0.0114 | 0.0436 | 0.0026 |
| N2-Y142A | 0.1079 | 0.0139 | 0.0250 | 0.0029 |
| N3V5 | 0.2427 | 0.0119 | 0.0577 | 0.0022 |
| N3V6 | 0.5665 | 0.0230 | 0.1849 | 0.0034 |
| N3V7 | 0.8830 | 0.0081 | 0.2485 | 0.0053 |
| N3V8 | 0.2536 | 0.0247 | 0.0585 | 0.0075 |
| N3-Y164A | 0.3370 | 0.0123 | 0.0742 | 0.0052 |
| N3-Y166A | 0.1793 | 0.0030 | 0.0475 | 0.0020 |
| N4V1 | 0.2415 | 0.0153 | 0.0587 | 0.0041 |
| N4V2 | 0.1311 | 0.0091 | 0.0361 | 0.0040 |
| N4V3 | 0.9933 | 0.0289 | 0.3581 | 0.0060 |
| N4V4 | 0.3196 | 0.0063 | 0.1217 | 0.0048 |
| N4V5 | 0.3593 | 0.0214 | 0.1291 | 0.0065 |
| N4V6 | 0.2651 | 0.0237 | 0.0838 | 0.0063 |
| N4V7 | 1.1946 | 0.0104 | 1.0324 | 0.0118 |
| N4V8 | 0.3752 | 0.0167 | 0.1662 | 0.0017 |
| N4-Y193A | 0.0574 | 0.0036 | 0.0166 | 0.0005 |
| N4-Y207A | 0.3814 | 0.0204 | 0.1544 | 0.0096 |
| N4-Y220A | 0.1474 | 0.0147 | 0.0467 | 0.0010 |
| N5V2 | 0.9947 | 0.0316 | 0.9101 | 0.0192 |
| N5V3 | 0.7067 | 0.0282 | 0.3204 | 0.0136 |
| N5V4 | 0.5716 | 0.0059 | 0.6033 | 0.0110 |
| N5V5 | 0.5197 | 0.0176 | 0.4348 | 0.0201 |
| N5V6 | 0.9243 | 0.0507 | 0.8826 | 0.0495 |
| N6V1 | 0.2098 | 0.0045 | 0.0428 | 0.0015 |
| N6V2 | 0.5046 | 0.0070 | 0.2333 | 0.0027 |
| N6V3 | 1.0075 | 0.0473 | 0.4041 | 0.0199 |
| N6V4 | 0.2384 | 0.0164 | 0.0589 | 0.0027 |
| N6V5 | 0.2539 | 0.0225 | 0.0463 | 0.0050 |
| N6V6 | 0.6378 | 0.0164 | 0.2087 | 0.0076 |
| N6-Y258A | 0.1685 | 0.0098 | 0.0340 | 0.0019 |
| N6-Y268A | 0.2055 | 0.0144 | 0.0337 | 0.0040 |
| N6-Y274A | 0.1093 | 0.0084 | 0.0431 | 0.0053 |
| N6-Y276A | 0.1765 | 0.0068 | 0.0268 | 0.0007 |
| N7V1 | 0.4020 | 0.0294 | 0.1129 | 0.0124 |
| N7V2 | 0.6559 | 0.0501 | 0.1955 | 0.0242 |
| N7V3 | 0.4149 | 0.0176 | 0.0678 | 0.0038 |
| N7V5 | 0.5322 | 0.0248 | 0.1516 | 0.0047 |
| N7V6 | 1.1491 | 0.0626 | 0.9620 | 0.0413 |
| N7V7 | 0.6734 | 0.0047 | 0.1279 | 0.0036 |
| N7-Y313A | 0.1675 | 0.0041 | 0.0414 | 0.0052 |
| N8V5 | 0.6363 | 0.0040 | 0.5539 | 0.0063 |
| N8V6 | 0.6094 | 0.0110 | 0.0607 | 0.0025 |
| N8V7 | 0.7593 | 0.0095 | 0.6963 | 0.0074 |
| N8V8 | 0.5880 | 0.0313 | 0.1175 | 0.0058 |
| N8-Y470A | 0.1578 | 0.0151 | 0.0333 | 0.0038 |
| N10V1 | 0.7056 | 0.0148 | 0.0883 | 0.0024 |
| N10V2 | 0.6709 | 0.0184 | 0.1958 | 0.0097 |
| N10V3 | 0.6918 | 0.0062 | 0.1564 | 0.0030 |
| N10V5 | 0.3373 | 0.0124 | 0.1240 | 0.0027 |
| N10V6 | 0.9103 | 0.0382 | 0.4576 | 0.0164 |
| N10V7 | 0.1631 | 0.0038 | 0.0421 | 0.0016 |
| N10V8 | 1.0088 | 0.0406 | 0.9699 | 0.0412 |
| N10-Y549A | 0.2203 | 0.0221 | 0.0355 | 0.0053 |
| N10-Y562A | 0.2138 | 0.0076 | 0.0585 | 0.0022 |
| N12V4 | 0.2121 | 0.0075 | 0.0622 | 0.0041 |
| N12V5 | 0.2084 | 0.0052 | 0.0559 | 0.0009 |
| N12V6 | 1.1298 | 0.0204 | 0.5600 | 0.0041 |
| N12V7 | 0.3140 | 0.0038 | 0.0877 | 0.0006 |
| N12-Y604A | 0.1026 | 0.0063 | 0.0295 | 0.0031 |
| N12-Y608A | 0.4104 | 0.0354 | 0.1335 | 0.0082 |
| N14V4 | 0.4622 | 0.0199 | 0.1333 | 0.0087 |
| N14V5 | 0.6140 | 0.0077 | 0.1030 | 0.0031 |
| N14V7 | 0.3355 | 0.0104 | 0.0715 | 0.0004 |
| N14V8 | 0.5707 | 0.0551 | 0.1178 | 0.0094 |
| N14-Y678A | 0.2015 | 0.0173 | 0.0533 | 0.0047 |
| N14-Y708A | 0.1704 | 0.0230 | 0.0398 | 0.0064 |
| N15V1 | 1.0982 | 0.0189 | 1.0183 | 0.0056 |
| N15V2 | 0.7958 | 0.0491 | 0.4995 | 0.0347 |
| N15V4 | 0.7434 | 0.0150 | 0.1105 | 0.0009 |
| N15V5 | 1.0056 | 0.0542 | 1.0385 | 0.0531 |
| N15V6 | 0.9459 | 0.0122 | 0.9455 | 0.0077 |
| N15V7 | 0.8743 | 0.0518 | 0.7983 | 0.0359 |
| N16V1 | 1.0441 | 0.1104 | 1.0276 | 0.0977 |
| N16V2 | 0.8223 | 0.0433 | 0.6325 | 0.0331 |
| N16V3 | 1.0045 | 0.0297 | 0.8040 | 0.0213 |
| N16V4 | 0.7497 | 0.0677 | 0.7392 | 0.0657 |
| N16V5 | 0.6495 | 0.0252 | 0.1833 | 0.0070 |
| N16V6 | 0.1595 | 0.0093 | 0.1385 | 0.0119 |
| N16V7 | 0.4297 | 0.0256 | 0.1954 | 0.0090 |
| N16V8 | 0.2295 | 0.0024 | 0.0254 | 0.0042 |
| N17V4 | 0.3182 | 0.0174 | 0.1440 | 0.0018 |
| N17V6 | 0.4076 | 0.0189 | 0.1893 | 0.0040 |
| N17V7 | 0.2092 | 0.0116 | 0.1455 | 0.0079 |
| N17V8 | 0.7403 | 0.0033 | 0.5776 | 0.0037 |
| N18V1 | 0.8710 | 0.0153 | 0.7702 | 0.0074 |
| N18V2 | 1.2026 | 0.0283 | 1.2085 | 0.0246 |
| N18V3 | 1.0737 | 0.0466 | 1.1575 | 0.0477 |
| N18V4 | 1.1469 | 0.0504 | 1.1692 | 0.0551 |
| N18V6 | 1.1131 | 0.0067 | 0.9995 | 0.0067 |
| N18V7 | 0.5502 | 0.0181 | 0.2434 | 0.0123 |
| N18V8 | 0.7309 | 0.0558 | 0.6676 | 0.0489 |
| N19V1 | 0.4616 | 0.0227 | 0.0838 | 0.0031 |
| N19V4 | 1.0292 | 0.0306 | 0.9443 | 0.0407 |
| N19V5 | 0.8482 | 0.0214 | 0.7707 | 0.0153 |
| N20V4 | 0.7122 | 0.0163 | 0.5587 | 0.0153 |
| N20V6 | 0.8172 | 0.0039 | 0.4597 | 0.0106 |
| N20V7 | 0.7701 | 0.0168 | 0.5945 | 0.0171 |
| N20-Y900A | 0.3975 | 0.0116 | 0.0266 | 0.0036 |
| N20-Y910A | 0.8119 | 0.0323 | 0.4698 | 0.0239 |
| N20-Y920A | 0.8056 | 0.0186 | 0.6471 | 0.0246 |
| N21V1 | 0.6641 | 0.0068 | 0.4461 | 0.0063 |
| N21V4 | 0.2457 | 0.0149 | 0.0741 | 0.0044 |
| N21V5 | 0.9092 | 0.0599 | 0.5882 | 0.0402 |
| N21V6 | 1.2034 | 0.0539 | 0.7407 | 0.0211 |
| N21V7 | 0.1188 | 0.0074 | 0.0249 | 0.0016 |
| N21-Y934A | 0.8336 | 0.0313 | 0.7271 | 0.0276 |
| WT | 0.1446 | 0.0030 | 0.0410 | 0.0022 |

After normalization of EGFP and mCherry fluorescence intensity by inactive dead Cas13d (dCas13d with R295A, H300A, R849A, and H854A mutations in HEPN domains), it was found that variants with mutation sites in N1, N2, N3, or N15, specially N1V7, N2V7, N2V8, N3V7, and N15V4, exhibited relatively low EGFP fluorescence intensity but high mCherry fluorescence intensity, indicating that these variants retained a high on-target activity but greatly reduced collateral activity (FIG. 17D).

Overall, these mutants exhibited less than 27.5% collateral effect (e.g., ≥72.5% mCherry⁺ cells), and ≥75% gRNA-guided cleavage (≤25% EGFP⁺ cells). They include: N1V7, N2V7, N2V8, N3V7, and N15V4, etc. (see above table and FIG. 17D). Based on FACS data (not shown), these mutants have significantly reduced collateral effect compared to wild-type.

Further, some of the Cas13d mutants exhibited low collateral effect (e.g., ≤27.5% collateral effect, or ≥72.5% mCherry⁺ cells), and intermediate gRNA-guided cleavage (e.g., 25%≤EGFP⁺ cells≤75%), including: N2V4, N2V5, N4V3, N6V3, N10V6, N15V2, N20V6, and N20-Y910A, etc. (see above table and FIG. 17D). The gRNA-guided cleavage efficiency for these mutants can be enhanced further by, for example, using multiple gRNA targeting different sites of the target sequence, and the collateral effect would remain low.

In other words, the invention has provided mutants having substantially retained (e.g., retaining at least about 50%, 60%, 70%, 80%, 85%, 90%, 95%, 96%, 97%, 98%, or 99%) wild-type level gRNA-guided cleavage, while substantially reducing/eliminating (at least about 72.5%, 75%, 80%, 85%, 90%, 95%, 96%, 97%, 98%, 99%, or 100%) Cas13d collateral effect.

Since N2V7 and N2V8 retained relatively high guide RNA-specific cleavage, with essentially eliminated Cas13d collateral effect, and the residues affected by these mutants are very close together, further mutagenesis study in the two regions of these mutants was conducted, by generating a number of additional mutants with single, double, triple, or quadruple combination mutations. The sequences of these mutants and the corresponding wild-type sequences (N2C) are listed below:

| | | SEQ | | SEQ |
|---|---|---|---|---|
| | | ID | | ID |
| Variants | Amino Acids | NO: | DNA | NO: |
| N2C | ILAEYITNAAYAVNNIS | 561 | ATCCTGGCTGAGTACATCACAAACGCCGCTTACGCCGTGAAC | 562 |
| | | | AACATCTCC | |
| I132A | ALAEYITNAAYAVNNIS | 563 | gccCTGGCTGAGTACATCACAAACGCCGCTTACGCCGTGAAC | 564 |
| | | | AACATCTCC | |
| L133A | IAAEYITNAAYAVNNIS | 565 | ATCgccGCTGAGTACATCACAAACGCCGCTTACGCCGTGAAC | 566 |
| | | | AACATCTCC | |
| A134V | ILVEYITNAAYAVNNIS | 567 | ATCCTGgccGAGTACATCACAAACGCCGCTTACGCCGTGAAC | 568 |
| | | | AACATCTCC | |
| E135A | ILAAYITNAAYAVNNIS | 569 | ATCCTGGCTgccTACATCACAAACGCCGCTTACGCCGTGAAC | 570 |
| | | | AACATCTCC | |
| I137A | ILAEYATNAAYAVNNIS | 571 | ATCCTGGCTGAGTACgccACAAACGCCGCTTACGCCGTGAAC | 572 |
| | | | AACATCTCC | |
| T138A | ILAEYIANAAYAVNNIS | 573 | ATCCTGGCTGAGTACATCgccAACGCCGCTTACGCCGTGAAC | 574 |
| | | | AACATCTCC | |
| N139A | ILAEYITAAAYAVNNIS | 575 | ATCCTGGCTGAGTACATCACAgccGCCGCTTACGCCGTGAAC | 576 |
| | | | AACATCTCC | |
| Al40V | ILAEYITNVAYAVNNIS | 577 | ATCCTGGCTGAGTACATCACAAACgtgGCTTACGCCGTGAAC | 578 |
| | | | AACATCTCC | |
| A141V | ILAEYITNAVYAVNNIS | 579 | ATCCTGGCTGAGTACATCACAAACGCCgtgTACGCCGTGAAC | 580 |
| | | | AACATCTCC | |
| Al43V | ILAEYITNAAYVVNNIS | 581 | ATCCTGGCTGAGTACATCACAAACGCCGCTTACgtgGTGAAC | 582 |
| | | | AACATCTCC | |
| V144A | ILAEYITNAAYAANNIS | 583 | ATCCTGGCTGAGTACATCACAAACGCCGCTTACGCCgccAAC | 584 |
| | | | AACATCTCC | |
| N145A | ILAEYITNAAYAVANIS | 585 | ATCCTGGCTGAGTACATCACAAACGCCGCTTACGCCGTGgcc | 586 |
| | | | AACATCTCC | |
| N146A | ILAEYITNAAYAVNAIS | 587 | ATCCTGGCTGAGTACATCACAAACGCCGCTTACGCCGTGAAC | 588 |
| | | | gccATCTCC | |
| I147A | ILAEYITNAAYAVNNAS | 589 | ATCCTGGCTGAGTACATCACAAACGCCGCTTACGCCGTGAAC | 590 |
| | | | AACgccTCC | |
| S148A | ILAEYITNAAYAVNNIA | 591 | ATCCTGGCTGAGTACATCACAAACGCCGCTTACGCCGTGAAC | 592 |
| | | | AACATCgcc | |
| N2D1 | ALAAYATNAAYAVNNIS | 593 | gccCTGGCTgccTACgccACAAACGCCGCTTACGCCGTGAAC | 594 |
| | | | AACATCTCC | |
| N2D2 | ALAAYITNAAYAVNNAS | 595 | gccCTGGCTgccTACATCACAAACGCCGCTTACGCCGTGAAC | 596 |
| | | | AACgccTCC | |
| N2D3 | ALAEYATNAAYAVNNAS | 597 | gccCTGGCTGAGTACgccACAAACGCCGCTTACGCCGTGAAC | 598 |
| | | | AACgccTCC | |
| N2D4 | ILAAYATNAAYAVNNAS | 599 | ATCCTGGCTgccTACgccACAAACGCCGCTTACGCCGTGAAC | 600 |
| | | | AACgccTCC | |
| N2D5 | ALAAYITNAAYAVNNIS | 601 | gccCTGGCTgccTACATCACAAACGCCGCTTACGCCGTGAAC | 602 |
| | | | AACATCTCC | |
| N2D6 | ALAEYATNAAYAVNNIS | 603 | gccCTGGCTGAGTACgccACAAACGCCGCTTACGCCGTGAAC | 604 |
| | | | AACATCTCC | |
| N2D7 | ALAEYITNAAYAVNNAS | 605 | gccCTGGCTGAGTACATCACAAACGCCGCTTACGCCGTGAAC | 606 |
| | | | AACgccTCC | |
| N2D8 | ILAAYATNAAYAVNNIS | 607 | ATCCTGGCTgccTACgccACAAACGCCGCTTACGCCGTGAAC | 608 |
| | | | AACATCTCC | |
| N2D9 | ILAAYITNAAYAVNNAS | 609 | ATCCTGGCTgccTACATCACAAACGCCGCTTACGCCGTGAAC | 610 |
| | | | AACgccTCC | |
| N2D10 | ILAEYATNAAYAVNNAS | 611 | ATCCTGGCTGAGTACgccACAAACGCCGCTTACGCCGTGAAC | 612 |
| | | | AACgccTCC | |
| N2D11 | ILVEYITNVVYAVNNIS | 613 | ATCCTGgtgGAGTACATCACAAACgtggtgTACGCCGTGAAC | 614 |
| | | | AACATCTCC | |
| N2D12 | ILVEYITNVAYVVNNIS | 615 | ATCCTGgtgGAGTACATCACAAACgtgGCTTACgtgGTGAAC | 616 |
| | | | AACATCTCC | |
| N2D13 | ILVEYITNAVYVVNNIS | 617 | ATCCTGgtgGAGTACATCACAAACGCCgtgTACgtgGTGAAC | 618 |
| | | | AACATCTCC | |
| N2D14 | ILAEYITNVVYVVNNIS | 619 | ATCCTGGCTGAGTACATCACAAACgtggtgTACgtgGTGAAC | 620 |
| | | | AACATCTCC | |
| N2D15 | ILVEYITNVAYAVNNIS | 621 | ATCCTGgtgGAGTACATCACAAACgtgGCTTACGCCGTGAAC | 622 |
| | | | AACATCTCC | |
| N2D16 | ILVEYITNAVYAVNNIS | 623 | ATCCTGgtgGAGTACATCACAAACGCCgtgTACGCCGTGAAC | 624 |
| | | | AACATCTCC | |
| N2D17 | ILVEYITNAAYVVNNIS | 625 | ATCCTGgtgGAGTACATCACAAACGCCGCTTACgtgGTGAAC | 626 |
| | | | AACATCTCC | |
| N2D18 | ILAEYITNVVYAVNNIS | 627 | ATCCTGGCTGAGTACATCACAAACgtggtgTACGCCGTGAAC | 628 |
| | | | AACATCTCC | |
| N2D19 | ILAEYITNVAYVVNNIS | 629 | ATCCTGGCTGAGTACATCACAAACgtgGCTTACgtgGTGAAC | 630 |
| | | | AACATCTCC | |
| N2D20 | ILAEYITNAVYVVNNIS | 631 | ATCCTGGCTGAGTACATCACAAACGCCgtgTACgtgGTGAAC | 632 |
| | | | AACATCTCC | |
| N2T | ALAAYITNAAYVVNNIS | 633 | gccCTGGCTgccTACATCACAAACGCCGCTTACgtgGTGAAC | 634 |
| | | | AACATCTCC | |
| N2Q | ALVAYITNAAYVVNNIS | 635 | gccCTGgtggccTACATCACAAACGCCGCTTACgtgGTGAAC | 636 |
| | | | AACATCTCC | |

Using the same assay above, and after normalizing the data with that of the dCas13d, mutants occupying the upper left corner of FIG. 17E were selected.

| Variants | % mCherry | S.E.M. | % EGFP | S.E.M. |
|---|---|---|---|---|
| dead | 1.0000 | 0.0153 | 1.0000 | 0.0305 |
| N2V7 | 1.0011 | 0.0539 | 0.0873 | 0.0076 |
| N2V8 | 0.9161 | 0.0259 | 0.0830 | 0.0039 |
| I132A | 0.6851 | 0.0050 | 0.0695 | 0.0065 |
| L133A | 0.1880 | 0.0048 | 0.0393 | 0.0004 |
| A134V | 0.3450 | 0.0136 | 0.0714 | 0.0060 |
| E135A | 0.4479 | 0.0280 | 0.0597 | 0.0057 |
| Y136A | 0.2225 | 0.0125 | 0.0454 | 0.0035 |
| I137A | 0.2187 | 0.0036 | 0.0418 | 0.0022 |
| T138A | 0.2702 | 0.0077 | 0.0426 | 0.0020 |
| N139A | 0.2152 | 0.0029 | 0.0346 | 0.0002 |
| A140V | 0.1912 | 0.0019 | 0.0355 | 0.0021 |
| A141V | 0.2454 | 0.0052 | 0.0472 | 0.0021 |
| Y142A | 0.1775 | 0.0029 | 0.0375 | 0.0043 |
| A143V | 0.5235 | 0.0087 | 0.0644 | 0.0057 |
| VI44 A | 0.2001 | 0.0027 | 0.0413 | 0.0036 |
| N145A | 0.3230 | 0.0152 | 0.0489 | 0.0032 |
| N146A | 0.1269 | 0.0013 | 0.0299 | 0.0012 |
| I147A | 0.1410 | 0.0067 | 0.0238 | 0.0026 |
| S148A | 0.1338 | 0.0042 | 0.0233 | 0.0007 |
| N2D1 | 0.8383 | 0.0187 | 0.3122 | 0.0250 |
| N2D2 | 0.7241 | 0.0239 | 0.0658 | 0.0056 |
| N2D3 | 0.1554 | 0.0064 | 0.0446 | 0.0039 |
| N2D4 | 0.0757 | 0.0072 | 0.0353 | 0.0010 |
| N2D5 | 0.8970 | 0.0302 | 0.1234 | 0.0112 |
| N2D6 | 0.3629 | 0.0264 | 0.0552 | 0.0079 |
| N2D7 | 0.1019 | 0.0100 | 0.0470 | 0.0058 |
| N2D8 | 0.1102 | 0.0040 | 0.0284 | 0.0039 |
| N2D9 | 0.0397 | 0.0050 | 0.0181 | 0.0017 |
| N2D10 | 0.0347 | 0.0054 | 0.0260 | 0.0016 |
| N2D11 | 0.1137 | 0.0153 | 0.0467 | 0.0023 |
| N2D12 | 0.9867 | 0.0286 | 0.2198 | 0.0047 |
| N2D13 | 0.4308 | 0.0376 | 0.0542 | 0.0066 |
| N2D14 | 0.1901 | 0.0189 | 0.0571 | 0.0019 |
| N2D15 | 0.0314 | 0.0023 | 0.0155 | 0.0003 |
| N2D16 | 0.0847 | 0.0035 | 0.0327 | 0.0016 |
| N2D17 | 0.8968 | 0.0271 | 0.1044 | 0.0088 |
| N2D18 | 0.0443 | 0.0022 | 0.0264 | 0.0016 |
| N2D19 | 0.5594 | 0.0338 | 0.0866 | 0.0103 |
| N2D20 | 0.1364 | 0.0084 | 0.0461 | 0.0014 |
| N2T | 0.7398 | 0.0150 | 0.5906 | 0.0122 |
| N2Q | 0.7333 | 0.0115 | 0.6117 | 0.0048 |
| WT | 0.0789 | 0.0070 | 0.0156 | 0.0036 |

Based on comprehensive analysis of all these mutants, N2V8 (carrying A134V, A140V, A141V, A143V) was believed to has superior characteristics, in that it retained relatively high guide RNA-specific cleavage, while essentially eliminated Cas13d collateral effect. See data above and FIGS. 17D and 17E. This mutant is sometimes referred to as cfCas13d (collateral free Cas13d) for further functional characterization.

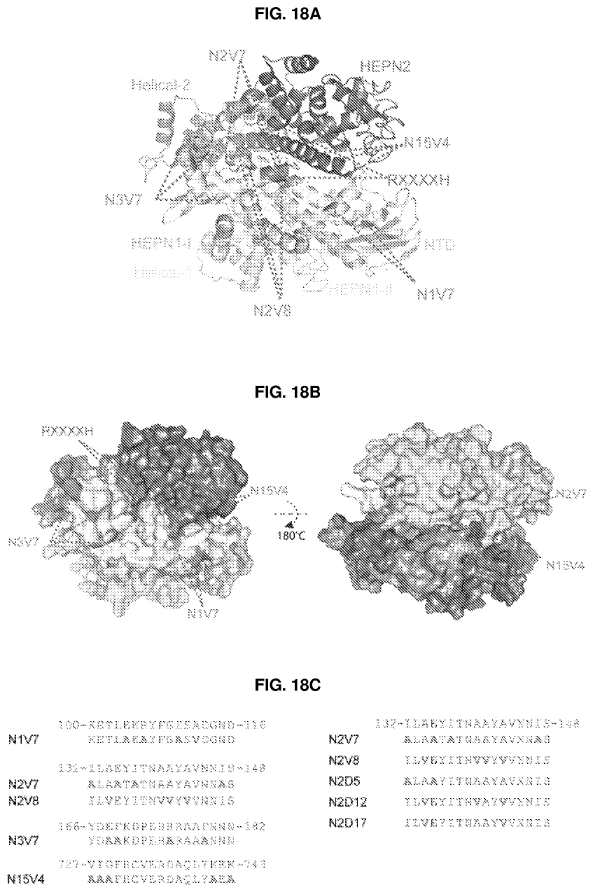

Based on the structure of Cas13d and PyMOL visualization, it was identified that the mutation sites of various effective variants were mainly located in a-helix proximal to catalytic sites of two HEPN domains (RXXXXH-1, RXXXXH-2) (FIGS. 18A-18C), especially for mutants N1V7, N2V7, N2V8, and N15V4. See FIGS. 18A-18C. It is believed that residues in these regions may have participated in binding between Cas13d to the target RNA and/or the non-specific RNA, and mutations in these residues had different/differential effects on Cas13d affinity towards different RNA targets, hence the cleavage efficiency towards these RNA targets.

The identified desired Cas13d mutants with reduced/eliminated collateral effects seem to share the following characteristics:

1. mutations are mainly located within the HEPN1-1 domain (e.g., residues 90-292), Helical2 domain (e.g., residues 536-690), and the HEPN2 domain (e.g., residues 690-967 in Cas13d).

2. in Cas13d, mutations are located within 170 residues of the RXXXXH motif.

3. most mutations, in 3D structure, are in the vicinity of the catalytic activity site formed by the RXXXXH motifs of HEPN1 and HEPN2 domains.

4. for each mutated residue, substitutions by residues other than Ala (especially Val, Gly, and Ile), are similarly effective to reduce/eliminate collateral effect.

Certain specific positions of the desired mutants in Cas13d are listed below:

| | | | SEQ |
|---|---|---|---|
| | | | ID |
| Variants | Mutations | Amino Acids | NO: |
| N1R | | QDMLGLKETLEKRYFGESA | 117 |
| N1V7 | E104A, R106A, E110A, A112V | QDMLGLKETLAKAYFGASV | 125 |
| N2L | | DGNDNICIQVIHNILDI | 129 |
| N2V4 | I120A, I123A, I126A, I129A | DGNDNICAQVAHNALDA | 137 |
| N2R | | EKILAEYITNAAYAVNNIS | 139 |
| N2V5 | E130A, K131A, T138A, N139A, S148A | AAILAEYIAAAAYAVNNIA | 141 |
| N2V7 | I132A, E135A, I137A, I147A | EKALAAYATNAAYAVNNAS | 145 |
| N2V8 | A134V, A140V, A141V, A143V | EKILVEYITNVVYVVNNIS | 147 |
| N3R | | YDEFKDPEHHRAAFNNNDK | 165 |
| N3V7 | E168A, F169A, H175A, F179A | YDAAKDPEHARAAANNNDK | 171 |
| N4L | | LINAIKAQYDEFDNFLD | 177 |
| N4V3 | I186A, I189A, F196A, L200A | LANAAKAQYDEADNFAD | 183 |
| N6L | | LYNLDKNLDNEYISTLN | 219 |
| N6V3 | L260A, L264A, E267A, I269A, L272A | LYNADKNADNAYASTAN | 225 |
| N10R | | AMYIDAIRILGTNLSYDEL | 321 |
| N10V6 | D551A, L556A, N559A, D563A | AMYIAAIRIAGTALSYAEL | 325 |
| N15L | | TVIYHILKNIVNINARY | 423 |
| N15V2 | V711A, I712A, H714A, L716A, V720A | TAAYAIAKNIANINARY | 427 |
| N15R | | VIGFHCVERDAQLYKEKGY | 431 |
| N15V4 | V727A, I728A, G729A, K741A, K743A | AAAFHCVERDAQLYAEAGY | 433 |
| N20R | | VSEYFDAVNDEKKYNDRLL | 527 |
| N20V6 | N915A, D916A, N921A, D922A | VSEYFDAVAAEKKYAARLL | 531 |
| N20-Y910A | Y910A | VSEAFDAVNDEKKYNDRLL | 537 |

Interestingly, the majority of variants exhibited either low dual cleavage activity (upper right in FIG. 17D) or high on-target cleavage activity but low collateral cleavage activity (upper left in FIG. 17D). However, there is almost no variants showing low on-target cleavage activity but high collateral cleavage activity (bottom right in FIG. 17D). These results suggest a distinct binding mechanism between on-target and collateral cleavage activity.

To confirm the elimination of collateral effects by cfCas13d, EGFP was targeted with other three different gRNAs, and substantial collateral effects was found to be induced by the wild-type Cas13d, but essentially no collateral effects were induced by cfCas13d (FIG. 17F).

Next, in vitro cleavage activities of purified Cas13d and cfCas13d proteins on targeted RNAs, in the presence of non-targeted single-strand RNA probes, were investigated. It was found that cfCas13d exhibited consistently efficient on-target activity with essentially no collateral cleavage, whereas wild-type Cas13d showed notable collateral activity (FIGS. 17G and 17H). These results further demonstrated that collateral effects were largely eliminated by cfCas13d.

On the other hand, the above screening also produced multiple mutants with significantly enhanced collateral effect, based on ≥87.5% collateral cleavage efficiency (e.g., ≤12.5% mCherry⁺ cells) and better gRNA-guided cleavage compared to wild-type (e.g., ≤4% EGFP⁺ cells). These mutants include: N2-Y142A, N4-Y193A, N12-Y604A, N21V7, etc. Among them, N2-Y142A is located in the Helical2 domain, extending towards the two HEPN domains in the 3D structure. Meanwhile, N4-Y193A and N21V7 are within the HEPN1 and HEPN2 domains, respectively, and are relatively far away from the catalytic active site. The residues involved in these mutants are listed below.

| | | | SEQ |
|---|---|---|---|
| | | | ID |
| Variants | Mutations | Amino Acids | NO |
| N2R | | EKILAEYITNAAYAVNNIS | 139 |
| N2-Y142A | Y142A | EKILAEYITNAAAAVNNIS | 151 |
| N4L | | LINAIKAQYDEFDNFLD | 177 |
| N4-Y193A | Y193A | LINAIKAQADEFDNFLD | 187 |
| N12L | | FHYLIRYGDPAHLHEIA | 355 |
| N12-Y604A | Y604A | FHALIRYGDPAHLHEIA | 363 |
| N21R | | SIEALFDRNEAAKFDKEKK | 551 |
| N21V7 | A946V, R950A, | SIEVLFDANEVVKFDKEKK | 559 |
| | A953V, A954V | | |

It should be understood that, although Ala was used in the mutagenesis studies herein, other substitutions at the same positions (especially those with small (alky) side chains such as Val or Ile, or Gly), also have similar effects as Ala substitution. These mutations are expressly contemplated and disclosed herein, and are within the scope of the invention.

### Example 5 Eliminating Collateral Effects of Cas13e through Mutagenesis

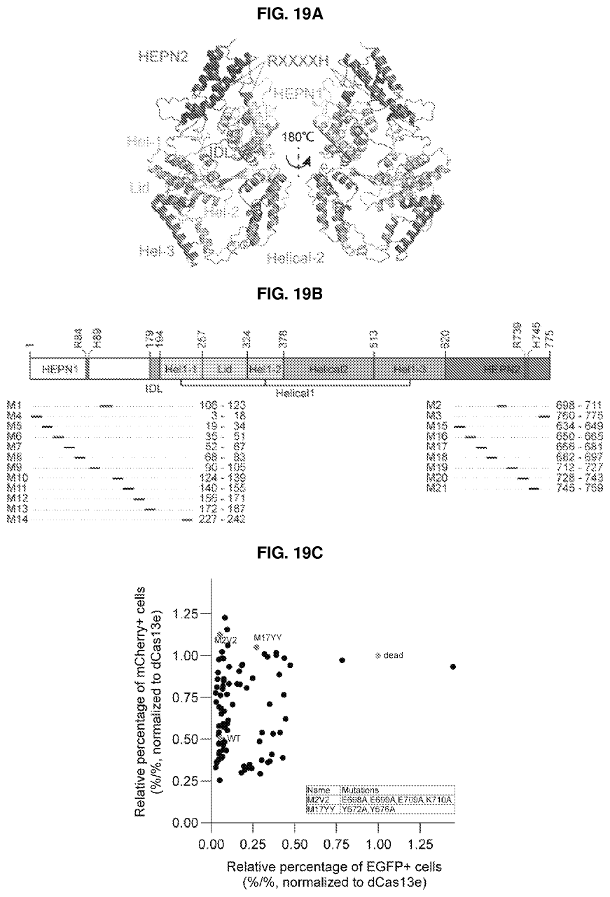

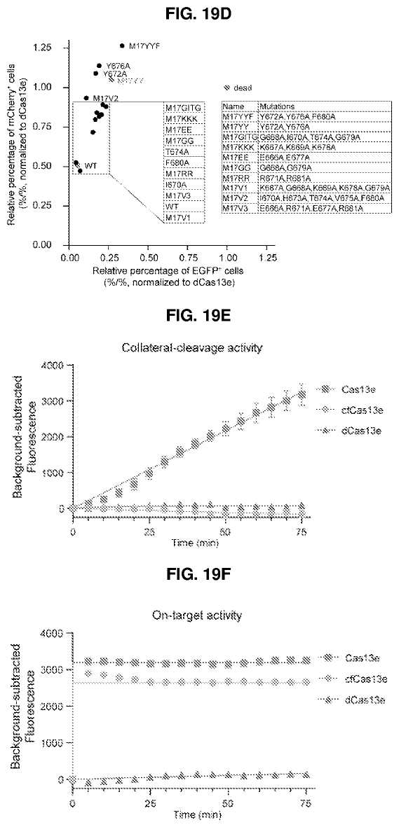

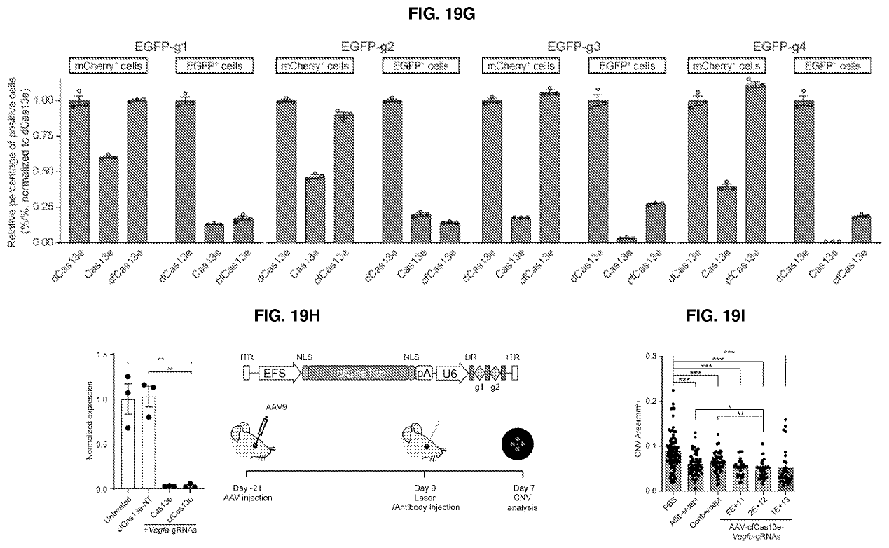

This example provides additional Cas13e mutants with reduced/eliminated collateral effect, based on knowledge of Cas13d mutants screening and simulated structural analysis of Cas13e (see FIG. 19A).

Specifically, a mutagenesis library was developed for Cas13e, covering HEPN1 and HEPN2 domains (FIG. 19B). At least 90 different mutants were constructed, each comprising 1-5 amino acid residue changes compared to the wild-type sequence. The various Cas13e mutants and the corresponding wild-type sequences (M1-M21) are listed below.

| | | SEQ | | SEQ |
|---|---|---|---|---|
| | | ID | | ID |
| | Amino Acids | NO: | DNA sequence | NO: |
| M1 | RTIMERAYERAIFECRRR | 637 | CGGACCATCATGGAGAGAGCCTATGAGCGGGCCATCTTCGA | 638 |
| | | | GTGCAGAAGAAGA | |
| M1V1 | ATIMEAAYEAAIFECAAR | 639 | gccACCATCATGGAGgccGCCTATGAGgccGCCATCTTCGA | 640 |
| | | | GTGCgccgccAGA | |
| M1V2 | RTIMARAYARAIFECRRA | 641 | CGGACCATCATGgccAGAGCCTATgccCGGGCCATCTTCgc | 642 |
| | | | cTGCAGAAGAgcc | |
| M1V3 | RAIMERAYERAIFEARRR | 643 | CGGgccATCgccGAGAGAGCCTATGAGCGGGCCATCgccGA | 644 |
| | | | GgccAGAAGAAGA | |
| M1V4 | RTAMERVYERVAFECRRR | 645 | CGGACCgccATGGAGAGAgtgTATGAGCGGgtggccTTCGA | 646 |
| | | | GTGCAGAAGAAGA | |
| M1-Y113A | RTIMERAAERAIFECRRR | 647 | CGGACCATCATGGAGAGAGCCgccGAGCGGGCCATCTTCGA | 648 |
| | | | GTGCAGAAGAAGA | |
| M2 | AFEEKVVKAKKMSEKE | 649 | GCTTTCGAAGAGAAGGTGGTGAAGGCCAAGAAGATGAGCGA | 650 |
| | | | GAAGGAA | |
| M2V1 | AFEEAVVAAAAMSEKE | 651 | GCTTTCGAAGAGgccGTGGTGgccGCCgccgccATGAGCGA | 652 |
| | | | GAAGGAA | |
| M2V2 | AFAAKVVKAKKMSAAE | 653 | GCTTTCgccgccAAGGTGGTGAAGGCCAAGAAGATGAGCgc | 654 |
| | | | cgccGAA | |
| M2V3 | AAEEKVVKAKKAAEKA | 655 | GCTgccGAAGAGAAGGTGGTGAAGGCCAAGAAGgccgccGA | 656 |
| | | | GAAGgcc | |
| M2V4 | VFEEKAAKVKKMSEKE | 657 | gtgTTCGAAGAGAAGgcegcCAAGgtgAAGAAGATGAGCGA | 658 |
| | | | GAAGGAA | |
| M3 | VMKKYGIEKEWKFPVK | 659 | GTGATGAAGAAGTACGGCATCGAGAAGGAATGGAAGTTCCC | 660 |
| | | | TGTCAAG | |
| M3V1 | VMAAYGIEAEWAFPVA | 661 | GTGATGgccgccTACGGCATCGAGgccGAATGGgccTTCCC | 662 |
| | | | TGTCgcc | |
| M3V2 | VAKKYGIAKAWKAAVK | 663 | GTGgccAAGAAGTACGGCATCgccAAGgccTGGAAGgccgc | 664 |
| | | | cGTCAAG | |
| M3V3 | AMKKYAAEKEAKFPAK | 665 | gccATGAAGAAGTACgccgccGAGAAGGAAgccAAGTTCCC | 666 |
| | | | TgccAAG | |
| M3-Y764A | VMKKAGIEKEWKFPVK | 667 | GTGATGAAGAAGgccGGCATCGAGAAGGAATGGAAGTTCCC | 668 |
| | | | TGTCAAG | |
| M4 | QVSKQTSKKRELSIDE | 669 | CAGGTGAGCAAGCAGACCTCCAAGAAGAGGGAGCTGAGCAT | 670 |
| | | | CGACGAG | |
| M4V1 | QVSAQTSAAAELSIDE | 671 | CAGGTGAGCgccCAGACCTCCgccgccgccGAGCTGAGCAT | 672 |
| | | | CGACGAG | |
| M4V2 | QVAKQTSKKRALSIAA | 673 | CAGGTGgccAAGCAGACCTCCAAGAAGAGGgccCTGAGCAT | 674 |
| | | | Cgccgcc | |
| M4V3 | AVSKQAAKKRELAIDE | 675 | gccGTGAGCAAGCAGgccgccAAGAAGAGGGAGCTGgccAT | 676 |
| | | | CGACGAG | |
| M4V4 | QASKATSKKREASADE | 677 | CAGgccAGCAAGgccACCTCCAAGAAGAGGGAGgccAGCgc | 678 |
| | | | cGACGAG | |
| M5 | YQGARKWCFTIAFNKA | 679 | TACCAGGGCGCCCGGAAGTGGTGCTTCACCATTGCCTTCAA | 680 |
| | | | CAAGGCC | |
| M5V1 | YQGAAAWCFAIAFAAA | 681 | TACCAGGGCGCCgccgccTGGTGCTTCgccATTGCCTTCgc | 682 |
| | | | cgccGCC | |
| M5V2 | YAGARKAAATIAANKA | 683 | TACgccGGCGCCCGGAAGgccgccgccACCATTGCCgccAA | 684 |
| | | | CAAGGCC | |
| M5V3 | YQAVRKWCFTAVFNKV | 685 | TACCAGgccgtgCGGAAGTGGTGCTTCACCgccgtgTTCAA | 686 |
| | | | CAAGgtg | |
| M5-Y19A | AQGARKWCFTIAFNKA | 687 | gccCAGGGCGCCCGGAAGTGGTGCTTCACCATTGCCTTCAA | 688 |
| | | | CAAGGCC | |
| M6 | LVNRDKNDGLFVESLLR | 16 | CTGGTGAACCGGGACAAGAACGACGGCCTGTTCGTGGAAAG | 18 |
| | | | CCTGCTGAGA | |
| M6V1 | LVNAAANAGLFVESLLA | 689 | CTGGTGAACgccgccgccAACgccGGCCTGTTCGTGGAAAG | 690 |
| | | | CCTGCTGgcc | |
| M6V2 | LVARDKADGLFVAALLR | 691 | CTGGTGgccCGGGACAAGgccGACGGCCTGTTCGTGgccgc | 692 |
| | | | cCTGCTGAGA | |
| M6V3 | LANRDKNDALAAESLLR | 693 | CTGgccAACCGGGACAAGAACGACgccCTGgccgccGAAAG | 694 |
| | | | CCTGCTGAGA | |
| M6V4 | AVNRDKNDGAFVESAAR | 695 | gccGTGAACCGGGACAAGAACGACGGCgccTTCGTGGAAAG | 696 |
| | | | CgccgccAGA | |
| M7 | HEKYSKHDWYDEDTRA | 20 | CACGAGAAGTACAGCAAGCACGACTGGTACGACGAAGATAC | 22 |
| | | | CCGGGCC | |
| M7V1 | AEAYSAADWYDEDTAA | 697 | gccGAGgccTACAGCgccgccGACTGGTACGACGAAGATAC | 698 |
| | | | CgccGCC | |
| M7V2 | HAKYSKHAWYAAATRA | 699 | CACgccAAGTACAGCAAGCACgccTGGTACgccgccgccAC | 700 |
| | | | CCGGGCC | |
| M7V3 | HEKYAKHDAYDEDARV | 701 | CACGAGAAGTACgCcAAGCACGACgccTACGACGAAGATgc | 702 |
| | | | cCGGgtg | |
| M7-Y55A | HEKASKHDWYDEDTRA | 703 | CACGAGAAGgccAGCAAGCACGACTGGTACGACGAAGATAC | 704 |
| | | | CCGGGCC | |
| M7-Y61A | HEKYSKHDWADEDTRA | 705 | CACGAGAAGTACAGCAAGCACGACTGGgccGACGAAGATAC | 706 |
| | | | CCGGGCC | |
| M8 | LIKCSTQAANAKAEAL | 707 | CTGATCAAGTGCAGCACCCAGGCCGCCAACGCCAAGGCTGA | 708 |
| | | | AGCCCTG | |
| M8V1 | LIACATQAANAAAAAL | 709 | CTGATCgccTGCgccACCCAGGCCGCCAACGCCgccGCTgc | 710 |
| | | | cGCCCTG | |
| M8V2 | LIKASAAAAAAKAEAL | 711 | CTGATCAAGgccAGCgccgccGCCGCCgccGCCAAGGCTGA | 712 |
| | | | AGCCCTG | |
| M8V3 | AAKCSTQVANAKAEAA | 713 | gccgccAAGTGCAGCACCCAGgtgGCCAACGCCAAGGCTGA | 714 |
| | | | AGCCgcc | |
| M8V4 | LIKCSTQAVNVKVEVL | 715 | CTGATCAAGTGCAGCACCCAGGCCgtgAACgtgAAGgtgGA | 716 |
| | | | AgtgCTG | |
| M9 | YRHSPGCLTFTAEDEL | 717 | TACCGGCATAGCCCTGGCTGCCTGACCTTCACCGCCGAGGA | 718 |
| | | | CGAACTG | |
| M9V1 | YAASPGCLTFTAAAAL | 719 | TACgccgccAGCCCTGGCTGCCTGACCTTCACCGCCgccgc | 720 |
| | | | cgccCTG | |
| M9V2 | YRHAPGALAAAAEDEL | 721 | TACCGGCATgccCCTGGCgccCTGgccgccgccGCCGAGGA | 722 |
| | | | CGAACTG | |
| M9V3 | YRHSAACATFTVEDEA | 723 | TACCGGCATAGCgccgccTGCgccACCTTCACCgtgGAGGA | 724 |
| | | | CGAAgcc | |
| M9-Y90A | ARHSPGCLTFTAEDEL | 725 | gccCGGCATAGCCCTGGCTGCCTGACCTTCACCGCCGAGGA | 726 |
| | | | CGAACTG | |
| M10 | ETEVIIEFPSLFEGDR | 727 | GAGACAGAGGTGATCATCGAGTTTCCCAGCCTGTTCGAGGG | 728 |
| | | | CGACCGG | |
| M10V1 | ATAVIIEFPSLFEGAA | 729 | gccACAgccGTGATCATCGAGTTTCCCAGCCTGTTCGAGGG | 730 |
| | | | Cgccgcc | |
| M10V2 | EAEVIIAFPALFAGDR | 731 | GAGgccGAGGTGATCATCgccTTTCCCgccCTGTTCgccGG | 732 |
| | | | CGACCGG | |
| M10V3 | ETEVIIEAASLAEADR | 733 | GAGACAGAGGTGATCATCGAGgccgccAGCCTGgccGAGgc | 734 |
| | | | cGACCGG | |
| M10V4 | ETEAAAEFPSAFEGDR | 735 | GAGACAGAGgccgccgccGAGTTTCCCAGCgccTTCGAGGG | 736 |
| | | | CGACCGG | |
| M11 | ITTAGVVFFVSFFVER | 737 | ATCACCACCGCCGGCGTGGTGTTTTTCGTGAGCTTTTTCGT | 738 |
| | | | GGAAAGA | |
| M11V1 | IATAGVVFFVAFFVAA | 739 | ATCgccACCGCCGGCGTGGTGTTTTTCGTGgccTTTTTCGT | 740 |
| | | | Ggccgcc | |
| M11V2 | ITAAGVVAAVSAFVER | 741 | ATCACCgccGCCGGCGTGGTGgccgccGTGAGCgccTTCGT | 742 |
| | | | GGAAAGA | |
| M11V3 | ITTAAAAFFVSFAVER | 743 | ATCACCACCGCCgccgccgccTTTTTCGTGAGCTTTgccGT | 744 |
| | | | GGAAAGA | |
| M11V4 | ATTVGVVFFASFFAER | 745 | gccACCACCgtgGGCGTGGTGTTTTTCgccAGCTTTTTCgc | 746 |
| | | | cGAAAGA | |
| M12 | RVLDRLYGAVSGLKKN | 24 | AGAGTGCTGGATCGGCTGTATGGAGCCGTGTCCGGCCTGAA | 26 |
| | | | GAAGAAT | |
| M12V1 | AVLAALYGAVSGLAAN | 747 | gccGTGCTGgccgccCTGTATGGAGCCGTGTCCGGCCTGgc | 748 |
| | | | cgccAAT | |
| M12V2 | RALDRLYAAVAALKKA | 749 | AGAgccCTGGATCGGCTGTATgccGCCGTGgccgccCTGAA | 750 |
| | | | GAAGgcc | |
| M12V3 | RVADRAYGVASGAKKN | 751 | AGAGTGgccGATCGGgccTATGGAgtggccTCCGGCgccAA | 752 |
| | | | GAAGAAT | |
| M12-Y162A | RVLDRLAGAVSGLKKN | 753 | AGAGTGCTGGATCGGCTGgccGGAGCCGTGTCCGGCCTGAA | 754 |
| | | | GAAGAAT | |
| M13 | EGQYKLTRKALSMYCL | 755 | GAGGGACAGTACAAGCTGACCCGGAAGGCCCTGAGCATGTA | 756 |
| | | | CTGCCTG | |
| M13V1 | AGQYALTAAALAMYCL | 757 | gccGGACAGTACgccCTGACCgccgccGCCCTGgccATGTA | 758 |
| | | | CTGCCTG | |
| M13V2 | EAAYKLARKALSAYAL | 759 | GAGgccgccTACAAGCTGgccCGGAAGGCCCTGAGCgccTA | 760 |
| | | | CgccCTG | |
| M13V3 | EGQYKATRKVASMYCA | 761 | GAGGGACAGTACAAGgccACCCGGAAGgtggccAGCATGTA | 762 |
| | | | CTGCgcc | |
| M13-Y175A | EGQAKLTRKALSMYCL | 763 | GAGGGACAGgccAAGCTGACCCGGAAGGCCCTGAGCATGTA | 764 |
| | | | CTGCCTG | |
| M13-Y185A | EGQYKLTRKALSMACL | 765 | GAGGGACAGTACAAGCTGACCCGGAAGGCCCTGAGCATGgc | 766 |
| | | | cTGCCTG | |
| Ml4 | DKKRANDNEGTNPKRH | 767 | GATAAGAAGAGAGCTAACGACAATGAGGGCACAAATCCCAA | 768 |
| | | | GCGGCAC | |
| M14V1 | DAARANDNEATNPARH | 769 | GATgccgccAGAGCTAACGACAATGAGgccACAAATCCCgc | 770 |
| | | | cCGGCAC | |
| M14V2 | AKKRAAANEGANPKRH | 771 | gccAAGAAGAGAGCTgccgccAATGAGGGCgccAATCCCAA | 772 |
| | | | GCGGCAC | |
| M14V3 | DKKRANDAAGTAPKRA | 773 | GATAAGAAGAGAGCTAACGACgccgccGGCACAgccCCCAA | 774 |
| | | | GCGGgcc | |
| M14V4 | DKKAVNDNEGTNAKAH | 775 | GATAAGAAGgccgtgAACGACAATGAGGGCACAAATgccAA | 776 |
| | | | GgccCAC | |
| M15 | KSIVFSVSDYGKLYVL | 777 | AAGAGCATCGTGTTCTCCGTGTCTGACTACGGCAAGCTGTA | 778 |
| | | | CGTGCTG | |
| M15V1 | AAIVFAVADYGALYVL | 779 | gccgccATCGTGTTCgccGTGgccGACTACGGCgccCTGTA | 780 |
| | | | CGTGCTG | |
| M15V2 | KSIAFSASAYAKLYAL | 781 | AAGAGCATCgccTTCTCCgccTCTgccTACgccAAGCTGTA | 782 |
| | | | CgccCTG | |
| M15V3 | KSAVASVSDYGKAYVA | 783 | AAGAGCgccGTGgccTCCGTGTCTGACTACGGCAAGgccTA | 784 |
| | | | CGTGgcc | |
| M15-Y643A | KSIVFSVSDAGKLYVL | 785 | AAGAGCATCGTGTTCTCCGTGTCTGACgccGGCAAGCTGTA | 786 |
| | | | CGTGCTG | |
| M15-Y647A | KSIVFSVSDYGKLAVL | 787 | AAGAGCATCGTGTTCTCCGTGTCTGACTACGGCAAGCTGgc | 788 |
| | | | CGTGCTG | |
| Ml6 | DDAEFLGRICEYFMPH | 789 | GACGATGCCGAATTCCTGGGCCGGATCTGCGAATACTTCAT | 790 |
| | | | GCCCCAC | |
| M16V1 | AAAEFAARIAEYFMPH | 791 | gccgccGCCGAATTCgccgccCGGATCgccGAATACTTCAT | 792 |
| | | | GCCCCAC | |
| M16V2 | DDAEALGRACEYAAPA | 793 | GACGATGCCGAAgccCTGGGCCGGgccTGCGAATACgccgc | 794 |
| | | | cCCCgcc | |
| M16V3 | DDVAFLGAICAYFMAH | 795 | GACGATgtggccTTCCTGGGCgccATCTGCgccTACTTCAT | 796 |
| | | | GgccCAC | |
| M16-Y661A | DDAEFLGRICEAFMPH | 797 | GACGATGCCGAATTCCTGGGCCGGATCTGCGAAgccTTCAT | 798 |
| | | | GCCCCAC | |
| Ml7 | EKGKIRYHTVYEKGFR | 28 | GAAAAGGGCAAGATCCGGTACCACACAGTGTACGAAAAGGG | 30 |
| | | | CTTTAGA | |
| M17V1 | EAAAIRYHTVYEAAFR | 799 | GAAgccgccgccATCCGGTACCACACAGTGTACGAAgccgc | 800 |
| | | | CTTTAGA | |
| M17V2 | EKGKARYAAAYEKGAR | 801 | GAAAAGGGCAAGgccCGGTACgccgccgccTACGAAAAGGG | 802 |
| | | | CgccAGA | |
| M17V3 | AKGKIAYHTVYAKGFA | 803 | gccAAGGGCAAGATCgccTACCACACAGTGTACgccAAGGG | 804 |
| | | | CTTTgcc | |
| Ml8 | AYNDLQKKCVEAVLAF | 805 | GCATACAACGACCTGCAGAAGAAGTGCGTGGAGGCCGTGCT | 806 |
| | | | GGCTTTC | |
| M18V1 | AYAALQAACAEAVLAF | 807 | GCATACgccgccCTGCAGgccgccTGCgccGAGGCCGTGCT | 808 |
| | | | GGCTTTC | |
| M18V2 | AYNDAAKKAVEAAAAF | 809 | GCATACAACGACgccgccAAGAAGgccGTGGAGGCCgccgc | 810 |
| | | | cGCTTTC | |
| M18V3 | VYNDLQKKCVAVVLVA | 811 | gtgTACAACGACCTGCAGAAGAAGTGCGTGgccgtgGTGCT | 812 |
| | | | Ggtggcc | |
| M18-Y683A | AANDLQKKCVEAVLAF | 813 | GCAgccAACGACCTGCAGAAGAAGTGCGTGGAGGCCGTGCT | 814 |
| | | | GGCTTTC | |
| Ml9 | GARYIDFREILAQTMC | 32 | GGCGCCCACTACATCGACTTCCGGGAGATCCTGGCCCAGAC | 34 |
| | | | CATGTGC | |
| M19-C727A | GARYIDFREILAQTMA | 815 | GGCGCCCACTACATCGACTTCCGGGAGATCCTGGCCCAGAC | 816 |
| | | | CATGgcC | |
| M19V1 | AARYIAFREIAAAAMC | 817 | gccGCCCACTACATCgccTTCCGGGAGATCgccGCCgccgc | 818 |
| | | | cATGTGC | |
| M19V2 | GAAYADFREALAQTAA | 819 | GGCGCCgccTACgccGACTTCCGGGAGgccCTGGCCCAGAC | 820 |
| | | | Cgccgcc | |
| M19V3 | GVHYIDAAAILVQTMC | 821 | GGCgtgCACTACATCGACgccgccgccATCCTGgtgCAGAC | 822 |
| | | | CATGTGC | |
| M19-G712A | AARYIDFREILAQTMC | 823 | GcCGCCCACTACATCGACTTCCGGGAGATCCTGGCCCAGAC | 824 |
| | | | CATGTGC | |
| M19-IA | GAHYADFREALAQTMC | 825 | GGCGCCCACTACgcCGACTTCCGGGAGgcCCTGGCCCAGAC | 826 |
| | | | CATGTGC | |
| M19-T725A | GAHYIDFREILAQAMC | 827 | GGCGCCCACTACATCGACTTCCGGGAGATCCTGGCCCAGgC | 828 |
| | | | CATGTGC | |
| M20 | KEAEKTAVNKVRRAFF | 829 | AAGGAGGCCGAAAAGACCGCAGTGAACAAGGTGAGACGCGC | 830 |
| | | | CTTCTTC | |
| M20V1 | AEAEAAAVNAVRRAFF | 831 | gccGAGGCCGAAgccgccGCAGTGAACgccGTGAGACGCGC | 832 |
| | | | CTTCTTC | |
| M20V2 | KEAEKTAAAKARRAAA | 833 | AAGGAGGCCGAAAAGACCGCAgccgccAAGgccAGACGCGC | 834 |
| | | | Cgccgcc | |
| M20V3 | KAVAKTVVNKVRRVFF | 835 | AAGgccgtggccAAGACCgtgGTGAACAAGGTGAGACGCgt | 836 |
| | | | gTTCTTC | |
| M20V4 | KEAEKTAVNKVAAAFF | 837 | AAGGAGGCCGAAAAGACCGCAGTGAACAAGGTGgccgccGC | 838 |
| | | | CTTCTTC | |
| M21 | HHLKFVIDEFGLFSD | 839 | CACCACCTGAAGTTCGTGATTGACGAGTTCGGCCTGTTCAG | 840 |
| | | | CGAC | |
| M21V1 | HHLAFVIAEFALFAA | 841 | CACCACCTGgccTTCGTGATTgccGAGTTCgccCTGTTCgc | 842 |
| | | | cgcc | |
| M21-HH | AALKFVIDEFGLFSD | 843 | gccgccCTGAAGTTCGTGATTGACGAGTTCGGCCTGTTCAG | 844 |
| | | | CGAC | |
| M21V2 | HHAKFAIDEFGAFSD | 845 | CACCACgccAAGTTCgccATTGACGAGTTCGGCgccTTCAG | 846 |
| | | | CGAC | |
| M21V3 | HHLKAVADAAGLASD | 847 | CACCACCTGAAGgccGTGgccGACgccgccGGCCTGgccAG | 848 |
| | | | CGAC | |

Using the EGFP-mCherry dual-fluorescence reporter system of the invention, these Cas13e mutants were functionally screened to assess their collateral vs. gRNA-guided cleavage activities. Specifically, according to standard cell culture methods, human HEK293 cells were grown in 24-well tissue culture plates to a suitable density before the cells were transfected with PEI reagents and plasmids that express each mutant Cas13e and the reporter system fluorescent proteins. Transfected cells were cultured at 37° C. in incubator under 5% CO₂ for about 48 hours, before measuring EGFP and mCherry signals in the cells with FACS. Mutants leading to low percentage of the gRNA-targeted EGFP signal (lower percentage of EGFP⁺ cells, as a readout for preserved gRNA-guided cleavage) and high percentage of non-targeted mCherry signal (higher percentage of mCherry⁺ cells, as a readout for lacking collateral effect) were selected.

In this experiment, dCas13e with no gRNA-guided cleavage was used as a negative control, and the results (mean±s.e.m.) were normalized against that of dCas13e and listed below. Cas13e mutants located at the upper left area of FIG. 19C had low collateral effect (high mCherry signal) and high gRNA-guided cleavage activity (low EGFP signal), and were selected as the desired low/no collateral effect mutants.

| Variants | % mCherry | S.E.M. | % EGFP | S.E.M. |
|---|---|---|---|---|
| dead | 1.0000 | 0.0172 | 1.0000 | 0.0191 |
| M1V1 | 0.7065 | 0.0068 | 0.1287 | 0.0048 |
| M1V2 | 0.4777 | 0.0143 | 0.0494 | 0.0072 |
| M1V3 | 0.6128 | 0.0217 | 0.1008 | 0.0087 |
| M1V4 | 0.9068 | 0.0114 | 0.1691 | 0.0086 |
| M1-Y113A | 0.5756 | 0.0173 | 0.0731 | 0.0062 |
| M2V1 | 0.5513 | 0.0135 | 0.0958 | 0.0050 |
| M2V2 | 1.1267 | 0.0068 | 0.0538 | 0.0010 |
| M2V3 | 0.8590 | 0.0138 | 0.0392 | 0.0025 |
| M2V4 | 0.8128 | 0.0177 | 0.0353 | 0.0006 |
| M3V1 | 0.4836 | 0.0050 | 0.0797 | 0.0056 |
| M3V2 | 0.7229 | 0.0072 | 0.0296 | 0.0023 |
| M3V3 | 0.5786 | 0.0021 | 0.0470 | 0.0035 |
| M3-Y764A | 0.6513 | 0.0114 | 0.0621 | 0.0021 |
| M4V1 | 0.4097 | 0.0131 | 0.3639 | 0.0191 |
| M4V2 | 0.3381 | 0.0185 | 0.1957 | 0.0125 |
| M4V3 | 0.3477 | 0.0061 | 0.2303 | 0.0077 |
| M4V4 | 0.2991 | 0.0131 | 0.1811 | 0.0101 |
| M5V1 | 0.9851 | 0.0023 | 0.0651 | 0.0012 |
| M5V2 | 0.5929 | 0.0161 | 0.0945 | 0.0071 |
| M5V3 | 0.4970 | 0.0269 | 0.0652 | 0.0077 |
| M5-Y19A | 0.5905 | 0.0247 | 0.0716 | 0.0034 |
| M6V1 | 0.5429 | 0.0243 | 0.0468 | 0.0023 |
| M6V2 | 0.8598 | 0.0194 | 0.0769 | 0.0073 |
| M6V3 | 0.9830 | 0.0055 | 0.0745 | 0.0049 |
| M6V4 | 1.1557 | 0.0131 | 0.0948 | 0.0077 |
| M7V1 | 1.2271 | 0.0061 | 0.0831 | 0.0038 |
| M7V2 | 0.7685 | 0.0201 | 0.0953 | 0.0054 |
| M7V3 | 1.0223 | 0.0279 | 0.0652 | 0.0028 |
| M7-Y55A | 0.7612 | 0.0293 | 0.0555 | 0.0015 |
| M7-Y61A | 0.9764 | 0.0268 | 0.0462 | 0.0045 |
| M8V1 | 0.3752 | 0.0237 | 0.3023 | 0.0185 |
| M8V2 | 0.3283 | 0.0129 | 0.2269 | 0.0118 |
| M8V3 | 0.3884 | 0.0040 | 0.4274 | 0.0041 |
| M8V4 | 0.7660 | 0.0164 | 0.4349 | 0.0279 |
| M9V1 | 1.0102 | 0.0091 | 0.3195 | 0.0045 |
| M9V2 | 0.3600 | 0.0097 | 0.3392 | 0.0210 |
| M9V3 | 0.2929 | 0.0199 | 0.2937 | 0.0220 |
| M9-Y90A | 0.5326 | 0.0092 | 0.3697 | 0.0075 |
| M10V1 | 0.3257 | 0.0184 | 0.2441 | 0.0093 |
| M10V2 | 0.3163 | 0.0089 | 0.2009 | 0.0125 |
| M10V3 | 0.9338 | 0.0095 | 1.4478 | 0.0212 |
| M10V4 | 0.7100 | 0.0126 | 0.3503 | 0.0175 |
| M11V1 | 0.8652 | 0.0040 | 0.2489 | 0.0073 |
| M11V2 | 0.9422 | 0.0200 | 0.4735 | 0.0159 |
| M11V3 | 0.9719 | 0.0059 | 0.7834 | 0.0087 |
| M11V4 | 0.4334 | 0.0156 | 0.0917 | 0.0088 |
| M12V1 | 0.5396 | 0.0160 | 0.4120 | 0.0076 |
| M12V2 | 0.3679 | 0.0114 | 0.3515 | 0.0160 |
| M12V3 | 1.0612 | 0.0218 | 0.0995 | 0.0064 |
| M12-Y162A | 0.4723 | 0.0138 | 0.0456 | 0.0033 |
| M13V1 | 1.0170 | 0.0187 | 0.3899 | 0.0246 |
| M13V2 | 0.9923 | 0.0137 | 0.3386 | 0.0124 |
| M13V3 | 0.9856 | 0.0112 | 0.4375 | 0.0112 |
| M13-Y175A | 0.5394 | 0.0126 | 0.3047 | 0.0122 |
| M13-Y185A | 0.4872 | 0.0144 | 0.2900 | 0.0106 |
| M14V1 | 0.3943 | 0.0053 | 0.0675 | 0.0026 |
| M14V2 | 0.3764 | 0.0022 | 0.0441 | 0.0010 |
| M14V3 | 0.4114 | 0.0187 | 0.0484 | 0.0030 |
| M14V4 | 0.4663 | 0.0190 | 0.0734 | 0.0006 |
| M15V1 | 0.8199 | 0.0384 | 0.0700 | 0.0026 |
| M15V2 | 0.8321 | 0.0204 | 0.1070 | 0.0039 |
| M15V3 | 1.0033 | 0.0118 | 0.3904 | 0.0055 |
| M15-Y643A | 0.9455 | 0.0359 | 0.1877 | 0.0106 |
| M15-Y647A | 0.8508 | 0.0023 | 0.0762 | 0.0023 |
| M16V1 | 0.8311 | 0.0185 | 0.1553 | 0.0029 |
| M16V2 | 0.9423 | 0.0194 | 0.1837 | 0.0046 |
| M16V3 | 0.3773 | 0.0054 | 0.0456 | 0.0026 |
| M16-Y661A | 0.4237 | 0.0193 | 0.0509 | 0.0043 |
| M17V1 | 0.4721 | 0.0165 | 0.0706 | 0.0013 |
| M17V2 | 0.9337 | 0.0121 | 0.1091 | 0.0055 |
| M17V3 | 0.5244 | 0.0312 | 0.0451 | 0.0036 |
| M18V1 | 0.2546 | 0.0060 | 0.0519 | 0.0017 |
| M18V2 | 0.8277 | 0.0224 | 0.1730 | 0.0006 |
| M18V3 | 0.8065 | 0.0300 | 0.2114 | 0.0069 |
| M18-Y683A | 0.4352 | 0.0193 | 0.0710 | 0.0050 |
| M19-C727A | 0.3308 | 0.0157 | 0.0280 | 0.0031 |
| M19V1 | 0.4785 | 0.0143 | 0.0604 | 0.0007 |
| M19V2 | 0.8989 | 0.0153 | 0.0408 | 0.0026 |
| M19V3 | 0.8012 | 0.0161 | 0.0679 | 0.0020 |
| M19-G712A | 0.3631 | 0.0131 | 0.0331 | 0.0020 |
| M19-IA | 0.7763 | 0.0052 | 0.0260 | 0.0025 |
| M19-T725A | 0.3600 | 0.0150 | 0.0353 | 0.0030 |
| M20V1 | 0.5719 | 0.0112 | 0.0812 | 0.0012 |
| M20V2 | 0.8873 | 0.0220 | 0.4079 | 0.0083 |
| M20V3 | 0.6858 | 0.0261 | 0.0598 | 0.0021 |
| M20V4 | 0.6208 | 0.0361 | 0.4449 | 0.0331 |
| M21-HH | 0.6930 | 0.0223 | 0.0489 | 0.0040 |
| M21V1 | 0.4833 | 0.0154 | 0.0608 | 0.0025 |
| M21V2 | 0.3888 | 0.0090 | 0.0632 | 0.0068 |
| M21V3 | 0.6676 | 0.0332 | 0.0785 | 0.0024 |
| M17YY | 1.0497 | 0.0035 | 0.2705 | 0.0211 |
| WT | 0.5065 | 0.0086 | 0.0552 | 0.0013 |

After screening from the mutagenesis library and further different combinations with single, double, triple or quadruple mutations, many mutants with reduced/eliminated collateral effect were identified. For example, Cas13e-M17YY (carrying Y672A, Y676A) exhibited similarly high level of EGFP knockdown and lower mCherry knockdown, compared with wild-type Cas13e (FIGS. 19C and 19D). Furthermore, with different EGFP gRNAs, or in vitro cleavage activities, similar results were observed for Cas13e-M17YY, named as cfCas13e (collateral free Cas13e), which showed effective on-target cleavage activities and considerably reduced collateral effects (FIGS. 19E-19G).

Overall, these mutants exhibited less than 25% collateral effect (e.g., ≥75% mCherry⁺ cells), and ≥75% gRNA-guided cleavage (≤25% EGFP⁺ cells). They include: M1V4, M2V2, M2V3, M2V4, M5V1, M6V2, M6V3, M6V4, M7V1, M7V2, M7V3, M7-Y55A, M7-Y61A, M11V1, M12V3, M15V1, M15V2, M15-Y643A, M15-Y647A, M16V1, M16V2, M17V2, M18V2, M18V3, M19V2, M19V3, M19-IA, etc. (see above table and FIG. 19C).

Further, some of the Cas13e mutants exhibited low collateral effect (e.g., ≤25% collateral effect, or ≥75% mCherry⁺ cells), and intermediate gRNA-guided cleavage (e.g., 25%≤EGFP⁺ cells≤75%), including: M17YY, M8V4, M9V1, M11V2, M11V3, M13V1, M13V2, M13V3, M15V3, M20V2, etc. (see above table and FIG. 19C). The gRNA-guided cleavage efficiency for these mutants can be enhanced further by, for example, using multiple gRNA targeting different sites of the target sequence, and the collateral effect would remain low.

In other words, the invention has provided mutants having substantially retained (e.g., retaining at least about 50%, 60%, 70%, 80%, 85%, 90%, 95%, 96%, 97%, 98%, or 99%) wild-type level gRNA-guided cleavage, while substantially reducing/eliminating (at least about 75%, 80%, 85%, 90%, 95%, 96%, 97%, 98%, 99%, or 100%) collateral effect.

While not wishing to be bound by any particular theory, the data presented herein seems to suggest the following mechanism for reduced/eliminated collateral effect, partly based on the analysis of the locations of the effective mutants in the 3D structure of the Cas13 effector enzyme based on PyMOL visualization. Specifically, it was found that most mutants with the desired effects (e.g., reduced/eliminated collateral effect) have mutations within the HEPN1/HEPN2 domains, usually near the RXXXXH catalytic active site. It is believed that residues in these regions may have participated in binding between Cas13e to the target RNA and/or the non-specific RNA, and mutations in these residues had different/differential effects on Cas13e affinity towards different RNA targets, hence the cleavage efficiency towards these RNA targets.

The identified desired Cas13e mutants with reduced/eliminated collateral effects seem to share the following characteristics:

1. mutations are located within the HEPN1 domain and the inter-domain linker (IDL) region (e.g., residues 1-194 in Cas13e), and the HEPN2 domain (e.g., residues 620-775 in Cas13e).

2. in Cas13e, mutations are located within 125 residues of the RXXXXH motif.

3. most mutations, in 3D structure, are in the vicinity of the catalytic activity site formed by the RXXXXH motifs of HEPN1 and HEPN2 domains.

4. for each mutated residue, substitutions by residues other than Ala (especially Val, Gly, and Ile), are similarly effective to reduce/eliminate collateral effect. These mutations are expressly contemplated and disclosed herein, and are within the scope of the invention.

Certain specific positions of the desired mutants in Cas13e are listed below:

| Variants | Mutations | Amino Acids | SEQ ID NO: |
|---|---|---|---|
| M1 | | RTIMERAYERAIFECRRR | 637 |
| M1V4 | I108A, A112V, A116V, I117A | RTAMERVYERVAFECRRR | 645 |
| M2 | | AFEEKVVKAKKMSEKE | 649 |
| M2V2 | E698A, E699A, E709A, K710A | AFAAKVVKAKKMSAAE | 653 |
| M2V3 | F697A, M709A, S708A, E711A | AAEEKVVKAKKAAEKA | 655 |
| M2V4 | A696V, V701A, V702A, A704V | VFEEKAAKVKKMSEKE | 657 |
| M15 | | KSIVFSVSDYGKLYVL | 777 |
| M5V1 | R23A, K24A, T28A, N32A, K33A | YQGAAAWCFAIAFAAA | 681 |
| M6 | | LVNRDKNDGLFVESLLR | 16 |
| M6V2 | N37A, N41A, E47A, S48A | LVARDKADGLFVAALLR | 691 |
| M6V3 | V36A, G43A, F45A, V46A | LANRDKNDALAAESLLR | 693 |
| M6V4 | L35A, L44A, L49A, L50A | AVNRDKNDGAFVESAAR | 695 |
| M7 | | HEKYSKHDWYDEDTRA | 20 |
| M7V1 | H52A, K54A, K57A, H58A, R66A | AEAYSAADWYDEDTAA | 697 |
| M7V2 | E53A, D59A, D62A, E63A, D64A | HAKYSKHAWYAAATRA | 699 |
| M7V3 | S56A, W60A, T65A, A67V | HEKYAKHDAYDEDARV | 701 |
| M7-Y55A | Y55A | HEKASKHDWYDEDTRA | 703 |
| M7-Y61A | Y61A | HEKYSKHDWADEDTRA | 705 |
| M8 | | LIKCSTQAANAKAEAL | 707 |
| M8V4 | A76V, A78V, A80V, A82V | LIKCSTQAVNVKVEVL | 715 |
| M9 | | YRHSPGCLTFTAEDEL | 717 |
| M9V1 | R91A, H92A, E102A, D103A, E104A | YAASPGCLTFTAAAAL | 719 |
| M11 | | ITTAGVVFFVSFFVER | 737 |
| M11V1 | T141A, S150A, E154A, R155A | IATAGVVFFVAFFVAA | 739 |
| M11V2 | T142A, F147A, F148A, F151A | ITAAGWAAVSAFVER | 741 |
| M11V3 | G144A, VI45A, V146A, F152A | ITTAAAAFFVSFAVER | 743 |
| M12 | | RVLDRLYGAVSGLKKN | 24 |
| M12V3 | L158A, L161A, A164V, V165A, L168A | RVADRAYGVASGAKKN | 751 |
| M13 | | EGQYKLTRKALSMYCL | 755 |
| M13V1 | E172A, K176A, RI79A, K180A, S183A | AGQYALTAAALAMYCL | 757 |
| M13V2 | G173A, Q174A, T178A, M184A, C186A | EAAYKLARKALSAYAL | 759 |
| M13V3 | L177A, A181V, L182A, L187A | EGQYKATRKVASMYCA | 761 |
| M15 | | KSIVFSVSDYGKLYVL | 777 |
| M15V1 | K634A, S635A, S639A, S641A, K645A | AAIVFAVADYGALYVL | 779 |
| M15V2 | V637A, V640A, D642A, G644A, V648A | KSIAFSASAYAKLYAL | 781 |
| M15V3 | I636A, F638A, L646A, L649A | KSAVASVSDYGKAYVA | 783 |
| M15-Y643A | Y643A | KSIVFSVSDAGKLYVL | 785 |
| M15-Y647A | Y647A | KSIVFSVSDYGKLAVL | 787 |
| M16 | | DDAEFLGRICEYFMPH | 789 |
| M16V1 | D650A, D651A, L655A, G656A, C659A | AAAEFAARIAEYFMPH | 791 |
| M16V2 | F654A, I658A, F662A, M663A, H665A | DDAEALGRACEYAAPA | 793 |
| M17 | | EKGKIRYHTVYEKGFR | 28 |
| M17V2 | I670A, H673A, T674A, V675A, F680A | EKGKARYAAAYEKGAR | 801 |
| M17YY | Y672A, Y676A | EKGKIRAHTVAEKGFR | 849 |
| M18 | | AYNDLQKKCVEAVLAF | 805 |
| M18V2 | L686A, Q687A, C690A, V694A, L695A | AYNDAAKKAVEAAAAF | 809 |
| M18V3 | A682V, E692A, A693V, A696V, F697A | VYNDLQKKCVAVVLVA | 811 |
| M19 | | GAHYIDFREILAQTMC | 32 |
| M19V2 | H714A, I716A, I721A, M726A, C727A | GAAYADFREALAQTAA | 819 |
| M19V3 | A713V, F718A, R719A, E720A, A723V | GVHYIDAAAILVQTMC | 821 |
| M19-IA | I716A, I721A | GAHYADFREALAQTMC | 825 |
| M20 | | KEAEKTAVNKVRRAFF | 829 |
| M20V2 | V735A, N736A, V738A, F742A, F743A | KEAEKTAAAKARRAAA | 833 |

One specific mutant, M17YY, to a large extent has reduced collateral effect compared to the previously identified M17.15-1 and M17.15-2 mutants (Y672A,Y676A) (see FIGS. 13-14). M17YY is sometimes referred to as cfCas13e (collateral free Cas13e) herein for further functional characterization.

On the other hand, the above screening also produced multiple mutants with significantly enhanced collateral effect, based on ≥60% collateral cleavage efficiency (e.g., ≤40% mCherry⁺ cells) and better gRNA-guided cleavage compared to wild-type (e.g., ≤5.5% EGFP⁺ cells). These mutants include: M14V2, M16V3, M18V1, M19-G712A, M19-T725A, M19-C727A, etc. These mutants are mainly located between the two catalytic active sites formed by the RXXXXH motifs. For example, M14V2 is located in the Helical1-1 domain, around the beta-turn towards the two HEPN domains in the 3D structure. Meanwhile, M16V3, M18V1, M19-G712A, M19-T725A, and M19-C727A have mutations in the HEPN2 domain, around/near the alpha-helic and the its flanking unstructured regions, all close to the catalytic active site. The residues involved in these mutants are listed below.

| Variants | Mutations | Amino Acids | SEQ ID NO |
|---|---|---|---|
| M14 | | DKKRANDNEGTNPKRH | 767 |
| M14V2 | D227A, N232A, D233A, T237A | AKKRAAANEGANPKRH | 771 |
| M16 | | DDAEFLGRICEYFMPH | 789 |
| M16V3 | A652V, E653A, R657A, E660A, P664A | DDVAFLGAICAYFMAH | 795 |
| M18 | | AYNDLQKKCVEAVLAF | 805 |
| M18V1 | N684A, D685A, K688A, K689A, V691A | AYAALQAACAEAVLAF | 807 |
| M19 | | GAHYIDFREILAQTMC | 32 |
| M19-G712A | G712A | AAHYIDFREILAQTMC | 823 |
| M19-1725A | T725A | GAHYIDFREILAQAMC | 827 |
| M19-C727A | C727A | GAHYIDFREILAQTMA | 815 |

It should be understood that, although Ala was used in the mutagenesis studies herein, other substitutions at the same positions (especially those with small (alky) side chains such as Val or Ile, or Gly), also have similar effects as Ala substitution. These mutations are expressly contemplated and disclosed herein, and are within the scope of the invention.

### Example 6 Functional Characterization of cfCas13d in Mammalian Cells

This experiment, based on using 4 different gRNA (g1-g4) targeting EGFP, demonstrates that cfCas13d has similarly high gRNA-guided target RNA cleavage as the wild-type Cas13d, yet exhibits no significant collateral effect. See FIG. 17F.

gRNA-1, g1:
(SEQ ID NO: 850)
GTCCTCCTTGAAGTCGATGCCCTTCAGCTC
gRNA-2, g2:
(SEQ ID NO: 3)
AGCACTGCACGCCGTAGGTCAGGGTGGTCA
gRNA-3, g3:
(SEQ ID NO: 851)
GCAGGACCATGTGATCGCGCTTCTCGTTGG
gRNA-4, g4:
(SEQ ID NO: 852)
GAACTTCAGGGTCAGCTTGCCGTAGGTGGC

Purified wild-type Cas13d, cfCas13d, and dCas13d were used to assess in vitro collateral effect as well as gRNA-guided target RNA cleavage. The results showed that cfCas13d did not exhibit any detectable collateral effect (FIG. 17G), while retained relatively high guide RNA directed target RNA cleavage (FIG. 17H).

The ssRNA target sequence and crRNA for determining gRNA-directed cleavage are:

ssRNA-cy5-Labeled: 5′-CY5-GGCCAGUGAAUUCGAGCUCGGUACCCGGGGAUCCUCUAGA AAUAUGGAUUACUUGGUAGAACAGCAAUCUACUCGACCUGCAGGCAUGCAAGCUUGGCGU-BHQ2-3′ (SEQ ID NO: 853), and Cas13d-crRNA (SEQ ID NO: 854).

The ssRNA target sequence and crRNA for determining collateral cleavage are: ssRNA (SEQ ID NO: 853), Cas13d-crRNA (SEQ ID NO: 854), and Collateral RNA-FMA-Labeled:

(SEQ ID NO: 856)
FAM-AAAGAUACGAGGGUGCUAUGUUUCCACGCUCC-BHQ1

### Example 7 Functional Characterization of cfCas13e in Mammalian Cells

This experiment, based on using 4 different gRNA (g1-g4) targeting EGFP, demonstrates that cfCas13e has similarly high gRNA-guided target RNA cleavage as the wild-type Cas13e, yet exhibits no significant collateral effect. See FIG. 19G.

gRNA-1, g1:
(SEQ ID NO: 3)
AGCACTGCACGCCGTAGGTCAGGGTGGTCA
gRNA-2, g2:
(SEQ ID NO: 850)
GTCCTCCTTGAAGTCGATGCCCTTCAGCTC
gRNA-3, g3:
(SEQ ID NO: 857)
TCGCCGTCCAGCTCGACCAGGATGGGCACC
gRNA-4, g4:
(SEQ ID NO: 858)
TTCGGGCATGGCGGACTTGAAGAAGTCGTG

Purified wild-type Cas13e, cfCas13e, and dCas13e were used to assess in vitro collateral effect as well as gRNA-guided target RNA cleavage. The results showed that cfCas13e did not exhibit any detectable collateral effect (FIG. 19E), while retained relatively high guide RNA directed target RNA cleavage (FIG. 19F).

The ssRNA target sequence and crRNA for determining gRNA-directed cleavage are:

ssRNA-cy5-Labeled: 5′-CY5-GGCCAGUGAAUUCGAGCUCGGUACCCGGGGAUCCUCUAG AAAUAUGGAUUACUUGGUAGAACAGCAAUCUACUCGACCUGCAGGCAUGCAAGCUUGGCGU-BHQ2-3′ (SEQ ID NO: 859), and Cas13e-crRNA (SEQ ID NO: 860).

The ssRNA target sequence and crRNA for determining collateral cleavage are: ssRNA (SEQ ID NO: 861), Cas13e-crRNA (SEQ ID NO: 862), and collateral RNA-FMA-Labeled:

(SEQ ID NO: 856)
FAM-AAAGAUACGAGGGUGCUAUGUUUCCACGCUCC-BHQ1

### Example 8 Efficacy and Specificity of cfCas13d in Mammalian Cells

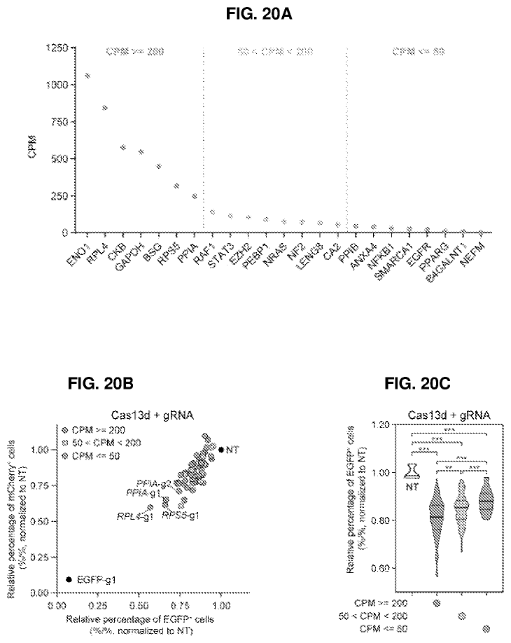

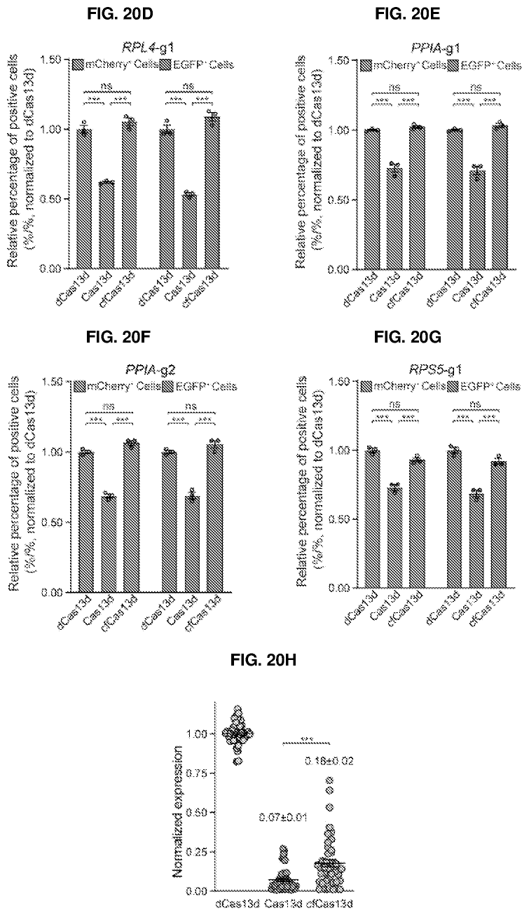

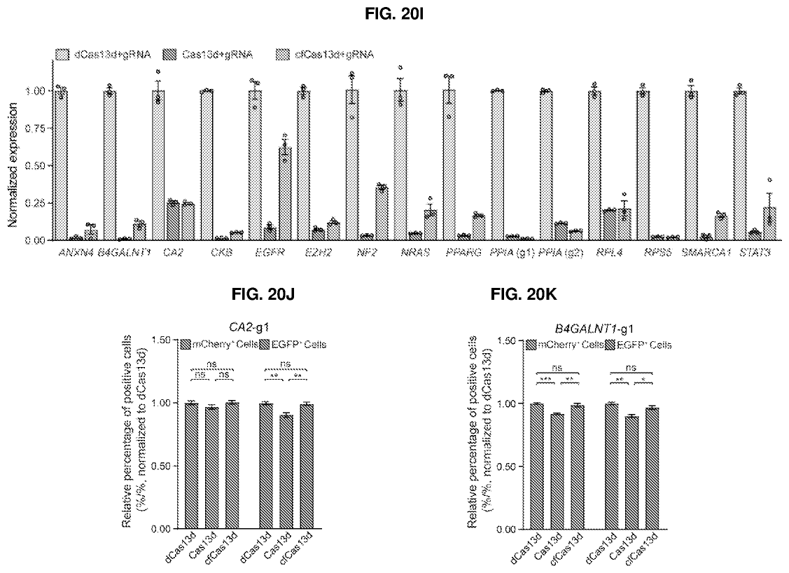

To evaluate whether the expression level of endogenous genes could affect the extend of collateral effects by Cas13d, a panel of 23 endogenous genes with diverse roles and differential expression levels in mammalian cells were selected. For each transcript, 1-6 gRNAs were then designed (FIG. 20A). Selected gRNA sequences for these target genes are listed below.

| | | | SEQ |
|---|---|---|---|
| Gene | gRNA | gRNA sequence | ID NO: |
| ANXA4 | g1 | TTAGGCAGCCCTCATCAGTGCCGGCTCCCT | 863 |
| B4GALNT1 | g1 | CCTCCTGACCAGAAGCTGCCTGAAGGCTCA | 864 |
| CA2 | g1 | AGGACAATCCAGGTCACACATTCCAGAAGA | 865 |
| CKB | 91 | GCAGCCGCTTAAGCACCTCCGAGAACTTCT | 866 |
| EGFR | g1 | GTTTCTGGCAGTTCTCCTCTCCTGCACCCC | 867 |
| EZH2 | g1 | CAAATGCTGGTAACACTGTGGTCCACAAGG | 868 |
| NF2 | g1 | CTTGGCCTGGACGGCGTAAGAAGCCAGGAG | 869 |
| NRAS | g1 | CTGTCTGGTCTTGGCTGAGGTTTCAATGAA | 870 |
| PPARG | g1 | CATTATGAGACATCCCCACTGCAAGGCATT | 871 |
| PPIA | g1 | AAACACCACATGCTTGCCATCCAACCACTC | 872 |
| PPIA | g2 | ATGCCAGGACCCGTATGCTTTAGGATGAAG | 873 |
| RPL4 | g1 | GTTGTGTTCACTCTACGATGCCAACGGCGC | 874 |
| RPL4 | g2 | GAAGTTCAGGAACTTCCTCAATACGATGAC | 875 |
| RPS5 | g1 | ACACATCCACAGCCTGTCGTCTCACAGTCC | 876 |
| SMARCA1 | g1 | CTGGTGAGGATTCCAGTCGCTGTCAAAAAT | 877 |
| STAT3 | g1 | ATCACAATTGGCTCGGCCCCCATTCCCACA | 878 |

HEK293 cells were transfected with an all-in-one construct containing Cas13d, EGFP, mCherry, non-target (NT) gRNA, or a gRNA targeting each endogenous gene, and another construct containing BFP driven by CAG promoter. BFP was used here for normalizing transfection efficiency. About 48 hours post-transfection, the EGFP and mCherry fluorescence intensity was examined for the collateral effects and target transcript level for RNA knockdown activity (FIG. 20B).

In general, increased expression level of the endogenous genes were associated with more prominent collateral effects induced by Cas13d (FIGS. 20B-20C). Specifically, obviously reduced dual fluorescence intensity was observed on genes with high expression level (ENO1, RPL4, CKB, BSG, RPS5, and PPIA, CPM>=200; CPM, counts per million), moderate but significant reduction was observed on genes with median expression level (RAF1, STAT3, EZH2, PEBEP1, NRAS, NF2, LENG8 and CA2, 50<CPM<200), and only slight decrease was observed on genes with low expression level (PPIB, ANXA4, NFKB1, SMARCA1, EGFR, PPARG, B4GALNT1, and NEFM, CPM<=50), compared with Cas13d using NT gRNA (FIG. 20B-20C).

Three individual highly expressed transcripts were selected, with four gRNAs from these endogenous genes for further characterization: RPL4-gRNA1, PPIA-gRNA1, PPIA-gRNA2, and RPS5-gRNA1. It was found that consistent notable reduced fluorescence intensity in Cas13d group but not in cfCas13d group, when compared with dCas13d group (FIGS. 20D-20G).

Meanwhile, for one medium expressed and one low expressed transcript with target gRNA: CA2-gRNA1 and B4GALNT1-gRNA1, reduced fluorescence intensity was slightly detectable in Cas13d group, but not in cfCas13d group (FIGS. 20J and 20K).

Consistently, both Cas13d and cfCas13d targeting exhibited robust knockdown of these genes, as confirmed by qPCR analysis (FIG. 20I).

These results indicate that collateral effects induced by Cas13-mediated knockdown were correlated with gene expression levels, and these collateral effects could be eliminated by cfCas13d.

To confirm that RNA interference activity by cfCas13d is still broadly applicable, cfCas13d and Cas13d were tested on randomly selected 14 endogenous transcripts in HEK293 cells. It was found that cfCas13d and Cas13d exhibited comparable efficient RNA knockdown activity (82±2% and 93±1%, respectively), indicating that cfCas13d retained high-level activity of RNA interference on most endogenous genes (FIGS. 20H and 20I).

Taken together, these results indicate that cfCas13d exhibits high RNA interference activity with rare collateral effects, which would maximize its applications.

On the other hand, multiple low-fidelity Cas13 variants exhibiting increased dual cleavage activity were obtained (bottom left in FIGS. 17D and 19C). These variants of Cas13 are better suited for nucleic acid detection applications such as SHERLOCK.

### Example 9 Elimination of Transcriptome-Wide Collateral Effects in cfCas13d

To comprehensively detect the collateral effects by Cas13d/cfCas13d-mediated knockdown, transcriptome-wide RNA sequencing (RNA-seq) was performed in Cas13d-, cfCas13d- or dCas13d-treated HEK293 cells.

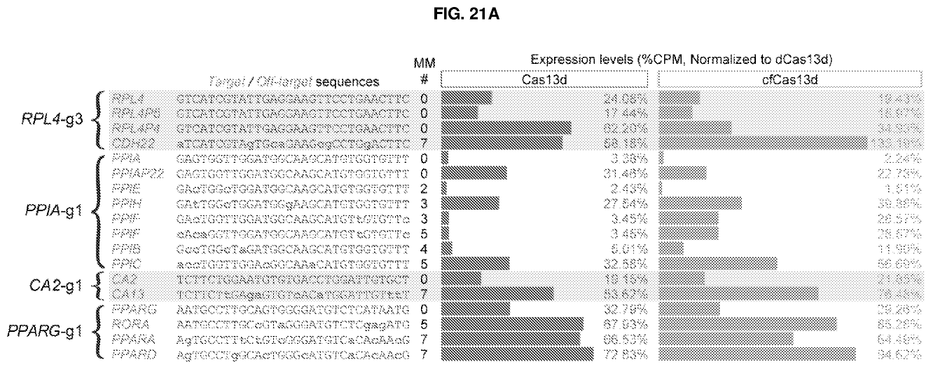

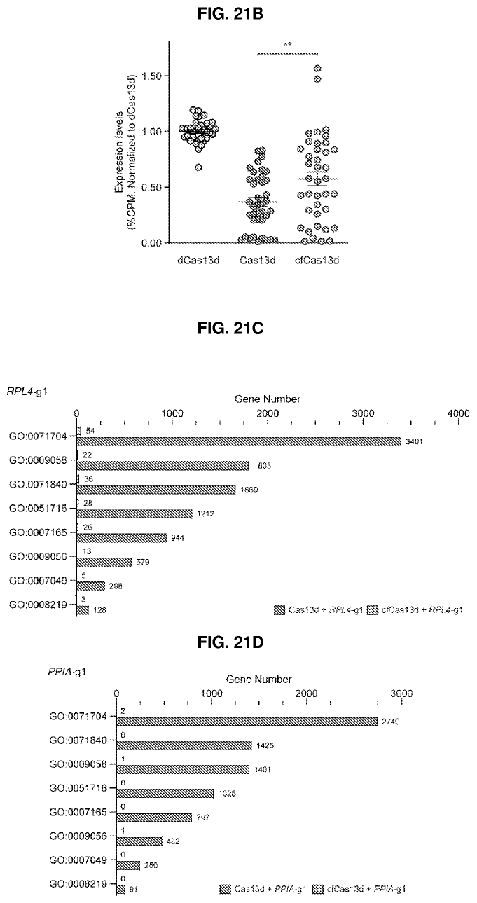

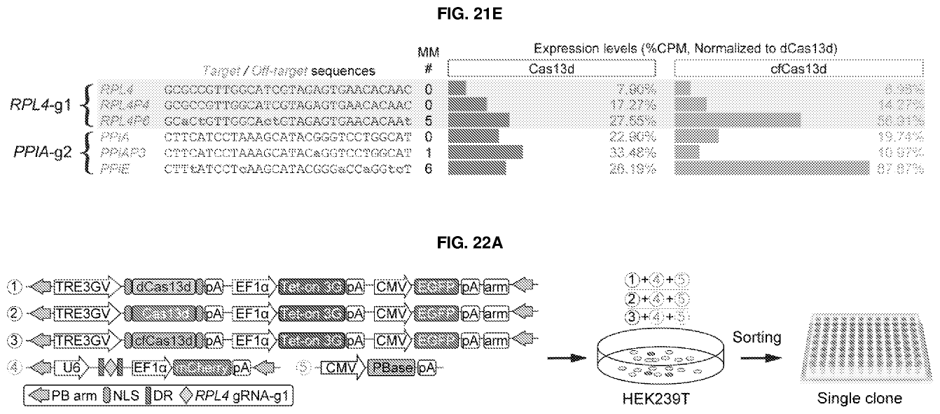

Significantly widespread off-target transcriptional changes were identified in cells that expressed Cas13d with RPL4 gRNA3 relative to dCas13d control (2007/6750 significant up/down-regulated genes, respectively), along with significant RPL4 on-target knockdown. Scatter plots of differential transcript levels between Cas13d and dCas13d-mediated RPL4, PPIA, CA2, or PPARG knockdown as determined by RNA sequencing (n=3) were not shown. Among these significant changes, 1 out of 11 predicted RPL4 gRNA-dependent off-target transcripts was identified (RPL4P5, a processed pseudogenes) (FIG. 21A). A similar pattern was observed when targeting RPL4 with a different gRNA (data not shown—Scatter plot of differential transcript levels induced by Cas13d-mediated knockdown with RPL4-g1 or PPIA-g2 as determined by RNA sequencing (n=3), compared with dCas13d).

Compared with dCas13d control, numerous off-target changes induced by Cas13d were found when targeting PPIA, CA2 or PPARG (FIGS. 21A and 21E).

Additionally, among those significantly down-regulated changes between Cas13d group and dCas13d group, targeting genes with relatively high expression level (RPL4, PPIA) induced more collateral cleavages than targeting genes with relatively low expression level (CA2, PPARG), and those collateral cleavages induced more RNA transcripts knockdown on high expressed genes than low expressed genes (data not shown—statisticalally reduced counts of down-regulated transcripts induced by Cas13d-mediated RPL4, PPIA knockdown, compared to dCas13d. Reduced counts were correlated to expression level of endogenous transcripts), in agreement with the previous results (FIGS. 20B and 20C).

Compared with Cas13d, cfCas13d remarkably reduced off-target changes when targeting RPL4 (down-regulated genes, 6750 vs. 39), PPIA (9289 vs. 8), CA2 (3519 vs. 18), and PPARG (1601 vs. 52). In addition, cfCas13d could also target predicted gRNA-dependent off-target sites as Cas13d, indicating mutations in cfCas13d decrease collateral off-target cleavage but not gRNA-dependent off-target cleavage (FIGS. 21A and 21E) (data not shown—Scatter plot of differential transcript levels induced by cfCas13d-mediated knockdown with RPL4-g1 or PPIA-g2 as determined by RNA sequencing (n=3), compared with dCas13d).

Those results suggest that cfCas13d almost eliminates off-target edits induced by Cas13d collateral activity, and those gRNA-dependent off-target could be eliminated via optimization of the design on gRNAs.

Further analysis showed that those down-regulated genes induced by CasRx targeting RPL4/PPIA gRNA were mostly distributed in metabolism, biosynthetic process, cell cycle and signal transduction pathways, while cfCasRx exhibited notable decreased off-target changes in these processes (FIGS. 21C and 21D).

When targeting RPL4, though some genes were similarly down-regulated (e.g., TP53BP2, ZMPSTE24 and FAM157C) or up-regulated (e.g., PPP1R3F), large number of unique genes were only changed in ether RPL4-g1 group or RPL4-g3 group.

Moreover, no overlaps of down-regulated or up-regulated genes were found between PPIA-g1 group and PPIA-g2 group when targeting PPIA. In addition, most of up-regulated genes from Cas13d targeting RPL4/PPIA were enriched in nucleosome assembly and gene expression pathways, related to cellular stress regulation after cleavage events (data not shown—bulk RNA-seq analysis of genes with differential expression level by Cas13d/cfCas13d targeting RPL4/PPIA, showing clustering analysis of genes with up-regulation induced by Cas13d targeting RPL4/PPIA).

Those suggested that collateral effects of Cas13d-mediated RNA reduction may inhibit cell growth, consistent with previous reports that massive host transcripts degradation induced by Cas13 result in cell retarded growth and dormancy.

These findings showed that cfCas13d maintained high specificity of on-target knockdown but collateral effects induced by Cas13d-mediated RNA knockdown were greatly reduced or even completely eliminated.

### Example 10 Elimination of Collateral Effect on Cell Growth

To further determine the cellular functional impact due to collateral effects induced by Cas13d-mediated RNA knockdown in vivo, stable cell lines were constructed by using the piggyBac transposon system with doxycycline (dox)-inducible Cas13d/cfCas13d/dCas13d expression targeting RPL4 (FIG. 22A).

Upon dox treatment, it was found that the cell clone carrying Cas13d had a significant retardation on cell growth and a notable decrease of RPL4 transcripts.

By contrast, the cell clone carrying cfCas13d exhibited no such changes on cell growth, along with a similar significant decrease of RPL4 transcripts (FIG. 22B).

These findings showed that collateral effects induced by Cas13d-mediated RNA knockdown in HEK293T cells could lead to severe cell growth retardation. Meanwhile, target RNA knockdown with a high-fidelity cfCas13d relieves cell growth stagnation.

### Example 11 Use of cfCas13e for Gene Therapy in Mouse AMD Model

Age-related macular degeneration (AMD), a progressive condition that is untreatable in up to 90% of patients, is a leading cause of blindness in the elderly worldwide. The two forms of AMD, wet and dry, are classified based on the presence or absence of blood vessels that have disruptively invaded the retina, respectively. Though wet AMD affects only 10-15% of AMD patients, it emerges abruptly, and rapidly progresses to blindness if left untreated. A detailed understanding of the molecular mechanisms underlying wet AMD has led to several robust FDA-approved therapies.

Wet AMD is typified by choroidal neovascularization (CNV), wherein newly immature blood vessels grow towards the outer retina from the underlying choroid, through a break in the Bruch membrane into the sub-retinal pigment epithelium (sub-RPE) or subretinal space. CNV is a major cause of visual loss.

Research in the late 1980s and early 1990s revealed the central role of VEGF in vascular biology, which lead to the development of the first FDA-approved anti-VEGF-A treatment for wet AMD—the monoclonal antibody Avastin (bevacizumab by Genentech). Most recently, in 2011, Eylea (VEGF-TRAP-Eye; aflibercept; Regeneron) received FDA approval for treatment of CNV. Aflibercept is a recombinant fusion protein consisting of VEGF-binding portions from the extracellular domains of human VEGF receptors 1 and 2, that are fused to the Fc portion of the human IgG1 immunoglobulin. It binds to circulating VEGFs and acts like a “VEGF trap” to inhibit the activity of VEGF-A and VEGF-B, as well as to placental growth factor (PGF), thus inhibiting the growth of new blood vessels in the choriocapillaris.

In late 2013, Chengdu Kanghong Pharmaceutical Group gained China Food and Drug Administration (CFDA) approval of Conbercept for the treatment of exudative macular degeneration. Like Conbercept is a recombinant fusion protein composed of the second Ig domain of VEGFR1 and the third and fourth Ig domains of VEGFR2 to the constant region (Fc) of human IgG1.

This example utilizes a mouse model of wet AMD to show that cfCas13e, just like wild-type Cas13e, can efficiently knock down VEGFA to reduce CNV.

Two VEGFA-targeting guide RNA molecules, gRNA-1 (g1) and gRNA-2 (g2), were previously identified to be able to direct high efficiency gRNA-guided VEGFA mRNA cleavage and expression knock down in mammalian cells, especially when they are used in combination (g1+g2). The corresponding DNA sequences of the gRNA are: gRNA-1 (g1) (SEQ ID NO: 879) and gRNA-2 (g2) (SEQ ID NO: 880).

In this experiment, coding sequence for cfCas13e (including two NLS sequences at the N- and C-terminus, under the EFS promoter) and the two gRNA's (g1+g2, under the control of the U6 promoter) were incorporated between the two ITR sequences of an AAV9 viral vector (with AAV9 serotype). Viral particles were injected directly into mouse subretinal space. After 21 days, laser light was used on the eyes of the experimental mouse to imitate UV-induced AMD. Seven days later, the extent of CNV in the experimental animals were determined (see FIGS. 19H and 19I).

In FIG. 19H, expression of VEGFA target mRNA was normalized against untreated control animals. It is apparent that, when only a non-targeting (NT) guide RNA was provided, cfCase13e did not affect VEGFA expression. In contrast, when both g1 and g2 guide RNA's were provided, cfCas13e efficiently knocked down VEGFA expression to the same extent as the wild-type Cas13e, and to nearly undetectable level (FIG. 19H).

As another control, certain control animals were also treated, at the time of laser treatment, either Aflibercept or Conbercept (FIG. 19H). The results in FIG. 19I showed that both treatments significantly reduced CNV area compared to PBS control. Notably, all three doses of cfCas13e treatments (5E11, 2E11, and 1E13 vg/kg) significantly reduced CNV (FIG. 19I). Compared to both Aflibercept and Conbercept treatments, the 2E11 dose achieved statisticalally significantly better (lower) CNV area (FIG. 19I).

In this experiment, the ITR sequence for the AAV9 viral vector is SEQ ID NO: 881, and the nucleotide sequence of the EFS promoter used to drive cfCas13e expression is SEQ ID NO: 882.

In summary, by combining analysis of 3D structure and protein sequence, Applicant has designed, constructed, and obtained by screening numerous mutant Cas13 variants with reduced or eliminated collateral effect (as well as variants with enhanced collateral effects). The guide RNA-mediated functions of these Cas13e and Cas13d mutants/variants have been verified by in vitro biochemical reactions, endogenous gene expression knock down in mammalian cells, as well as gene therapy in an in vivo mouse model of AMD.

These results demonstrate that the collateral effects of the Cas13 family proteins, including but not limited to Cas13d and Cas13e, can be engineered according to the methods and examples of the invention by, for example, introducing point mutations in and around the RXXXXH catalytic active sites within the HEPN domains (HEPN1 and HEPN2). These introduced mutations may not affect binding between the respective cfCas13 protein and the cognate gRNA, such that the cfCas13 mutants can still be activated to cleave target RNA in a gRNA-dependent manner. Meanwhile, the cfCas13 mutants have greatly reduced collateral effect compared to the corresponding wild-type Cas13, thus eliminating one significant risk of using Cas13 in gene therapy. A possible (non-limiting) mechanism of how cfCas13 mutants operate is illustrated in FIG. 22C.

Materials and Methods for the examples are provided below.

### Construction of Plasmids.

The Cas13d (CasRx) gene and gRNA backbone sequences were synthesized by a commercial source. Vectors CAG-Cas13d-p2A-GFP and U6-DR-BpiI-BpiI-DR-EF1α-mCherry were generated to knockdown target genes by transient transfection. The gRNA oligos were annealed and ligated into BpiI sites. The gRNA sequences were listed below.

| gRNA Name | gRNA sequence | SEQ ID NO |
|---|---|---|
| Cas13d mCherry gRNA-g1 | ACTTGATGTTGACGTTGTAGGCGCCGGGCA | 883 |
| Cas13d mCherry gRNA-g2 | CACGTAGGCCTTGGAGCCGTACATGAACTG | 884 |
| Cas13d mCherry gRNA-g3 | GCAGCTTCACCTTGTAGATGAACTCGCCGT | 885 |
| Cas13d non-target gRNA-NT | CGTCTGGCCTTCCTGTAGCCAGCTTTCATC | 886 |
| Cas13a mCherry gRNA-g1 | ACTTGATGTTGACGTTGTAGGCGCCGGGCA | 883 |
| Cas13a mCherry gRNA-g2 | CACGTAGGCCTTGGAGCCGTACATGAACTG | 884 |
| Cas13a mCherry gRNA-g3 | GCAGCTTCACCTTGTAGATGAACTCGCCGT | 885 |
| Cas13a non-target gRNA-NT | CGTCTGGCCTTCCTGTAGCCAGCTTTCATC | 886 |
| Human RPL4 Cas13d gRNA-g1 | GTTGTGTTCACTCTACGATGCCAACGGCGC | 874 |
| Human RPL4 Cas13d gRNA-g2 | CTTTAGACATGACCAGTGCTGGTAGGGCTG | 887 |
| Human RPL4 Cas13d gRNA-g3 | GAAGTTCAGGAACTTCCTCAATACGATGAC | 875 |
| Human RPL4 Cas13d gRNA-g4 | GGTTTCTCATTTTGCCTTTGCCAGCTCTCA | 888 |
| Human PKM Cas13d gRNA-g1 | GGCTCCCTTCTTCAGCTCCACCTCTGCAGT | 889 |
| Human PKM Cas13d gRNA-g2 | GTAGGCGTTATCCAGCGTGATTTTGAGAGT | 890 |
| Human PKM Cas13d gRNA-g3 | GTTCTTGTAGTCCAGCCACAGGATGTTCTC | 891 |
| Human PKM Cas13d gRNA-g4 | GTAGATCTTGCTGCCCACTTCCACCACCTT | 892 |
| Human PFN1 Cas13d gRNA-g1 | GGTCTTTGCCAACCAGGACACCCACCTCAG | 893 |
| Human PFN1 Cas13d gRNA-g2 | GCAGTGAGTCCCGGATCACCGAACATTTCT | 894 |
| Human PFN1 Cas13d gRNA-g3 | GTGCTCTTGGTACGAAGATCCATGCTAAAT | 895 |
| Human PFN1 Cas13d gRNA-g4 | GTCAGTCTTGGTGACAGTGACATTGAAGGT | 896 |
| Cas13d EGFPgRNA-g1 | GTCCTCCTTGAAGTCGATGCCCTTCAGCTC | 850 |
| Cas13d EGFPgRNA-g2 | AGCACTGCACGCCGTAGGTCAGGGTGGTCA | 3 |
| Cas13d EGFPgRNA-g3 | GCAGGACCATGTGATCGCGCTTCTCGTTGG | 851 |
| Cas13d EGFPgRNA-g4 | GAACTTCAGGGTCAGCTTGCCGTAGGTGGC | 852 |
| Human BSG Cas13d gRNA-g1 | ACTCGTAAGTGCCCGTGTCCTCCTCCACGA | 897 |
| Human BSG Cas13d gRNA-g2 | GTCATTCAAGGAGCAGGTGAGGAGTATCTT | 898 |
| Human BSG Cas13d gRNA-g3 | TCTGACGACTTCACAGCCTTCACTCTGGGA | 899 |
| Human BSG Cas13d gRNA-g4 | CTTGTCCTCAGAGTCAGTGATCTTGTACCA | 900 |
| Human BSG Cas13d gRNA-g5 | GTGCAGAGCCGGCGTCGTCATCATCCAGGA | 901 |
| Human CA2 Cas13d gRNA-g1 | AGCACAATCCAGGTCACACATTCCAGAAGA | 865 |
| Human CA2 Cas13d gRNA-g2 | TATGCCAGTGCTCAGGTCCGTTGTGTTTGC | 902 |
| Human CA2 Cas13d gRNA-g3 | CAGGGAAGTTGCTTGATCATAGGAAACAGA | 903 |
| Human CA2 Cas13d gRNA-g4 | AAACTGAATCAATCTGTAAGTGCCATCCAG | 904 |
| Human CA2 Cas13d gRNA-g5 | TTTATCCACAGTATGCTCTGAACCTTGTCC | 905 |
| Human CA2 Cas13d gRNA-g6 | GAATCCAGCACATCAACAACTTTCTGAAGG | 906 |
| Human CA2 Cas13d gRNA-g7 | GCGCCAGTTGTCCACCATCAGTTCTTCGGG | 907 |
| Human CKB Cas13d gRNA-g1 | GCAGCCGCTTAAGCACCTCCGAGAACTTCT | 866 |
| Human CKB Cas13d gRNA-g2 | TGGCCCGGGTTGTCCACGCCTGTCTGGATG | 908 |
| Human CKB Cas13d gRNA-g3 | CGCCCGCCACGCAGCCCACGGTCATGATGT | 909 |
| Human CKB Cas13d gRNA-g4 | CTGCTGCTGCTCCGCCTCCGTCATGCTCTT | 910 |
| Human CKB Cas13d gRNA-g5 | GTCTTATTGTCATTGTGCCAGATACCGCGG | 911 |
| Human ENO1 Cas13d gRNA-g1 | ATATAGCGAGTCTTATCATTGTCCCGGAGC | 912 |
| Human ENO1 Cas13d gRNA-g2 | ATCATCAGTTTGTCAATCTTCTCTTGTTCT | 913 |
| Human ENO1 Cas13d gRNA-g3 | CGCCATTGATGACATTGAACGCCGGGACTG | 914 |
| Human EN01 Cas13d gRNA-g4 | TCTTTCCCATATTTCTCCTTGATGACATTC | 915 |
| Human ENO1 Cas13d gRNA-g5 | CCTTATCAGTGTAGCCAGCTTTCCCAATAG | 916 |
| Human ENO1 Cas13d gRNA-g6 | ACTACCTGGATTCCTGCACTGGCTGTGAAC | 917 |
| Human LENG8 Cas13d gRNA-g1 | CCACCATGCTGTACTGAGAAGACCAATCTG | 918 |
| Human LENG8 Cas13d gRNA-g2 | GCAGGTTCGGTGTAGGTGTGTGGCCCATAG | 919 |
| Human LENG8 Cas13d gRNA-g3 | TGCGGTCCTTGTCCTCCTCCGACTCACAGG | 920 |
| Human LENG8 Cas13d gRNA-g4 | TTGTCCTTCATGAAGACGTTGCGGTTGCCA | 921 |
| Human LENG8 Cas13d gRNA-g5 | CCTCACACTCCAGCGCCGCCATCTTCTTTC | 922 |
| Human LENG8 Cas13d gRNA-g6 | AACGCGTAGTCCTGCTTCTCTTTCCAGTGG | 923 |
| Human NEFM Cas13d gRNA-g1 | GACCACGACTGCGAGCGGAAGCCACTGGAC | 924 |
| Human NEFM Cas13d gRNA-g2 | TTATAGGAGGAGGACACGGTGCTGGGCGAG | 925 |
| Human NEFM Cas13d gRNA-g3 | ATCTCCGCCTCAATCTCCTTATTCTGCTGC | 926 |
| Human NEFM Cas13d gRNA-g4 | CTCCACCTTGACCAGCGACGCCTCCTCGAT | 927 |
| Human NEFM Cas13d gRNA-g5 | CTTGGCGTAGCGGCATTTGAACCACTCTTC | 928 |
| Human NEFM Cas13d gRNA-g6 | TCCTGCAAATGTGCTAAATCTAGTCTCTTC | 929 |
| Human PEBP1 Cas13d gRNA-g1 | TCAGCACTTTGCCCAGCTCGTCCACCGCCG | 930 |
| Human PEBP1 Cas13d gRNA-g2 | TATTCTTAACCTGGGTGGGCGTCAGCACTT | 931 |
| Human PEBP1 Cas13d gRNA-g3 | CTTCCCTGAATCAAGACCATCCCACGAAAT | 932 |
| Human PEBP1 Cas13d gRNA-g4 | ATGCCATTCTCTGTATTTGGGATCCTTCCT | 933 |
| Human PEBP1 Cas13d gRNA-g5 | ATGTTGACCACCAGGAAATGATGCCATTCT | 934 |
| Human PEBP1 Cas13d gRNA-g6 | CCACTGCTGATGTCATTGCCCTTCATGTTG | 935 |
| Human PPIA Cas13d gRNA-g1 | AAACACCACATGCTTGCCATCCAACCACTC | 872 |
| Human PPIA Cas13d gRNA-g2 | ATGCCAGGACCCGTATGCTTTAGGATGAAG | 873 |
| Human PPIA Cas13d gRNA-g3 | CAAACAGCTCAAAGGAGACGCGGCCCAAGG | 936 |
| Human PPIA Cas13d gRNA-g4 | AACCCTTATAACCAAATCCTTTCTCTCCAG | 937 |
| Human PPIA Cas13d gRNA-g5 | CACCCTGACACATAAACCCTGGAATAATTC | 938 |
| Human PPIA Cas13d gRNA-g6 | CCTCCACAATATTCATGCCTTCTTTCACTT | 939 |
| Human RPS5 Cas13d gRNA-g1 | ACACATCCACAGCCTGTCGTCTCACAGTCC | 876 |
| Human RPS5 Cas13d gRNA-g2 | ATACTTCTCCTTCACTGCAATGTAATCCTG | 940 |
| Human RPS5 Cas13d gRNA-g3 | GAGTTAGTGAGGCGCTCCACAATGGGACAC | 941 |
| Human ANXA4 Cas13d gRNA-g1 | TTAGGCAGCCCTCATCAGTGCCGGCTCCCT | 863 |
| Human B4GALNT1 Cas13d gRNA-g1 | CCTCCTGACCAGAAGCTGCCTGAAGGCTCA | 864 |
| Human EGFR Cas13d gRNA-g1 | GTTTCTGGCAGTTCTCCTCTCCTGCACCCC | 867 |
| Human EZH2 Cas13d gRNA-g1 | CAAATGCTGGTAACACTGTGGTCCACAAGG | 868 |
| Human NF2 Cas13d gRNA-g1 | CTTGGCCTGGACGGCGTAAGAAGCCAGGAG | 869 |
| Human NFKB1 Cas13d gRNA-g1 | CTCATAGTTGTCCATAAGTGTTTTGGAAGG | 942 |
| Human NRASCas13d gRNA-g1 | CTGTCTGGTCTTGGCTGAGGTTTCAATGAA | 870 |
| Human PPARG Cas13d gRNA-g1 | CATTATGAGACATCCCCACTGCAAGGCATT | 871 |
| Human PPIB Cas13d gRNA-g1 | GGCCCGTAGTGCTTCAGTTTGAAGTTCTCA | 943 |
| Human RAF1 Cas13d gRNA-g1 | CTCAATCATCCTGCTGTCCACAGGCAGGGT | 944 |
| Human SMARCA1 Cas13d gRNA-g1 | CTGGTGAGGATTCCAGTCGCTGTCAAAAAT | 877 |
| Human STAT3 Cas13d gRNA-g1 | ATCACAATTGGCTCGGCCCCCATTCCCACA | 878 |
| Human RPL4 Cas13d gRNA-g5 | TCAGTCCAAATGCAGAAACGTCCCACATGC | 945 |
| Human RPL4 Cas13d gRNA-g6 | CAATACGATGACCTTTAGACATGACCAGTG | 946 |
| Cas13e EGFPgRNA-g1 | AGCACTGCACGCCGTAGGTCAGGGTGGTCA | 3 |
| Cas13e EGFPgRNA-g2 | GTCCTCCTTGAAGTCGATGCCCTTCAGCTC | 850 |
| Cas13e EGFPgRNA-g3 | TCGCCGTCCAGCTCGACCAGGATGGGCACC | 857 |
| Cas13e EGFPgRNA-g4 | TTCGGGCATGGCGGACTTGAAGAAGTCGTG | 858 |
| VEGFA Cas13egRNA-g1 | GTGCTGTAGGAAGCTCATCTCTCCTATGTG | 879 |
| VEGFA Cas13egRNA-g2 | GGTACTCCTGGAAGATGTCCACCAGGGTCT | 880 |

### Cell Culture, Transfection and Flow Cytometry Analysis

HEK293T cell lines were purchased from Stem Cell Bank, Chinese Academy of Sciences. HEK293T cell lines were cultured with DMEM (Gibco) supplemented with 10% fetal bovine serum (Gibco), 1% penicillin/streptomycin (Thermo Fisher Scientific) and 0.1 mM non-essential amino acids (Gibco) in an incubator at 37° C. with 5% CO₂. When cells reached 90% confluence, HEK293T cells were passaged at a ratio of 1:4 to 12-well plates. After 12 hr, 2 μg/well plasmids were transfected into cells with Lipofectamine 3000 (Thermo Fisher Scientific) using the standard protocol. 48 hr after transfection, 50,000 of both EGFP and mCherry positive cells were sorted by BD FACS Aria II for RNA extraction. For the groups of mCherry knockdown, total cells of the 12-well plate were collected for RNA extraction. Flow cytometry results were analyzed with FlowJo V10.5.3. For transgene cell lines, cells were expanded cultivation for dox (1 μg/mL) induction.

### Harvest of Total RNA and Quantitative PCR.

Total RNA was extracted by adding 500 μL Trizol (Invitrogen), 200 μL chloroform to the cells. After centrifuge at 12,000 rpm for 15 min at 4° C., the supernatant was transferred to a 1.5 mL RNase-free tube. 100% isopropanol and 75% alcohol were added to precipitate and purify the RNA. cDNA was prepared using HiScript Q RT SuperMix for qPCR (Vazyme, Biotech) according to manufacturer's instructions.

qPCR reactions were performed with AceQ qPCR SYBR Green Master Mix (Vazyme, Biotech). All of the reagents were precooled in advance. qPCR results were analyzed with—ΔΔCt method.

### Design and Construct of Cas13d Mutants

Unbiased all-in-one vectors CAG-Cas13d-U6-DR-gRNA-SV40-EGFP-SV40-mCherry and CMV-Cas13e-SV40-EGFP-SV40-mCherry-U6-DR-gRNA-DR, of which the gRNA target EGFP, were generated firstly. Then, 21 BpiI-harbouring Cas13 mutants, each spanning 36 amino acids, were introduced via site-directed mutagenesis by PCR and Gibson Assembly method using NEBuilder HiFi DNA Assembly Master Mix (New England BioLabs).

For Cas13d, to cover all the mutable regions, over a hundred of mutants with four or five random amino acid substitutions (replacing all non-alanine to alanine, X>A, and alanine to valine, A>V) were designed and generated by ligating two phosphorylated oligos (one wild-type oligo and the other mutant oligo) into corresponding BpiI-digested backbones.

To identify roles of amino acids within or nearby mutant N2V8 and N2V7, one more 17-amino-acid-span BpiI-harbouring Cas13 mutants N2R was generated, then single, double, triple or quadruple mutations were introduced by ligating annealed mutant oligos into corresponding BpiI-digested backbones.

For Cas13e, rationally designed mutants with four or five random amino acid substitutions in two regions (M17 and M18) were generated by ligating annealed mutant oligos into corresponding BpiI-digested backbones.

I-TASSER were used to perform the protein structure prediction.

### Screening for High-Fidelity Cas13d Using Flow Cytometry Analysis

Cas13 mutants screening was conducted in 48-well plates, and consolidation performed in 24-well plates. The day before transfection for screening, plate 3×10⁴ cells per well in 0.25 mL of complete growth medium. After 12 hours., 0.5 μg plasmids were transfected into HEK293 cells with 1.25 μg PEI (DNA:PEI=1:2.5).

For 24-well plates, 1×10⁵ cells were plated per well in 0.5 mL of complete growth medium, 0.8 μm plasmids were transfected into HEK293 cells with 2.5 μg PEI. 48 hours after transfection, cells were analyzed by BD FACS Aria II. Flow cytometry results were analyzed with FlowJo V10.5.3.

### Protein Purification of Cas13

Cas13 protein purification was performed according to protocol as previously described. The humanized codon-optimized gene for Cas13d/cfCas13d/Cas13e/cfCas13e was synthesized (Huagene) and cloned into a bacterial expression vector (pC013-Twinstrep-SUMO-huLwCas13a, Plasmid #90097) after the plasmid digestion by BamHI and NotI with NEBuilder HiFi DNA Assembly Cloning Kit (New England Biolabs).

The expression constructs were transformed into BL21 (DE3) (TIANGEN) cells. One liter of LB Broth growth media (Tryptone 10.0 g; Yeast Extract 5.0 g; NaCl 10.0 g, Sangon Biotech) was inoculated with ten mL of 12 hr growing culture. Cells were then grown to a cell density A600 of 0.6 at 37° C., and then SUMO-Cas13 proteins expression was induced by supplementing with 500 mM IPTG. The induced cells were grown at 16° C. for 16-18 hours before harvest by centrifuge (4,000 rpm, 20 min). Collected cells were resuspended in Buffer W (Strep-Tactin Purification Buffer Set, IBA) and lysed using ultrasonic homogenizer (Scientz).

Cell debris was removed by centrifugation and the clear lysate was loaded onto StrepTactin Sepharose High Performance Column (StrepTrap HP, GE Healthcare). The non-specific binding protein and contaminants were flowed through. The target proteins were eluted with Elution Buffer (Strep-Tactin Purification Buffer Set, IBA). The N-terminal 6× His/Twinstrep-SUMO tag (“6× His” disclosed as SEQ ID NO: 947) was removed by SUMO protease (4° C., >20 hours). Then target proteins were subjected to a final polishing step by gel filtration (S200, GEHealthcare). The purity of >95% was assessed by SDS-PAGE.

Cas13 on-Target and Collateral Cleavage Activity Assay

Fluorescent labeled ssRNA reporter assay for Cas13 nuclease activity was performed as previously described. For on-target cleavage activity analysis, assays were performed with 45 nM purified Cas13d/cfCas13d/Cas13e/cfCas13e, 22.5 nM crRNA, 125 nM quenched fluorescent RNA reporter (Sangon Biotech), 1 μL murine RNase inhibitor (New England Biolabs), 100 ng of background total human RNA (purified from HEK293T cell culture), and varying amounts of input nucleic acid target, unless otherwise indicated, in nuclease assay buffer (40 mM Tris-HCl including 25 mM Tris-HCl, pH7.5 and 25 mM Tris-HCl, pH7.0, 60 mM NaCl, 6 mM MgCl₂, pH 7.3). Reactions were allowed to proceed for 1-3 hr at 37° C. on a fluorescent plate reader (Analytik Jena) with fluorescent kinetics measured every 5 min.

### RNA-Seq and Analysis

For transcriptome sequencing, 35 μg all-in-one plasmids were transfected into HEK293 cells cultured in 10-cm dishes. Then 600,000 dual-positive EGFP⁺/mCherry⁺ (top 15%) cells were sorted out to make a pool for sequencing. Total RNA was extracted with TRIZOL-based method, fragmented and reverse transcribed to cDNAs with HiScript Q RT SuperMix for qPCR (Vazyme, Biotech) according to manufacturer's instructions. RNA-seq library was generated and quality was assessed using Illumina Hiseq X-ten platform in Novogene. Differential analysis among cell groups (RPL4 gRNA1, RPL4 gRNA3, PPIA gRNA1, PPIA gRNA2, CA2 gRNA1, and PPARG gRNA1) was done by a count-based method limma, which is implemented in R and voom is involved for normalization. Significantly expressed genes were first screened by BH-adjusted P value 0.05, further filtered with 2 fold-change. After enrichment analysis with GSEA v3.0 (Broad Institute, PreRanked mode), and the t-statistical output from limma as the metrics for ranking, 1,000 gene sets permutations were set as default, and gene sets were obtained through collecting pathways from KEGG and biological processes from GO. A gene set with an FDR P value<0.05 will be considered as significant enrichment.

Growth curve

Single cell clones with dCas13d/Cas13d/cfCas13d and RPL4 gRNA were plated on a 24-well plate at 2×10⁵ cells/mL with or without dox treated (1 μg/mL). Cell were collected at 24, 48, 72, 96 and 120 hrs. Cell number was counted by an automated cell counter (C10311, Invitrogen). Experiments were performed for three replicates.

### Determination of Cell Proliferation.

Cell proliferation was assessed by using a colorimetric thiazolyl blue (MTT) assay. Briefly, single cell clones with dCas13d/Cas13d/cfCas13d and RPL4 gRNA were treated with or without dox treated (1 μg/mL) for 0, 24, 48, 72, 96 or 120 hrs. Then each group of cells was collected and further plated on a 24-well plate at 2×10⁵ cells/mL with or without dox treated (1 μg/mL). After an incubation period of 24 hrs at 37° C., the tetrazolium salt MTT (Sigma-Chemie) was added to a final concentration of 2 μg/mL, and incubation was continued for 4 hrs. Cells were washed 3 times and finally lysed with dimethyl sulfoxide. Metabolization of MTT directly correlates with the cell number and was quantitated by measuring the absorbance at 550 nm (reference wavelength, 690 nm) by using a microplate reader (type 7500; Cambridge Technology, Watertown, Mass.). Experiments were performed for five replicates.

### Statisticalal Analysis

Statisticalal tests performed by Graphpad Prism 8 included the two-tailed unpaired two-sample t-test or the log-rank Mantel-Cox test. The respective statisticalal test used for each figure is noted in the corresponding figure legends and significant statisticalal differences are noted as *P<0.05, **P<0.01, ***P<0.001. All values are reported as mean±s.e.m.

### Example 12 Eliminating Collateral Effects of Cas13f through Mutagenesis

Collateral RNA degradation by the Cas13 family of effector enzymes has previously been found in glioma cells, flies and mammalian cells. Based on the fast and sensitive dual-fluorescence reporter system for detecting collateral effects as described herein, this example demonstrates that Cas13f could indeed induce substantial collateral effects in HEK293T cells. The example also demonstrates that the collateral effects of other Cas13f can also be diminished (if not eliminated) via mutagenesis, based on the finding that changing RNA-binding cleft proximal to catalytic sites RXXXXH in HEPN domains may selectively decrease promiscuous RNA binding and non-target cleavage, while maintaining on-target RNA cleavage.

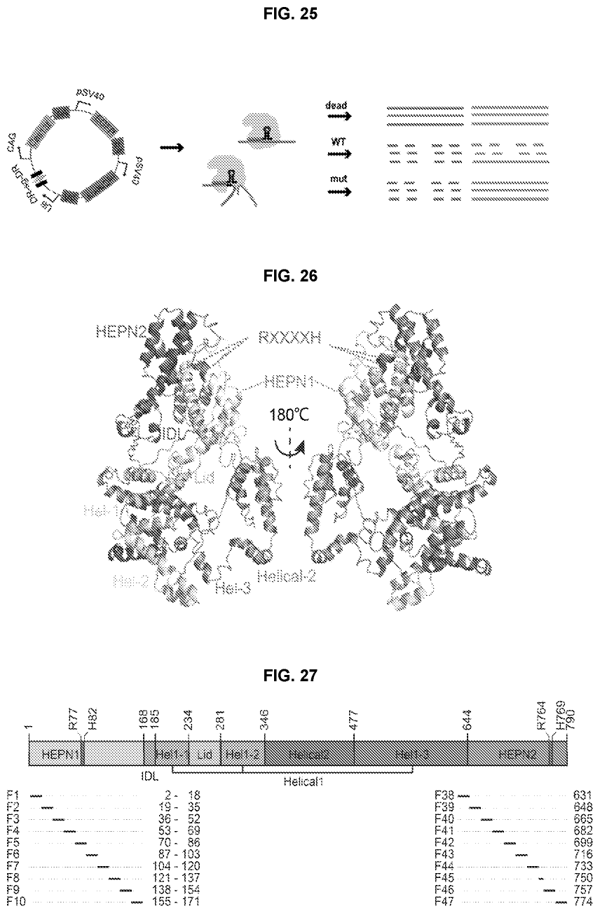

Specifically, to evaluate the collateral effects of Cas13f in mammalian cells, different Cas13f variants were co-transfected with EGFP and mCherry coding sequences, together with targeted (against EGFP) guide RNA (gRNA) into HEK293T cells. Expression levels of the targeted EGFP and the non-targeted mCherry were measured 48 hrs after transfection (FIG. 25).

A publically available online tool TASSER was used to predict the 3D structure of Cas13f, and the predicted structure was visualized with PyMOL in order to determine the position of the various structual domains in 3D (see FIG. 26).

Then an unbiased screening system was designed based on the dual-fluorescence system described herein, in which coding sequences for EGFP, mCherry, EGFP-targeting gRNA, together with each Cas13 variants, were inserted into a plasmid for expression in 293T cells. In this system, expression of EGFP and expression of mCherry were driven by the same SV40 promoter, in order to ensure roughly equally stable expression of the reporter genes in the transfected host cell. The gRNA was chosen to be specific for EGFP mRNA. Each coding sequence for Cas13f and variants has an N-terminal and a C-terminal nuclear localization signal (NLS), and expression of Cas13f and variants/mutants was driven by the strong CAG promoter.

The EGFP and mCherry coding sequences are SEQ ID NOs: 1 and 2, respectively. The corresponding DNA sequence of the gRNA is SEQ ID NO: 3. The SV40 promoter sequence is SEQ ID NO: 104. The wild-type Cas13f protein sequence is SEQ ID NO: 52. The CAG promoter sequence is SEQ ID NO: 103.

The HEPN1, HEPN2, Helical1 and Helical2 domains of Cas13f were chosen for generating a Cas13f mutagenesis library. First, these regions were divided into 47 small segments (F1-F47), each with about 17 residues (FIG. 27).

To facilitate subsequent selection, a BpiI restriction enzyme recognition site (GTCTTC, corresponding to encoded residues VF; reverse complement GAAGAC, corresponding to encoded residues ED) was introduced at each end of the segments. When producing mutants, all non-Ala residues were substituted by Ala, and all Ala residues were substituted by Val (e.g., replacing all non-alanine to alanine, X>A, and alanine to valine, A>V). About 4-5 total mutations were introduced between the two BpiI sites flanking each segment. The various mutants so generated and their corresponding wild-type sequences are provided below.

| | | SEQ | | SEQ |
|---|---|---|---|---|
| Variants | Amino Acids | ID NO: | DNA sequence | ID NO: |
| F1V1 | AAIELAAEEAAFAFNQA | 1027 | gccgccATCGAGCTGgccgccGAAGAAGCCGCCTTCgccTTCAATCAGGCC | 1211 |
| F1V2 | NGAEAKKEEAAFYFAAA | 1028 | AATGGCgccGAGgccAAGAAGGAAGAAGCCGCCTTCTACTTCgccgccGCC | 1212 |
| F1V3 | NGIALKKAEAAAYANQA | 1029 | AATGGCATCgccCTGAAGAAGgccGAAGCCGCCgccTACgccAATCAGGCC | 1213 |
| F1V4 | NGIELKKEAVVFYFNQV | 1030 | AATGGCATCGAGCTGAAGAAGGAAgccgtggtgTTCTACTTCAATCAGgtg | 1214 |
| F2V1 | ELALAAIEANIFAAERR | 1031 | GAGCTGgccCTGgccGCCATTGAGgccAACATCTTCgccgccGAGAGAAGA | 1215 |
| F2V2 | EANAKAAEDAIFDKERR | 1032 | GAGgccAACgccAAGGCCgccGAGGACgccATCTTCGACAAGGAGAGAAGA | 1216 |
| F2V3 | ALNLKAIADNAADKERR | 1033 | gccCTGAACCTGAAGGCCATTgccGACAACgccgccGACAAGGAGAGAAGA | 1217 |
| F2V4 | ELNLKVIEDNIFDKAAA | 1034 | GAGCTGAACCTGAAGgtgATTGAGGACAACATCTTCGACAAGgccgccgcc | 1218 |
| F3V1 | AALLAAPQILAAMENFI | 1035 | gccgccCTGCTGgccgccCCCCAGATCCTGGCCgccATGGAGAACTTTATC | 1219 |
| F3V2 | KTAANNPAILAKMEAFI | 1036 | AAGACAgccgccAACAACCCCgccATCCTGGCCAAGATGGAGgccTTTATC | 1220 |
| F3V3 | KTLLNNPQAAAKAENFA | 1037 | AAGACACTGCTGAACAACCCCCAGgccgccGCCAAGgccGAGAACTTTgcc | 1221 |
| F3V4 | KTLLNNAQILVKMANAI | 1038 | AAGACACTGCTGAACAACgccCAGATCCTGgtgAAGATGgccAACgccATC | 1222 |
| F4V1 | FNFRAVAANAAAEIDCL | 1039 | TTCAATTTCCGGgccGTGgccgccAACGCCgccgccGAAATCGACTGCCTG | 1223 |
| F4V2 | FAFRDATKAAKGEIACL | 1040 | TTCgccTTCCGGGACgccACCAAGgccGCCAAGGGCGAAATCgccTGCCTG | 1224 |
| F4V3 | ANFRDVTKNAKGEADAA | 1041 | gccAATTTCCGGGACGTGACCAAGAACGCCAAGGGCGAAgccGACgccgcc | 1225 |
| F4V4 | FNAADVTKNVKGAIDCL | 1042 | TTCAATgccgccGACGTGACCAAGAACgtgAAGGGCgccATCGACTGCCTG | 1226 |
| F5V1 | ALALRELRNFYSHAAHA | 1043 | gccCTGgccCTGAGAGAGCTGcggaacttttacagccacgccgccCACgcc | 1227 |
| F5V2 | LAKAREARNFYSHYVAK | 1044 | CTGgccAAGgccAGAGAGgcccggaacttttacagccacTACGTGgccAAG | 1228 |
| F5V3 | LLKLAALRNFYSHYVHK | 1045 | CTGCTGAAGCTGgccgccCTGcggaacttttacagccacTACGTGCACAAG | 1229 |
| F6V1 | RDVRELAAAEAPILEAY | 1046 | CGGGACGTCAGAGAACTGgccgccgccGAGgccCCGATCCTGGAGgccTAC | 1230 |
| F6V2 | RAAREASKGEKPILEKA | 1047 | CGGgccgccAGAGAAgccAGCAAGGGCGAGAAGCCGATCCTGGAGAAGgcc | 1231 |
| F6V3 | RDVRALSKGAKPAAEKY | 1048 | CGGGACGTCAGAgccCTGAGCAAGGGCgccAAGCCGgccgccGAGAAGTAC | 1232 |
| F6V4 | ADVAELSKGEKAILAKY | 1049 | gccGACGTCgccGAACTGAGCAAGGGCGAGAAGgccATCCTGgccAAGTAC | 1233 |
| F7V1 | YQFAIEAAAAENVALEI | 1050 | TACCAGTTCGCCATCGAAgccgccgccgccGAGAACGTGgccCTCGAAATC | 1234 |
| F7V2 | AAFAIESTGSEAAKLEI | 1051 | gccgccTTCGCCATCGAATCCACCGGCTCTGAGgccgccAAGCTCGAAATC | 1235 |
| F7V3 | YQAAAESTGSENVKAEA | 1052 | TACCAGgccGCCgccGAATCCACCGGCTCTGAGAACGTGAAGgccGAAgcc | 1236 |
| F7V4 | YQFVIASTGSANVKLAI | 1053 | TACCAGTTCgtgATCgccTCCACCGGCTCTgccAACGTGAAGCTCgccATC | 1237 |
| F8V1 | IEAAAWLAAAAALFFLC | 1054 | ATCGAAgccgccGCCTGGCTGGCCgccGCCgccgccCTGTTCTTCCTGTGC | 1238 |
| F8V2 | IENDAWAADAGVAFFAA | 1055 | ATCGAAAACGACGCCTGGgccGCCGACGCCGGCGTGgccTTCTTCgccgcc | 1239 |
| F8V3 | AANDAWLADAGVLAALC | 1056 | gccgccAACGACGCCTGGCTGGCCGACGCCGGCGTGCTGgccgccCTGTGC | 1240 |
| F8V4 | IENDVALVDVGVLFFLC | 1057 | ATCGAAAACGACgtggccCTGgtgGACgtgGGCGTGCTGTTCTTCCTGTGC | 1241 |
| F9V1 | IFLAAAQANALIAGISG | 1058 | ATCTTCCTGgccgccgccCAGGCAAACgccCTGATCgccGGCATCAGCGGC | 1242 |
| F9V2 | IFLKKSQAAKLISAIAA | 1059 | ATCTTCCTGAAGAAGAGCCAGGCAgccAAGCTGATCAGCgccATCgccgcc | 1243 |
| F9V3 | AFAKKSAANKAISGISG | 1060 | gccTTCgccAAGAAGAGCgccGCAAACAAGgccATCAGCGGCATCAGCGGC | 1244 |
| F9V4 | IALKKSQVNKLASGASG | 1061 | ATCgccCTGAAGAAGAGCCAGgtgAACAAGCTGgccAGCGGCgccAGCGGC | 1245 |
| F10V1 | FARNADAAQPRRNLFAY | 1062 | TTCgccAGAAACgccGACgccgccCAGCCTCGGAGAAACCTGTTCgccTAC | 1246 |
| F10V2 | FKRADATGQPRRALFTA | 1063 | TTCAAGAGAgccGACgccACCGGCCAGCCTCGGAGAgccCTGTTCACCgcc | 1247 |
| F10V3 | AKRNDDTGAPRRNAATY | 1064 | gccAAGAGAAACGACGACACCGGCgccCCTCGGAGAAACgccgccACCTAC | 1248 |
| F10V4 | FKANDDTGQAAANLFTY | 1065 | TTCAAGgccAACGACGACACCGGCCAGgccgccgccAACCTGTTCACCTAC | 1249 |
| F11V1 | FAIREAAAVVPEMQAHF | 1066 | TTCgccATCCGGGAGgccgccgccGTGGTGCCCGAAATGCAGgccCACTTC | 1250 |
| F11V2 | FSIREGYKAAPEMAKAF | 1067 | TTCTCCATCCGGGAGGGCTACAAGgccgccCCCGAAATGgccAAGgccTTC | 1251 |
| F11V3 | ASAREGYKVVPEAQKHA | 1068 | gccTCCgccCGGGAGGGCTACAAGGTGGTGCCCGAAgccCAGAAGCACgcc | 1252 |
| F11V4 | FSIAAGYKVVAAMQKHF | 1069 | TTCTCCATCgccgccGGCTACAAGGTGGTGgccgccATGCAGAAGCACTTC | 1253 |
| F12V1 | LLFALVNHLANQAAAIE | 1070 | CTGCTGTTCgccCTGGTGAACCACCTGgccAACCAGgccgccgccATCGAA | 1254 |
| F12V2 | LLFSLAAHLSAADDYIE | 1071 | CTGCTGTTCTCCCTGgccgccCACCTGAGCgccgccGACGATTATATCGAA | 1255 |
| F12V3 | AAFSAVNHASNQDDYIE | 1072 | gccgccTTCTCCgccGTGAACCACgccAGCAACCAGGACGATTATATCGAA | 1256 |
| F12V4 | LLASLVNALSNQDDYAA | 1073 | CTGCTGgccTCCCTGGTGAACgccCTGAGCAACCAGGACGATTATgccgcc | 1257 |
| F13V1 | AAHQPAAIAEALFFHRI | 1074 | gccGCCCACCAGCCCgccgccATCgccGAGgccCTCTTCTTCCACCGGATT | 1258 |
| F13V2 | KAAAPYDIGEGAFFARI | 1075 | AAGGCCgccgccCCCTACGACATCGGCGAGGGCgccTTCTTCgccCGGATT | 1259 |
| F13V3 | KAHQPYDAGEGLAAHRA | 1076 | AAGGCCCACCAGCCCTACGACgccGGCGAGGGCCTCgccgccCACCGGgcc | 1260 |
| F13V4 | KVHQAYDIGAGLFFHAI | 1077 | AAGgtgCACCAGgccTACGACATCGGCgccGGCCTCTTCTTCCACgccATT | 1261 |
| F14V1 | AAAFLNIAAILRNMAFY | 1078 | GCCgccgccTTCCTGAACATCgccgccATCCTGAGAAACATGgccTTCTAC | 1262 |
| F14V2 | ASTFAAISGILRAMKFA | 1079 | GCCAGCACCTTCgccgccATCTCCGGAATCCTGAGAgccATGAAGTTCgcc | 1263 |
| F14V3 | ASTFLNASGAARNAKFY | 1080 | GCCAGCACCTTCCTGAACgccTCCGGAgccgccAGAAACgccAAGTTCTAC | 1264 |
| F14V4 | VSTALNISGILANMKAY | 1081 | gtgAGCACCgccCTGAACATCTCCGGAATCCTGgccAACATGAAGgccTAC | 1265 |
| F15V1 | AYQAARLVEQRAELARE | 1082 | gccTATCAGgccgccAGACTGGTGGAGCAGAGAgccGAGCTGgccCGGGAA | 1266 |
| F15V2 | TAASKRLAEARGELKRE | 1083 | ACCgccgccAGCAAGAGACTGgccGAGgccAGAGGCGAGCTGAAGCGGGAA | 1267 |
| F15V3 | TYQSKRAVAQRGAAKRE | 1084 | ACCTATCAGAGCAAGAGAgccGTGgccCAGAGAGGCgccgccAAGCGGGAA | 1268 |
| F15V4 | TYQSKALVEQAGELKAA | 1085 | ACCTATCAGAGCAAGgccCTGGTGGAGCAGgccGGCGAGCTGAAGgccgcc | 1269 |
| F16V1 | AAIFAWEEPFQANAAFE | 1086 | gccgccATCTTCGCCTGGGAAGAACCGTTTCAGgccAATgccgccTTTGAG | 1270 |
| F16V2 | KDAAAWEEPFAGASYFE | 1087 | AAGGACgccgccGCCTGGGAAGAACCGTTTgccGGCgccTCCTACTTTGAG | 1271 |
| F16V3 | KDIFAWAAPAQGNSYAE | 1088 | AAGGACATCTTCGCCTGGgccgccCCGgccCAGGGCAATTCCTACgccGAG | 1272 |
| F16V4 | KDIFVAEEAFQGNSYFA | 1089 | AAGGACATCTTCgtggccGAAGAAgccTTTCAGGGCAATTCCTACTTTgcc | 1273 |
| F17V1 | INAHAAVIAEDELAELC | 1090 | ATCAACgccCACgccgccGTGATTgccGAGGACGAGCTGgccGAGCTGTGC | 1274 |
| F17V2 | IAGHKGAIGEAEAKELC | 1091 | ATCgccGGCCACAAGGGCgccATTGGCGAGgccGAGgccAAGGAGCTGTGC | 1275 |
| F17V3 | ANGAKGVIGEDELKEAA | 1092 | gccAACGGCgccAAGGGCGTGATTGGCGAGGACGAGCTGAAGGAGgccgcc | 1276 |
| F17V4 | INGHKGVAGADALKALC | 1093 | ATCAACGGCCACAAGGGCGTGgccGGCgccGACgccCTGAAGgccCTGTGC | 1277 |
| F18V1 | AAFLIANQAANAVEARI | 1094 | gccGCCTTCCTGATCgccAACCAGgccGCCAACgccGTGGAGgccCGGATC | 1278 |
| F18V2 | YAFLIGAADAAKAEGRI | 1095 | TACGCCTTCCTGATCGGCgccgccGACGCCgccAAGgccGAGGGCCGGATC | 1279 |
| F18V3 | YAAAAGNQDANKVEGRA | 1096 | TACGCCgccgccgccGGCAACCAGGACGCCAACAAGGTGGAGGGCCGGgcc | 1280 |
| F18V4 | YVFLIGNQDVNKVAGAI | 1097 | TACgtgTTCCTGATCGGCAACCAGGACgtgAACAAGGTGgccGGCgccATC | 1281 |
| F19V1 | AQFLEAFRAANAVQQVA | 1098 | gccCAGTTCCTGGAGgccTTCAGAgccGCCAACgccGTGCAGCAGGTGgcc | 1282 |
| F19V2 | TAFLEKFRNAASAQQAK | 1099 | ACCgccTTCCTGGAGAAGTTCAGAAACGCCgccAGCgccCAGCAGgccAAG | 1283 |
| F19V3 | TQAAEKFRNANSVAAVK | 1100 | ACCCAGgccgccGAGAAGTTCAGAAACGCCAACAGCGTGgccgccGTGAAG | 1284 |
| F19V4 | TQFLAKAANVNSVQQVK | 1101 | ACCCAGTTCCTGgccAAGgccgccAACgtgAACAGCGTGCAGCAGGTGAAG | 1285 |
| F20V1 | AAEMLAPEAFPANAFAE | 1102 | gccgccGAGATGCTGgccCCTGAAgccTTCCCCGCCAACgccTTTGCCGAG | 1286 |
| F20V2 | DDEAAKPEYAPAAYFAE | 1103 | GACGACGAGgccgccAAGCCTGAATATgccCCCGCCgccTACTTTGCCGAG | 1287 |
| F20V3 | DDAMLKPAYFPANYAAA | 1104 | GACGACgccATGCTGAAGCCTgccTATTTCCCCGCCAACTACgccGCCgcc | 1288 |
| F20V4 | DDEMLKAEYFAVNYFVE | 1105 | GACGACGAGATGCTGAAGgccGAATATTTCgccgtgAACTACTTTgtgGAG | 1289 |
| F21V1 | AAVARIADRVLNRLNAA | 1106 | gccgccGTGgccCGGATCgccGACCGGGTGCTGAACAGACTGAACgccGCC | 1290 |
| F21V2 | SGAGRIKARVLARLAKA | 1107 | AGCGGCgccGGCCGGATCAAGgccCGGGTGCTGgccAGACTGgccAAGGCC | 1291 |
| F21V3 | SGVGRAKDRAANRANKA | 1108 | AGCGGCGTGGGCCGGgccAAGGACCGGgccgccAACAGAgccAACAAGGCC | 1292 |
| F21V4 | SGVGAIKDAVLNALNKV | 1109 | AGCGGCGTGGGCgccATCAAGGACgccGTGCTGAACgccCTGAACAAGgtg | 1293 |
| F22V1 | IASNAAAAGEIIAYDAM | 1110 | ATCgccAGCAACgccGCCgccgccGGCGAGATCATCGCCTATGACgccATG | 1294 |
| F22V2 | IKANKAKKAEIIAAAKM | 1111 | ATCAAGgccAACAAGGCCAAGAAGgccGAGATCATCGCCgccgccAAGATG | 1295 |
| F22V3 | AKSAKAKKGEAIAYDKA | 1112 | gccAAGAGCgccAAGGCCAAGAAGGGCGAGgccATCGCCTATGACAAGgcc | 1296 |
| F22V4 | IKSNKVKKGAIAVYDKM | 1113 | ATCAAGAGCAACAAGgtgAAGAAGGGCgccATCgccgtgTATGACAAGATG | 1297 |
| F23V1 | REVMAFIAAALPVAEAL | 1114 | AGAGAAGTGATGGCTTTCATCgccgccgccCTGCCCGTGgccGAGgccCTG | 1298 |
| F23V2 | REAMAFINNSAPADEKA | 1115 | AGAGAAgccATGGCTTTCATCAATAACTCTgccCCCgccGACGAGAAGgcc | 1299 |
| F23V3 | RAVAAAANNSLPVDEKL | 1116 | AGAgccGTGgccGCTgccgccAATAACTCTCTGCCCGTGGACGAGAAGCTG | 1300 |
| F23V4 | AEVMVFINNSLAVDAKL | 1117 | gccGAAGTGATGgtgTTCATCAATAACTCTCTGgccGTGGACgccAAGCTG | 1301 |
| F24V1 | APAAYARYLAMVRFWDR | 1118 | gccCCCgccgccTACgccAGATACCTGgccATGGTGAGATTCTGGGATAGA | 1302 |
| F24V2 | KPKDAKRALGMARFWAR | 1119 | AAGCCCAAGGATgccAAGAGAgccCTGGGCATGgccAGATTCTGGgccAGA | 1303 |
| F24V3 | KAKDYKRYAGAVRAWDR | 1120 | AAGgccAAGGATTACAAGAGATACgccGGCgccGTGAGAgccTGGGATAGA | 1304 |
| F24V4 | KPKDYKAYLGMVAFADA | 1121 | AAGCCCAAGGATTACAAGgccTACCTGGGCATGGTGgccTTCgccGATgcc | 1305 |
| F25V1 | EADNIAREFETAEWAAY | 1122 | GAAgccGACAATATCgccCGCGAGTTCGAAACGgccGAGTGGgccgccTAT | 1306 |
| F25V2 | EKAAIKREFEAKEWSKA | 1123 | GAAAAGgccgccATCAAGCGCGAGTTCGAAgccAAGGAGTGGAGCAAGgcc | 1307 |
| F25V3 | AKDNAKRAAETKEWSKY | 1124 | gccAAGGACAATgccAAGCGCgccgccGAAACGAAGGAGTGGAGCAAGTAT | 1308 |
| F25V4 | EKDNIKAEFATKAASKY | 1125 | GAAAAGGACAATATCAAGgccGAGTTCgccACGAAGgccgccAGCAAGTAT | 1309 |
| F26V1 | LPANFWAAANLERVAAL | 1126 | CTGCCCgccAACTTCTGGgccGCCgccAACCTGGAGAGAGTGgccgccCTG | 1310 |
| F26V2 | APSAFWTAKALERAYGL | 1127 | gccCCCTCCgccTTCTGGACCGCCAAGgccCTGGAGAGAgccTACGGACTG | 1311 |
| F26V3 | LPSNAWTAKNAARVYGA | 1128 | CTGCCCTCCAACgccTGGACCGCCAAGAACgccgccAGAGTGTACGGAgcc | 1312 |
| F26V4 | LASNFATVKNLEAVYGL | 1129 | CTGgccTCCAACTTCgccACCgtgAAGAACCTGGAGgccGTGTACGGACTG | 1313 |
| F27V1 | AREAAAELFNALAAAVE | 1130 | GCCCGGGAAgccgccGCAGAGCTGTTTAACgccCTGgccGCCgccGTGGAG | 1314 |
| F27V2 | AREKNAEAFAKAKADAE | 1131 | GCCCGGGAAAAGAACGCAGAGgccTTTgccAAGgccAAGGCCGACgccGAG | 1315 |
| F27V3 | ARAKNAALANKLKADVA | 1132 | GCCCGGgccAAGAACGCAgccCTGgccAACAAGCTGAAGGCCGACGTGgcc | 1316 |
| F27V4 | VAEKNVELFNKLKVDVE | 1133 | gtggccGAAAAGAACgtgGAGCTGTTTAACAAGCTGAAGgtgGACGTGGAG | 1317 |
| F28V1 | AMAERELEAYQAINDAA | 1134 | gccATGgccGAAAGAGAGCTGGAAgccTATCAGgccATCAACGACGCCgcc | 1318 |
| F28V2 | KMDERELEKAAKIAAAK | 1135 | AAGATGGACGAAAGAGAGCTGGAAAAGgccgccAAGATCgccgccGCCAAG | 1319 |
| F28V3 | KADAREAEKYQKANDAK | 1136 | AAGgccGACgccAGAGAGgccGAAAAGTATCAGAAGgccAACGACGCCAAG | 1320 |
| F28V4 | KMDEAALAKYQKINDVK | 1137 | AAGATGGACGAAgccgccCTGgccAAGTATCAGAAGATCAACGACgtgAAG | 1321 |
| F29V1 | ALANLRRLAAAFAVAWE | 1138 | gccCTGGCCAACCTGCGGCGGCTGGCCgccgccTTCgccGTGgccTGGGAG | 1322 |
| F29V2 | DAAAARRLASDFGAKWE | 1139 | GATgccGCCgccgccCGGCGGCTGGCCAGCGACTTCGGAgccAAGTGGGAG | 1323 |
| F29V3 | DLVNLRRAASDAGVKWA | 1140 | GATCTGgtgAACCTGCGGCGGgccGCCAGCGACgccGGAGTGAAGTGGgcc | 1324 |
| F29V4 | DLANLAALVSDFGVKAE | 1141 | GATCTGGCCAACCTGgccgccCTGgtgAGCGACTTCGGAGTGAAGgccGAG | 1325 |
| F30V1 | EADWDEYAAQIAAQITD | 1142 | GAGgccGATTGGGACGAGTACgccgccCAGATCgccgccCAGATCACAGAT | 1326 |
| F30V2 | EKAWAEYSGQIKKQIAA | 1143 | GAGAAGgccTGGgccGAGTACTCCGGCCAGATCAAGAAGCAGATCgccgcc | 1327 |
| F30V3 | EKDWDEASGAAKKAITD | 1144 | GAGAAGGATTGGGACGAGgccTCCGGCgccgccAAGAAGgccATCACAGAT | 1328 |
| F30V4 | AKDADAYSGQIKKQATD | 1145 | gccAAGGATgccGACgccTACTCCGGCCAGATCAAGAAGCAGgccACAGAT | 1329 |
| F31V1 | AQALTIMAQRITAGLAA | 1146 | gccCAGgccCTGACCATCATGgccCAGAGAATCACAGCCGGCCTGgccgcc | 1330 |
| F31V2 | SAKLAIMKQRIAAALKK | 1147 | TCCgccAAGCTGgccATCATGAAGCAGAGAATCgccGCCgccCTGAAGAAG | 1331 |
| F31V3 | SQKATIAKARITAGAKK | 1148 | TCCCAGAAGgccACCATCgccAAGgccAGAATCACAGCCGGCgccAAGAAG | 1332 |
| F31V4 | SQKLTAMKQAATVGLKK | 1149 | TCCCAGAAGCTGACCgccATGAAGCAGgccgccACAgtgGGCCTGAAGAAG | 1333 |
| F32V1 | AHAIENLNLRIAIAINA | 1150 | gccCACgccATCGAAAACCTGAACCTGAGGATCgccATCgccATCAACgcc | 1334 |
| F32V2 | KHGIEAAALRITIDIAK | 1151 | AAGCACGGCATCGAAgccgccgccCTGAGGATCACCATCGACATCgccAAG | 1335 |
| F32V3 | KAGAENLNARATIDINK | 1152 | AAGgccGGCgccGAAAACCTGAACgccAGGgccACCATCGACATCAACAAG | 1336 |
| F32V4 | KHGIANLNLAITADANK | 1153 | AAGCACGGCATCgccAACCTGAACCTGgccATCACCgccGACgccAACAAG | 1337 |
| F33V1 | ARAAVLARIAIPRAFVA | 1154 | gccAGAgccGCCGTGCTGgccCGGATCGCCATCCCCAGAgccTTTGTGgcc | 1338 |
| F33V2 | SRKAAANRAAIPRGFAK | 1155 | TCCAGAAAGGCCgccgccAATCGGgccGCCATCCCCAGAGGATTTgccAAG | 1339 |
| F33V3 | SRKVVLNRIAAARGAVK | 1156 | TCCAGAAAGgtgGTGCTGAATCGGATCGCCgccgccAGAGGAgccGTGAAG | 1340 |
| F33V4 | SAKAVLNAIVIPAGFVK | 1157 | TCCgccAAGGCCGTGCTGAATgccATCgtgATCCCCgccGGATTTGTGAAG | 1341 |
| F34V1 | RHILGWQEAEAVAAAIR | 1158 | CGGCACATCCTGGGCTGGCAGGAAgccGAGgccGTGgccgccgccATCAGA | 1342 |
| F34V2 | RHIAAWAESEKASKKIR | 1159 | CGGCACATCgccgccTGGgccGAATCCGAGAAGgccAGCAAGAAGATCAGA | 1343 |
| F34V3 | RAALGWQASEKVSKKAR | 1160 | CGGgccgccCTGGGCTGGCAGgccTCCGAGAAGGTGAGCAAGAAGgccAGA | 1344 |
| F34V4 | AHILGAQESAKVSKKIA | 1161 | gccCACATCCTGGGCgccCAGGAATCCgccAAGGTGAGCAAGAAGATCgcc | 1345 |
| F35V1 | EAECEILLAAEAEELAA | 1162 | GAAGCCGAATGCGAGATTCTGCTGgccgccGAGgccGAGGAGCTGgccgcc | 1346 |
| F35V2 | EAEAEIAASKEYEEASK | 1163 | GAAGCCGAAgCCGAGATTgCCgCCAGCAAGGAGTACGAGGAGgCCAGCAAG | 1347 |
| F35V3 | AAACAALLSKEYEELSK | 1164 | gccGCCgccTGCgccgccCTGCTGAGCAAGGAGTACGAGGAGCTGAGCAAG | 1348 |
| F35V4 | EVECEILLSKAYAALSK | 1165 | GAAgtgGAATGCGAGATTCTGCTGAGCAAGgccTACgccgccCTGAGCAAG | 1349 |
| F36V1 | QFFQAADYDAMARINAL | 1166 | CAGTTCTTTCAGgccgccGACTACGACgccATGgccCGCATCAACgccCTG | 1350 |
| F36V2 | QFFQSKAAAKMTRIAGL | 1167 | CAGTTCTTTCAGAGCAAGgccgccgccAAGATGACCCGCATCgccGGCCTG | 1351 |
| F36V3 | AFFASKDYDKATRINGA | 1168 | gccTTCTTTgccAGCAAGGACTACGACAAGgccACCCGCATCAACGGCgcc | 1352 |
| F36V4 | QAAQSKDYDKMTAANGL | 1169 | CAGgccgccCAGAGCAAGGACTACGACAAGATGACCgccgccAACGGCCTG | 1353 |
| F37V1 | AEANALIALMAVALMAQ | 1170 | gccGAGgccAATgccCTGATCGCCCTGATGGCCGTGgccCTGATGgccCAG | 1354 |
| F37V2 | YEKAKAIALMAAYLMGA | 1171 | TACGAGAAGgccAAGgccATCGCCCTGATGGCCgccTATCTGATGGGGgcc | 1355 |
| F37V3 | YEKNKLIAAAAVYAAGQ | 1172 | TACGAGAAGAATAAGCTGATCGCCgccgccGCCGTGTATgccgccGGGCAG | 1356 |
| F37V4 | YAKNKLAVLMVVYLMGQ | 1173 | TACgccAAGAATAAGCTGgccgtgCTGATGgtgGTGTATCTGATGGGGCAG | 1357 |
| F38V1 | LRILFAEHAALDDIAAT | 1174 | CTGAGAATCCTGTTCgccGAGCACgccgccCTGGACGACATCgccgccACC | 1358 |
| F38V2 | ARILFKEHTKLAAITKA | 1175 | gccAGAATCCTGTTCAAGGAGCACACCAAGCTGgccgccATCACCAAGgcc | 1359 |
| F38V3 | LRAAFKEATKADDITKT | 1176 | CTGAGAgccgccTTCAAGGAGgccACCAAGgccGACGACATCACCAAGACC | 1360 |
| F38V4 | LAILAKAHTKLDDATKT | 1177 | CTGgccATCCTGgccAAGgccCACACCAAGCTGGACGACgccACCAAGACC | 1361 |
| F39V1 | TVDFAIADAVTVAIPFA | 1178 | ACCGTGGATTTCgccATCgccGACgccGTGACCGTGgccATCCCCTTCgcc | 1362 |
| F39V2 | AVAFKISAKVAVKIPFS | 1179 | gccGTGgccTTCAAGATCAGCgccAAGGTGgccGTGAAGATCCCCTTCTCC | 1363 |
| F39V3 | TADFKASDKATAKIPFS | 1180 | ACCgccGATTTCAAGgccAGCGACAAGgccACCgccAAGATCCCCTTCTCC | 1364 |
| F39V4 | TVDAKISDKVTVKAAAS | 1181 | ACCGTGGATgccAAGATCAGCGACAAGGTGACCGTGAAGgccgccgccTCC | 1365 |
| F40V1 | NYPALVYAMAAAYVDNI | 1182 | AACTATCCCgccCTGGTGTACgccATGgccgccgccTACGTGGACAATATC | 1366 |
| F40V2 | NAPSLVATMSSKAVANI | 1183 | AACgccCCCTCCCTGGTGgccACCATGAGCAGCAAGgccGTGgccAATATC | 1367 |
| F40V3 | AYPSLAYTMSSKYADAI | 1184 | gccTATCCCTCCCTGgccTACACCATGAGCAGCAAGTACgccGACgccATC | 1368 |
| F40V4 | NYASAVYTASSKYVDNA | 1185 | AACTATgccTCCgccGTGTACACCgccAGCAGCAAGTACGTGGACAATgcc | 1369 |
| F41V1 | GNYGFANADADAPILGA | 1186 | GGCAACTACGGCTTCgccAACgccGACgccGATgccCCCATTCTGGGCgcc | 1370 |
| F41V2 | ANYAFSNKAKDKPILAK | 1187 | gccAACTACgccTTCAGCAACAAGgccAAGGATAAGCCCATTCTGgccAAG | 1371 |
| F41V3 | GAAGFSAKDKAKPILGK | 1188 | GGCgccgccGGCTTCAGCgccAAGGACAAGgccAAGCCCATTCTGGGCAAG | 1372 |
| F41V4 | GNYGASNKDKDKAAAGK | 1189 | GGCAACTACGGCgccAGCAACAAGGACAAGGATAAGgccgccgccGGCAAG | 1373 |
| F42V1 | IAAIEAQRMEFIAEVLA | 1190 | ATCgccgccATCGAGgccCAGCGGATGGAGTTTATCgccGAGGTGCTGgcc | 1374 |
| F42V2 | IDVIEKARAEFIKEAAG | 1191 | ATCGACGTGATCGAGAAGgccCGGgccGAGTTTATCAAGGAGgccgccGGA | 1375 |
| F42V3 | ADVAEKQRMEAAKEVLG | 1192 | gccGACGTGgccGAGAAGCAGCGGATGGAGgccgccAAGGAGGTGCTGGGA | 1376 |
| F42V4 | IDVIAKQAMAFIKAVLG | 1193 | ATCGACGTGATCgccAAGCAGgccATGgccTTTATCAAGgccGTGCTGGGA | 1377 |
| F43V1 | FEAYLFDDAIIDAAAFA | 1194 | TTCGAGgccTACCTGTTTGACGATgccATCATCGACgccgccgccTTCGCC | 1378 |
| F43V2 | FEKALFAAKIIAKSKFA | 1195 | TTCGAGAAGgccCTGTTTgccgccAAGATCATCgccAAGAGCAAGTTCGCC | 1379 |
| F43V3 | AEKYAFDDKAADKSKFA | 1196 | gccGAGAAGTACgccTTTGACGATAAGgccgccGACAAGAGCAAGTTCGCC | 1380 |
| F43V4 | FAKYLADDKIIDKSKAV | 1197 | TTCgccAAGTACCTGgccGACGATAAGATCATCGACAAGAGCAAGgccgtg | 1381 |
| F44V1 | AAAAHIAFAEIAEELVE | 1198 | gccgccGCCgccCACATCgccTTTGCCGAAATCgccGAAGAACTGGTGGAG | 1382 |
| F44V2 | DTATAASFAEIVEEAAE | 1199 | GACACCGCCACCgccgccAGCTTTGCCGAAATCGTGGAAGAAgccgccGAG | 1383 |
| F44V3 | DTATHISAAAAVAELVE | 1200 | GACACCGCCACCCACATCAGCgccGCCgccgccGTGgccGAACTGGTGGAG | 1384 |
| F44V4 | DTVTHISFVEIVEALVA | 1201 | GACACCgtgACCCACATCAGCTTTgtgGAAATCGTGGAAgccCTGGTGgcc | 1385 |
| F45V1 | AAWAADRLA | 1202 | gccgccTGGgccgccGACCGGCTGgcc | 1386 |
| F45V2 | KGADKAAAT | 1203 | AAGGGCgccGACAAGgccgccgccACG | 1387 |
| F46V1 | LAALAAARNKALHAEIL | 1204 | CTGgccgccCTGgccgccGCCcggaacaaggccctgcacgccGAGATCCTG | 1388 |
| F46V2 | ATKAKDARNKALHGEAA | 1205 | gccACGAAGgccAAGGATGCCcggaacaaggccctgcacGGCGAGgccgcc | 1389 |
| F46V3 | LTKLKDVRNKALHGAIL | 1206 | CTGACGAAGCTGAAGGATgtgcggaacaaggccctgcacGGCgccATCCTG | 1390 |
| F47V1 | TGTAFDETAALINELAA | 1207 | ACCGGCACCgccTTCGACGAGACAgccgccCTGATCAACGAGCTGgccgcc | 1391 |
| F47V2 | AAASFDEAKSLINELKK | 1208 | gccgccgccAGCTTCGACGAGgccAAGTCCCTGATCAACGAGCTGAAGAAG | 1392 |
| F47V3 | TGTSFAETKSAIAEAKK | 1209 | ACCGGCACCAGCTTCgccGAGACAAAGTCCgccATCgccGAGgccAAGAAG | 1393 |
| F47V4 | TGTSADATKSLANALKK | 1210 | ACCGGCACCAGCgCCGACgCCACAAAGTCCCTGgCCAACgCCCTGAAGAAG | 1394 |

Using the EGFP-mCherry dual-fluorescence reporter system of the invention, these Cas13f mutants were functionally screened to assess their collateral vs. gRNA-guided cleavage activities. Specifically, according to standard cell culture methods, human HEK293 cells were grown in 24-well tissue culture plates to a suitable density before the cells were transfected with PEI reagents and plasmids that express each mutant Cas13f and the reporter system fluorescent proteins. Transfected cells were cultured at 37° C. in incubator under 5% CO₂ for about 48 hours, before measuring EGFP and mCherry signals in the cells with FACS. Mutants leading to low percentage of the gRNA-targeted EGFP signal (lower percentage of EGFP⁺ cells, as a readout for preserved gRNA-guided cleavage) and high percentage of non-targeted mCherry signal (higher percentage of mCherry⁺ cells, as a readout for lacking collateral effect) were selected.

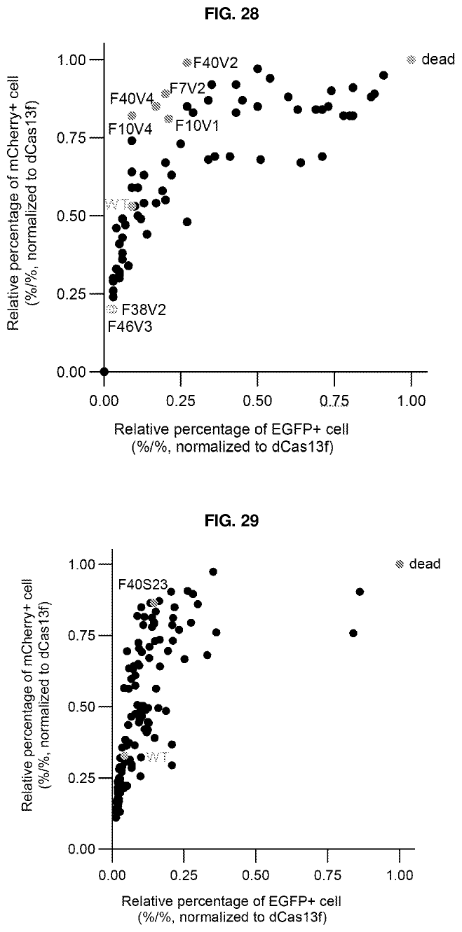

In this experiment, dCas13f with no gRNA-guided cleavage was used as a negative control, and the results (mean±s.e.m.) were normalized against that of dCas13f and listed below. Cas13f mutants/variants located at the upper left area of FIG. 28 had low collateral effect (high mCherry signal) and high gRNA-guided cleavage activity (low EGFP signal), and were selected as the desired low/no collateral effect mutants.

| | % mCherry | S.E.M. | % EGFP | S.E.M. |
|---|---|---|---|---|
| dead | 1.0000 | 0.0089 | 1.0000 | 0.0040 |
| WT | 0.5278 | 0.0124 | 0.0944 | 0.0035 |
| F2V1 | 0.8439 | 0.0205 | 0.6276 | 0.0123 |
| F2V2 | 0.8266 | 0.0220 | 0.4340 | 0.0103 |
| F2V3 | 0.4429 | 0.0017 | 0.1445 | 0.0033 |
| F2V4 | 0.6268 | 0.0094 | 0.2191 | 0.0016 |
| F3V1 | 0.5784 | 0.0045 | 0.1915 | 0.0047 |
| F3V2 | 0.9749 | 0.0113 | 0.4988 | 0.0093 |
| F3V3 | 0.7297 | 0.0216 | 0.2525 | 0.0102 |
| F3V4 | 0.5909 | 0.0071 | 0.1112 | 0.0078 |
| F4V1 | 0.6783 | 0.0092 | 0.3402 | 0.0141 |
| F4V2 | 0.9468 | 0.0096 | 0.9054 | 0.0078 |
| F4V3 | 0.3446 | 0.0102 | 0.0775 | 0.0030 |
| F4V4 | 0.9046 | 0.0260 | 0.7416 | 0.0141 |
| F5V1 | 0.9385 | 0.0143 | 0.5379 | 0.0094 |
| F5V2 | 0.5352 | 0.0108 | 0.1281 | 0.0025 |
| F5V3 | 0.5405 | 0.0127 | 0.1688 | 0.0074 |
| F6V1 | 0.8309 | 0.0077 | 0.2858 | 0.0053 |
| F6V2 | 0.6913 | 0.0091 | 0.3636 | 0.0075 |
| F6V3 | 0.3426 | 0.0028 | 0.0829 | 0.0017 |
| F6V4 | 0.6262 | 0.0143 | 0.1283 | 0.0025 |
| F7V1 | 0.5315 | 0.0096 | 0.0960 | 0.0019 |
| F7V2 | 0.8915 | 0.0086 | 0.1956 | 0.0100 |
| F7V3 | 0.6861 | 0.0153 | 0.4122 | 0.0042 |
| F7V4 | 0.4794 | 0.0023 | 0.2748 | 0.0031 |
| F7V4 | 0.8393 | 0.0086 | 0.6918 | 0.0117 |
| F8V1 | 0.8171 | 0.0068 | 0.7974 | 0.0122 |
| F8V2 | 0.8228 | 0.0034 | 0.7836 | 0.0024 |
| F8V3 | 0.8180 | 0.0083 | 0.8101 | 0.0093 |
| F8V4 | 0.3162 | 0.0040 | 0.0494 | 0.0020 |
| F9V1 | 0.8656 | 0.0120 | 0.4549 | 0.0124 |
| F9V2 | 0.4951 | 0.0023 | 0.1051 | 0.0019 |
| F9V3 | 0.6949 | 0.0557 | 0.7116 | 0.0375 |
| F9V4 | 0.6677 | 0.0017 | 0.6370 | 0.0052 |
| F10V1 | 0.8131 | 0.0050 | 0.2123 | 0.0102 |
| F10V2 | 0.3165 | 0.0091 | 0.0470 | 0.0023 |
| F10V3 | 0.8360 | 0.0082 | 0.7123 | 0.0091 |
| F10V4 | 0.8215 | 0.0088 | 0.0929 | 0.0018 |
| F38V1 | 0.3261 | 0.0046 | 0.0381 | 0.0019 |
| F38V2 | 0.2031 | 0.0040 | 0.0350 | 0.0007 |
| F38V3 | 0.3078 | 0.0062 | 0.0526 | 0.0011 |
| F38V4 | 0.5860 | 0.0101 | 0.0904 | 0.0069 |
| F39V1 | 0.4731 | 0.0220 | 0.0736 | 0.0043 |
| F39V2 | 0.4639 | 0.0044 | 0.0386 | 0.0026 |
| F39V3 | 0.9212 | 0.0257 | 0.3547 | 0.0187 |
| F39V4 | 0.9168 | 0.0279 | 0.4272 | 0.0062 |
| F40V1 | 0.6440 | 0.0283 | 0.0856 | 0.0052 |
| F40V2 | 0.9857 | 0.0083 | 0.2711 | 0.0053 |
| F40V3 | 0.2644 | 0.0085 | 0.0341 | 0.0031 |
| F40V4 | 0.8524 | 0.0109 | 0.1698 | 0.0068 |
| F41V1 | 0.5281 | 0.0089 | 0.0963 | 0.0057 |
| F41V2 | 0.3567 | 0.0149 | 0.0644 | 0.0040 |
| F41V3 | 0.7446 | 0.0137 | 0.0886 | 0.0055 |
| F41V4 | 0.8726 | 0.0120 | 0.3435 | 0.0193 |
| F42V1 | 0.2398 | 0.0069 | 0.0306 | 0.0004 |
| F42V2 | 0.6810 | 0.0327 | 0.5106 | 0.0245 |
| F42V3 | 0.8821 | 0.0002 | 0.8702 | 0.0032 |
| F42V4 | 0.6718 | 0.0222 | 0.2016 | 0.0114 |
| F43V1 | 0.5508 | 0.0189 | 0.1999 | 0.0111 |
| F43V2 | 0.2909 | 0.0072 | 0.0293 | 0.0009 |
| F43V3 | 0.8538 | 0.0147 | 0.7331 | 0.0183 |
| F43V4 | 0.9133 | 0.0152 | 0.8146 | 0.0136 |
| F44V1 | 0.4936 | 0.0106 | 0.0585 | 0.0020 |
| F44V2 | 0.8519 | 0.0183 | 0.2728 | 0.0106 |
| F44V3 | 0.8813 | 0.0144 | 0.5960 | 0.0070 |
| F44V3 | 0.9420 | 0.0104 | 0.8856 | 0.0150 |
| F44V4 | 0.2871 | 0.0161 | 0.0262 | 0.0019 |
| F45V1 | 0.4907 | 0.0173 | 0.1229 | 0.0062 |
| F45V2 | 0.3045 | 0.0085 | 0.0459 | 0.0029 |
| F46V1 | 0.4139 | 0.0096 | 0.0477 | 0.0020 |
| F46V2 | 0.8899 | 0.0091 | 0.8797 | 0.0066 |
| F46V3 | 0.2017 | 0.0084 | 0.0199 | 0.0003 |
| F47V1 | 0.8500 | 0.0091 | 0.4965 | 0.0026 |
| F47V1 | 0.4331 | 0.0100 | 0.0602 | 0.0009 |
| F47V2 | 0.2973 | 0.0138 | 0.0347 | 0.0035 |
| F47V3 | 0.3790 | 0.0179 | 0.0607 | 0.0049 |
| F47V4 | 0.8356 | 0.0064 | 0.7086 | 0.0107 |

After normalization of EGFP and mCherry fluorescence intensity by inactive dead Cas13f (dCas13f with R77A, H82A, R764A, and H769A mutations in HEPN domains), it was found that variants with mutation sites in F10, F38, F40, or F46, specially F10V1, F10V4, F38V2, F40V2, F40V4, F46V1 and F46V3, exhibited relatively low EGFP fluorescence intensity but much higher (or lower) mCherry fluorescence intensity compared to wild-type, indicating that these variants retained a high on-target activity but greatly reduced (or enhanced) collateral activity (FIG. 28).

Further mutagenesis study in or nearby these regions (F10V1, F10V4, F38V2, F40V2, F40V4, F46V1 and F46V3) of these mutants was conducted, by generating a number of additional mutants with single or multiple (e.g., double, triple, or quadruple) combination mutations. The sequences of these mutants/variants are listed below:

| | | SEQ | | SEQ |
|---|---|---|---|---|
| Variants | Amino Acids | ID NO: | DNA sequense | ID NO: |
| F10S1 | AKRNDDTGQPRRNLFTY | 1395 | gccAAGAGAAACGACGACACCGGCCAGCCTCGGAGAAACCTGTTCACCTAC | 1515 |
| F10S2 | FARNDDTGQPRRNLFTY | 1396 | TTCgccAGAAACGACGACACCGGCCAGCCTCGGAGAAACCTGTTCACCTAC | 1516 |
| F10S3 | FKANDDTGQPRRNLFTY | 1397 | TTCAAGgccAACGACGACACCGGCCAGCCTCGGAGAAACCTGTTCACCTAC | 1517 |
| F10S4 | FKRADDTGQPRRNLFTY | 1398 | TTCAAGAGAgccGACGACACCGGCCAGCCTCGGAGAAACCTGTTCACCTAC | 1518 |
| F10S5 | FKRNADTGQPRRNLFTY | 1399 | TTCAAGAGAAACgccGACACCGGCCAGCCTCGGAGAAACCTGTTCACCTAC | 1519 |
| F10S6 | FKRNDATGQPRRNLFTY | 1400 | TTCAAGAGAAACGACgccACCGGCCAGCCTCGGAGAAACCTGTTCACCTAC | 1520 |
| F10S7 | FKRNDDAGQPRRNLFTY | 1401 | TTCAAGAGAAACGACGACgccGGCCAGCCTCGGAGAAACCTGTTCACCTAC | 1521 |
| F10S8 | FKRNDDTAQPRRNLFTY | 1402 | TTCAAGAGAAACGACGACACCgccCAGCCTCGGAGAAACCTGTTCACCTAC | 1522 |
| F10S9 | FKRNDDTGAPRRNLFTY | 1403 | TTCAAGAGAAACGACGACACCGGCgccCCTCGGAGAAACCTGTTCACCTAC | 1523 |
| F10S10 | FKRNDDTGQARRNLFTY | 1404 | TTCAAGAGAAACGACGACACCGGCCAGgccCGGAGAAACCTGTTCACCTAC | 1524 |
| F10S11 | FKRNDDTGQPARNLFTY | 1405 | TTCAAGAGAAACGACGACACCGGCCAGCCTgccAGAAACCTGTTCACCTAC | 1525 |
| F10S12 | FKRNDDTGQPRANLFTY | 1406 | TTCAAGAGAAACGACGACACCGGCCAGCCTCGGgccAACCTGTTCACCTAC | 1526 |
| F10S13 | FKRNDDTGQPRRALFTY | 1407 | TTCAAGAGAAACGACGACACCGGCCAGCCTCGGAGAgccCTGTTCACCTAC | 1527 |
| F10S14 | FKRNDDTGQPRRNAFTY | 1408 | TTCAAGAGAAACGACGACACCGGCCAGCCTCGGAGAAACgccTTCACCTAC | 1528 |
| F10S15 | FKRNDDTGQPRRNLATY | 1409 | TTCAAGAGAAACGACGACACCGGCCAGCCTCGGAGAAACCTGgccACCTAC | 1529 |
| F10S16 | FKRNDDTGQPRRNLFAY | 1410 | TTCAAGAGAAACGACGACACCGGCCAGCCTCGGAGAAACCTGTTCgccTAC | 1530 |
| F10S17 | FKRNDDTGQPRRNLFTA | 1411 | TTCAAGAGAAACGACGACACCGGCCAGCCTCGGAGAAACCTGTTCACCgcc | 1531 |
| F38S1 | ARILFKEHTKLDDITKT | 1412 | gccAGAATCCTGTTCAAGGAGCACACCAAGCTGGACGACATCACCAAGACC | 1532 |
| F38S2 | LAILFKEHTKLDDITKT | 1413 | CTGgccATCCTGTTCAAGGAGCACACCAAGCTGGACGACATCACCAAGACC | 1533 |
| F38S3 | LRALFKEHTKLDDITKT | 1414 | CTGAGAgccCTGTTCAAGGAGCACACCAAGCTGGACGACATCACCAAGACC | 1534 |
| F38S4 | LRIAFKEHTKLDDITKT | 1415 | CTGAGAATCgccTTCAAGGAGCACACCAAGCTGGACGACATCACCAAGACC | 1535 |
| F38S5 | LRILAKEHTKLDDITKT | 1416 | CTGAGAATCCTGgCCAAGGAGCACACCAAGCTGGACGACATCACCAAGACC | 1536 |
| F38S6 | LRILFAEHTKLDDITKT | 1417 | CTGAGAATCCTGTTCgccGAGCACACCAAGCTGGACGACATCACCAAGACC | 1537 |
| F38S7 | LRILFKAHTKLDDITKT | 1418 | CTGAGAATCCTGTTCAAGgccCACACCAAGCTGGACGACATCACCAAGACC | 1538 |
| F38S8 | LRILFKEATKLDDITKT | 1419 | CTGAGAATCCTGTTCAAGGAGgccACCAAGCTGGACGACATCACCAAGACC | 1539 |
| F38S9 | LRILFKEHAKLDDITKT | 1420 | CTGAGAATCCTGTTCAAGGAGCACgccAAGCTGGACGACATCACCAAGACC | 1540 |
| F38S10 | LRILFKEHTALDDITKT | 1421 | CTGAGAATCCTGTTCAAGGAGCACACCgccCTGGACGACATCACCAAGACC | 1541 |
| F38S11 | LRILFKEHTKADDITKT | 1422 | CTGAGAATCCTGTTCAAGGAGCACACCAAGgccGACGACATCACCAAGACC | 1542 |
| F38S12 | LRILFKEHTKLADITKT | 1423 | CTGAGAATCCTGTTCAAGGAGCACACCAAGCTGgccGACATCACCAAGACC | 1543 |
| F38S13 | LRILFKEHTKLDAITKT | 1424 | CTGAGAATCCTGTTCAAGGAGCACACCAAGCTGGACgccATCACCAAGACC | 1544 |
| F38S14 | LRILFKEHTKLDDATKT | 1425 | CTGAGAATCCTGTTCAAGGAGCACACCAAGCTGGACGACgccACCAAGACC | 1545 |
| F38S15 | LRILFKEHTKLDDIAKT | 1426 | CTGAGAATCCTGTTCAAGGAGCACACCAAGCTGGACGACATCgccAAGACC | 1546 |
| F38S16 | LRILFKEHTKLDDITAT | 1427 | CTGAGAATCCTGTTCAAGGAGCACACCAAGCTGGACGACATCACCgccACC | 1547 |
| F38S17 | LRILFKEHTKLDDITKA | 1428 | CTGAGAATCCTGTTCAAGGAGCACACCAAGCTGGACGACATCACCAAGgcc | 1548 |
| F40S1 | AYPSLVYTMSSKYVDNI | 1429 | gccTATCCCTCCCTGGTGTACACCATGAGCAGCAAGTACGTGGACAATATC | 1549 |
| F40S2 | NAPSLVYTMSSKYVDNI | 1430 | AACgccCCCTCCCTGGTGTACACCATGAGCAGCAAGTACGTGGACAATATC | 1550 |
| F40S3 | NYASLVYTMSSKYVDNI | 1431 | AACTATgccTCCCTGGTGTACACCATGAGCAGCAAGTACGTGGACAATATC | 1551 |
| F40S4 | NYPALVYTMSSKYVDNI | 1432 | AACTATCCCgccCTGGTGTACACCATGAGCAGCAAGTACGTGGACAATATC | 1552 |
| F40S5 | NYPSAVYTMSSKYVDNI | 1433 | AACTATCCCTCCgccGTGTACACCATGAGCAGCAAGTACGTGGACAATATC | 1553 |
| F40S6 | NYPSLAYTMSSKYVDNI | 1434 | AACTATCCCTCCCTGgccTACACCATGAGCAGCAAGTACGTGGACAATATC | 1554 |
| F40S7 | NYPSLVATMSSKYVDNI | 1435 | AACTATCCCTCCCTGGTGgccACCATGAGCAGCAAGTACGTGGACAATATC | 1555 |
| F40S8 | NYPSLVYAMSSKYVDNI | 1436 | AACTATCCCTCCCTGGTGTACgccATGAGCAGCAAGTACGTGGACAATATC | 1556 |
| F40S9 | NYPSLVYTASSKYVDNI | 1437 | AACTATCCCTCCCTGGTGTACACCgccAGCAGCAAGTACGTGGACAATATC | 1557 |
| F40S10 | NYPSLVYTMASKYVDNI | 1438 | AACTATCCCTCCCTGGTGTACACCATGgccAGCAAGTACGTGGACAATATC | 1558 |
| F40S11 | NYPSLVYTMSAKYVDNI | 1439 | AACTATCCCTCCCTGGTGTACACCATGAGCgccAAGTACGTGGACAATATC | 1559 |
| F40S12 | NYPSLVYTMSSAYVDNI | 1440 | AACTATCCCTCCCTGGTGTACACCATGAGCAGCgccTACGTGGACAATATC | 1560 |
| F40S13 | NYPSLVYTMSSKAVDNI | 1441 | AACTATCCCTCCCTGGTGTACACCATGAGCAGCAAGgccGTGGACAATATC | 1561 |
| F40S14 | NYPSLVYTMSSKYADNI | 1442 | AACTATCCCTCCCTGGTGTACACCATGAGCAGCAAGTACgccGACAATATC | 1562 |
| F40S15 | NYPSLVYTMSSKYVANI | 1443 | AACTATCCCTCCCTGGTGTACACCATGAGCAGCAAGTACGTGgccAATATC | 1563 |
| F40S16 | NYPSLVYTMSSKYVDAI | 1444 | AACTATCCCTCCCTGGTGTACACCATGAGCAGCAAGTACGTGGACgccATC | 1564 |
| F40S17 | NYPSLVYTMSSKYVDNA | 1445 | AACTATCCCTCCCTGGTGTACACCATGAGCAGCAAGTACGTGGACAATgcc | 1565 |
| F46S1 | ATKLKDARNKALHGEIL | 1446 | gccACGAAGCTGAAGGATGCCcggaacAAGGCCCTGcacGGCGAGATCCTG | 1566 |
| F46S2 | LAKLKDARNKALHGEIL | 1447 | CTGgccAAGCTGAAGGATGCCcggaacAAGGCCCTGcacGGCGAGATCCTG | 1567 |
| F46S3 | LTALKDARNKALHGEIL | 1448 | CTGACGgccCTGAAGGATGCCcggaacAAGGCCCTGcacGGCGAGATCCTG | 1568 |
| F46S4 | LTKAKDARNKALHGEIL | 1449 | CTGACGAAGgccAAGGATGCCcggaacAAGGCCCTGcacGGCGAGATCCTG | 1569 |
| F46S5 | LTKLADARNKALHGEIL | 1450 | CTGACGAAGCTGgccGATGCCcggaacAAGGCCCTGcacGGCGAGATCCTG | 1570 |
| F46S6 | LTKLKAARNKALHGEIL | 1451 | CTGACGAAGCTGAAGgccGCCcggaacAAGGCCCTGcacGGCGAGATCCTG | 1571 |
| F46S7 | LTKLKDVRNKALHGEIL | 1452 | CTGACGAAGCTGAAGGATgtgcggaacAAGGCCCTGcacGGCGAGATCCTG | 1572 |
| F46S10 | LTKLKDARNAALHGEIL | 1453 | CTGACGAAGCTGAAGGATGCCcggaacgccGCCCTGcacGGCGAGATCCTG | 1573 |
| F46S11 | LTKLKDARNKVLHGEIL | 1454 | CTGACGAAGCTGAAGGATGCCcggaacAAGgtgCTGcacGGCGAGATCCTG | 1574 |
| F46S12 | LTKLKDARNKAAHGEIL | 1455 | CTGACGAAGCTGAAGGATGCCcggaacAAGGCCgcccacGGCGAGATCCTG | 1575 |
| F46S14 | LTKLKDARNKALHAEIL | 1456 | CTGACGAAGCTGAAGGATGCCcggaacAAGGCCCTGcacgccGAGATCCTG | 1576 |
| F46S15 | LTKLKDARNKALHGAIL | 1457 | CTGACGAAGCTGAAGGATGCCcggaacAAGGCCCTGcacGGCgccATCCTG | 1577 |
| F46S16 | LTKLKDARNKALHGEAL | 1458 | CTGACGAAGCTGAAGGATGCCcggaacAAGGCCCTGcacGGCGAGgccCTG | 1578 |
| F46S17 | LTKLKDARNKALHGEIA | 1459 | CTGACGAAGCTGAAGGATGCCcggaacAAGGCCCTGcacGGCGAGATCgcc | 1579 |
| F10S18 | FARNADAAQPRRNLFTY | 1460 | TTCgccAGAAACgccGACgccgccCAGCCTCGGAGAAACCTGTTCACCTAC | 1580 |
| F10S19 | FARNADAGQPRRNLFAY | 1461 | TTCgccAGAAACgccGACgccGGCCAGCCTCGGAGAAACCTGTTCgccTAC | 1581 |
| F10S20 | FARNADTAQPRRNLFAY | 1462 | TTCgccAGAAACgccGACACCgccCAGCCTCGGAGAAACCTGTTCgccTAC | 1582 |
| F10S21 | FARNDDAAQPRRNLFAY | 1463 | TTCgccAGAAACGACGACgccgccCAGCCTCGGAGAAACCTGTTCgccTAC | 1583 |
| F10S22 | FKRNADAAQPRRNLFAY | 1464 | TTCAAGAGAAACgccGACgccgccCAGCCTCGGAGAAACCTGTTCgccTAC | 1584 |
| F10S23 | FARNADAGQPRRNLFTY | 1465 | TTCgccAGAAACgccGACgccGGCCAGCCTCGGAGAAACCTGTTCACCTAC | 1585 |
| F10S24 | FARNADTAQPRRNLFTY | 1466 | TTCgccAGAAACgccGACACCgccCAGCCTCGGAGAAACCTGTTCACCTAC | 1586 |
| F10S25 | FARNADTGQPRRNLFAY | 1467 | TTCgccAGAAACgccGACACCGGCCAGCCTCGGAGAAACCTGTTCgccTAC | 1587 |
| F10S26 | FARNDDAAQPRRNLFTY | 1468 | TTCgccAGAAACGACGACgccgccCAGCCTCGGAGAAACCTGTTCACCTAC | 1588 |
| F10S27 | FARNDDAGQPRRNLFAY | 1469 | TTCgccAGAAACGACGACgccGGCCAGCCTCGGAGAAACCTGTTCgccTAC | 1589 |
| F10S28 | FARNDDTAQPRRNLFAY | 1470 | TTCgccAGAAACGACGACACCgccCAGCCTCGGAGAAACCTGTTCgccTAC | 1590 |
| F10S29 | FKRNADAAQPRRNLFTY | 1471 | TTCAAGAGAAACgccGACgccgccCAGCCTCGGAGAAACCTGTTCACCTAC | 1591 |
| F10S30 | FKRNADAGQPRRNLFAY | 1472 | TTCAAGAGAAACgccGACgccGGCCAGCCTCGGAGAAACCTGTTCgccTAC | 1592 |
| F10S31 | FKRNADTAQPRRNLFAY | 1473 | TTCAAGAGAAACgccGACACCgccCAGCCTCGGAGAAACCTGTTCgccTAC | 1593 |
| F10S32 | FKRNDDAAQPRRNLFAY | 1474 | TTCAAGAGAAACGACGACgccgccCAGCCTCGGAGAAACCTGTTCgccTAC | 1594 |
| F10S33 | FARNADTGQPRRNLFTY | 1475 | TTCgccAGAAACgccGACACCGGCCAGCCTCGGAGAAACCTGTTCACCTAC | 1595 |
| F10S34 | FARNDDAGQPRRNLFTY | 1476 | TTCgccAGAAACGACGACgccGGCCAGCCTCGGAGAAACCTGTTCACCTAC | 1596 |
| F10S35 | FARNDDTAQPRRNLFTY | 1477 | TTCgccAGAAACGACGACACCgccCAGCCTCGGAGAAACCTGTTCACCTAC | 1597 |
| F10S36 | FARNDDTGQPRRNLFAY | 1478 | TTCgccAGAAACGACGACACCGGCCAGCCTCGGAGAAACCTGTTCgccTAC | 1598 |
| F10S37 | FKRNADAGQPRRNLFTY | 1479 | TTCAAGAGAAACgccGACgccGGCCAGCCTCGGAGAAACCTGTTCACCTAC | 1599 |
| F10S38 | FKRNADTAQPRRNLFTY | 1480 | TTCAAGAGAAACgccGACACCgccCAGCCTCGGAGAAACCTGTTCACCTAC | 1600 |
| F10S39 | FKRNADTGQPRRNLFAY | 1481 | TTCAAGAGAAACgccGACACCGGCCAGCCTCGGAGAAACCTGTTCgccTAC | 1601 |
| F10S40 | FKRNDDAAQPRRNLFTY | 1482 | TTCAAGAGAAACGACGACgccgccCAGCCTCGGAGAAACCTGTTCACCTAC | 1602 |
| F10S41 | FKRNDDAGQPRRNLFAY | 1483 | TTCAAGAGAAACGACGACgccGGCCAGCCTCGGAGAAACCTGTTCgccTAC | 1603 |
| F10S42 | FKRNDDTAQPRRNLFAY | 1484 | TTCAAGAGAAACGACGACACCgccCAGCCTCGGAGAAACCTGTTCgccTAC | 1604 |
| F10S43 | FKANDDTGQAARNLFTY | 1485 | TTCAAGgccAACGACGACACCGGCCAGgccgccAGAAACCTGTTCACCTAC | 1605 |
| F10S44 | FKANDDTGQARANLFTY | 1486 | TTCAAGgccAACGACGACACCGGCCAGgccCGGgccAACCTGTTCACCTAC | 1606 |
| F10S45 | FKANDDTGQPAANLFTY | 1487 | TTCAAGgccAACGACGACACCGGCCAGCCTgccgccAACCTGTTCACCTAC | 1607 |
| F10S46 | FKRNDDTGQAAANLFTY | 1488 | TTCAAGAGAAACGACGACACCGGCCAGgccgccgccAACCTGTTCACCTAC | 1608 |
| F10S47 | FKANDDTGQARRNLFTY | 1489 | TTCAAGgccAACGACGACACCGGCCAGgccCGGAGAAACCTGTTCACCTAC | 1609 |
| F10S48 | FKANDDTGQPARNLFTY | 1490 | TTCAAGgccAACGACGACACCGGCCAGCCTgccAGAAACCTGTTCACCTAC | 1610 |
| F10S49 | FKANDDTGQPRANLFTY | 1491 | TTCAAGgccAACGACGACACCGGCCAGCCTCGGgccAACCTGTTCACCTAC | 1611 |
| F10S50 | FKRNDDTGQAARNLFTY | 1492 | TTCAAGAGAAACGACGACACCGGCCAGgccgccAGAAACCTGTTCACCTAC | 1612 |
| F10S51 | FKRNDDTGQARANLFTY | 1493 | TTCAAGAGAAACGACGACACCGGCCAGgccCGGgccAACCTGTTCACCTAC | 1613 |
| F10S52 | FKRNDDTGQPAANLFTY | 1494 | TTCAAGAGAAACGACGACACCGGCCAGCCTgccgccAACCTGTTCACCTAC | 1614 |
| F40S18 | NAPSLVATMSSKAVDNI | 1495 | AACgccCCCTCCCTGGTGgccACCATGAGCAGCAAGgccGTGGACAATATC | 1615 |
| F40S19 | NAPSLVATMSSKYVANI | 1496 | AACgccCCCTCCCTGGTGgccACCATGAGCAGCAAGTACGTGgccAATATC | 1616 |
| F40S20 | NAPSLVYTMSSKAVANI | 1497 | AACgccCCCTCCCTGGTGTACACCATGAGCAGCAAGgccGTGgccAATATC | 1617 |
| F40S21 | NYPSLVATMSSKAVANI | 1498 | AACTATCCCTCCCTGGTGgccACCATGAGCAGCAAGgccGTGgccAATATC | 1618 |
| F40S22 | NAPSLVATMSSKYVDNI | 1499 | AACgccCCCTCCCTGGTGgccACCATGAGCAGCAAGTACGTGGACAATATC | 1619 |
| F40S23 | NAPSLVYTMSSKAVDNI | 1500 | AACgccCCCTCCCTGGTGTACACCATGAGCAGCAAGgccGTGGACAATATC | 1620 |
| F40S24 | NAPSLVYTMSSKYVANI | 1501 | AACgccCCCTCCCTGGTGTACACCATGAGCAGCAAGTACGTGgccAATATC | 1621 |
| F40S25 | NYPSLVATMSSKAVDNI | 1502 | AACTATCCCTCCCTGGTGgccACCATGAGCAGCAAGgccGTGGACAATATC | 1622 |
| F40S26 | NYPSLVATMSSKYVANI | 1503 | AACTATCCCTCCCTGGTGgccACCATGAGCAGCAAGTACGTGgccAATATC | 1623 |
| F40S27 | NYPSLVYTMSSKAVANI | 1504 | AACTATCCCTCCCTGGTGTACACCATGAGCAGCAAGgccGTGgccAATATC | 1624 |
| F40S28 | NYASAVYTASSKYVDNI | 1505 | AACTATgccTCCgccGTGTACACCgccAGCAGCAAGTACGTGGACAATATC | 1625 |
| F40S29 | NYASAVYTMSSKYVDNA | 1506 | AACTATgccTCCgccGTGTACACCATGAGCAGCAAGTACGTGGACAATgcc | 1626 |
| F40S30 | NYASLVYTASSKYVDNA | 1507 | AACTATgccTCCCTGGTGTACACCgccAGCAGCAAGTACGTGGACAATgcc | 1627 |
| F40S31 | NYPSAVYTASSKYVDNA | 1508 | AACTATCCCTCCgccGTGTACACCgccAGCAGCAAGTACGTGGACAATgcc | 1628 |
| F40S32 | NYASAVYTMSSKYVDNI | 1509 | AACTATgccTCCgccGTGTACACCATGAGCAGCAAGTACGTGGACAATATC | 1629 |
| F40S33 | NYASLVYTASSKYVDNI | 1510 | AACTATgccTCCCTGGTGTACACCgccAGCAGCAAGTACGTGGACAATATC | 1630 |
| F40S34 | NYASLVYTMSSKYVDNA | 1511 | AACTATgccTCCCTGGTGTACACCATGAGCAGCAAGTACGTGGACAATgcc | 1631 |
| F40S35 | NYPSAVYTASSKYVDNI | 1512 | AACTATCCCTCCgccGTGTACACCgccAGCAGCAAGTACGTGGACAATATC | 1632 |
| F40S36 | NYPSAVYTMSSKYVDNA | 1513 | AACTATCCCTCCgccGTGTACACCATGAGCAGCAAGTACGTGGACAATgcc | 1633 |
| F40S37 | NYPSLVYTASSKYVDNA | 1514 | AACTATCCCTCCCTGGTGTACACCgccAGCAGCAAGTACGTGGACAATgcc | 1634 |

In this experiment, dCas13f with no gRNA-guided cleavage was used as a negative control, and the results (mean±s.e.m.) were normalized against that of dCas13f and listed below. Cas13f mutants located at the upper left area of FIG. 29 had low collateral effect (high mCherry signal) and high gRNA-guided cleavage activity (low EGFP signal), and were selected as the desired low/no collateral effect mutants.

| | % mCherry | S.E.M. | % EGFP | S.E.M. |
|---|---|---|---|---|
| dead | 1 | 0.023131 | 1 | 0.01545 |
| WT | 0.328068 | 0.001057 | 0.042958 | 0.000813 |
| F10V1 | 0.761218 | 0.005948 | 0.362324 | 0.000881 |
| F10V4 | 0.691621 | 0.003172 | 0.103638 | 0.002507 |
| F38V2 | 0.221726 | 0.00152 | 0.032981 | 0.001559 |
| F40V2 | 0.972985 | 0.002644 | 0.351174 | 0.010436 |
| F40V4 | 0.735119 | 0.011235 | 0.165258 | 0.002711 |
| F46V1 | 0.466461 | 0.009847 | 0.103756 | 0.002033 |
| F46V3 | 0.141941 | 0.003569 | 0.013439 | 0.00044 |
| F38S2 | 0.213141 | 0.00423 | 0.02993 | 6.78E-05 |
| F38S3 | 0.315018 | 0.007798 | 0.045305 | 0.000271 |
| F38S4 | 0.160027 | 0.000661 | 0.021596 | 0.000542 |
| F38S5 | 0.213255 | 0.001124 | 0.028521 | 0.00061 |
| F38S6 | 0.206616 | 0.002181 | 0.02338 | 0.000217 |
| F38S7 | 0.176969 | 0.00185 | 0.022887 | 0.001152 |
| F38S8 | 0.196085 | 0.00033 | 0.020164 | 0.00164 |
| F38S9 | 0.199748 | 0.002049 | 0.025822 | 0.000949 |
| F38S10 | 0.138851 | 0.002445 | 0.018545 | 0.000542 |
| F38S11 | 0.177999 | 0.008922 | 0.022418 | 0.001423 |
| F38S12 | 0.135302 | 0.001322 | 0.017019 | 0.001559 |
| F38S13 | 0.19826 | 0.001454 | 0.027817 | 0.000474 |
| F38S15 | 0.172161 | 0.000661 | 0.017758 | 0.000861 |
| F38S16 | 0.194025 | 0.000727 | 0.0227 | 0.000854 |
| F38S17 | 0.20158 | 0.003503 | 0.020305 | 0.000339 |
| F40S1 | 0.230197 | 0.002842 | 0.025704 | 0.000203 |
| F40S2 | 0.213828 | 0.010971 | 0.018897 | 0.00061 |
| F40S3 | 0.178915 | 0.004296 | 0.02007 | 0.001016 |
| F40S4 | 0.163347 | 0.001917 | 0.019836 | 0.00061 |
| F40S5 | 0.226648 | 0.000264 | 0.033216 | 0.000203 |
| F40S6 | 0.203755 | 0.000529 | 0.024061 | 6.78E-05 |
| F40S7 | 0.632669 | 0.016192 | 0.072887 | 0.001288 |
| F40S8 | 0.22951 | 0.001917 | 0.027277 | 0.001111 |
| F40S9 | 0.505266 | 0.029872 | 0.087559 | 0.002846 |
| F40S11 | 0.502404 | 0.006939 | 0.096596 | 0.000339 |
| F40S12 | 0.488095 | 0.002776 | 0.11608 | 6.78E-05 |
| F40S13 | 0.485234 | 0.000991 | 0.186972 | 0.001559 |
| F40S14 | 0.445971 | 0.001586 | 0.123826 | 0.005489 |
| F40S15 | 0.322001 | 0.00271 | 0.100235 | 0.000813 |
| F40S16 | 0.255952 | 0.017183 | 0.097887 | 0.000949 |
| F40S17 | 0.495765 | 0.016853 | 0.125352 | 0.002168 |
| F40S18 | 0.293842 | 0.013152 | 0.208451 | 0.004201 |
| F40S19 | 0.39011 | 0.022338 | 0.148239 | 0.002778 |
| F40S20 | 0.367674 | 0.011764 | 0.208099 | 0.00332 |
| F40S21 | 0.906593 | 0.002644 | 0.262324 | 0.000474 |
| F40S22 | 0.811928 | 0.004164 | 0.138498 | 0.003659 |
| F40S25 | 0.68109 | 0.033705 | 0.330282 | 0.000271 |
| F40S26 | 0.87065 | 0.021413 | 0.163263 | 0.00576 |
| F40S28 | 0.597756 | 0.006212 | 0.066526 | 0.000759 |
| F40S29 | 0.503205 | 0.001454 | 0.107981 | 0.001762 |
| F40S30 | 0.641598 | 0.005882 | 0.166901 | 0.003659 |
| F40S31 | 0.859661 | 0.001983 | 0.298122 | 0.002033 |
| F40S32 | 0.465545 | 0.006146 | 0.066549 | 0.000203 |
| F40S33 | 0.372253 | 0.013614 | 0.058685 | 0.002033 |
| F40S34 | 0.30506 | 0.004957 | 0.044484 | 0.001694 |
| F40S35 | 0.573832 | 0.000859 | 0.080164 | 0.001559 |
| F40S36 | 0.84913 | 0.009252 | 0.217371 | 0.002033 |
| F40S37 | 0.670673 | 0.031656 | 0.12946 | 0.00576 |
| F46S1 | 0.213713 | 0.000727 | 0.041315 | 0.000678 |
| F46S2 | 0.758013 | 0.030401 | 0.83885 | 0.025412 |
| F46S4 | 0.222184 | 0.000859 | 0.051878 | 0.000407 |
| F46S5 | 0.356227 | 0.004494 | 0.035446 | 0.001762 |
| F46S6 | 0.153159 | 0.005948 | 0.021009 | 0.00061 |
| F46S7 | 0.21875 | 0.003899 | 0.024061 | 0.000474 |
| F46S10 | 0.213599 | 0.00119 | 0.030869 | 0.001152 |
| F46S11 | 0.474359 | 0.01216 | 0.080047 | 0.001355 |
| F46S12 | 0.2856 | 0.013152 | 0.067371 | 0.002846 |
| F46S14 | 0.167468 | 0.008525 | 0.023709 | 0.000271 |
| F46S15 | 0.110577 | 0.002115 | 0.013146 | 0.000542 |
| F10S1 | 0.478709 | 0.004626 | 0.093192 | 0.000813 |
| F10S2 | 0.609547 | 0.000859 | 0.080845 | 0.002114 |
| F10S3 | 0.280105 | 0.005089 | 0.024613 | 0.001376 |
| F10S4 | 0.137477 | 6.61E-05 | 0.017723 | 0.000339 |
| F10S5 | 0.130952 | 0.008459 | 0.026995 | 0.000271 |
| F10S6 | 0.130609 | 0.005882 | 0.014789 | 0.001084 |
| F10S8 | 0.287202 | 0.002577 | 0.026056 | 0.000678 |
| F10S9 | 0.165865 | 0.002313 | 0.014812 | 0.000108 |
| F10S10 | 0.235462 | 0.000727 | 0.019683 | 0.001105 |
| F10S12 | 0.642399 | 0.012689 | 0.075129 | 0.001227 |
| F10S13 | 0.290636 | 0.002974 | 0.035211 | 0.000678 |
| F10S17 | 0.297276 | 0.001124 | 0.067488 | 0.000474 |
| F10S18 | 0.709936 | 0.00608 | 0.130399 | 0.001288 |
| F10S19 | 0.794414 | 0.010574 | 0.274413 | 0.013146 |
| F10S21 | 0.769345 | 0.00033 | 0.232629 | 0.004066 |
| F10S22 | 0.442193 | 0.005353 | 0.12723 | 0.004608 |
| F10S23 | 0.730426 | 0.00033 | 0.149178 | 0.000881 |
| F10S24 | 0.779304 | 0.012689 | 0.139319 | 0.002778 |
| F10S26 | 0.795215 | 0.012359 | 0.145775 | 0.000813 |
| F10S27 | 0.786287 | 0.007336 | 0.209038 | 0.006167 |
| F10S28 | 0.731456 | 0.015729 | 0.21115 | 0.002236 |
| F10S29 | 0.363439 | 0.009186 | 0.050822 | 0.001152 |
| F10S30 | 0.418613 | 0.000463 | 0.124296 | 0.000474 |
| F10S31 | 0.563187 | 0.006609 | 0.153169 | 0.001423 |
| F10S32 | 0.31353 | 0.011962 | 0.061854 | 6.78E-05 |
| F10S33 | 0.833562 | 0.011499 | 0.151526 | 0.006031 |
| F10S34 | 0.786516 | 0.017513 | 0.108099 | 0.003727 |
| F10S35 | 0.815018 | 0.00727 | 0.112559 | 0.001559 |
| F10S36 | 0.810897 | 0.003833 | 0.212207 | 0.006641 |
| F10S37 | 0.322688 | 0.007468 | 0.043192 | 0.00393 |
| F10S38 | 0.444254 | 0.006014 | 0.093075 | 0.002507 |
| F10S39 | 0.495192 | 0.004626 | 0.161033 | 0.011791 |
| F10S40 | 0.320169 | 0.004957 | 0.028239 | 0.00042 |
| F10S41 | 0.36424 | 0.006873 | 0.078286 | 0.000203 |
| F10S42 | 0.456731 | 0.010442 | 0.096009 | 0.00122 |
| F10S43 | 0.634043 | 0.002842 | 0.059272 | 0.002643 |
| F10S44 | 0.704556 | 0.01606 | 0.093897 | 0.002033 |
| F10S45 | 0.902701 | 0.009252 | 0.204812 | 0.002778 |
| F10S46 | 0.790179 | 0.005221 | 0.146244 | 0.002033 |
| F10S47 | 0.562729 | 0.005155 | 0.057864 | 0.003591 |
| F10S48 | 0.849245 | 0.02214 | 0.101526 | 0.005624 |
| F10S49 | 0.863897 | 0.012755 | 0.132629 | 0.001491 |
| F10S50 | 0.724359 | 0.010574 | 0.09162 | 0.002534 |
| F10S51 | 0.644116 | 0.000198 | 0.094836 | 0.005557 |
| F10S52 | 0.695513 | 0.004097 | 0.194249 | 0.005353 |
| F10S7 | 0.249313 | 0.006741 | 0.024308 | 0.000603 |
| F10S11 | 0.650069 | 0.006014 | 0.089977 | 0.000122 |
| F10S14 | 0.279075 | 0.009252 | 0.033568 | 0.001084 |
| F10S15 | 0.421016 | 0.008459 | 0.113615 | 0.004472 |
| F10S16 | 0.410027 | 0.016126 | 0.119836 | 0.006031 |
| F10S20 | 0.667353 | 0.012557 | 0.251526 | 0.00698 |
| F10S25 | 0.895147 | 0.010574 | 0.280869 | 0.005895 |
| F10S43 | 0.694712 | 0.003899 | 0.051479 | 0.000718 |
| F38S1 | 0.214744 | 0.000264 | 0.01912 | 0.000332 |
| F38S13 | 0.246223 | 0.011169 | 0.02723 | 0.000678 |
| F40S10 | 0.384272 | 0.010244 | 0.04554 | 0.000678 |
| F40S23 | 0.863782 | 0.005551 | 0.144836 | 0.000407 |
| F40S24 | 0.565247 | 0.007666 | 0.04142 | 0.000996 |
| F40S27 | 0.818109 | 0.016853 | 0.087676 | 0.001559 |
| F46S2 | 0.244391 | 0.000727 | 0.025822 | 0.000407 |
| F46S3 | 0.903159 | 0.018108 | 0.861502 | 0.023717 |
| F46S16 | 0.43544 | 0.016787 | 0.055516 | 0.000881 |
| F46S17 | 0.270833 | 0.004891 | 0.033216 | 0.000745 |
| F40S27 | 0.8181 | 0.0169 | 0.0877 | 0.0016 |
| F10V4 | 0.8215 | 0.0088 | 0.0929 | 0.0018 |
| F10S48 | 0.8492 | 0.0221 | 0.1015 | 0.0056 |
| F10S34 | 0.7865 | 0.0175 | 0.1081 | 0.0037 |
| F10S35 | 0.8150 | 0.0073 | 0.1126 | 0.0016 |
| F10S49 | 0.8639 | 0.0128 | 0.1326 | 0.0015 |
| F40S22 | 0.8119 | 0.0042 | 0.1385 | 0.0037 |
| F10S24 | 0.7793 | 0.0127 | 0.1393 | 0.0028 |
| F40S23 | 0.8638 | 0.0056 | 0.1448 | 0.0004 |
| F10S26 | 0.7952 | 0.0124 | 0.1458 | 0.0008 |
| F10S46 | 0.7902 | 0.0052 | 0.1462 | 0.0020 |
| F10S33 | 0.8336 | 0.0115 | 0.1515 | 0.0060 |
| F40S26 | 0.8707 | 0.0214 | 0.1633 | 0.0058 |
| F40V4 | 0.8524 | 0.0109 | 0.1698 | 0.0068 |
| F7V2 | 0.8915 | 0.0086 | 0.1956 | 0.0100 |
| F10S45 | 0.9027 | 0.0093 | 0.2048 | 0.0028 |
| F10S27 | 0.7863 | 0.0073 | 0.2090 | 0.0062 |
| F10S36 | 0.8109 | 0.0038 | 0.2122 | 0.0066 |
| F10V1 | 0.8131 | 0.0050 | 0.2123 | 0.0102 |
| F40S36 | 0.8491 | 0.0093 | 0.2174 | 0.0020 |
| F10S21 | 0.7693 | 0.0003 | 0.2326 | 0.0041 |

Overall, some of the Cas13f mutants exhibited low collateral effect (e.g., ≤25% collateral effect, or ≥75% mCherry⁺ cells), and high (e.g., EGFP⁺ cells≤25%) to intermediate gRNA-guided cleavage (e.g., 25%≤EGFP⁺ cells≤75%) including: F40S23 ((Y666A,Y677A), SEQ ID NO: 1635) and F40S27, etc (see below table and FIG. 28 and FIG. 29). Based on FACS data (not shown), these mutants have significantly reduced collateral effect compared to wild-type.

Other mutants/variants retained high gRNA-guided cleavage (e.g., EGFP⁺ cells≤25%), but also exhibited higher than wild-type level collateral activity (e.g., ≤25% mCherry⁺ cells). See tables above. These mutants/variants may be useful for better/more sensitivity detection methods such as SHERLOCK.

1. An engineered Clustered Regularly Interspaced Short Palindromic Repeat (CRISPR)-Cas13 effector enzyme, wherein the engineered Cas13:
(1) comprises a mutation in a region spatially close to an endonuclease catalytic domain (e.g., a HEPN domain) of the corresponding wild-type Cas13 effector enzyme;
(2) substantially preserves (e.g., retains at least 50%, 60%, 70%, 72.5%, 75%, 80%, 85%, 87.5%, 90%, 95%, 96%, 97%, 97.5%, 98%, 99% or more of) guide sequence-specific endonuclease cleavage activity of the wild-type Cas13 towards a target RNA complementary to the guide sequence; and,
(3) substantially lacks (e.g., retains less than 50%, 40%, 35%, 30%, 27.5%, 25%, 22.5%, 20%, 17.5%, 15%, 12.5%, 10%, 7.5%, 5%, 4%, 3%, 2.5%, 2%, 1% or less of) guide sequence-independent collateral endonuclease cleavage activity of the wild-type Cas13 towards a non-target RNA that does not bind to the guide sequence.

2. The engineered Cas13 of claim 1, wherein the Cas13 is a Cas13e, a Cas13f, a Cas13d (including CasRx), a Cas13a, a Cas13b, or a Cas13c.

3. The engineered Cas13 of claim 2, wherein the Cas13e has the amino acid sequence of SEQ ID NO: 4, and/or wherein the Cas13f has the amino acid sequence of SEQ ID NO: 52, and/or wherein the Cas13d has the amino acid sequence of SEQ ID NO: 101.

4. The engineered Cas13 of any one of claims 1-3, wherein the region includes residues within 130, 125, 120, 110, 100, 90, 80, 70, 60, 50, 40, 30, 20, or 10 amino acids from any residues of the endonuclease catalytic domain (e.g., an RXXXXH domain) in the primary sequence of the Cas13e; residues within 170, 160, 150, 140, 130, 120, 110, 100, 90, 80, 70, 60, 50,40, 30, 20, or 10 amino acids from any residues of the endonuclease catalytic domain (e.g., an RXXXXH domain) in the primary sequence of the Cas13d; or residues within 140, 130, 120, 110, 100, 90, 80, 70, 60, 50, 40, 30, 20, or 10 amino acids from any residues of the endonuclease catalytic domain (e.g., an RXXXXH domain) in the primary sequence of the Cas13f.

5. The engineered Cas13 of any one of claims 1-3, wherein the region includes residues more than 100, 110, 120, or 130 residues away from any residues of the endonuclease catalytic domain in the primary sequence of the Cas13, but are spatially within 1-10 or 5 Ångström of a residue of the endonuclease catalytic domain.

6. The engineered Cas13 of any one of claims 1-5, wherein the endonuclease catalytic domain is a HEPN domain, optionally a HEPN domain comprising an RXXXXH motif.

7. The engineered Cas13 of claim 6, wherein the RXXXXH motif comprises a R{N/H/K/Q/R}X₁X₂X₃H sequence (SEQ ID NO: 1024).

8. The engineered Cas13 of claim 7, wherein in the R{N/H/K/Q/R}X₁X₂X₃H sequence (SEQ ID NO: 1025), X₁ is R, S, D, E, Q, N, G, or Y; X₂ is I, S, T, V, or L; and X₃ is L, F, N, Y, V, I, S, D, E, or A.

9. The engineered Cas13 of claim 6, wherein the RXXXXH motif is an N-terminal RXXXXH motif comprising an RNXXXH sequence, such as an RN{Y/F}{F/Y}SH sequence (SEQ ID NO: 64).

10. The engineered Cas13 of claim 9, wherein the N-terminal RXXXXH motif has a RNYFSH sequence (SEQ ID NO: 65).

11. The engineered Cas13 of claim 9, wherein the N-terminal RXXXXH motif has a RNFYSH sequence (SEQ ID NO: 66).

12. The engineered Cas13 of claim 9, wherein the RXXXXH motif is a C-terminal RXXXXH motif comprising an R{N/A/R}{A/K/S/F}{A/L/F}{F/H/L}H sequence (SEQ ID NO: 1026).

13. The engineered Cas13 of claim 12, wherein the C-terminal RXXXXH motif has a RN(A/K)ALH sequence (SEQ ID NO: 67).

14. The engineered Cas13 of claim 12, wherein the C-terminal RXXXXH motif has a RAFFHH (SEQ ID NO: 68) or RRAFFH sequence (SEQ ID NO: 69).

15. The engineered Cas13 of any one of claims 1-14, wherein said region comprises, consists essentially of, or consists of:
(i) residues corresponding to residues between residues 1-194, 2-187, 227-242, 620-775, or 634-755 of SEQ ID NO: 4;
(ii) residues corresponding to the HEPN1-1 domain (e.g., residues 90-292), Helical2 domain (e.g., residues 536-690), and the HEPN2 domain (e.g., residues 690-967) of SEQ ID NO: 101; or,
(iii) residues corresponding to the HEPN1 domain (e.g., residues 1-168), Helical1 domain, Helical2 domain (e.g., residues 346-477), and the HEPN2 domain (e.g., residues 644-790) of SEQ ID NO: 52.

16. The engineered Cas13 of any one of claims 1-15, wherein said region comprises, consists essentially of, or consists of residues corresponding to residues between residues 35-51, 52-67, 156-171, 666-682, or 712-727 of SEQ ID NO: 4.

17. The engineered Cas13 of any one of claims 1-16, wherein said mutation comprises, consists essentially of, or consists of substitutions, within a stretch of 15-20 consecutive amino acids within the region, (a) one or more charged, nitrogen-containing side chain group, bulky (such as F or Y), aliphatic, and/or polar residues to a charge-neutral short chain aliphatic residue (such as A, V, or I); (b) one or more I/L to A substitution(s); and/or (c) one or more A to V substitution(s).

18. The engineered Cas13 of claim 17, wherein said stretch is about 16 or 17 residues.

19. The engineered Cas13 of claim 17 or 18, wherein substantially all, except for up to 1, 2, or 3, charged and polar residues within the stretch are substituted.

20. The engineered Cas13 of any one of claims 17-19, wherein a total of about 7, 8, 9, or 10 charged and polar residues within the stretch are substituted.

21. The engineered Cas13 of any one of claims 17-20, wherein the N- and C-terminal 2 residues of the stretch are substituted to amino acids the coding sequences of which contain a restriction enzyme recognition sequence.

22. The engineered Cas13 of claim 21, wherein the N-terminal two residues are VF, and the C-terminal 2 residues are ED, and the restriction enzyme is BpiI.

23. The engineered Cas13 of any one of claims 17-22, wherein the one or more charged or polar residues comprise N, Q, R, K, H, D, E, Y, S, and T residues.

24. The engineered Cas13 of claim 23, wherein the one or more charged or polar residues comprise R, K, H, N, Y, and/or Q residues.

25. The engineered Cas13 of claim 17 or 18, wherein one or more Y residue(s) within said stretch is substituted.

26. The engineered Cas13 of claim 25, wherein said one or more Y residues(s) correspond to Y672, Y676, and/or Y715 of wild-type Cas13e.1 (SEQ ID NO: 4).

27. The engineered Cas13 of claim 25, wherein said stretch is residues 35-51, 52-67, 156-171, 666-682, or 712-727 of SEQ ID NO: 4.

28. The engineered Cas13 of claim 17 or 18, wherein the mutation comprises Ala substitution(s) corresponding to any one or more of SEQ ID NOs: 37-39, 45, and 48.

29. The engineered Cas13 of any one of claims 17-28, wherein the charge-neutral short chain aliphatic residue is Ala (A).

30. The engineered Cas13 of any one of claims 17-29, wherein said mutation comprises, consists essentially of, or consists of:
(a) substitutions within 1, 2, 3, 4, or 5 of said stretches of 15-20 consecutive amino acids within the region;
(b) a mutation corresponds to a Cas13d mutation (e.g., that of Example 4) that retains at least about 75% of guide RNA-specific cleavage of wild-type Cas13d (such as SEQ ID NO: 101), and exhibits less than about 27.5% collateral effect of wild-type Cas13d (such as SEQ ID NO: 101);
(c) a mutation corresponds to the N1V7, N2V7, N2V8 (cfCas13d), N3V7, or N15V4 mutation of Cas13d mutation;
(d) a mutation corresponds to a Cas13d mutation (e.g., that of Example 4) that retains between about 25-75% of guide RNA-specific cleavage of wild-type Cas13d (such as SEQ ID NO: 101), and exhibits less than about 27.5% collateral effect of wild-type Cas13d (such as SEQ ID NO: 101);
(e) a mutation corresponds to the N2V4, N2V5, N4V3, N6V3, N10V6, N15V2, N20V6, or N20-Y910A mutation of Cas13d mutation;
(f) a mutation corresponds to a Cas13e mutation (e.g., that of Example 1, 2, or 5) that retains at least about 75% of guide RNA-specific cleavage of wild-type Cas13e (such as SEQ ID NO: 4), and exhibits less than about 25% collateral effect of wild-type Cas13e (such as SEQ ID NO: 4);
(g) a mutation corresponds to the M1V4, M2V2, M2V3, M2V4, M5V1, M6V2, M6V3, M6V4, M7V1, M7V2, M7V3, M7-Y55A, M7-Y61A, M11V1, M12V3, M15V1, M15V2, M15-Y643A, M15-Y647A, M16V1, M16V2, M17V2, M18V2, M18V3, M19V2, M19V3, or M19-IA mutation of Cas13e mutation;
(h) a mutation corresponds to a Cas13e mutation (e.g., that of Example 5) that retains between about 25-75% of guide RNA-specific cleavage of wild-type Cas13e (such as SEQ ID NO: 4), and exhibits less than about 25% collateral effect of wild-type Cas13e (such as SEQ ID NO: 4);
(i) a mutation corresponds to the M17YY (cfCas13e), M8V4, M9V1, M11V2, M11V3, M13V1, M13V2, M13V3, M15V3, or M20V2 mutation of Cas13e mutation;
(j) a mutation corresponds to a Cas13f mutation (e.g., that of Example 12) that retains at least about 75% of guide RNA-specific cleavage of wild-type Cas13f (such as SEQ ID NO: 52), and exhibits less than about 25 or 27.5% collateral effect of wild-type Cas13f (such as SEQ ID NO: 52);
(k) a mutation corresponds to the F7V2, F10V1, F10V4, F40V2, F40V4, F44V2, F10S19, F10S21, F10S24, F10S26, F10S27, F10S33, F10S34, F10S35, F10S36, F10S45, F10S46, F10S48, F10S49, F40S22, F40S23, F40S26, F40S27, or F40S36 mutation of Cas13f mutation;
(l) a mutation corresponds to a Cas13f mutation (e.g., that of Example 12) that retains between about 50-75% of guide RNA-specific cleavage of wild-type Cas13f (such as SEQ ID NO: 52), and exhibits less than about 25 or 27.5% collateral effect of wild-type Cas13f (such as SEQ ID NO: 52); and/or
(m) a mutation corresponds to the F2V4, F3V1, F3V3, F3V4, F5V2, F5V3, F6V4, F7V1, F38V4, F40V1, F41V1, F41V3, F42V4, F43V1, F10S2, F10S11, F10S12, F10S18, F10S20, F10S23, F10S25, F10S28, F10S43, F10S44, F10S47, F10S50, F10S51, F10S52, F40S7, F40S9, F40S11, F40S21, F40S22, F40S24, F40S28, F40S29, F40S30, F40S35, or F40S37 or mutation of Cas13f mutation.

31. The engineered Cas13 of any one of claims 1-30, wherein the engineered Cas13 preserves at least about 50%, 60%, 70%, 72.5%, 75%, 80%, 85%, 87.5%, 90%, 95%, 96%, 97%, 97.5%, 98%, 99% or more of the guide sequence-specific endonuclease cleavage activity of the wild-type Cas13 towards the target RNA.

32. The engineered Cas13 of any one of claims 1-31, wherein the engineered Cas13 lacks at least about 70%, 72.5%, 75%, 77.5%, 80%, 82.5%, 85%, 87.5%, 90%, 92.5%, 95%, 96%, 97%, 98%, 99%, or 100% of the guide sequence-independent collateral endonuclease cleavage activity of the wild-type Cas13 towards the non-target RNA.

33. The engineered Cas13 of any one of claims 1-32, wherein the engineered Cas13 preserves at least about 80-90% of the guide sequence-specific endonuclease cleavage activity of the wild-type Cas13 towards the target RNA, and lacks at least about 95-100% of the guide sequence-independent collateral endonuclease cleavage activity of the wild-type Cas13 towards the non-target RNA.

34. The engineered Cas13 of any one of claims 1-33, having an amino acid sequence at least 80%, 85%, 90%, 95%, 96%, 97%, 98%, 99%, 99.1%, 99.2%, 99.3%, 99.4%, 99.5%, 99.6%, 99.7%, 99.8%, or 99.86% identical to any one of SEQ ID NOs: 6-10 and Cas13d (e.g., SEQ ID NO: 101), excluding any one or more of the regions defined by SEQ ID NOs: 16, 20, 24, 28, and 32, and any of the mutation regions in Example 4 or 5.

35. The engineered Cas13 of claim 34, wherein said amino acid sequence contains up to 1, 2, 3, 4, or 5 differences (a) in each of one or more regions defined by SEQ ID NO: 16, 20, 24, 28, and 32, as compared to SEQ ID NOs: 17, 21, 25, 29, and 33, respectively, or (b) in any of the mutations in claim 30.

36. The engineered Cas13 of any one of claims 1-35, having the amino acid sequence of any one of SEQ ID NOs: 6-10.

37. The engineered Cas13 of any one of claims 1-36, having the amino acid sequence of SEQ ID NO: 9 or 10.

38. The engineered Cas13 of any one of claims 1-37, further comprising a nuclear localization signal (NLS) sequence or a nuclear export signal (NES).

39. The engineered Cas13 of claim 38, comprising an N- and/or a C-terminal NLS.

40. A polynucleotide encoding the engineered Cas13 of any one of claims 1-39.

41. The polynucleotide of claim 40, which is codon-optimized for expression in a eukaryote, a mammal, such as a human or a non-human mammal, a plant, an insect, a bird, a reptile, a rodent (e.g., mouse, rat), a fish, a worm/nematode, or a yeast.

42. A polynucleotide having (i) one or more (e.g., 1, 2, 3, 4, 5, 6, 7, 8, 9 or 10) nucleotides additions, deletions, or substitutions compared to the polynucleotide of claim 40 or 41; (ii) at least 50%, 60%, 70%, 80%, 90%, 95%, or 97% sequence identity to e polynucleotide of claim 40 or 41; (iii) hybridize under stringent conditions with the polynucleotide of claim 40 or 41 or any of (i) and (ii); or (iv) is a complement of any of (i)-(iii).

43. A vector comprising the polynucleotide of any one of claims 40-42.

44. The vector of claim 43, wherein the polynucleotide is operably linked to a promoter and optionally an enhancer.

45. The vector of claim 44, wherein the promoter is a constitutive promoter, an inducible promoter, a ubiquitous promoter, or a tissue specific promoter.

46. The vector of any one of claims 43-45, which is a plasmid.

47. The vector of any one of claims 43-45, which is a retroviral vector, a phage vector, an adenoviral vector, a herpes simplex viral (HSV) vector, an AAV vector, or a lentiviral vector.

48. The vector of claim 47, wherein the AAV vector is a recombinant AAV vector of the serotype AAV1, AAV2, AAV4, AAV5, AAV6, AAV7, AAVrh74, AAV8, AAV9, AAV10, AAV 11, AAV 12, or AAV 13.

49. A delivery system comprising (1) a delivery vehicle, and (2) the engineered Cas13 of any one of claims 1-39, the polynucleotide of any one of claims 40-42, or the vector of any one of claims 43-48.

50. The delivery system of claim 49, wherein the delivery vehicle is a nanoparticle, a liposome, an exosome, a microvesicle, or a gene-gun.

51. A cell or a progeny thereof, comprising the engineered Cas13 of any one of claims 1-39, the polynucleotide of any one of claims 40-42, or the vector of any one of claims 43-48.

52. The cell or progeny thereof of claim 51, which is a eukaryotic cell (e.g., a non-human mammalian cell, a human cell, or a plant cell) or a prokaryotic cell (e.g., a bacteria cell).

53. A non-human multicellular eukaryote comprising the cell of claim 51 or 52.

54. The non-human multicellular eukaryote of claim 53, which is an animal (e.g., rodent or primate) model for a human genetic disorder.

55. A method of modifying a target RNA, the method comprising contacting the target RNA with a CRISPR-Cas13 complex comprising the engineered Cas13 of any one of claims 1-39, and a spacer sequence complementary to at least 15 nucleotides of the target RNA; wherein upon binding of the complex to the target RNA through the spacer sequence, engineered Cas13 modifies the target RNA.

56. The method of claim 55, wherein the target RNA is modified by cleavage by the engineered Cas13.

57. The method of claim 55 or 56, wherein the target RNA is an mRNA, a tRNA, an rRNA, a non-coding RNA, an lncRNA, or a nuclear RNA.

58. The method of any one of claims 55-57, wherein upon binding of the complex to the target RNA, the engineered Cas13 does not exhibit substantial (or detectable) collateral RNase activity.

59. The method of any one of claims 55-58, wherein the target RNA is within a cell.

60. The method of claim 59, wherein the cell is a cancer cell.

61. The method of claim 59, wherein the cell is infected with an infectious agent.

62. The method of claim 61, wherein the infectious agent is a virus, a prion, a protozoan, a fungus, or a parasite.

63. The method of claim 59, wherein the cell is a neuronal cell (e.g., astrocyte, glial cell (e.g., Muller glia cell, oligodendrocyte, ependymal cell, Schwan cell, NG2 cell, or satellite cell)).

64. The method of any one of claims 59-63, wherein the CRISPR-Cas13 complex is encoded by a first polynucleotide encoding the engineered Cas13 of any one of claims 1-39, and a second polynucleotide comprising or encoding a spacer RNA capable of binding to the target RNA, wherein the first and the second polynucleotides are introduced into the cell.

65. The method of claim 64, wherein the first and the second polynucleotides are introduced into the cell by the same vector.

66. The method of any one of claims 59-65, which causes one or more of: (i) in vitro or in vivo induction of cellular senescence; (ii) in vitro or in vivo cell cycle arrest; (iii) in vitro or in vivo cell growth inhibition and/or cell growth inhibition; (iv) in vitro or in vitro induction of anergy; (v) in vitro or in vitro induction of apoptosis; and (vi) in vitro or in vitro induction of necrosis.

67. A method of treating a condition or disease in a subject in need thereof, the method comprising administering to the subject a composition comprising a CRISPR-Cas complex comprising the engineered Cas13 of any one of claims 1-39 or a polynucleotide encoding the same; and a spacer sequence complementary to at least 15 nucleotides of a target RNA associated with the condition or disease; wherein upon binding of the complex to the target RNA through the spacer sequence, the engineered Cas13 cleaves the target RNA, thereby treating the condition or disease in the subject.

68. The method of claim 67, wherein the condition or disease is a neurological condition, a cancer or an infectious disease.

69. The method of claim 68, wherein the cancer is Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, or urinary bladder cancer.

70. The method of claim 68, wherein the neurological condition is glaucoma, age-related RGC loss, optic nerve injury, retinal ischemia, Leber's hereditary optic neuropathy, a neurological condition associated with degeneration of RGC neurons, a neurological condition associated with degeneration of functional neurons in the striatum of a subject in need thereof, Parkinson's disease, Alzheimer's disease, Huntington's disease, Schizophrenia, depression, drug addiction, movement disorder such as chorea, choreoathetosis, and dyskinesias, bipolar disorder, Autism spectrum disorder (ASD), or dysfunction.

71. The method of any one of claims 67-70, which is an in vitro method, an in vivo method, or an ex vivo method.

72. A CRISPR-Cas complex comprising the engineered Cas13 of any one of claims 1-39, a guide RNA comprising a DR sequence that binds the engineered Cas13 and a spacer sequence designed to be complementary to and binds a target RNA.

73. The CRISPR-Cas complex of claim 72, wherein the target RNA is encoded by a eukaryotic DNA.

74. The CRISPR-Cas complex of claim 73, wherein the eukaryotic DNA is a non-human mammalian DNA, a non-human primate DNA, a human DNA, a plant DNA, an insect DNA, a bird DNA, a reptile DNA, a rodent DNA, a fish DNA, a worm/nematode DNA, a yeast DNA.

75. The CRISPR-Cas complex of any one of claims 72-74, wherein the target RNA is an mRNA.

76. The CRISPR-Cas complex of any one of claims 72-75, further comprising a target RNA comprising a sequence capable of hybridizing to the spacer sequence.

77. A method of identifying an engineered CRISPR/Cas effector enzyme of a corresponding wild-type Cas effector enzyme, wherein the engineered Cas substantially maintains guide-sequence-specific endonuclease activity and substantially lacks guide-sequence-independent collateral endonuclease activity, the method comprising:
(1) in each of one or more regions of 15-20 consecutive polynucleotides (a) within 80, 90, 100, 110, 120, 130, 140, 150, 160, 170, or 180 residues of any residues of a endonuclease catalytic domain of the wild-type Cas effector enzyme or (b) spatially within 1-10 Ångström of any residues of the endonuclease catalytic domain of the wild-type Cas effector enzyme, substituting one or more (e.g., substantially all, except for up to 1, 2, 3, 4, or 5) polar and charged residues with a charge neutral aliphatic side-chain residue (such as A); and,
(2) identifying engineered Cas substantially maintains guide-sequence-specific endonuclease activity and substantially lacks guide-sequence-independent collateral endonuclease activity compared to the corresponding wild-type Cas.

78. The method of claim 77, wherein the wild-type Cas effector enzyme is a Cas13.

79. The method of claim 78, wherein the Cas13 is a Cas13a, a Cas13b, a Cas13c, a Cas13d (e.g., CasRx), a Cas13e, or a Cas13f.

80. The method of claim 79, wherein the Cas13e has the amino acid sequence of SEQ ID NO: 4; or wherein the Cas13d has the amino acid sequence of SEQ ID NO: 101; or wherein the Cas13f has the amino acid sequence of SEQ ID NO: 52.

81. A method of identifying an engineered Clustered Regularly Interspaced Short Palindromic Repeat (CRISPR)-Cas13 effector enzyme with altered guide sequence-independent collateral nuclease activity, the method comprising: in a region spatially close to an endonuclease catalytic domain of the corresponding wild-type Cas13 effector enzyme, substituting one or more charged or polar residues to a charge-neutral short chain aliphatic residue (such as A), to determine whether the resulting variant Cas13 effector enzyme:
(1) has substantially preserved guide sequence-specific endonuclease cleavage activity of the wild-type Cas13 towards a target RNA complementary to the guide sequence; and,
(2) either substantially lacks or has enhanced guide sequence-independent collateral endonuclease cleavage activity of the wild-type Cas13 towards a non-target RNA that does not bind to the guide sequence,
thereby identifying said engineered Cas13 effector enzyme with altered guide sequence-independent collateral nuclease activity.

82. The method of claim 81, wherein the engineered Cas13 effector enzyme substantially lacks guide sequence-independent collateral nuclease activity.

83. The method of claim 81, wherein the engineered Cas13 effector enzyme has enhanced guide sequence-independent collateral nuclease activity.

84. The method of any one of claims 81-83, wherein said one or more charged or polar residues are within a stretch of 15-20 (e.g., 16 or 17) consecutive amino acids within the region.

85. The method of claim 84, wherein said one or more charged or polar residues comprise, consist essentially of, or consist of one or more (or all) Tyr (Y) residue(s) within the stretch.
